(19) United States
(12) Patent Application Publication
(10) Pub. No.: US 2026/0159528 A1
(43) Pub. Date: Jun. 11, 2026

(54) HETEROCYCLIC COMPOUNDS AS TRIGGERING RECEPTOR EXPRESSED ON MYELOID CELLS 2 AGONISTS AND METHODS OF USE

(71) Applicant: Vigil Neuroscience, Inc., Watertown, MA (US)

(72) Inventors: Jonathan B. HOUZE, Watertown, MA (US); Bhaumik PANDYA, Watertown, MA (US); Alan P. KAPLAN, Watertown, MA (US); Maxence BOS, Saint-Laurent (CA); John MANCUSO, Saint-Laurent (CA); Ivan FRANZONI, Saint-Laurent (CA)

(73) Assignee: Vigil Neuroscience, Inc., Watertown, MA (US)

(21) Appl. No.: 18/707,486

(22) PCT Filed: Nov. 9, 2022
(86) PCT No.: PCT/US2022/079517

§ 371 Date: May 3, 2024

Related U.S. Application Data
(60) Provisional application No. 63/263,814, filed on Nov. 9, 2021.
(60) Provisional application No. 63/375,125, filed on Sep. 9, 2022.

(51) Int. Cl. C07D 513/04; A61K 31/4439; A61K 31/519; A61K 31/52; A61K 31/5377; C07D 405/14; C07D 471/04; C07D 473/00; C07D 473/32; C07D 487/04; C07D 498/04

(52) U.S. Cl. CPC C07D 513/04; A61K 31/4439; A61K 31/519; A61K 31/52; A61K 31/5377; C07D 405/14; C07D 471/04; C07D 473/00; C07D 473/32; C07D 487/04; C07D 498/04

(57) ABSTRACT

The present disclosure provides compounds of Formula I, useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

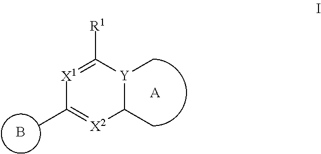

### CROSS REFERENCE TO RELATED APPLICATION

This application claims the benefit of U.S. Provisional Application No. 63/263,814, filed Nov. 9, 2021, and U.S. Provisional Application No. 63/375,125, filed Sep. 9, 2022, the entirety of each of which is incorporated herein by reference.

### FIELD

The present disclosure provides compounds useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

### BACKGROUND

Microglia are resident innate immune cells in the brain and are important for the maintenance of homeostatic conditions in the central nervous system (Hickman et al. Nat Neurosci 2018, Li and Barres, Nat Rev Immunol., 2018). These resident macrophages express a variety of receptors that allow them to sense changes in their microenvironment and alter their phenotypes to mediate responses to invading pathogens, proteotoxic stress, cellular injury, and other infarcts that can occur in health and disease. Id. Microglia reside in the parenchyma of the brain and spinal cord where they interact with neuronal cell bodies (Cserep et al. Science, 2019), neuronal processes (Paolicelli et al. Science, 2011, Ikegami et al. Neruopathology, 2019) in addition to other types of glial cells (Domingues et al. Front Cell Dev Biol, 2016; Liddelow et al. Nature, 2017, Shinozaki et al. Cell Rep., 2017), playing roles in a multitude of physiological processes. With the ability to rapidly proliferate in response to stimuli, microglia characteristically exhibit myeloid cell functions such as phagocytosis, cytokine/chemokine release, antigen presentation, and migration (Colonna and Butovsky, Annu Rev Immunol, 2017). More specialized functions of microglia include the ability to prune synapses from neurons and directly communicate with their highly arborized cellular processes that survey the area surrounding the neuronal cell bodies (Hong et al. Curr Opin Neurobiol, 2016; Sellgren et al. Nat Neurosci, 2019).

The plasticity of microglia and their diverse states as described through single-cells RNASeq profiling are thought to arise through the integration of signaling from a diverse array of cell surface receptors (Hickman et al. Nat Neurosci 2013). Collectively known as the microglial “sensome,” these receptors are responsible for transducing activating or activation-suppressing intracellular signaling and include protein families such as Sialic acid-binding immunoglobulin-type lectins (“SIGLEC”), Toll-like receptors (“TLR”), Fc receptors, nucleotide-binding oligomerization domain (“NOD”) and purinergic G protein-coupled receptors. Doens and Fernandez 2014, Madry and Attwell 2015, Hickman and El Khoury 2019. Similar to other cells of the myeloid lineage, the composition of microglial sensomes is dynamically regulated and acts to recognize molecular pattern that direct phenotypic responses to homeostatic changes in the central nervous system (“CNS”). Id. One of the receptors selectively expressed by brain microglia is TREM2, composed of a single-pass transmembrane domain, an extracellular stalk region, and extracellular immunoglobulin variable (“IgV”)-like domain responsible for ligand interaction (Kleinberger et al. Sci Transl Med, 2014). As TREM2 does not possess intracellular signal transduction-mediating domains, biochemical analysis has illustrated that interaction with adaptor proteins DAP10 and DAP12 mediate downstream signal transduction following ligand recognition (Peng et al. Sci Signal 2010; Jay et al. Mol Neurodegener, 2017). TREM2/DAP12 complexes in particular act as a signaling unit that can be characterized as pro-activation on microglial phenotypes in addition to peripheral macrophages and osteoclasts (Otero et al. J Immunol, 2012; Kobayashi et al. J Neurosci, 2016; Jaitin et al., Cell, 2019. In the CNS, signaling through TREM2 has been studied in the context of ligands such as phospholipids, cellular debris, apolipoproteins, and myelin (Wang et al. Cell, 2015; Kober and Brett, J Mol Biol, 2017; Shirotani et al., Sci Rep, 2019). In mice lacking functional TREM2 expression or expressing a mutated form of the receptor, a core observation is blunted microglial responses to insults such as oligodendrocyte demyelination, stroke-induced tissue damage in the brain, and proteotoxic inclusions in vivo (Cantoni et al., Acta Neuropathol, 2015, Wu et al., Mol Brain, 2017).

Coding variants in the TREM12 locus has been associated with late onset Alzheimer's disease (“LOAD”) in human genome-wide association studies, linking a loss-of-receptor function to a gain in disease risk (Jonsson et al. N Engl J Med 2013, Sims et al. Nat Genet 2017). Genetic variation of other genes selectively expressed by microglia in the CNS, for example, CD33, PLCg2 and MS4A4A/6A have reached genome-wide significance for their association with LOAD risk (Hollingworth et al. Nat Genet 2011, Sims et al. Nat Genet 2017, Deming et al. Sci Transl Med 2019). Together, these genetic findings link together in a putative biochemical circuit that highlights the importance of microglial innate immune function in LOAD. Additionally, increase or elevation in the soluble form of TREM2 (“sTREM2”) in the cerebrospinal fluid (CSF) of human subjects is associated with disease progression and emergence of pathological hallmarks of LOAD including phosphorylated Tau (Suarez-Calvet et al. Mol Neurodegener 2019). Furthermore, natural history and human biology studies indicate that baseline sTREM2 levels in the CSF can stratify the rate of temporal lobe volume loss and episodic memory decline in longitudinally monitored cohorts (Ewers et al. Sci Transl Med 2019).

In addition to human genetic evidence supporting a role of TREM2 in LOAD, homozygous loss-of-function mutations in TREM12 are causal for an early onset dementia syndrome known as Polycystic lipomembranous osteodysplasia with sclerosing leukoencephalopathy (“PLOSL”) or Nasu-Hakola disease (“NHD”) (Golde et al. Alzheimers Res Ther 2013, Dardiotis et al. Neurobiol Aging 2017). This progressive neurodegenerative disease typically manifests in the 3^{rd} decade of life and is pathologically characterized by loss of myelin in the brain concomitant with gliosis, unresolved neuroinflammation, and cerebral atrophy. Typical neuropsychiatric presentations are often preceded by osseous abnormalities, such as bone cysts and loss of peripheral bone density (Bianchin et al. Cell Mol Neurobiol 2004; Madry et al. Clin Orthop Relat Res 2007, Bianchin et al. Nat Rev Neurol 2010). Given that osteoclasts of the myeloid lineage are also known to express TREM2, the PLOSL-related symptoms of wrist and ankle pain, swelling, and fractures indicate that TREM2 may act to regulate bone homeostasis through defined signaling pathways that parallel the microglia in the CNS (Paloneva et al. J Exp Med 2003, Otero et al. J Immunol 2012). The link between TREM2 function and PLOSL has illustrated the importance of the receptor in sustaining key physiological aspects of myeloid cell function in the human body.

Efforts have been made to model the biology of TREM2 in mice prompting the creation of TREM2 knock out (“KO”) mice in addition to the LOAD-relevant TREM2 R47H loss-of-function mutant transgenic mice (Ulland et al. Cell, 2017, Kang et al. Hum Mol Genet 2018). Although unable to recapitulate the neurological manifestations of PLOSL, TREM12 KO mice show abnormalities in bone ultrastructure (Otero et al. J Immunol 2012). When the TREM2 KO or mutant mice have been crossed onto familial Alzheimer's disease transgenic mouse background such as the 5×FAD amyloidogenic mutation lines, marked phenotypes have been observed (Ulrich et al. Neuron, 2017). These in vivo phenotypes of TREM2 loss-of-function in the CNS include elevated the plaque burden and lower levels of secreted microglial factors SPP1 and Osteopontin that are characteristic of the microglial response to amyloid pathology (Ulland et al. Cell, 2017). Other rodent studies have demonstrated that loss of TREM2 leads to decreased microglial clustering around plaques and emergence of less compact plaque morphology in familial AD amyloid models (Parhizkar et al. Nat Neurosci 2019). With regards to the Tau protein pathology that is observed in LOAD, familial tauopathy models in mice demonstrated an enhanced spreading of pathological human Tau aggregates from point of injection into mouse brain in TREM2 KO mice (Leyns et al. Nat Neurosci 2019). Furthermore, single-cell RNASeq studies with the TREM2 KO mice in aged scenarios, 5×FAD familial Alzheimer's disease model mice, and Amyotrophic Lateral Sclerosis SOD1 mutant mouse backgrounds indicate that TREM2 receptor function is critical for a conserved set of phenotypic transformations within microglial populations in response to CNS pathology (Keren-Shaul et al. Cell 2017).

In rodent models where TREM2 expression levels are elevated, brain amyloid pathology in the 5×FAD transgenic mice displayed reduced plaque volume and altered morphology (Lee et al. Neuron, 2018). The changes in immunohistological markers relating to brain amyloid pathology were also accompanied by an attenuated presence of dystrophic neurites when TREM12 was overexpressed. Id. Therefore, the pharmacological activation of TREM2 is a target of interest for treating or preventing neurological, neurodegenerative and other diseases. Despite many attempts to alter disease progression by targeting the pathological hallmarks of LOAD through anti-amyloid and anti-Tau therapeutics, there is a need for activators of TREM2 to address the genetics-implicated neuroimmune aspects of, for example, LOAD. Such TREM2 activators may be suitable for use as therapeutic agents and remain in view of the significant continuing societal burden that remains unmitigated for diseases, such as Alzheimer's disease.

### SUMMARY

Provided herein is a compound of Formula I

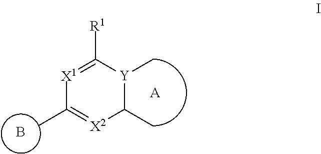

or a tautomer thereof, or a pharmaceutically acceptable salt of said compound or said tautomer, wherein
R¹ is an optionally substituted C₁₋₆ aliphatic group, OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula

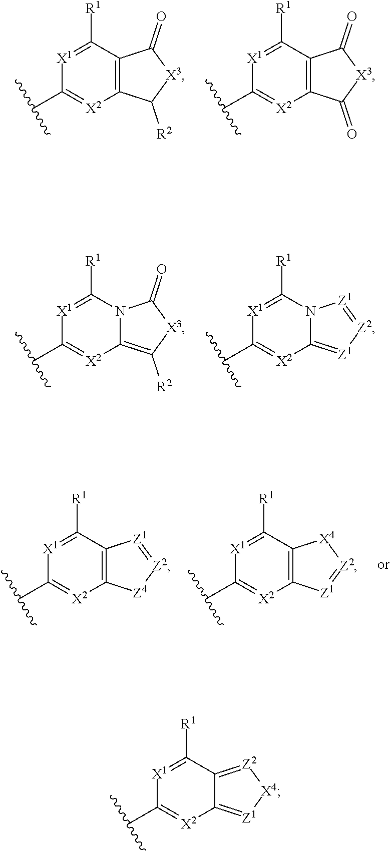

Y is C or N, as required by the bicyclic ring system formed by Ring A;
X³ is CHR³, or NR⁴;
X⁴ is CHR³, NR⁴, O or S;
each Z¹ is independently CR² or N;
Z² is CR³ or N;
R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R⁴ is hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
R³ and R⁴ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
Ring B is

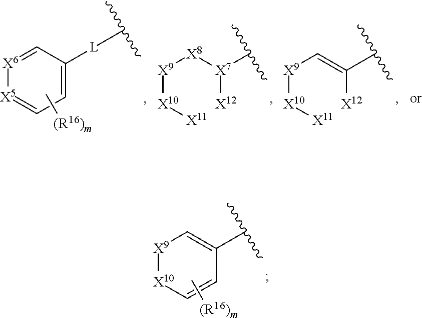

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR, C(R)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³ and R¹⁴ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

Also provided herein is a pharmaceutical composition comprising a compound of Formula I, or a tautomer thereof, or a pharmaceutically acceptable salt of said compound or said tautomer, and a pharmaceutically acceptable excipient.

Also provided herein is a compound of Formula I, or a tautomer thereof, or a pharmaceutically acceptable salt of said compound or said tautomer, or a pharmaceutical composition as described hereinabove, for use in treating or preventing a condition associated with a loss of function of human TREM2.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

In one aspect, provided herein is a compound of Formula I.

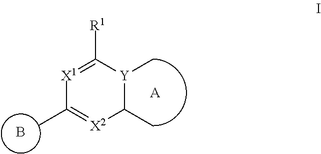

or a tautomer thereof, or a pharmaceutically acceptable salt of said compound or said tautomer, wherein
R¹ is an optionally substituted C₁₋₆ aliphatic group, C₁₋₆haloalkyl, optionally substituted OCH₂—(C₃₋₆cycloalkyl), optionally substituted 0-phenyl, or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula

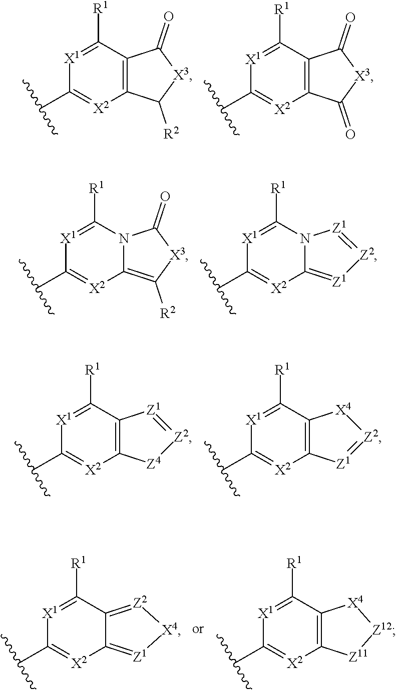

Y is C or N, as required by the bicyclic ring system formed by Ring A;
X³ is CHR³, or NR⁴;
X⁴ is CHR³, NR⁴, or S;
each Z¹ is independently CR² or N;
Z² is CR³ or N;
Z¹¹ is CHR³, C(R³)₂, or NR⁴;
Z¹² is CHR², C(R²)₂, NR⁴, or C(═N—R⁴);
R² and R³ are each independently selected from hydrogen, an optionally substituted C₁₋₆ aliphatic group, halogen, —OR, —CN, —NR₂, —C(═O)R, —NR—C(O)—R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R⁴ is hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
R³ and R⁴ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
Ring B is

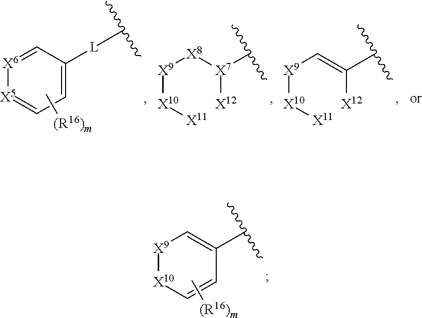

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR, C(R)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³ and R¹⁴ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

Provided herein is a compound of Formula I

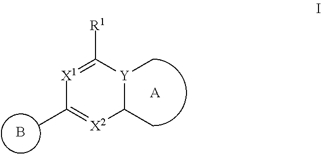

or a tautomer thereof, or a pharmaceutically acceptable salt of said compound or said tautomer, wherein
R¹ is an optionally substituted C₁₋₆ aliphatic group, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula

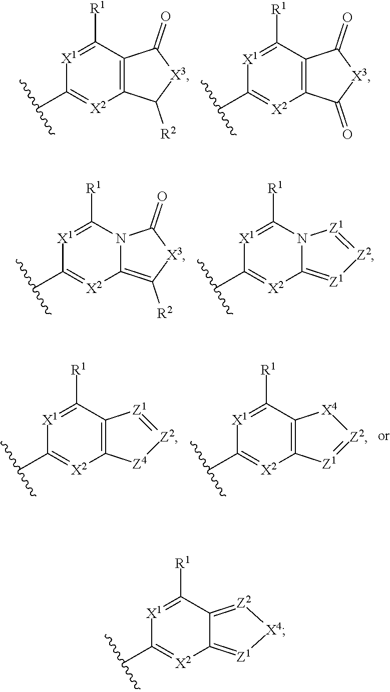

Y is C or N, as required by the bicyclic ring system formed by Ring A;
X³ is CHR³, or NR⁴;
X⁴ is CHR³, NR⁴, or S;
each Z¹ is independently CR² or N;
Z² is CR³ or N;
R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R⁴ is hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
R³ and R⁴ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
Ring B is

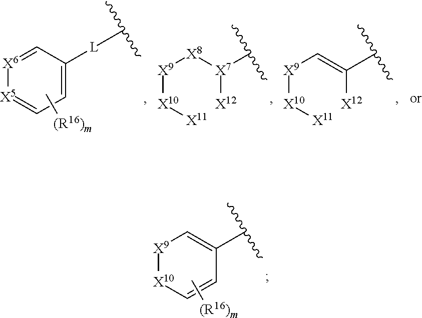

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR^{B}, C(R)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³ and R¹⁴ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

In some embodiments, the compound is a compound of Formula IIa:

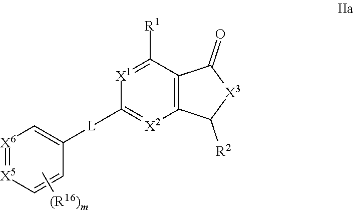

or a tautomer thereof, or a pharmaceutically acceptable salt of said compound or said tautomer, wherein each variable is as defined above and described in embodiments herein both singly and in combination.

In some embodiments, the compound is a compound of Formula IIb:

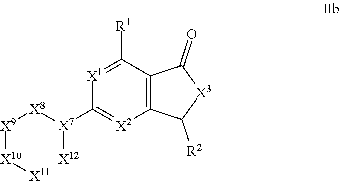

or a tautomer thereof, or a pharmaceutically acceptable salt of said compound or said tautomer, wherein each variable is as defined above and described in embodiments herein both singly and in combination.

In some embodiments, the compound is a compound of Formula IIc:

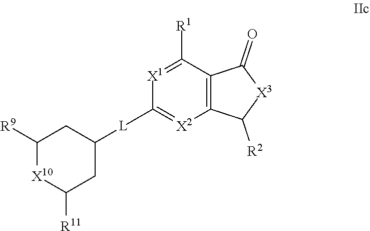

or a tautomer thereof, or a pharmaceutically acceptable salt of said compound or said tautomer, wherein each variable is as defined above and described in embodiments herein both singly and in combination.

In some embodiments, the compound is a compound of Formula IIIa:

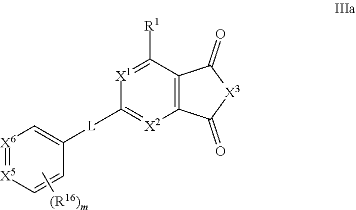

or a tautomer thereof, or a pharmaceutically acceptable salt of said compound or said tautomer, wherein each variable is as defined above and described in embodiments herein both singly and in combination.

In some embodiments, the compound is a compound of Formula IIIb:

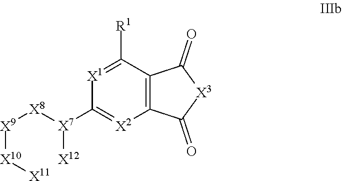

or a tautomer thereof, or a pharmaceutically acceptable salt of said compound or said tautomer, wherein each variable is as defined above and described in embodiments herein both singly and in combination.

In some embodiments, the compound is a compound of Formula IIIc:

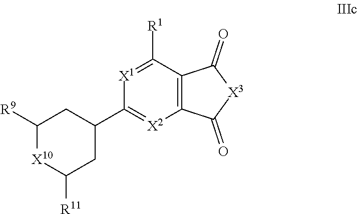

or a tautomer thereof, or a pharmaceutically acceptable salt of said compound or said tautomer, wherein each variable is as defined above and described in embodiments herein both singly and in combination.

In some embodiments, the compound is a compound of Formula IVa:

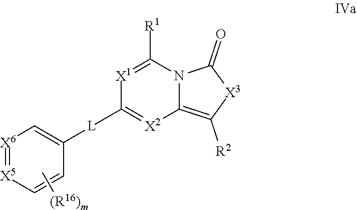

or a tautomer thereof, or a pharmaceutically acceptable salt of said compound or said tautomer, wherein each variable is as defined above and described in embodiments herein both singly and in combination.

In some embodiments, the compound is a compound of Formula IVb:

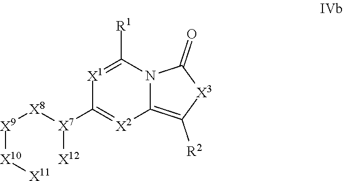

or a tautomer thereof, or a pharmaceutically acceptable salt of said compound or said tautomer, wherein each variable is as defined above and described in embodiments herein both singly and in combination.

In some embodiments, the compound is a compound of Formula IVc:

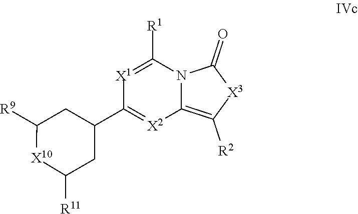

or a tautomer thereof, or a pharmaceutically acceptable salt of said compound or said tautomer, wherein each variable is as defined above and described in embodiments herein both singly and in combination.

In some embodiments, the compound is a compound of Formula Va:

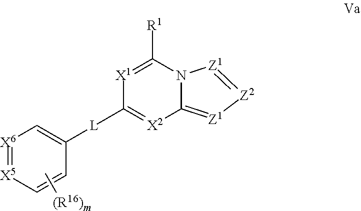

or a tautomer thereof, or a pharmaceutically acceptable salt of said compound or said tautomer, wherein each variable is as defined above and described in embodiments herein both singly and in combination.

In some embodiments, the compound is a compound of Formula Vb:

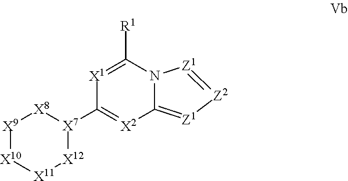

or a tautomer thereof, or a pharmaceutically acceptable salt of said compound or said tautomer, wherein each variable is as defined above and described in embodiments herein both singly and in combination.

In some embodiments, the compound is a compound of Formula Vc:

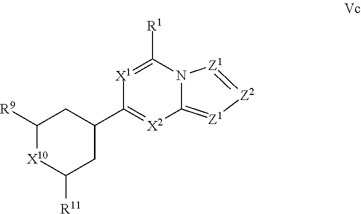

or a tautomer thereof, or a pharmaceutically acceptable salt of said compound or said tautomer, wherein each variable is as defined above and described in embodiments herein both singly and in combination.

In some embodiments, the compound is a compound of Formula VIa:

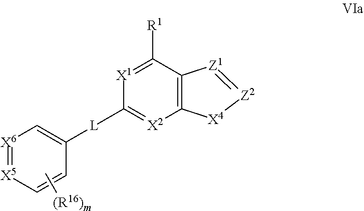

or a tautomer thereof, or a pharmaceutically acceptable salt of said compound or said tautomer, wherein each variable is as defined above and described in embodiments herein both singly and in combination.

In some embodiments, the compound is a compound of Formula VIb:

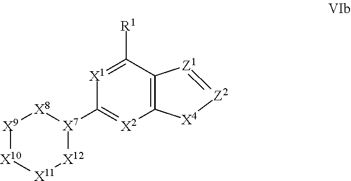

or a tautomer thereof, or a pharmaceutically acceptable salt of said compound or said tautomer, wherein each variable is as defined above and described in embodiments herein both singly and in combination.

In some embodiments, the compound is a compound of Formula VIc:

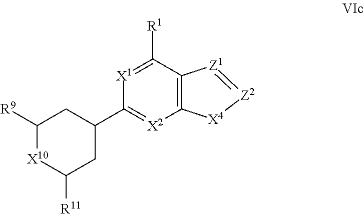

or a tautomer thereof, or a pharmaceutically acceptable salt of said compound or said tautomer, wherein each variable is as defined above and described in embodiments herein both singly and in combination.

In some embodiments, the compound is a compound of Formula Vila:

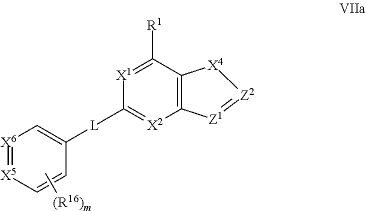

or a tautomer thereof, or a pharmaceutically acceptable salt of said compound or said tautomer, wherein each variable is as defined above and described in embodiments herein both singly and in combination.

In some embodiments, the compound is a compound of Formula VIIb:

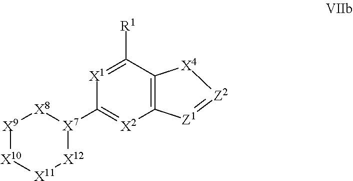

or a tautomer thereof, or a pharmaceutically acceptable salt of said compound or said tautomer, wherein each variable is as defined above and described in embodiments herein both singly and in combination.

In some embodiments, the compound is a compound of Formula VIIc:

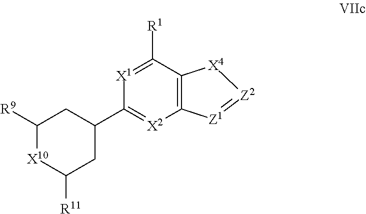

or a tautomer thereof, or a pharmaceutically acceptable salt of said compound or said tautomer, wherein each variable is as defined above and described in embodiments herein both singly and in combination.

In some embodiments, the compound is a compound of Formula VIIIa:

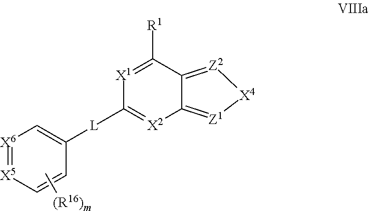

or a tautomer thereof, or a pharmaceutically acceptable salt of said compound or said tautomer, wherein each variable is as defined above and described in embodiments herein both singly and in combination.

In some embodiments, the compound is a compound of Formula VIIIb:

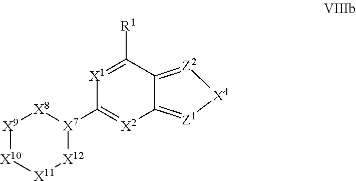

or a tautomer thereof, or a pharmaceutically acceptable salt of said compound or said tautomer, wherein each variable is as defined above and described in embodiments herein both singly and in combination.

In some embodiments, the compound is a compound of Formula VIIIc:

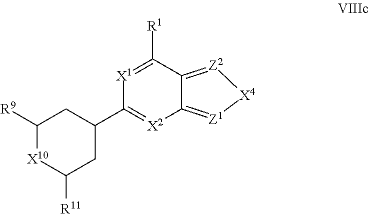

or a tautomer thereof, or a pharmaceutically acceptable salt of said compound or said tautomer, wherein each variable is as defined above and described in embodiments herein both singly and in combination.

In some embodiments, the compound is a compound of Formula VIIb-1 to VIIb-11:

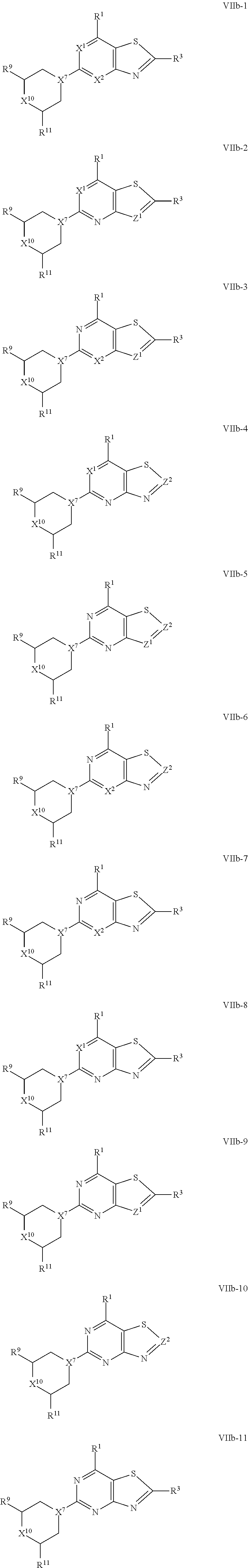

or a tautomer thereof, or a pharmaceutically acceptable salt of said compound or said tautomer, wherein each variable is as defined above and described in embodiments herein both singly and in combination.

In some embodiments, the compound is a compound of Formula VIIb′-1 to VIIb′-11:

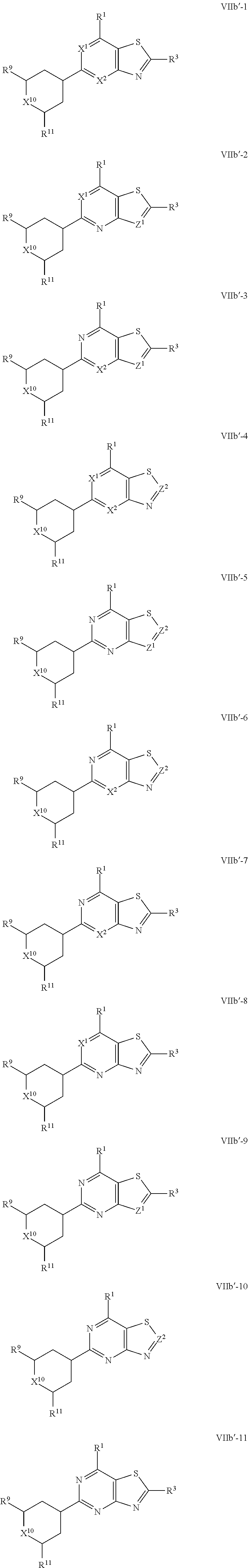

or a tautomer thereof, or a pharmaceutically acceptable salt of said compound or said tautomer, wherein each variable is as defined above and described in embodiments herein both singly and in combination.

In some embodiments, the compound is a compound of Formula VIIb″-1 to VIIb″-11:

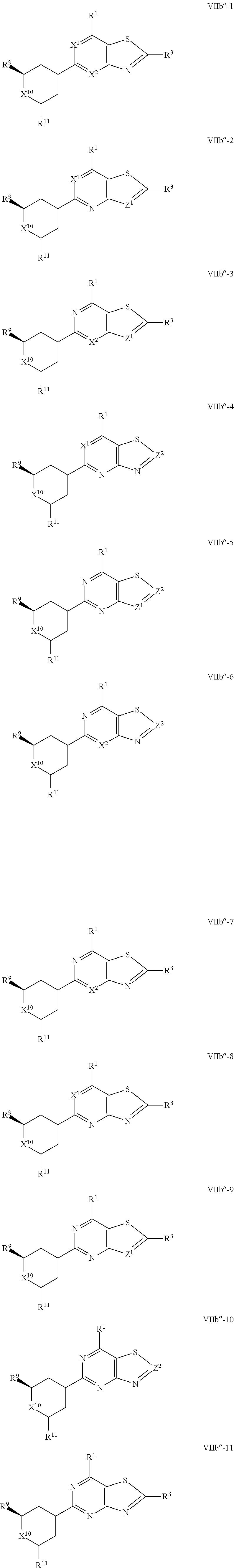

or a tautomer thereof, or a pharmaceutically acceptable salt of said compound or said tautomer, wherein each variable is as defined above and described in embodiments herein both singly and in combination.

As defined generally above, R¹ is an optionally substituted C₁₋₆ aliphatic group, C₁₋₆haloalkyl, optionally substituted OCH₂—(C₃₋₆cycloalkyl), or a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted. In some embodiments, R¹ is optionally substituted 0-phenyl.

In some embodiments, R¹ is an optionally substituted C₁₋₆ aliphatic group. In some embodiments, R¹ is —OR. In some embodiments, R¹ is —NR₂. In some embodiments, R¹ is —C(═O)R. In some embodiments, R¹ is —C(═O)OR. In some embodiments, R¹ is —C(═O)NR₂. In some embodiments, R² is —SO₂R. In some embodiments, R¹ is —SO₂NR₂. In some embodiments, R¹ is C₁₋₆haloalkyl. In some embodiments, R¹ is an optionally substituted OCH₂—(C₃₋₆cycloalkyl). In some embodiments, R¹ is an optionally substituted 3-8 membered saturated or partially unsaturated monocyclic carbocyclic ring. In some embodiments, R¹ is an optionally substituted 5-12 membered saturated or partially unsaturated bridged carbocyclic ring. In some embodiments, R¹ is an optionally substituted 7-12 membered saturated or partially unsaturated bicyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 3-8 membered saturated or partially unsaturated monocyclic heterocyclic ring (having 1-2 heteroatoms independently selected from nitrogen, oxygen, and sulfur). In some embodiments, R¹ is an optionally substituted 6-12 membered saturated or partially unsaturated bridged heterocyclic ring (having 1-4 heteroatoms independently selected from nitrogen, oxygen, and sulfur). In some embodiments, R¹ is an optionally substituted 7-12 membered saturated or partially unsaturated bicyclic heterocyclic ring (having 1-4 heteroatoms independently selected from nitrogen, oxygen, and sulfur). In some embodiments, R¹ is an optionally substituted 5-6 membered monocyclic heteroaromatic ring (having 1-4 heteroatoms independently selected from nitrogen, oxygen, and sulfur). In some embodiments, R¹ is an optionally substituted 8-10 membered bicyclic heteroaromatic ring (having 1-5 heteroatoms independently selected from nitrogen, oxygen, and sulfur).

In some embodiments, R¹ is a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹ is phenyl, optionally substituted with 1-3 substituents independently selected from halogen, C₁₋₆ aliphatic, —OR^{∘}, or C₁₋₆haloalkyl. In some embodiments, R¹ is phenyl, optionally substituted with 1-3 halogen. In some embodiments, R¹ is a 5-12 membered saturated or partially unsaturated bridged carbocyclic ring, optionally substituted with 1-3 substituents independently selected from halogen, C₁₋₆ aliphatic, —OR^{∘}, or C₁₋₆haloalkyl. In some embodiments, R¹ is a C₅₋₈tricycloalkyl ring, optionally substituted with 1-3 substituents independently selected from halogen, C₁₋₆ aliphatic, —OR^{∘}, or C₁₋₆haloalkyl. In some embodiments, R¹ is 5-6 membered monocyclic heteroaromatic ring (having 1-4 heteroatoms independently selected from nitrogen, oxygen, and sulfur), optionally substituted with 1-3 substituents independently selected from halogen, C₁₋₆ aliphatic, —OR^{∘}, or C₁₋₆haloalkyl. In some embodiments, R¹ is 5-6 membered monocyclic heteroaromatic ring (having 1-4 heteroatoms independently selected from nitrogen, oxygen, and sulfur), optionally substituted with 1-3 halogen.

In some embodiments, R¹ is optionally substituted C₃₋₆cycloalkyl, optionally substituted spiro[3.3]heptanyl, optionally substituted spiro[5.2]octanyl, optionally substituted

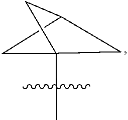 [SMILES: *C12CC(C1)C2]

optionally substituted cyclopent-1-en-1-yl, optionally substituted cyclohex-1-en-1-yl, optionally substituted phenyl, optionally substituted pyridinyl, optionally substituted aziridine-1-yl, optionally substituted pyrrolidine-1-yl, optionally substituted azabicyclo[3.1.0]hexan-3-yl, optionally substituted piperidine-1-yl, or optionally substituted —OCH₂—(C₃₋₄cycloalkyl). In some embodiments, R¹ is optionally substituted C₃₋₆cycloalkyl. In some embodiments, R¹ is optionally substituted spiro[3.3]heptanyl. In some embodiments, R¹ is optionally substituted spiro[5.2]octanyl. In some embodiments, R¹ is optionally substituted

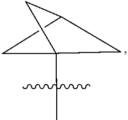 [SMILES: *C12CC(C1)C2]

In some embodiments, R¹ is optionally substituted cyclopent-1-en-1-yl. In some embodiments, R¹ is optionally substituted cyclohex-1-en-1-yl. In some embodiments, R¹ is optionally substituted phenyl. In some embodiments, R¹ is optionally substituted pyridinyl. In some embodiments, R¹ is optionally substituted aziridine-1-yl. In some embodiments, R¹ is optionally substituted pyrrolidine-1-yl. In some embodiments, R¹ is optionally substituted azabicyclo[3.1.0]hexan-3-yl. In some embodiments, R¹ is optionally substituted piperidine-1-yl. In some embodiments, R¹ is optionally substituted —OCH₂—(C₃-4cycloalkyl).

In some embodiments, R¹ is optionally substituted with 1-3 groups that are independently halogen; —(CH₂)₀₋₆R^{∘}; —(CH₂)₀₋₆OR^{∘}; —O(CH₂)₀₋₆R^{∘}, —O—(CH₂)₀₋₆C(O)OR^{∘}; —(CH₂)₀₋₆CH(OR^{∘})₂; —(CH₂)₀₋₆SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂; —CN; —N₃; —(CH₂)₀₋₆N(R^{∘})₂; —(CH₂)₀₋₆N(R^{∘})C(O)R^{∘}; —N(R^{∘})C(S)R^{∘}; —(CH₂)₀₋₆N(R^{∘})C(O)NR^{∘}₂; —N(R^{∘})C(S)NR^{∘}₂; —(CH₂)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆C(O)R^{∘}; —C(S)R^{∘}; —(CH₂)₀₋₆C(O)OR^{∘}; —(CH₂)₀₋₆C(O)SR^{∘}; —(CH₂)₀₋₆C(O)OSiR^{∘}₃; —(CH₂)₀₋₆OC(O)R^{∘}; —OC(O)(CH₂)₀₋₆SR^{∘}, —(CH₂)₀₋₆SC(O)R^{∘}; —(CH₂)₀₋₆C(O)NR^{∘}₂; —C(S)NR^{∘}₂; —C(S)SR^{∘}; —SC(S)SR^{∘}, —(CH₂)₀₋₆OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₆SSR^{∘}; —(CH₂)₀₋₆S(O)₂R^{∘}; —(CH₂)₀₋₆S(O)₂OR^{∘}; —(CH₂)₀₋₆OS(O)₂R^{∘}; —S(O)₂NR^{∘}₂; —(CH₂)₀₋₆S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined elsewhere herein and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur). In some embodiments, R¹ is optionally substituted with one or more —SF₅ groups.

In some embodiments, R¹ is —CH₂CH₂CF₃,

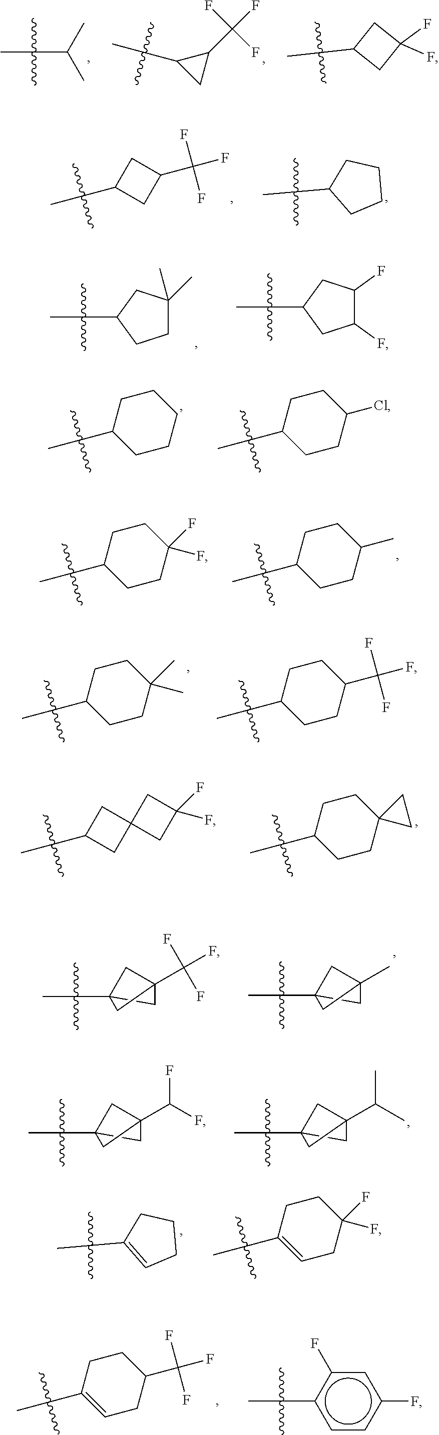

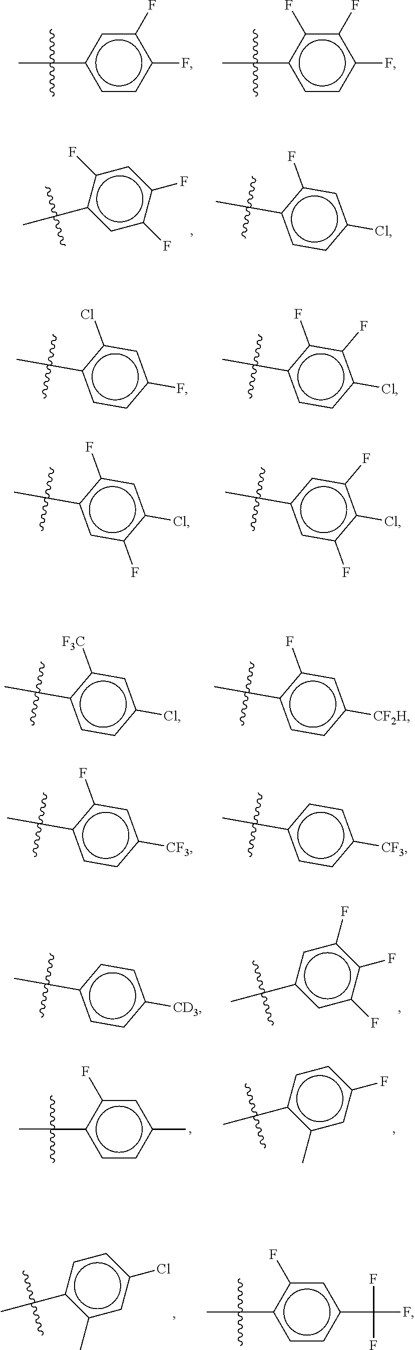

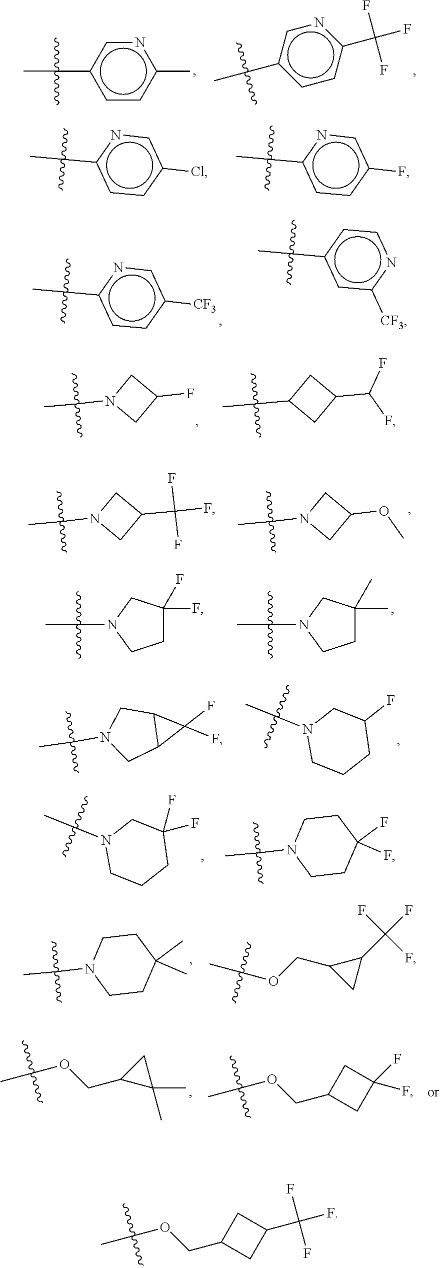

In some embodiments, R¹ is

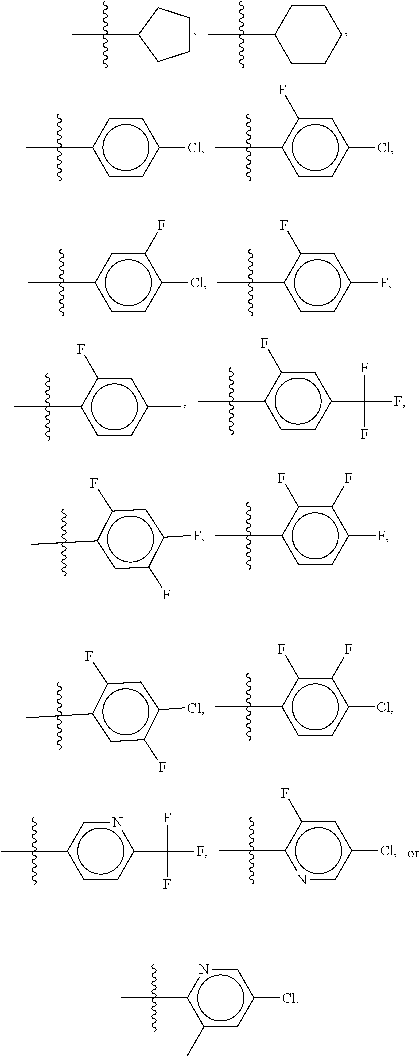

In some embodiments, R¹ is a substituent selected from those shown below:

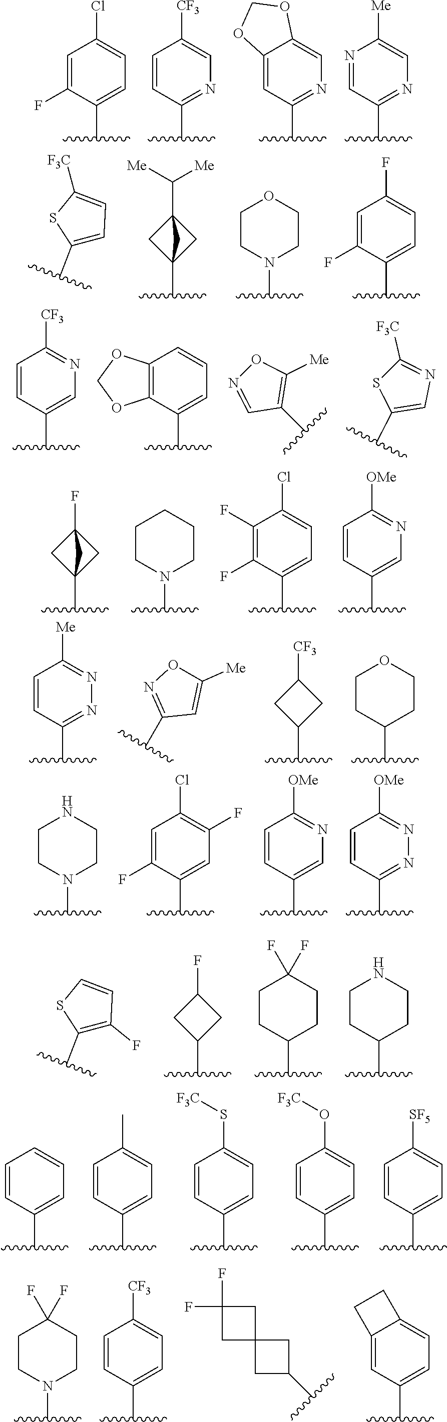

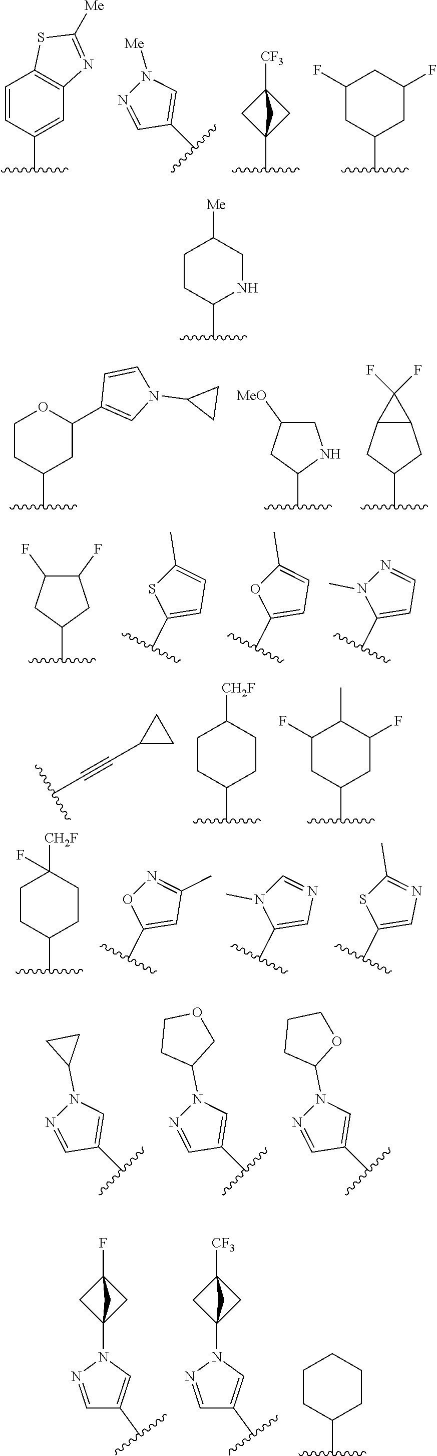

In some embodiments, R¹ is

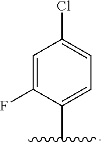 [SMILES: *c1ccc(Cl)cc1F]

In some embodiments, R¹ is

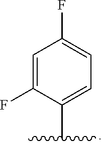 [SMILES: *c1ccc(F)cc1F]

In some embodiments, R¹ is

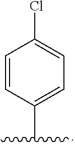 [SMILES: *c1ccc(Cl)cc1]

In some embodiments, R¹ is

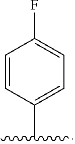 [SMILES: *c1ccc(F)cc1]

In some embodiments, R¹ is selected from those depicted in Table A below. In some embodiments, R¹ is selected from those depicted in Table A2 below.

As defined generally above, X¹ is CR¹³, CH or N. In some embodiments, X¹ is CH or N. In some embodiments, X¹ is CH. In some embodiments, X² is CR¹³. In some embodiments, X¹ is N. In some embodiments, X¹ is selected from those depicted in Table A below. In some embodiments, X¹ is selected from those depicted in Table A2 below.

As defined generally above, X² is CR¹⁴, CH or N. In some embodiments, X² is CH or N. In some embodiments, X² is CH. In some embodiments, X² is CR¹⁴. In some embodiments, X² is N. In some embodiments, X² is selected from those depicted in Table A below. In some embodiments, X² is selected from those depicted in Table A2 below.

As defined generally above, R¹³ and R¹⁴ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy.

In some embodiments, R¹³ is hydrogen. In some embodiments, R³ is an optionally substituted C₁₋₆ aliphatic group. In some embodiments, R¹³ is halogen. In some embodiments, R¹³ is —OR. In some embodiments, R¹³ is —CN. In some embodiments, R¹³ is —NR₂. In some embodiments, R¹³ is —C(═O)R. In some embodiments, R¹³ is —C(═O)OR. In some embodiments, R¹³ is —C(═O)NR₂. In some embodiments, R¹³ is —SO₂R. In some embodiments, R¹³ is —SO₂NR₂. In some embodiments, R¹³ is C₁₋₆haloalkyl. In some embodiments, R¹³ is C₁₋₆haloalkoxy. In some embodiments, R¹³ is methyl.

In some embodiments, R¹⁴ is hydrogen. In some embodiments, R¹⁴ is an optionally substituted C₁₋₆ aliphatic group. In some embodiments, R¹⁴ is halogen. In some embodiments, R¹⁴ is —OR. In some embodiments, R¹⁴ is —CN. In some embodiments, R¹⁴ is —NR₂. In some embodiments, R¹⁴ is —C(═O)R. In some embodiments, R¹⁴ is —C(═O)OR. In some embodiments, R¹⁴ is —C(═O)NR₂. In some embodiments, R¹⁴ is —SO₂R. In some embodiments, R¹⁴ is —SO₂NR₂. In some embodiments, R¹⁴ is C₁₋₆haloalkyl. In some embodiments, R¹⁴ is C₁₋₆haloalkoxy. In some embodiments, R¹⁴ is methyl.

As defined generally above, Ring A together with the 6-membered ring system to which it is fused forms a bicyclic ring system of formula

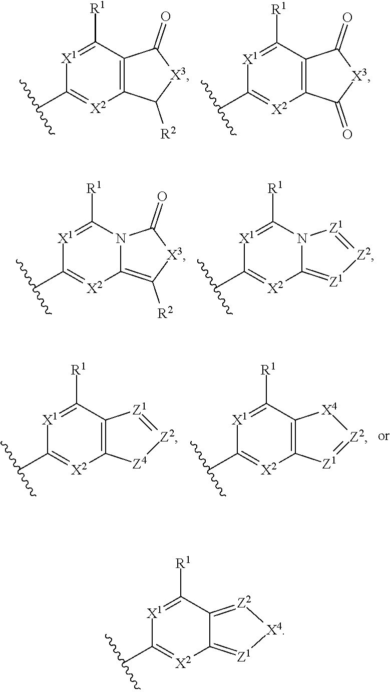

In some embodiments, Ring A together with the 6-membered ring system to which it is fused forms a bicyclic ring system of formula

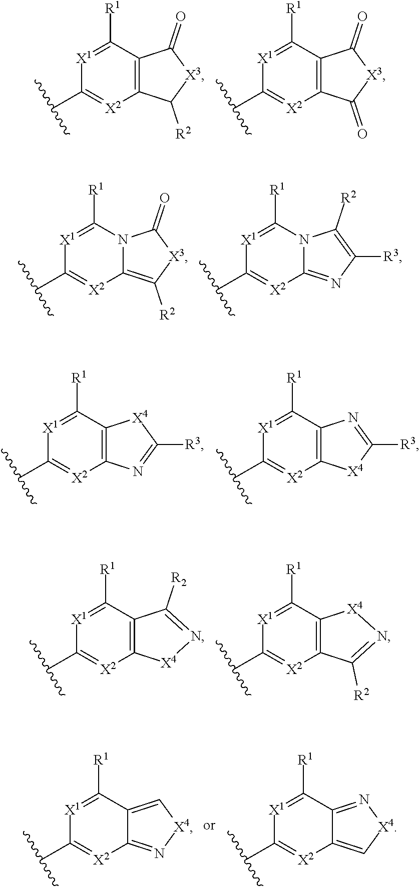

In some embodiments, Ring A together with the 6-membered ring system to which it is fused forms a bicyclic ring system of formula

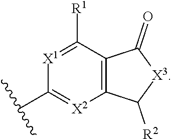

In some embodiments, Ring A together with the 6-membered ring system to which it is fused forms a bicyclic ring system of formula

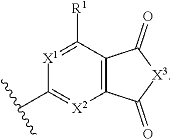

In some embodiments, Ring A together with the 6-membered ring system to which it is fused forms a bicyclic ring system of formula

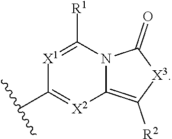

In some embodiments, Ring A together with the 6-membered ring system to which it is fused forms a bicyclic ring system of formula

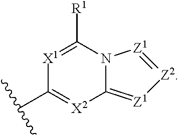

In some embodiments, Ring A together with the 6-membered ring system to which it is fused forms a bicyclic ring system of formula

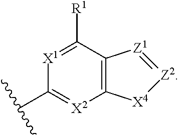

In some embodiments, Ring A together with the 6-membered ring system to which it is fused forms a bicyclic ring system of formula

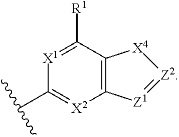

In some embodiments, Ring A together with the 6-membered ring system to which it is fused forms a bicyclic ring system of formula

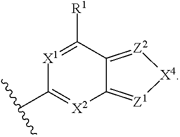

In some embodiments, Ring A together with the 6-membered ring system to which it is fused forms a bicyclic ring system of formula

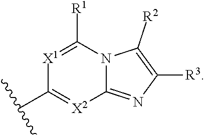

In some embodiments, Ring A together with the 6-membered ring system to which it is fused forms a bicyclic ring system of formula

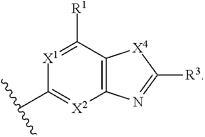

In some embodiments, Ring A together with the 6-membered ring system to which it is fused forms a bicyclic ring system of formula

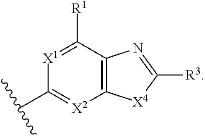

In some embodiments, Ring A together with the 6-membered ring system to which it is fused forms a bicyclic ring system of formula

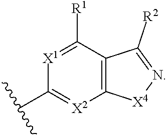

In some embodiments, Ring A together with the 6-membered ring system to which it is fused forms a bicyclic ring system of formula

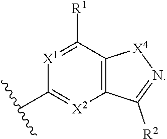

In some embodiments, Ring A together with the 6-membered ring system to which it is fused forms a bicyclic ring system of formula

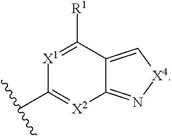

In some embodiments, Ring A together with the 6-membered ring system to which it is fused forms a bicyclic ring system of formula

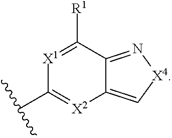

In some embodiments, Ring A together with the 6-membered ring system to which it is fused forms a bicyclic ring system of formula

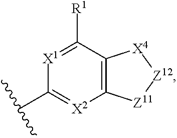

wherein Z¹¹ is CHR³, C(R³)₂, or NR⁴; and Z¹² is CHR², C(R²)₂, NR⁴, or C(═N—R⁴), wherein each variable is independently as defined herein and as described in embodiments herein.

In some embodiments, Ring A together with the 6-membered ring system to which it is fused forms a bicyclic ring system selected from:

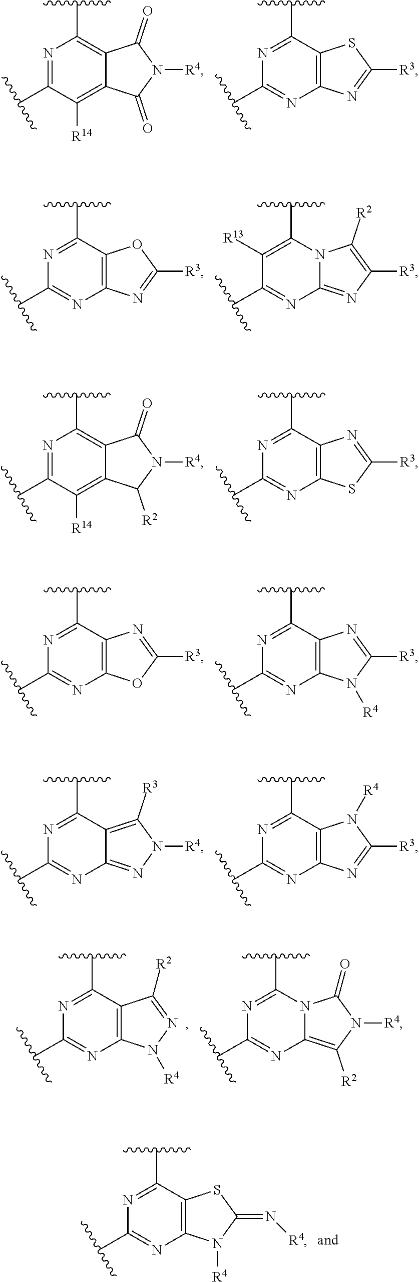

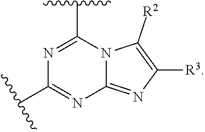

In some embodiments, Ring A together with the 6-membered ring system to which it is fused forms a bicyclic ring system selected from those depicted in Table A below. In some embodiments, Ring A together with the 6-membered ring system to which it is fused forms a bicyclic ring system selected from those depicted in Table A2 below.

As defined generally above, X³ is CHR³, or NR⁴. In some embodiments X³ is CHR³. In some embodiments, X³ is NR⁴. In some embodiments, X³ is NH. In some embodiments, X³ is NMe. In some embodiments, X³ is NCH(CH₃)₂.

As defined generally above, X⁴ is CHR³, NR⁴, O or S. In some embodiments X⁴ is CHR³. In some embodiments, X⁴ is NR⁴. In some embodiments, X⁴ is O. In some embodiments, X⁴ is S. In some embodiments, X⁴ is NH. In some embodiments, X⁴ is NMe. In some embodiments, X⁴ is NCH(CH₃)₂.

As defined generally above, each Z¹ is independently CR² or N. In some embodiments, Z¹ is CR². In some embodiments, Z¹ is N.

As defined generally above, each Z² is independently CR³ or N. In some embodiments, Z² is CR³. In some embodiments, Z¹ is N.

As defined generally above, Z¹¹ is CHR³, C(R³)₂, or NR⁴. In some embodiments, Z¹¹ is CHR³. In some embodiments, Z¹¹ is C(R³)₂. In some embodiments, Z¹¹ is NR⁴.

As defined generally above, Z¹² is CHR², C(R²)₂, NR⁴, or C(═N—R⁴). In some embodiments, Z¹² is CHR². In some embodiments, Z¹² is C(R²)₂. In some embodiments, Z¹² is NR⁴. In some embodiments, Z¹² is C(═N—R⁴).

As defined generally above, R² and R³ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted, provided that at least one of R² and R³ is not hydrogen; or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted. In some embodiments, R² and R³ are each independently —NR—C(O)—R.

In some embodiments, R² is hydrogen. In some embodiments, R² is an optionally substituted C₁₋₆ aliphatic group. In some embodiments, R² is halogen. In some embodiments, R² is —OR. In some embodiments, R² is —NR₂. In some embodiments, R² is —C(═O)R. In some embodiments, R² is —C(═O)OR. In some embodiments, R² is —C(═O)NR₂. In some embodiments, R² is —SO₂R. In some embodiments, R² is —SO₂NR₂. In some embodiments, R² is C₁₋₆haloalkyl. In some embodiments, R² is C₁₋₆haloalkoxy. In some embodiments, R² is an optionally substituted 3-8 membered saturated or partially unsaturated monocyclic carbocyclic ring. In some embodiments, R² is an optionally substituted 6-12 membered saturated or partially unsaturated bridged carbocyclic ring. In some embodiments, R² is an optionally substituted 7-12 membered saturated or partially unsaturated bicyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 3-8 membered saturated or partially unsaturated monocyclic heterocyclic ring (having 1-2 heteroatoms independently selected from nitrogen, oxygen, and sulfur). In some embodiments, R² is an optionally substituted 6-12 membered saturated or partially unsaturated bridged heterocyclic ring (having 1-4 heteroatoms independently selected from nitrogen, oxygen, and sulfur). In some embodiments, R² is an optionally substituted 7-12 membered saturated or partially unsaturated bicyclic heterocyclic ring (having 1-4 heteroatoms independently selected from nitrogen, oxygen, and sulfur). In some embodiments, R² is an optionally substituted 5-6 membered monocyclic heteroaromatic ring (having 1-4 heteroatoms independently selected from nitrogen, oxygen, and sulfur). In some embodiments, R² is an optionally substituted 8-10 membered bicyclic heteroaromatic ring (having 1-5 heteroatoms independently selected from nitrogen, oxygen, and sulfur). In some embodiments, R² is selected from those depicted in Table A below. In some embodiments, R² is selected from those depicted in Table A2 below.

In some embodiments, R³ is hydrogen. In some embodiments, R³ is an optionally substituted C₁₋₆ aliphatic group. In some embodiments, R³ is halogen. In some embodiments, R³ is —OR. In some embodiments, R³ is —NR₂. In some embodiments, R³ is —NR—C(O)—R. In some embodiments, R³ is —C(═O)R. In some embodiments, R³ is —C(═O)OR. In some embodiments, R³ is —C(═O)NR₂. In some embodiments, R³ is —SO₂R. In some embodiments, R³ is —SO₂NR₂. In some embodiments, R³ is C₁₋₆haloalkyl. In some embodiments, R³ is C₁₋₆haloalkoxy. In some embodiments, R³ is an optionally substituted 3-8 membered saturated or partially unsaturated monocyclic carbocyclic ring. In some embodiments, R³ is an optionally substituted 6-12 membered saturated or partially unsaturated bridged carbocyclic ring. In some embodiments, R³ is an optionally substituted 7-12 membered saturated or partially unsaturated bicyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 3-8 membered saturated or partially unsaturated monocyclic heterocyclic ring (having 1-2 heteroatoms independently selected from nitrogen, oxygen, and sulfur). In some embodiments, R³ is an optionally substituted 6-12 membered saturated or partially unsaturated bridged heterocyclic ring (having 1-4 heteroatoms independently selected from nitrogen, oxygen, and sulfur). In some embodiments, R³ is an optionally substituted 7-12 membered saturated or partially unsaturated bicyclic heterocyclic ring (having 1-4 heteroatoms independently selected from nitrogen, oxygen, and sulfur). In some embodiments, R³ is an optionally substituted 5-6 membered monocyclic heteroaromatic ring (having 1-4 heteroatoms independently selected from nitrogen, oxygen, and sulfur). In some embodiments, R³ is an optionally substituted 8-10 membered bicyclic heteroaromatic ring (having 1-5 heteroatoms independently selected from nitrogen, oxygen, and sulfur). In some embodiments, R³ is selected from those depicted in Table A below. In some embodiments, R³ is selected from those depicted in Table A2 below.

In some embodiments, R² is hydrogen. In some embodiments, R² is methyl. In some embodiments, R² is Cl. In some embodiments, R² is isopropyl. In some embodiments, R² is a C₁₋₃ haloalkyl. In some embodiments, R² is 3-8 membered saturated monocyclic heterocyclic ring (having 1-2 heteroatoms independently selected from nitrogen, oxygen, and sulfur). In some embodiments, R² is an azetidinyl group. In some embodiments, R² is optionally substituted ethyl. In some embodiments, R² is methoxy. In some embodiments, R² is —CH₂F. In some embodiments, R² is —OCH₂F. In some embodiments, R² is -CD₃.

In some embodiments, R³ is hydrogen. In some embodiments, R³ is methyl. In some embodiments, R³ is Cl. In some embodiments, R³ is isopropyl. In some embodiments, R³ is a C₁₋₃ haloalkyl. In some embodiments, R³ is 3-8 membered saturated monocyclic heterocyclic ring (having 1-2 heteroatoms independently selected from nitrogen, oxygen, and sulfur). In some embodiments, R³ is an azetidinyl group. In some embodiments, R³ is optionally substituted ethyl. In some embodiments, R³ is methoxy. In some embodiments, R³ is —CH₂F. In some embodiments, R³ is —OCH₂F. In some embodiments, R³ is -CD₃. In some embodiments, R³ is —N(CH₃)—C(O)—CH₃. In some embodiments, R³ is —N(CH₃)₂. In some embodiments R³ is —NH(CH₃). In some embodiments, R³ is

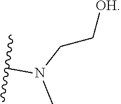 [SMILES: *N(C)CCO]

In some embodiments, R³ is

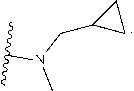 [SMILES: *N(C)CC1CC1]

In some embodiments, R³ is

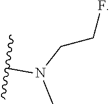 [SMILES: *N(C)CCF]

In some embodiments, R³ is

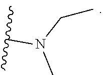 [SMILES: *N(C)CC]

In some embodiments, R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R² and R³ are taken together with their intervening atoms to form an optionally substituted 3-8 membered saturated or partially unsaturated monocyclic carbocyclic ring. In some embodiments, R² and R³ are taken together with their intervening atoms to form an optionally substituted 6-12 membered saturated or partially unsaturated bridged carbocyclic ring. In some embodiments, R² and R³ are taken together with their intervening atoms to form an optionally substituted 7-12 membered saturated or partially unsaturated bicyclic carbocyclic ring. In some embodiments, R² and R are taken together with their intervening atoms to form an optionally substituted phenyl. In some embodiments, R² and R³ are taken together with their intervening atoms to form an optionally substituted 8-10 membered bicyclic aromatic carbocyclic ring. In some embodiments, R² and R are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R² and R³ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R² and R³ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R² and R are taken together with their intervening atoms to form an optionally substituted 5-6 membered monocyclic heteroaromatic ring (having 1-4 heteroatoms independently selected from nitrogen, oxygen, and sulfur). In some embodiments, R² and R³ are taken together with their intervening atoms to form an optionally substituted 8-10 membered bicyclic heteroaromatic ring (having 1-5 heteroatoms independently selected from nitrogen, oxygen, and sulfur).

In some embodiments, R² and R³ are taken together with their intervening atoms to form a cyclopentane ring. In some embodiments, R² and R are taken together with their intervening atoms to form a pyrrolidine ring.

As defined generally above, R⁴ is hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or R³ and R⁴ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁴ is hydrogen. In some embodiments, R⁴ is an optionally substituted C₁₋₆ aliphatic group. In some embodiments, R⁴ is an optionally substituted phenyl. In some embodiments, R⁴ is an optionally substituted 3-7 membered saturated or partially unsaturated carbocyclic ring. In some embodiments, R⁴ is an optionally substituted 3-7 membered saturated or partially unsaturated heterocyclic ring (having 1-2 heteroatoms independently selected from nitrogen, oxygen, and sulfur). In some embodiments, R⁴ is an optionally substituted 5-6 membered heteroaryl ring (having 1-4 heteroatoms independently selected from nitrogen, oxygen, and sulfur).

In some embodiments, R and R⁴ are taken together with their intervening atoms to form an optionally substituted 3-8 membered saturated or partially unsaturated monocyclic carbocyclic ring. In some embodiments, R³ and R⁴ are taken together with their intervening atoms to form an optionally substituted 7-12 membered saturated or partially unsaturated bicyclic carbocyclic ring, phenyl. In some embodiments, R³ and R⁴ are taken together with their intervening atoms to form an optionally substituted 8-10 membered bicyclic aromatic carbocyclic ring. In some embodiments, R and R⁴ are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R and R⁴ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R³ and R⁴ are taken together with their intervening atoms to form an optionally substituted 5-6 membered monocyclic heteroaromatic ring (having 1-4 heteroatoms independently selected from nitrogen, oxygen, and sulfur). In some embodiments, R³ and R⁴ are taken together with their intervening atoms to form an optionally substituted 8-10 membered bicyclic heteroaromatic ring (having 1-5 heteroatoms independently selected from nitrogen, oxygen, and sulfur.

In some embodiments, R and R⁴ are taken together with their intervening atoms to form a cyclopentane ring. In some embodiments, R and R⁴ are taken together with their intervening atoms to form a pyrrolidine ring.

As defined generally above, Ring B is

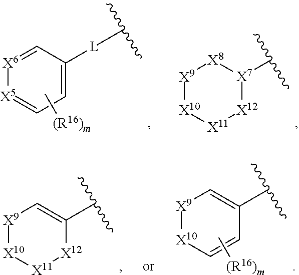

In some embodiments, Ring B is

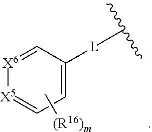

In some embodiments, Ring B is

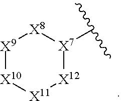

In some embodiments, Ring B is

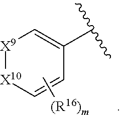

In some embodiments, Ring B is

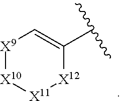

As defined generally above, L is a bond or an optionally substituted straight chain or branched C₁₋₆ alkylene. In some embodiments, L is a bond. In some embodiments, L is an optionally substituted straight chain or branched C₁₋₆ alkylene. In some embodiments, L is optionally substituted ethylene. In some embodiments, L is optionally substituted methylene. In some embodiments, L is selected from those depicted in Table A below. In some embodiments, L is selected from those depicted in Table A2 below.

As defined generally above, X⁵ is CH, N or CR⁵. In some embodiments, X⁵ is CH. In some embodiments, X⁵ is N. In some embodiments, X⁵ is CR⁵. In some embodiments, X⁵ is selected from those depicted in Table A below. In some embodiments, X⁵ is selected from those depicted in Table A2 below.

As defined generally above, X⁶ is CH, N or CR⁶. In some embodiments, X⁶ is CH. In some embodiments, X⁶ is N. In some embodiments, X⁶ is CR⁶. In some embodiments, X⁶ is selected from those depicted in Table A below. In some embodiments, X⁶ is selected from those depicted in Table A2 below.

In some embodiments, X⁵ is N and X⁶ is CH. In some embodiments, X⁵ is N and X⁶ is CR⁶. In some embodiments, X⁵ is CH and X⁶ is N. In some embodiments, X⁵ is CR⁵ and X⁶ is N. In some embodiments, X⁵ is CH and X⁶ is CH. In some embodiments, X⁵ is CH and X⁶ is CR⁶. In some embodiments, X⁵ is CR⁵ and X⁶ is CH.

As defined generally above, R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R¹⁶ is hydrogen. In some embodiments, R¹⁶ is an optionally substituted C₁₋₆ aliphatic group. In some embodiments, R¹⁶ is halogen. In some embodiments, R¹³ is —OR. In some embodiments, R¹⁶ is —CN. In some embodiments, R¹⁶ is —NR₂. In some embodiments, R¹⁶ is —C(═O)R. In some embodiments, R¹⁶ is —C(═O)OR. In some embodiments, R¹⁶ is —C(═O)NR₂. In some embodiments, R¹⁶ is —SO₂R. In some embodiments, R¹⁶ is —SO₂NR₂. In some embodiments, R¹⁶ is C₁₋₆haloalkyl. In some embodiments, R¹⁶ is C₁₋₆haloalkoxy. In some embodiments, R¹⁶ is -CD₃. In some embodiments, R¹⁶ is selected from those depicted in Table A below. In some embodiments, R¹⁶ is selected from those depicted in Table A2 below.

As defined generally above, m is 0, 1 or 2. In some embodiments, m is 0. In some embodiments, m is 1. In some embodiments, m is 2.

In some embodiments, Ring B is

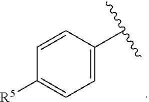

In some embodiments, Ring B is

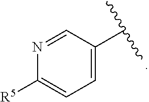

In some embodiments, Ring B is

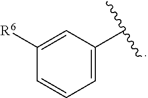

In some embodiments, Ring B is

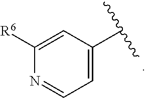

In some embodiments, Ring B is

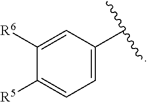

As defined generally above, R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁵ is an optionally substituted C₁₋₆ aliphatic group. In some embodiments, R⁵ is —OR. In some embodiments, R is —NR₂. In some embodiments, R is —C(═O)R. In some embodiments, R⁵ is —C(═O)OR. In some embodiments, R⁵ is —C(═O)NR₂. In some embodiments, R is —SO₂R. In some embodiments, R⁵ is —SO₂NR₂. In some embodiments, R⁵ is halogen. In some embodiments, R⁵ is C₁₋₆haloalkyl. In some embodiments, R⁵ is C₁₋₆haloalkoxy. In some embodiments, R is an optionally substituted 3-8 membered saturated or partially unsaturated monocyclic carbocyclic ring. In some embodiments, R is an optionally substituted 6-12 membered saturated or partially unsaturated bridged carbocyclic ring. In some embodiments, R⁵ is an optionally substituted 7-12 membered saturated or partially unsaturated bicyclic carbocyclic ring. In some embodiments, R⁵ is an optionally substituted phenyl. In some embodiments, R⁵ is an optionally substituted 8-10 membered bicyclic aromatic carbocyclic ring. In some embodiments, R is an optionally substituted 3-8 membered saturated or partially unsaturated monocyclic heterocyclic ring (having 1-2 heteroatoms independently selected from nitrogen, oxygen, and sulfur). In some embodiments, R⁵ is an optionally substituted 6-12 membered saturated or partially unsaturated bridged heterocyclic ring (having 1-4 heteroatoms independently selected from nitrogen, oxygen, and sulfur). In some embodiments, R⁵ is an optionally substituted 7-12 membered saturated or partially unsaturated bicyclic heterocyclic ring (having 1-4 heteroatoms independently selected from nitrogen, oxygen, and sulfur). In some embodiments, R is an optionally substituted 5-6 membered monocyclic heteroaromatic ring (having 1-4 heteroatoms independently selected from nitrogen, oxygen, and sulfur). In some embodiments, R⁵ is an optionally substituted 8-10 membered bicyclic heteroaromatic ring (having 1-5 heteroatoms independently selected from nitrogen, oxygen, and sulfur).

In some embodiments, R⁵ is F. In some embodiments, R⁵ is Cl. In some embodiments, R is —OCF₃. In some embodiments, R is cyclopropyl. In some embodiments, R is selected from those depicted in Table A below. In some embodiments, R⁵ is selected from those depicted in Table A2 below.

In some embodiments, R⁶ is an optionally substituted C₁₋₆ aliphatic group. In some embodiments, R⁶ is —OR. In some embodiments, R⁶ is —NR₂. In some embodiments, R⁶ is —C(═O)R. In some embodiments, R⁶ is —C(═O)OR. In some embodiments, R⁶ is —C(═O)NR₂. In some embodiments, R⁶ is —SO₂R. In some embodiments, R⁶ is —SO₂NR₂. In some embodiments, R⁶ is halogen. In some embodiments, R⁶ is C₁₋₆haloalkyl. In some embodiments, R⁶ is C₁₋₆haloalkoxy. In some embodiments, R⁶ is an optionally substituted 3-8 membered saturated or partially unsaturated monocyclic carbocyclic ring. In some embodiments, R⁶ is an optionally substituted 6-12 membered saturated or partially unsaturated bridged carbocyclic ring. In some embodiments, R⁶ is an optionally substituted 7-12 membered saturated or partially unsaturated bicyclic carbocyclic ring. In some embodiments, R⁶ is an optionally substituted phenyl. In some embodiments, R⁶ is an optionally substituted 8-10 membered bicyclic aromatic carbocyclic ring. In some embodiments, R⁶ is an optionally substituted 3-8 membered saturated or partially unsaturated monocyclic heterocyclic ring (having 1-2 heteroatoms independently selected from nitrogen, oxygen, and sulfur). In some embodiments, R⁶ is an optionally substituted 6-12 membered saturated or partially unsaturated bridged heterocyclic ring (having 1-4 heteroatoms independently selected from nitrogen, oxygen, and sulfur). In some embodiments, R⁶ is an optionally substituted 7-12 membered saturated or partially unsaturated bicyclic heterocyclic ring (having 1-4 heteroatoms independently selected from nitrogen, oxygen, and sulfur). In some embodiments, R⁶ is an optionally substituted 5-6 membered monocyclic heteroaromatic ring (having 1-4 heteroatoms independently selected from nitrogen, oxygen, and sulfur). In some embodiments, R⁶ is an optionally substituted 8-10 membered bicyclic heteroaromatic ring (having 1-5 heteroatoms independently selected from nitrogen, oxygen, and sulfur).

In some embodiments, R⁶ is F. In some embodiments, R⁶ is Cl. In some embodiments, R⁶ is —OCF₃. In some embodiments, R⁶ is cyclopropyl. In some embodiments, R⁶ is cyclobutyl. In some embodiments, R⁶ is optionally substituted pyrazolyl. In some embodiments, R⁶ is optionally substituted pyridinyl. In some embodiments, R⁶ is optionally substituted pyrimidinyl. In some embodiments, R⁶ is optionally substituted pyridazinyl. In some embodiments, R⁶ is optionally substituted imidazolyl. In some embodiments, R⁶ is optionally substituted triazolyl. In some embodiments, R⁶ is optionally substituted oxazolyl. In some embodiments, R⁶ is optionally substituted thiazolyl. In some embodiments, R⁶ is optionally substituted oxadiazolyl. In some embodiments, R⁶ is optionally substituted thiadiazolyl. In some embodiments, R⁶ is optionally substituted oxetanyl. In some embodiments, R⁶ is optionally substituted azetidinyl. In some embodiments, R⁶ is optionally substituted piperidinyl. In some embodiments, R⁶ is optionally substituted piperazinyl. In some embodiments, R⁶ is selected from those depicted in Table A below. In some embodiments, R⁶ is selected from those depicted in Table A2 below.

In some embodiments, R⁵ and R⁶ are independently a substituent selected from hydrogen and those shown below:

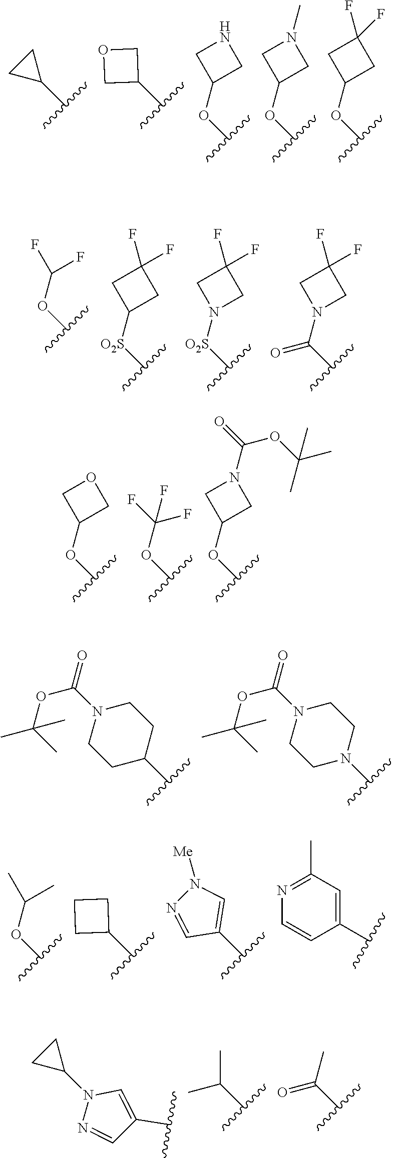

In some embodiments, R⁵ and R⁶ taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R and R⁶ are taken together with their intervening atoms to form an optionally substituted 3-8 membered saturated or partially unsaturated monocyclic carbocyclic ring. In some embodiments, R⁵ and R⁶ are taken together with their intervening atoms to form an optionally substituted 6-12 membered saturated or partially unsaturated bridged carbocyclic ring. In some embodiments, R⁵ and R⁶ are taken together with their intervening atoms to form an optionally substituted 7-12 membered saturated or partially unsaturated bicyclic carbocyclic ring. In some embodiments, R and R are taken together with their intervening atoms to form an optionally substituted phenyl. In some embodiments, R⁵ and R⁶ are taken together with their intervening atoms to form an optionally substituted 8-10 membered bicyclic aromatic carbocyclic ring. In some embodiments, R⁵ and R⁶ are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R and R⁶ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R⁵ and R⁶ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R and R⁶ are taken together with their intervening atoms to form an optionally substituted 5-6 membered monocyclic heteroaromatic ring (having 1-4 heteroatoms independently selected from nitrogen, oxygen, and sulfur). In some embodiments, R⁵ and R⁶ are taken together with their intervening atoms to form an optionally substituted 8-10 membered bicyclic heteroaromatic ring (having 1-5 heteroatoms independently selected from nitrogen, oxygen, and sulfur).

In some embodiments, R⁵ and R⁶ are taken together with their intervening atoms to form a dioxole ring.

As defined generally above, X⁷ is N, CH, or CR⁷. In some embodiments, X⁷ is N. In some embodiments, X⁷ is CH. In some embodiments, X⁷ is CR⁷. In some embodiments, X⁷ is CCH₃. In some embodiments, X⁷ is COH. In some embodiments, X⁷ is CF. In some embodiments, X⁷ is selected from those depicted in Table A below. In some embodiments, X⁷ is selected from those depicted in Table A2 below.

As defined generally above, X⁸ is O, NR^{B}, C(R^{B})₂, CHR⁸, SO₂, or C═O. In some embodiments, X⁸ is O. In some embodiments, X⁸ is NR^{B}. In some embodiments, X⁸ is C(R^{B})₂. In some embodiments, X⁸ is CHR^{B}. In some embodiments, X⁸ is SO₂. In some embodiments, X⁸ is CH₂. In some embodiments, X⁸ is C═O. In some embodiments, X⁸ is selected from those depicted in Table A below. In some embodiments, X⁸ is selected from those depicted in Table A2 below.

As defined generally above, X⁹ is O, NR⁹, C(R⁹)₂, CHR⁹, SO₂, or C═O. In some embodiments, X⁹ is O. In some embodiments, X⁹ is NR⁹. In some embodiments, X⁹ is C(R⁹)₂. In some embodiments, X⁹ is CHR⁹. In some embodiments, X⁹ is SO₂. In some embodiments, X⁹ is CH₂. In some embodiments, X⁹ is C═O. In some embodiments, X⁹ is selected from those depicted in Table A below. In some embodiments, X⁹ is selected from those depicted in Table A2 below.

As defined generally above, X¹⁰ is O, NR¹⁰, C(R¹⁰)₂, CHR¹⁰, SO₂, or C═O. In some embodiments, X¹⁰ is O. In some embodiments, X¹⁰ is NR¹⁰. In some embodiments, X¹⁰ is C(R¹⁰)₂. In some embodiments, X¹⁰ is CHR¹⁰. In some embodiments, X¹⁰ is SO₂. In some embodiments, X¹⁰ is C═O. In some embodiments, X¹⁰ is CH₂, CF₂, or O. In some embodiments, X¹⁰ is CH₂. In some embodiments, X¹⁰ is NR¹⁰, or O. In some embodiments, X¹⁰ is NMe, NH, or O. In some embodiments, X¹⁰ is selected from those depicted in Table A below. In some embodiments, X¹⁰ is selected from those depicted in Table A2 below.

As defined generally above, X¹¹ is O, NR¹¹, C(R¹¹)₂, CHR¹¹, SO₂, or C═O. In some embodiments, X¹¹ is O. In some embodiments, X¹¹ is NR¹¹. In some embodiments, X¹¹ is C(R¹¹)₂. In some embodiments, X¹¹ is CHR¹¹. In some embodiments, X¹¹ is SO₂. In some embodiments, X¹¹ is CH₂. In some embodiments, X¹¹ is C═O. In some embodiments, X¹¹ is selected from those depicted in Table A below. In some embodiments, X¹¹ is selected from those depicted in Table A2 below.

As defined generally above, X¹² is a direct bond, O, NR¹², C(R¹²)₂, CHR¹², —CH₂CH₂—, —OCH₂—, SO₂, or C═O. In some embodiments, X¹² is O. In some embodiments, X¹² is NR¹². In some embodiments, X¹² is C(R¹²)₂. In some embodiments, X¹² is CHR¹². In some embodiments, X¹² is CH₂. In some embodiments, X¹² is SO₂. In some embodiments, X¹² is C═O. In some embodiments, X¹² is —CH₂CH₂—. In some embodiments, X¹² is —OCH₂—. In some embodiments, X¹² is a direct bond. In some embodiments, X¹² is selected from those depicted in Table A below. In some embodiments, X¹² is selected from those depicted in Table A2 below.

In some embodiments, when any of X⁷, X⁸, X⁹, X¹⁰, X¹¹, or X¹² is N, O or SO₂, then neither of the neighboring positions in Ring B are N, O or SO₂.

In some embodiments, when any one of X⁸, X⁹, X¹⁰, X¹¹, or X¹² is C═O, then neither of the neighboring positions in Ring B are C═O or SO₂.

As defined generally above, R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R⁷ is an optionally substituted aliphatic group. In some embodiments, R⁷ is halogen. In some embodiments, R⁷ is —OR. In some embodiments, R⁷ is —NR₂. In some embodiments, R⁷ is —C(═O)R. In some embodiments, R⁷ is —C(═O)OR. In some embodiments, R⁷ is —C(═O)NR₂. In some embodiments, R⁷ is —SO₂R. In some embodiments, R⁷ is —SO₂NR₂. In some embodiments, R⁷ is C₁₋₆haloalkyl. In some embodiments, R⁷ is C₁₋₆haloalkoxy. In some embodiments, R⁷ is methyl. In some embodiments, R⁷ is OH. In some embodiments, R⁷ is F. In some embodiments, R⁷ is selected from those depicted in Table A below. In some embodiments, R⁷ is selected from those depicted in Table A2 below.

As defined generally above, each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁸ is hydrogen. In some embodiments, R⁸ is an optionally substituted C₁₋₆ aliphatic group. In some embodiments, R⁸—OR. In some embodiments, R⁸ is —NR₂. In some embodiments, R⁸ is —C(═O)R. In some embodiments, R⁸ is —C(═O)OR. In some embodiments, R⁸ is —C(═O)NR₂. In some embodiments, R⁸ is —SO₂R. In some embodiments, R⁸ is —SO₂NR₂. In some embodiments, R⁸ is C₁₋₆haloalkyl. In some embodiments, R⁸ is C₁₋₆haloalkoxy. In some embodiments, R⁸ is an optionally substituted 3-8 membered saturated or partially unsaturated monocyclic carbocyclic ring. In some embodiments, R⁸ is an optionally substituted 7-12 membered saturated or partially unsaturated bicyclic carbocyclic ring. In some embodiments, R⁸ is an optionally substituted phenyl. In some embodiments, R⁸ is an optionally substituted 8-10 membered bicyclic aromatic carbocyclic ring. In some embodiments, R⁸ is an optionally substituted 3-8 membered saturated or partially unsaturated monocyclic heterocyclic ring (having 1-2 heteroatoms independently selected from nitrogen, oxygen, and sulfur). In some embodiments, R⁸ is an optionally substituted 7-12 membered saturated or partially unsaturated bicyclic heterocyclic ring (having 1-4 heteroatoms independently selected from nitrogen, oxygen, and sulfur). In some embodiments, R⁸ is an optionally substituted 5-6 membered monocyclic heteroaromatic ring (having 1-4 heteroatoms independently selected from nitrogen, oxygen, and sulfur). In some embodiments, R⁸ is an optionally substituted 8-10 membered bicyclic heteroaromatic ring (having 1-5 heteroatoms independently selected from nitrogen, oxygen, and sulfur). In some embodiments, R⁸ is methyl. In some embodiments, R⁸ is —OH. In some embodiments, R⁸ is F. In some embodiments, R⁸ is methoxy. In some embodiments, R⁸ is —CH₂OH. In some embodiments, wherein X⁸ is C(R)₂, each R⁸ is independently selected from any of the aforementioned substituents. In some embodiments, wherein X⁸ is C(R⁸)₂, both R⁸ are the same. In some embodiments, R⁸ is selected from those depicted in Table A below. In some embodiments, R⁸ is selected from those depicted in Table A2 below.

In some embodiments, R⁹ is hydrogen. In some embodiments, R⁹ is an optionally substituted C₁₋₆ aliphatic group. In some embodiments, R⁹—OR. In some embodiments, R⁹ is —NR₂. In some embodiments, R⁹ is —C(═O)R. In some embodiments, R⁹ is —C(═O)OR. In some embodiments, R⁹ is —C(═O)NR₂. In some embodiments, R⁹ is —SO₂R. In some embodiments, R⁹ is —SO₂NR₂. In some embodiments, R⁹ is C₁₋₆haloalkyl. In some embodiments, R⁹ is C₁₋₆haloalkoxy. In some embodiments, R⁹ is an optionally substituted 3-8 membered saturated or partially unsaturated monocyclic carbocyclic ring. In some embodiments, R⁹ is an optionally substituted 7-12 membered saturated or partially unsaturated bicyclic carbocyclic ring. In some embodiments, R⁹ is an optionally substituted phenyl. In some embodiments, R⁹ is an optionally substituted 8-10 membered bicyclic aromatic carbocyclic ring. In some embodiments, R⁹ is an optionally substituted 3-8 membered saturated or partially unsaturated monocyclic heterocyclic ring (having 1-2 heteroatoms independently selected from nitrogen, oxygen, and sulfur). In some embodiments, R⁹ is an optionally substituted 7-12 membered saturated or partially unsaturated bicyclic heterocyclic ring (having 1-4 heteroatoms independently selected from nitrogen, oxygen, and sulfur). In some embodiments, R⁹ is an optionally substituted 5-6 membered monocyclic heteroaromatic ring (having 1-4 heteroatoms independently selected from nitrogen, oxygen, and sulfur). In some embodiments, R⁹ is an optionally substituted 8-10 membered bicyclic heteroaromatic ring (having 1-5 heteroatoms independently selected from nitrogen, oxygen, and sulfur). In some embodiments, R⁹ is methyl. In some embodiments, R⁹ is —OH. In some embodiments, R⁹ is F. In some embodiments, R⁹ is methoxy. In some embodiments, R⁹ is —CH₂OH. In some embodiments, wherein X⁹ is C(R⁹)₂, each R⁹ is independently selected from any of the aforementioned substituents. In some embodiments, wherein X⁹ is C(R⁹)₂, both R⁹ are the same. In some embodiments, R⁹ is selected from those depicted in Table A below. In some embodiments, R⁹ is selected from those depicted in Table A2 below.

In some embodiments, R⁹ is optionally substituted pyrazolyl. In some embodiments, R⁹ is optionally substituted pyridinyl. In some embodiments, R⁹ is optionally substituted pyrimidinyl. In some embodiments, R⁹ is optionally substituted pyridazinyl. In some embodiments, R⁹ is optionally substituted imidazolyl. In some embodiments, R⁹ is optionally substituted triazolyl. In some embodiments, R⁹ is optionally substituted oxazolyl. In some embodiments, R⁹ is optionally substituted thiazolyl. In some embodiments, R⁹ is optionally substituted oxadiazolyl. In some embodiments, R⁹ is optionally substituted thiadiazolyl. In some embodiments, R⁹ is optionally substituted oxetanyl. In some embodiments, R⁹ is optionally substituted azetidinyl. In some embodiments, R⁹ is optionally substituted piperidinyl. In some embodiments, R⁹ is optionally substituted piperazinyl.

In some embodiments, R⁹ is substituted with an optionally substituted 3-6 membered saturated or partially unsaturated monocyclic carbocyclic ring. In some embodiments, R⁹ is substituted with an optionally substituted 5-8 membered saturated or partially unsaturated bicyclic carbocyclic ring. In some embodiments, R⁹ is substituted with an optionally substituted 3-6 membered saturated or partially unsaturated monocyclic heterocyclic ring. In some embodiments, R⁹ is substituted with an optionally substituted C₁₋₆ aliphatic group. In some embodiments, R⁹ is substituted with a methyl group. In some embodiments, R⁹ is substituted with a -CD₃ group. In some embodiments, R⁹ is substituted with a methoxy group. In some embodiments, R⁹ is substituted with a cyclopropyl group. In some embodiments, R⁹ is substituted with an optionally substituted

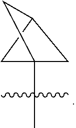 [SMILES: *C12CC(C1)C2]

In some embodiments, R⁹ is —OR, wherein R is an optionally substituted 5-6 membered heteroaryl ring (having 1-4 heteroatoms independently selected from nitrogen, oxygen, and sulfur). In some embodiments, R⁹ is —NHR, wherein R is an optionally substituted 5-6 membered heteroaryl ring (having 1-4 heteroatoms independently selected from nitrogen, oxygen, and sulfur). In some embodiments, R⁹ is —N(CH₃)R, wherein R is an optionally substituted 5-6 membered heteroaryl ring (having 1-4 heteroatoms independently selected from nitrogen, oxygen, and sulfur). In some embodiments, R⁹ is —C(═O)N(CH₃)R, wherein R is an optionally substituted 5-6 membered heteroaryl ring (having 1-4 heteroatoms independently selected from nitrogen, oxygen, and sulfur). In some embodiments, R⁹ is —C(═O)NHR, wherein R is an optionally substituted 5-6 membered heteroaryl ring (having 1-4 heteroatoms independently selected from nitrogen, oxygen, and sulfur).

In some embodiments, R⁹ is a substituent selected from those shown below:

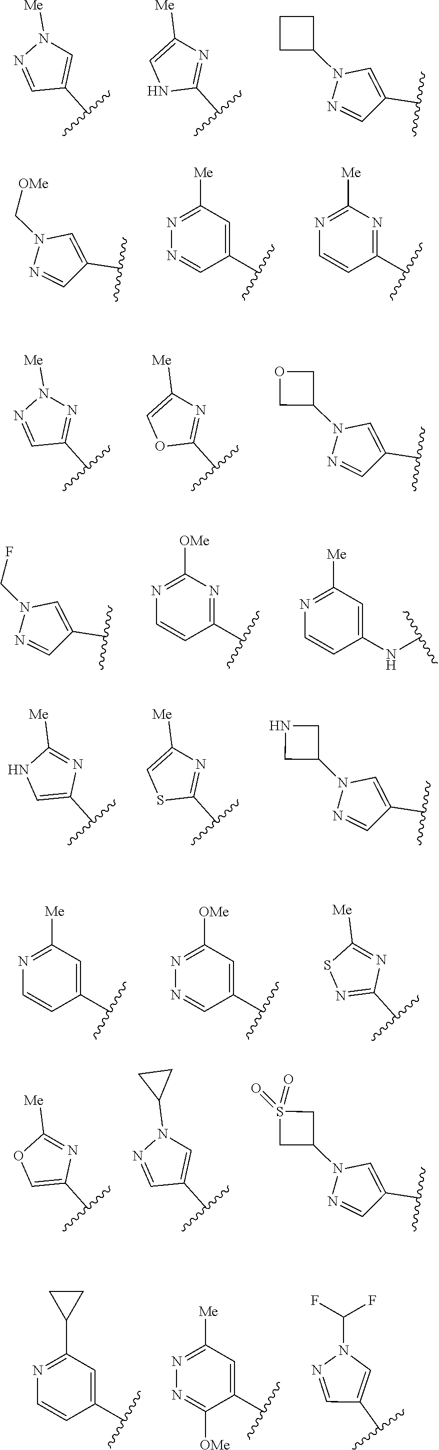

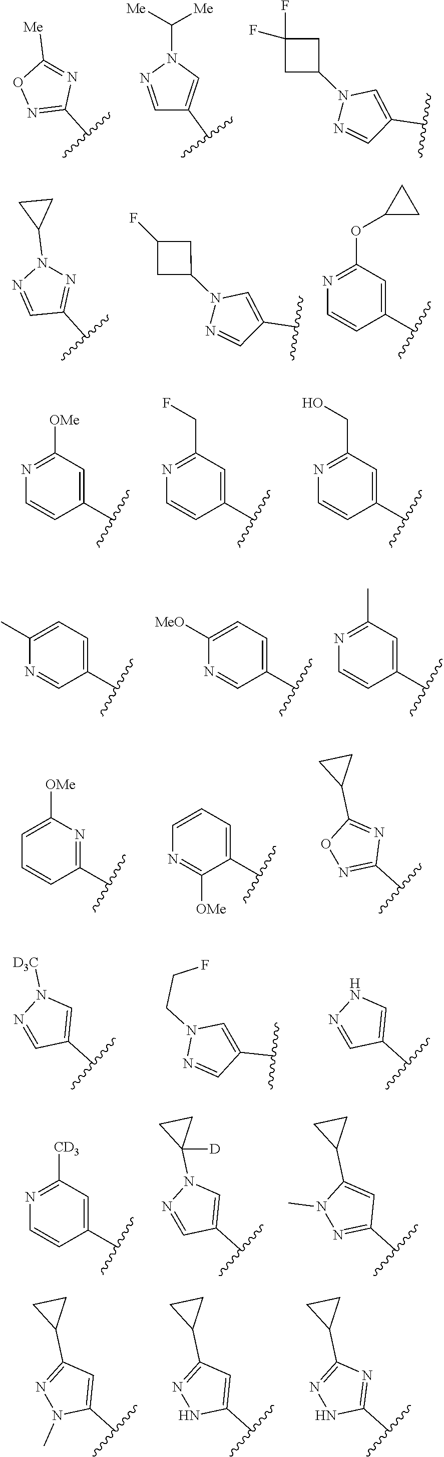

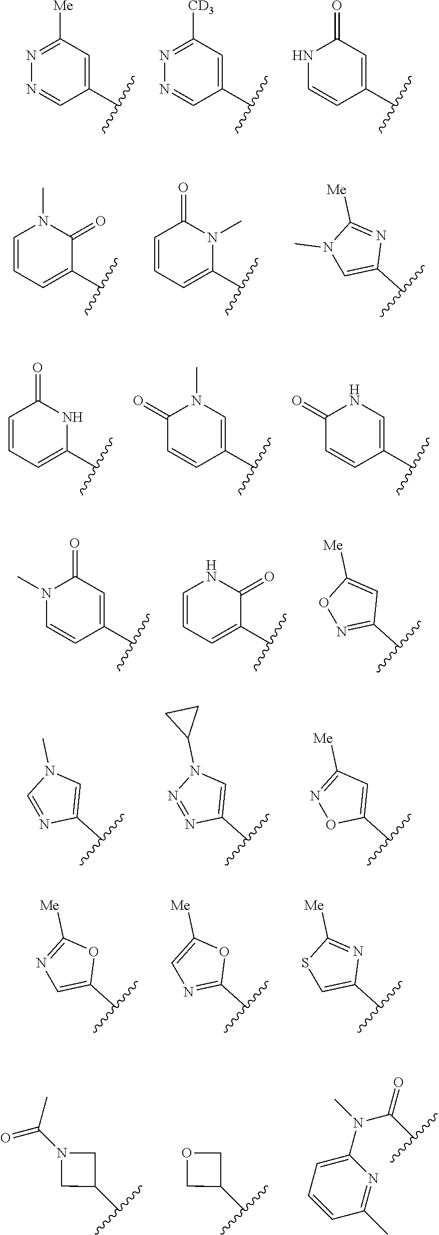

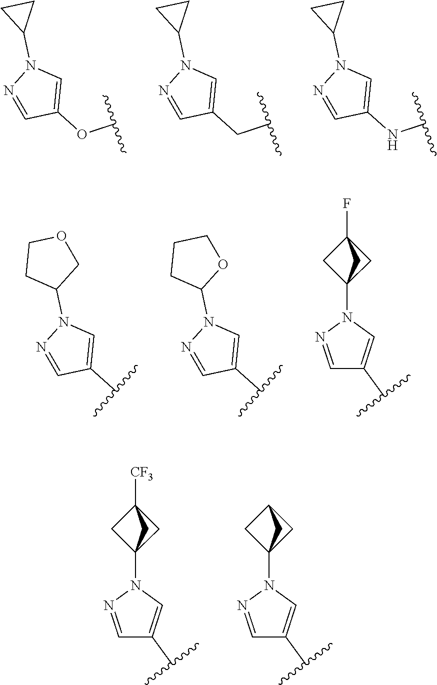

In some embodiments, R⁹ is methyl, tetrahydrofuran-3-yl,

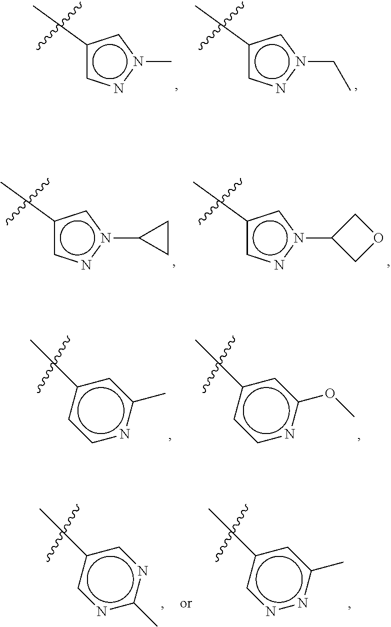

In some embodiments, R⁹ is methyl, tetrahydrofuran-3-yl,

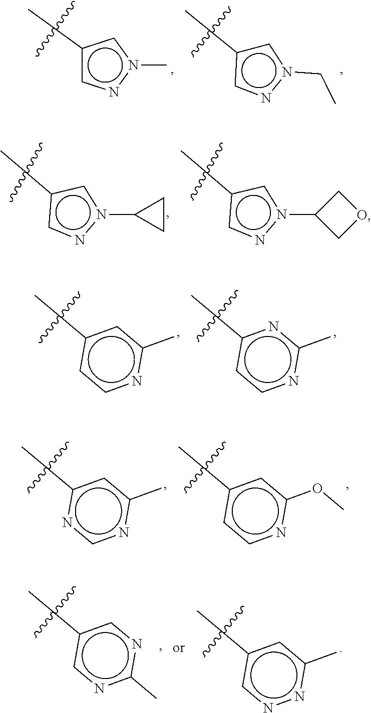

In some embodiments, R⁹ is

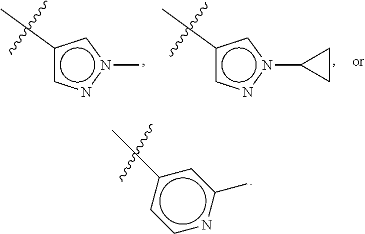

In some embodiments, R⁹ is

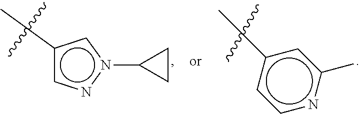

In some embodiments, R⁹ is

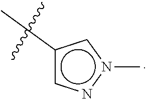

In some embodiments, R⁹ is

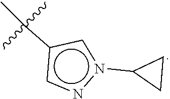

In some embodiments, R⁹ is

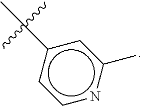

In some embodiments, R¹⁰ is hydrogen. In some embodiments, R¹⁰ is an optionally substituted C₁₋₆ aliphatic group. In some embodiments, R¹⁰—OR. In some embodiments, R¹⁰ is —NR₂. In some embodiments, R¹⁰ is —C(═O)R. In some embodiments, R¹⁰ is —C(═O)OR. In some embodiments, R¹⁰ is —C(═O)NR₂. In some embodiments, R¹⁰ is —SO₂R. In some embodiments, R¹⁰ is —SO₂NR₂. In some embodiments, R¹⁰ is C₁₋₆haloalkyl. In some embodiments, R¹⁰ is C₁₋₆haloalkoxy. In some embodiments, R¹⁰ is an optionally substituted 3-8 membered saturated or partially unsaturated monocyclic carbocyclic ring. In some embodiments, R¹⁰ is an optionally substituted 7-12 membered saturated or partially unsaturated bicyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 3-8 membered saturated or partially unsaturated monocyclic heterocyclic ring (having 1-2 heteroatoms independently selected from nitrogen, oxygen, and sulfur). In some embodiments, R¹⁰ is an optionally substituted 7-12 membered saturated or partially unsaturated bicyclic heterocyclic ring (having 1-4 heteroatoms independently selected from nitrogen, oxygen, and sulfur). In some embodiments, R¹⁰ is an optionally substituted 5-6 membered monocyclic heteroaromatic ring (having 1-4 heteroatoms independently selected from nitrogen, oxygen, and sulfur). In some embodiments, R¹⁰ is an optionally substituted 8-10 membered bicyclic heteroaromatic ring (having 1-5 heteroatoms independently selected from nitrogen, oxygen, and sulfur). In some embodiments, R¹⁰ is methyl. In some embodiments, R¹⁰ is —OH. In some embodiments, R¹⁰ is F. In some embodiments, R¹⁰ is methoxy. In some embodiments, R¹⁰ is —CH₂OH. In some embodiments, wherein X¹⁰ is C(R¹⁰)₂, each R¹⁰ is independently selected from any of the aforementioned substituents. In some embodiments, wherein X¹⁰ is C(R¹⁰)₂, both R¹⁰ are the same. In some embodiments, R¹⁰ is selected from those depicted in Table A below. In some embodiments, R¹⁰ is selected from those depicted in Table A2 below.

In some embodiments, R¹¹ is hydrogen. In some embodiments, R¹¹ is an optionally substituted C₁₋₆ aliphatic group. In some embodiments, R¹¹—OR. In some embodiments, R¹¹ is —NR₂. In some embodiments, R¹¹ is —C(═O)R. In some embodiments, R¹¹ is —C(═O)OR. In some embodiments, R¹¹ is —C(═O)NR₂. In some embodiments, R¹¹ is —SO₂R. In some embodiments, R¹¹ is —SO₂NR₂. In some embodiments, R¹¹ is C₁₋₆haloalkyl. In some embodiments, R¹¹ is C₁₋₆haloalkoxy. In some embodiments, R¹¹ is an optionally substituted 3-8 membered saturated or partially unsaturated monocyclic carbocyclic ring. In some embodiments, R¹¹ is an optionally substituted 7-12 membered saturated or partially unsaturated bicyclic carbocyclic ring. In some embodiments, R¹¹ is an optionally substituted phenyl. In some embodiments, R¹¹ is an optionally substituted 8-10 membered bicyclic aromatic carbocyclic ring. In some embodiments, R¹¹ is an optionally substituted 3-8 membered saturated or partially unsaturated monocyclic heterocyclic ring (having 1-2 heteroatoms independently selected from nitrogen, oxygen, and sulfur). In some embodiments, R¹¹ is an optionally substituted 7-12 membered saturated or partially unsaturated bicyclic heterocyclic ring (having 1-4 heteroatoms independently selected from nitrogen, oxygen, and sulfur). In some embodiments, R¹¹ is an optionally substituted 5-6 membered monocyclic heteroaromatic ring (having 1-4 heteroatoms independently selected from nitrogen, oxygen, and sulfur). In some embodiments, R¹¹ is an optionally substituted 8-10 membered bicyclic heteroaromatic ring (having 1-5 heteroatoms independently selected from nitrogen, oxygen, and sulfur). In some embodiments, R¹¹ is methyl. In some embodiments, R¹¹ is —OH. In some embodiments, R¹¹ is F. In some embodiments, R¹¹ is methoxy. In some embodiments, R¹¹ is —CH₂OH. In some embodiments, wherein X¹¹ is C(R¹¹)₂, each R¹¹ is independently selected from any of the aforementioned substituents. In some embodiments, wherein X¹¹ is C(R¹¹)₂, both R¹¹ are the same. In some embodiments, R¹¹ is selected from those depicted in Table A below. In some embodiments, R¹¹ is selected from those depicted in Table A2 below.

In some embodiments, R¹² is hydrogen. In some embodiments, R¹² is an optionally substituted C₁₋₆ aliphatic group. In some embodiments, R¹²—OR. In some embodiments, R¹² is —NR₂. In some embodiments, R¹² is —C(═O)R. In some embodiments, R¹² is —C(═O)OR. In some embodiments, R¹² is —C(═O)NR₂. In some embodiments, R¹² is —SO₂R. In some embodiments, R¹² is —SO₂NR₂. In some embodiments, R¹² is C₁₋₆haloalkyl. In some embodiments, R¹² is C₁₋₆haloalkoxy. In some embodiments, R¹² is an optionally substituted 3-8 membered saturated or partially unsaturated monocyclic carbocyclic ring. In some embodiments, R¹² is an optionally substituted 7-12 membered saturated or partially unsaturated bicyclic carbocyclic ring. In some embodiments, R¹² is an optionally substituted phenyl. In some embodiments, R¹² is an optionally substituted 8-10 membered bicyclic aromatic carbocyclic ring. In some embodiments, R¹² is an optionally substituted 3-8 membered saturated or partially unsaturated monocyclic heterocyclic ring (having 1-2 heteroatoms independently selected from nitrogen, oxygen, and sulfur). In some embodiments, R¹² is an optionally substituted 7-12 membered saturated or partially unsaturated bicyclic heterocyclic ring (having 1-4 heteroatoms independently selected from nitrogen, oxygen, and sulfur). In some embodiments, R¹² is an optionally substituted 5-6 membered monocyclic heteroaromatic ring (having 1-4 heteroatoms independently selected from nitrogen, oxygen, and sulfur). In some embodiments, R¹² is an optionally substituted 8-10 membered bicyclic heteroaromatic ring (having 1-5 heteroatoms independently selected from nitrogen, oxygen, and sulfur). In some embodiments, R¹² is methyl. In some embodiments, R¹² is —OH. In some embodiments, R¹² is F. In some embodiments, R¹² is methoxy. In some embodiments, R¹² is —CH₂OH. In some embodiments, wherein X¹² is C(R¹²)₂, each R¹² is independently selected from any of the aforementioned substituents. In some embodiments, wherein X¹² is C(R¹²)₂, both R¹² are the same. In some embodiments, R¹² is selected from those depicted in Table A below. In some embodiments, R¹² is selected from those depicted in Table A2 below.

In some embodiments, Ring B is a substituent selected from those shown below:

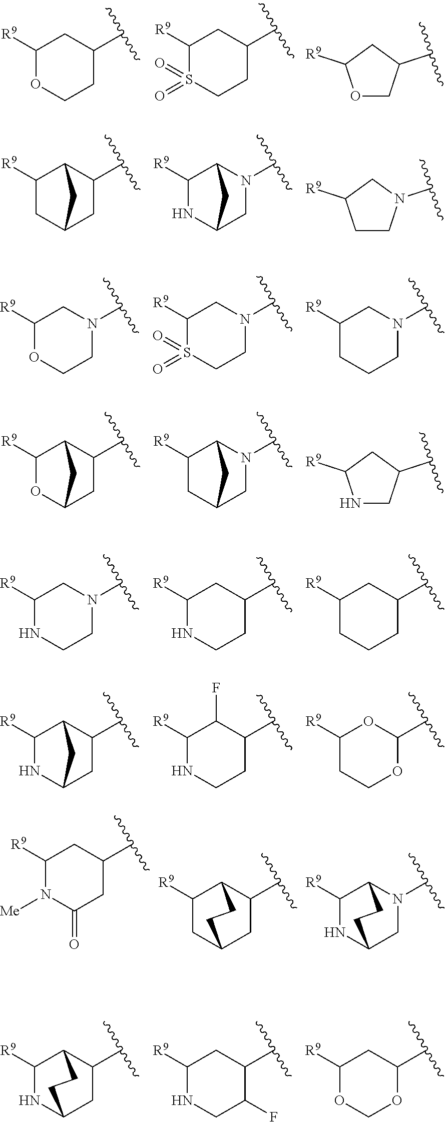

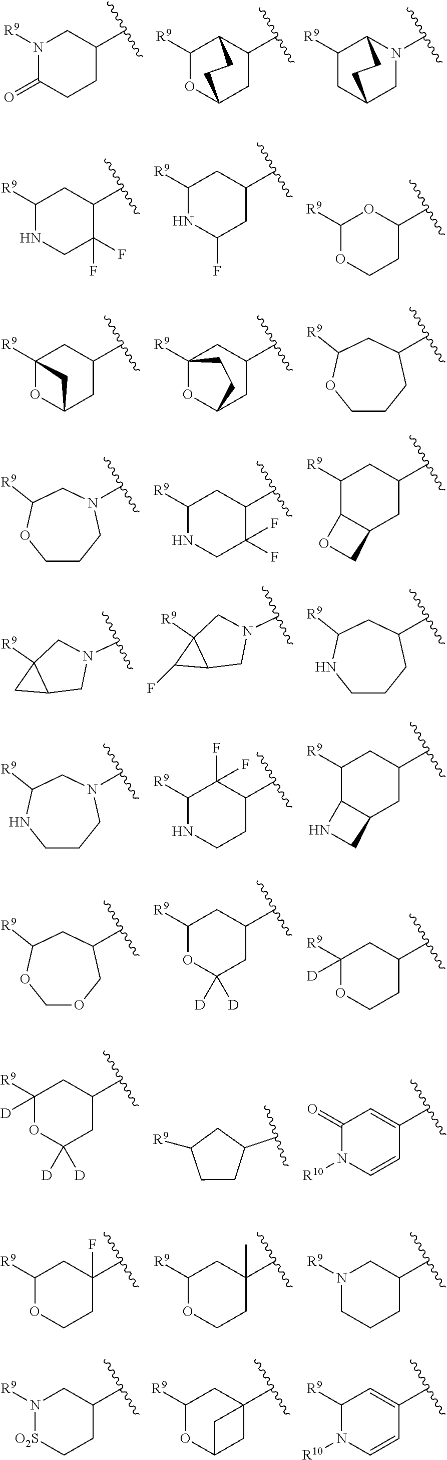

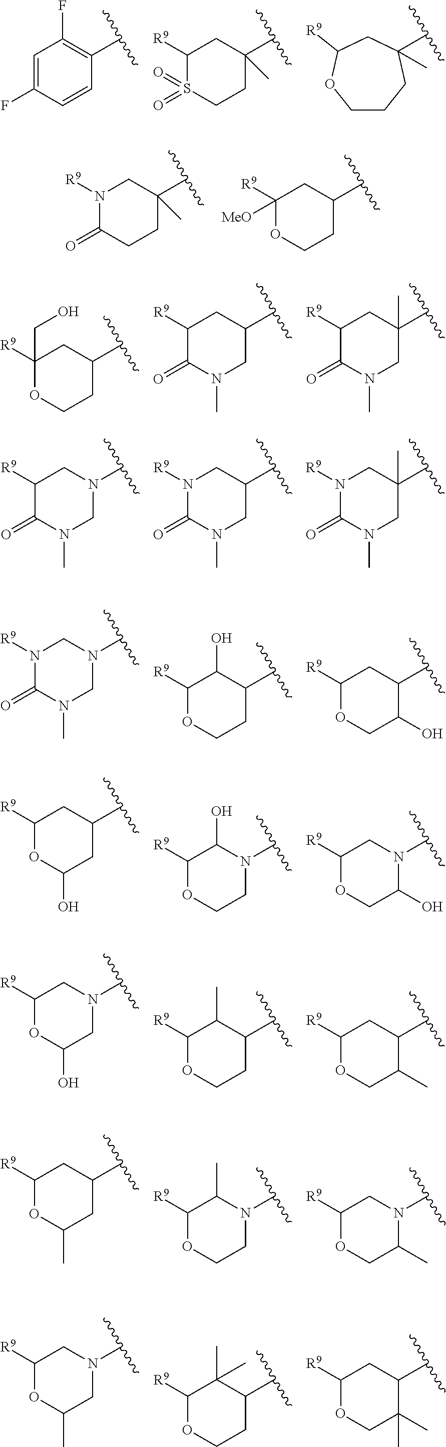

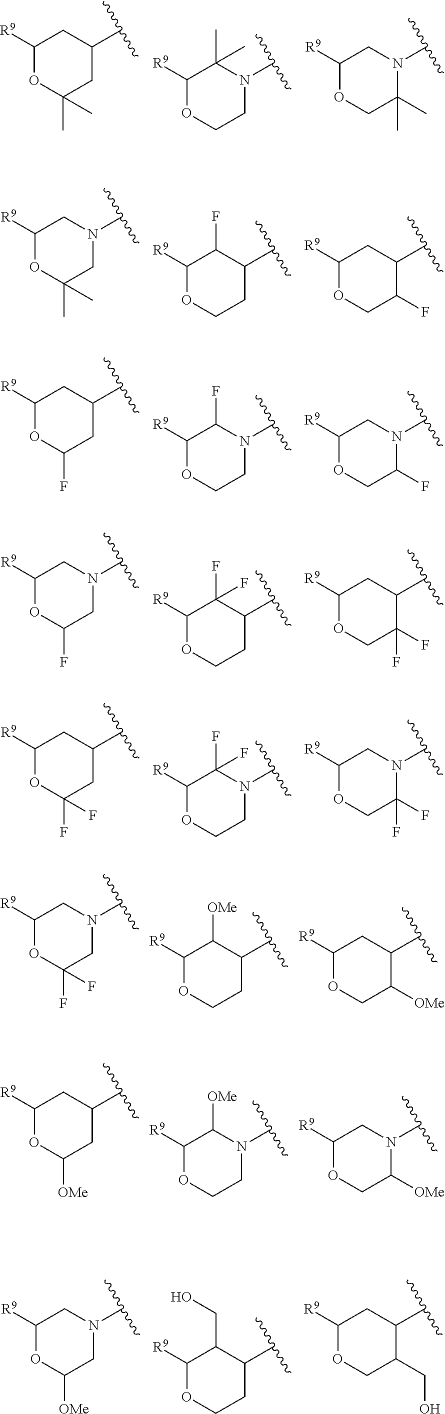

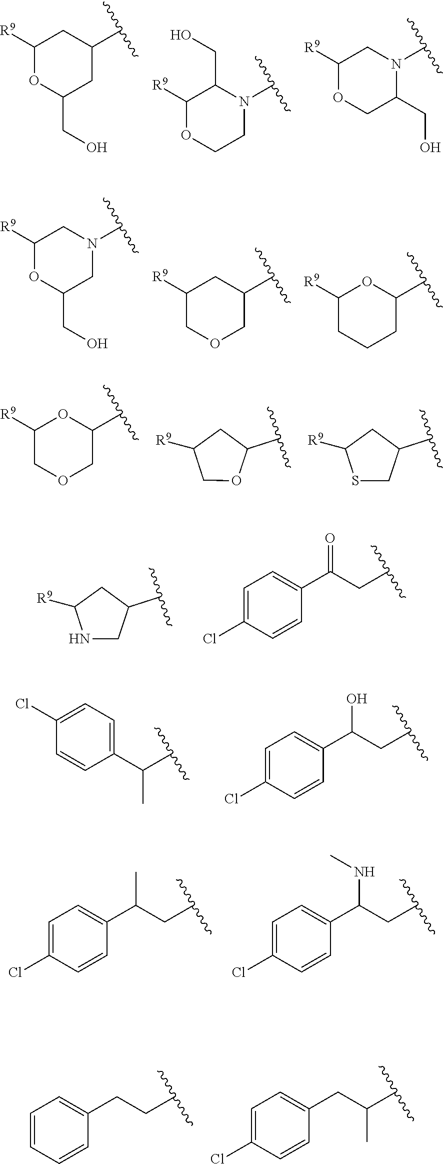

In some embodiments, Ring B is

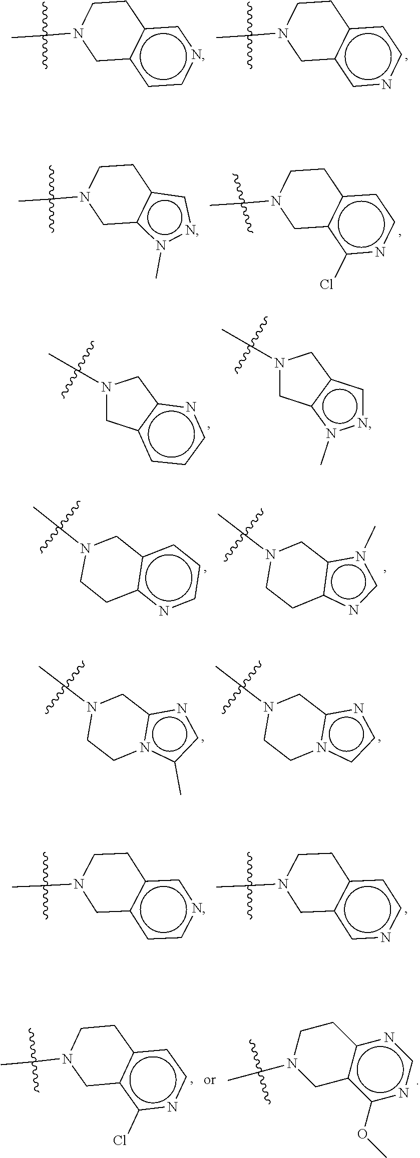

In some embodiments, Ring B is

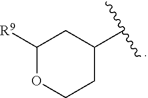

In some embodiments, Ring B is

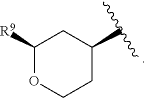

In some embodiments, Ring B is

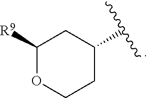

In some embodiments, Ring B is

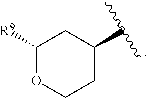

In some embodiments, Ring B is

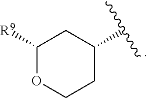

In some embodiments, Ring B is

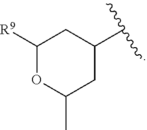

In some embodiments, Ring B is

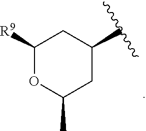

In some embodiments, Ring B is

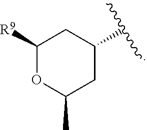

In some embodiments, Ring B is

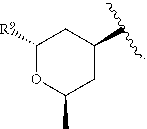

In some embodiments, Ring B is

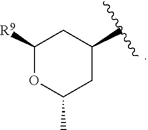

In some embodiments, Ring B is

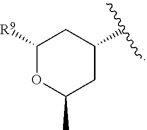

In some embodiments, Ring B is

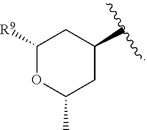

In some embodiments, Ring B is

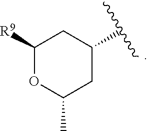

In some embodiments, Ring B is

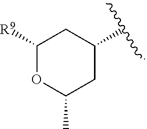

In some embodiments, Ring B is

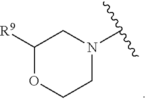

In some embodiments, Ring B is

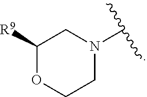

In some embodiments, Ring B is

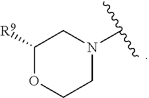

In some embodiments, Ring B is

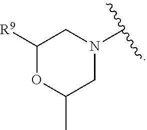

In some embodiments, Ring B is

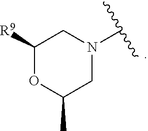

In some embodiments, Ring B is

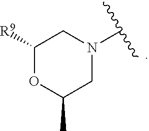

In some embodiments, Ring B is

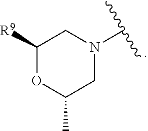

In some embodiments, Ring B is

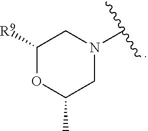

In some embodiments, Ring B is

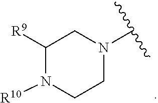

In some embodiments, Ring B is

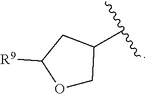

In some embodiments, Ring B is

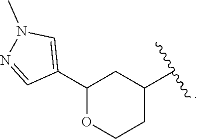 [SMILES: *C1CCOC(c2cnn(C)c2)C1]

In some embodiments, Ring B is

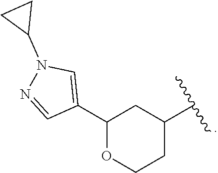 [SMILES: *C1CCOC(c2cnn(C3CC3)c2)C1]

In some embodiments, Ring B is

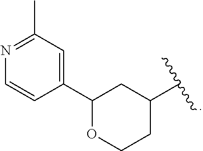 [SMILES: *C1CCOC(c2ccnc(C)c2)C1]

In some embodiments, Ring B is

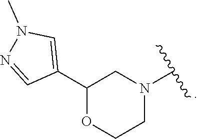 [SMILES: *N1CCOC(c2cnn(C)c2)C1]

In some embodiments, Ring B is

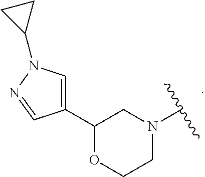 [SMILES: *N1CCOC(c2cnn(C3CC3)c2)C1]

In some embodiments, Ring B is

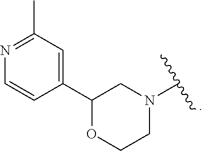 [SMILES: *N1CCOC(c2ccnc(C)c2)C1]

In some embodiments, Ring B is

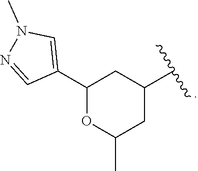 [SMILES: *C1CC(C)OC(c2cnn(C)c2)C1]

In some embodiments, Ring B is

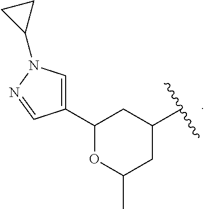 [SMILES: *C1CC(C)OC(c2cnn(C3CC3)c2)C1]

In some embodiments, Ring B is

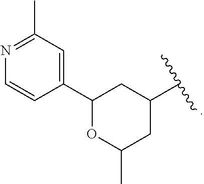 [SMILES: *C1CC(C)OC(c2ccnc(C)c2)C1]

In some embodiments, Ring B is

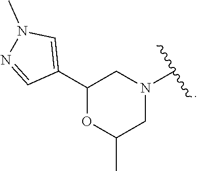 [SMILES: *N1CC(C)OC(c2cnn(C)c2)C1]

In some embodiments, Ring B is

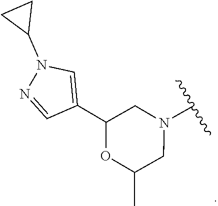 [SMILES: *N1CC(C)OC(c2cnn(C3CC3)c2)C1]

In some embodiments, Ring B is

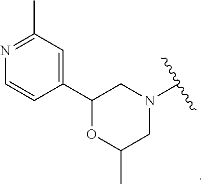 [SMILES: *N1CC(C)OC(c2ccnc(C)c2)C1]

In some embodiments, Ring B is

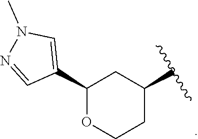 [SMILES: *[C@H]1CCO[C@@H](c2cnn(C)c2)C1]

In some embodiments, Ring B is

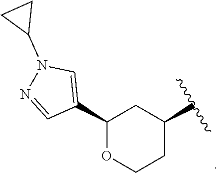 [SMILES: *[C@H]1CCO[C@@H](c2cnn(C3CC3)c2)C1]

In some embodiments, Ring B is

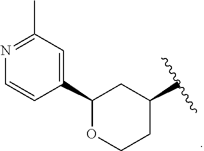 [SMILES: *[C@H]1CCO[C@@H](c2ccnc(C)c2)C1]

In some embodiments, Ring B is

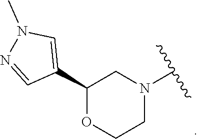 [SMILES: *N1CCO[C@@H](c2cnn(C)c2)C1]

In some embodiments, Ring B is

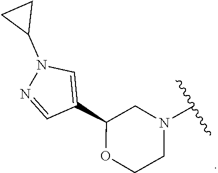 [SMILES: *N1CCO[C@@H](c2cnn(C3CC3)c2)C1]

In some embodiments, Ring B is

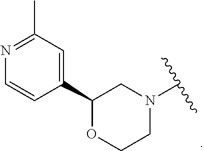 [SMILES: *N1CCO[C@@H](c2ccnc(C)c2)C1]

In some embodiments, Ring B is

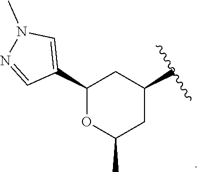 [SMILES: *[C@H]1C[C@@H](C)O[C@@H](c2cnn(C)c2)C1]

In some embodiments, Ring B is

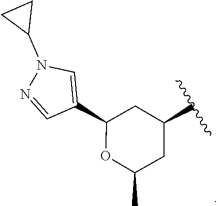 [SMILES: *[C@H]1C[C@@H](C)O[C@@H](c2cnn(C3CC3)c2)C1]

In some embodiments, Ring B is

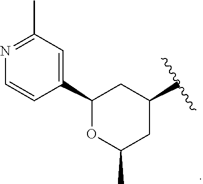 [SMILES: *[C@H]1C[C@@H](C)O[C@@H](c2ccnc(C)c2)C1]

In some embodiments, Ring B is

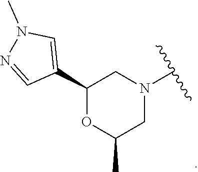 [SMILES: *N1C[C@@H](C)O[C@@H](c2cnn(C)c2)C1]

In some embodiments, Ring B is

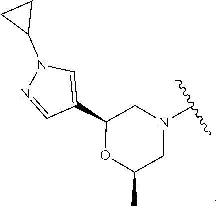 [SMILES: *N1C[C@@H](C)O[C@@H](c2cnn(C3CC3)c2)C1]

In some embodiments, Ring B is

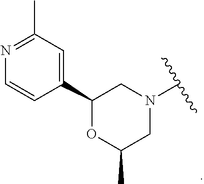 [SMILES: *N1C[C@@H](C)O[C@@H](c2ccnc(C)c2)C1]

In some embodiments, at least one hydrogen atom of the compound is a deuterium atom. In some embodiments, at least one C₁-C₆ aliphatic group of the compound is substituted with at least one deuterium atom. In some embodiments, at least one C₁-C₆alkyl group of the compound is substituted with at least one deuterium atom. In some embodiments, R^{Z} is -CD₃. In some embodiments, R³ is -CD₃. In some embodiments, R² and R³ are both -CD₃. In some embodiments, R⁴ is -CD₃.

Exemplary compounds of the invention are set forth in Table A, below. In some embodiments, the compound is a compound set forth in Table A, or a pharmaceutically acceptable salt thereof.

TABLE A
Exemplary Compounds

| I-# | Structure |
|---|---|
| I-1 | 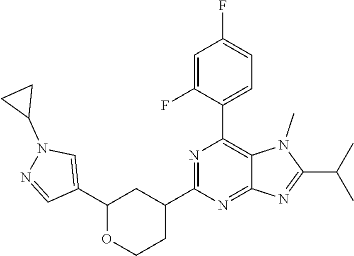 [SMILES: CC(C)c1nc2nc(C3CCOC(c4cnn(C5CC5)c4)C3)nc(-c3ccc(F)cc3F)c2n1C] |
| I-2 | 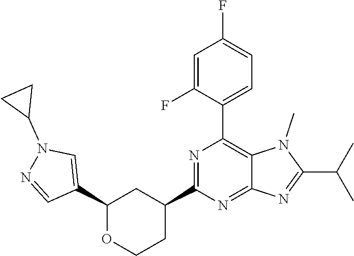 [SMILES: CC(C)c1nc2nc([C@H]3CCO[C@@H](c4cnn(C5CC5)c4)C3)nc(-c3ccc(F)cc3F)c2n1C] |
| I-3 | 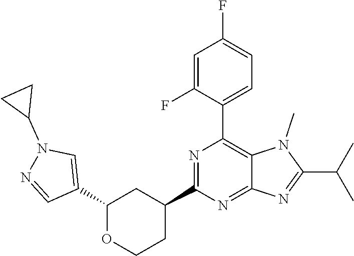 [SMILES: CC(C)c1nc2nc([C@H]3CCO[C@H](c4cnn(C5CC5)c4)C3)nc(-c3ccc(F)cc3F)c2n1C] |
| I-4 | 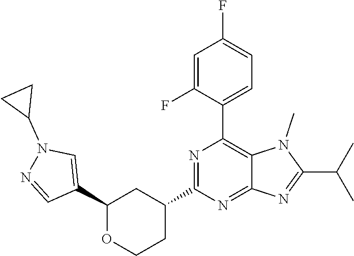 [SMILES: CC(C)c1nc2nc([C@@H]3CCO[C@@H](c4cnn(C5CC5)c4)C3)nc(-c3ccc(F)cc3F)c2n1C] |
| I-5 | 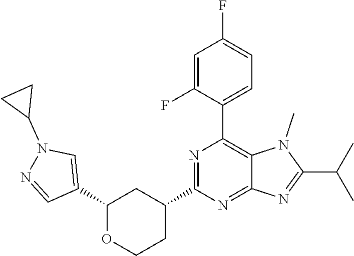 [SMILES: CC(C)c1nc2nc([C@@H]3CCO[C@H](c4cnn(C5CC5)c4)C3)nc(-c3ccc(F)cc3F)c2n1C] |
| I-6 | 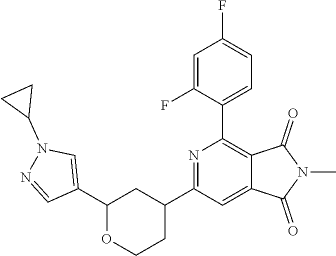 [SMILES: CN1C(=O)c2cc(C3CCOC(c4cnn(C5CC5)c4)C3)nc(-c3ccc(F)cc3F)c2C1=O] |
| I-7 | 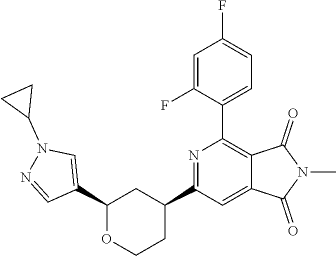 [SMILES: CN1C(=O)c2cc([C@H]3CCO[C@@H](c4cnn(C5CC5)c4)C3)nc(-c3ccc(F)cc3F)c2C1=O] |
| I-8 | 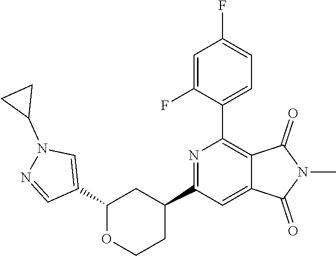 [SMILES: CN1C(=O)c2cc([C@H]3CCO[C@H](c4cnn(C5CC5)c4)C3)nc(-c3ccc(F)cc3F)c2C1=O] |
| I-9 | 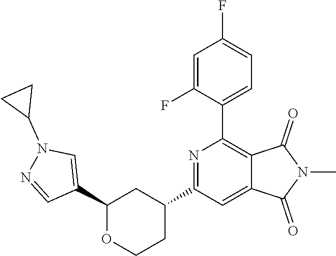 [SMILES: CN1C(=O)c2cc([C@@H]3CCO[C@@H](c4cnn(C5CC5)c4)C3)nc(-c3ccc(F)cc3F)c2C1=O] |
| I-10 | 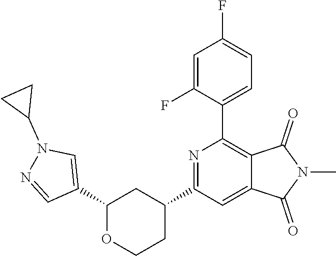 [SMILES: CN1C(=O)c2cc([C@@H]3CCO[C@H](c4cnn(C5CC5)c4)C3)nc(-c3ccc(F)cc3F)c2C1=O] |
| I-11 | 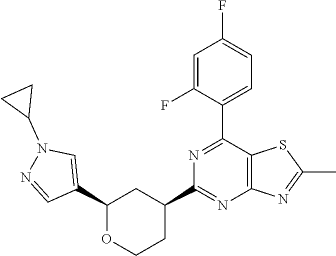 [SMILES: Cc1nc2nc([C@H]3CCO[C@@H](c4cnn(C5CC5)c4)C3)nc(-c3ccc(F)cc3F)c2s1] |
| I-12 | 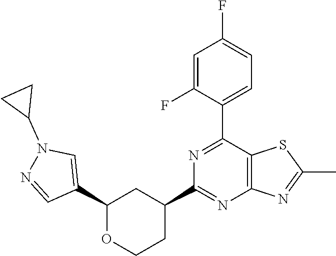 [SMILES: Cc1nc2nc([C@H]3CCO[C@@H](c4cnn(C5CC5)c4)C3)nc(-c3ccc(F)cc3F)c2s1] |
| I-13 | 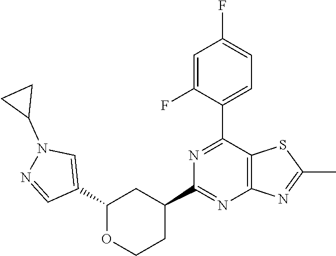 [SMILES: Cc1nc2nc([C@H]3CCO[C@H](c4cnn(C5CC5)c4)C3)nc(-c3ccc(F)cc3F)c2s1] |
| I-14 | 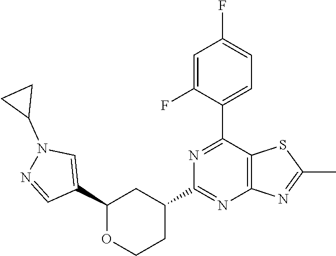 [SMILES: Cc1nc2nc([C@@H]3CCO[C@@H](c4cnn(C5CC5)c4)C3)nc(-c3ccc(F)cc3F)c2s1] |
| I-15 | 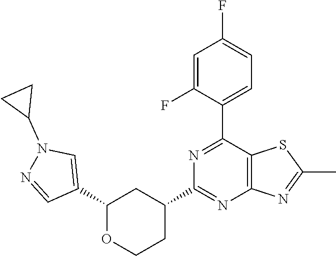 [SMILES: Cc1nc2nc([C@@H]3CCO[C@H](c4cnn(C5CC5)c4)C3)nc(-c3ccc(F)cc3F)c2s1] |
| I-16 | 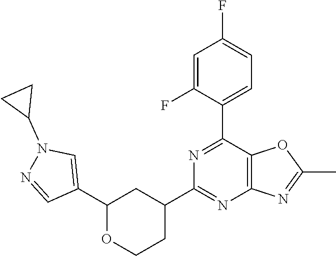 [SMILES: Cc1nc2nc(C3CCOC(c4cnn(C5CC5)c4)C3)nc(-c3ccc(F)cc3F)c2o1] |
| I-17 | 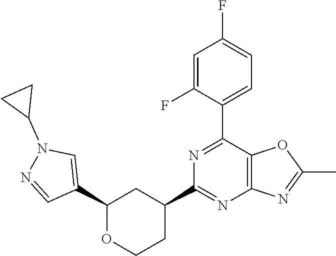 [SMILES: Cc1nc2nc([C@H]3CCO[C@@H](c4cnn(C5CC5)c4)C3)nc(-c3ccc(F)cc3F)c2o1] |
| I-18 | 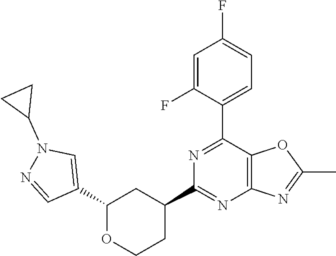 [SMILES: Cc1nc2nc([C@H]3CCO[C@H](c4cnn(C5CC5)c4)C3)nc(-c3ccc(F)cc3F)c2o1] |
| I-19 | 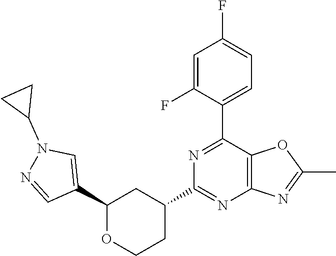 [SMILES: Cc1nc2nc([C@@H]3CCO[C@@H](c4cnn(C5CC5)c4)C3)nc(-c3ccc(F)cc3F)c2o1] |
| I-20 | 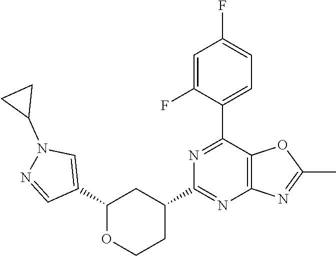 [SMILES: Cc1nc2nc([C@@H]3CCO[C@H](c4cnn(C5CC5)c4)C3)nc(-c3ccc(F)cc3F)c2o1] |
| I-21 | 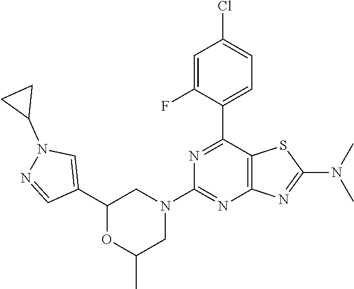 [SMILES: CC1CN(c2nc(-c3ccc(Cl)cc3F)c3sc(N(C)C)nc3n2)CC(c2cnn(C3CC3)c2)O1] |
| I-22 | 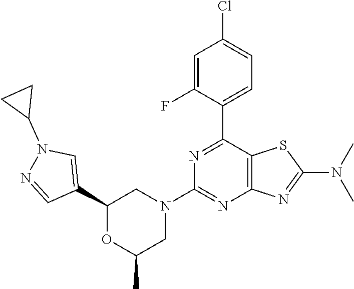 [SMILES: C[C@@H]1CN(c2nc(-c3ccc(Cl)cc3F)c3sc(N(C)C)nc3n2)C[C@H](c2cnn(C3CC3)c2)O1] |
| I-23 | 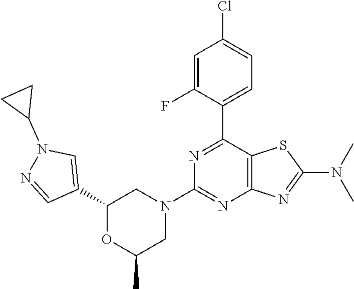 [SMILES: C[C@@H]1CN(c2nc(-c3ccc(Cl)cc3F)c3sc(N(C)C)nc3n2)C[C@@H](c2cnn(C3CC3)c2)O1] |
| I-24 | 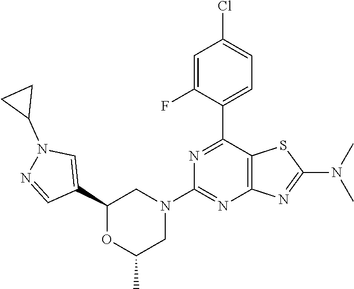 [SMILES: C[C@H]1CN(c2nc(-c3ccc(Cl)cc3F)c3sc(N(C)C)nc3n2)C[C@H](c2cnn(C3CC3)c2)O1] |
| I-25 | 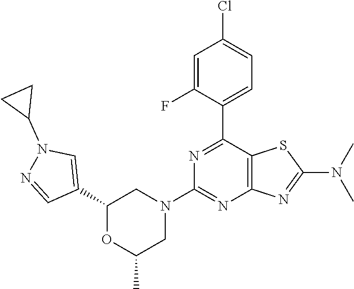 [SMILES: C[C@H]1CN(c2nc(-c3ccc(Cl)cc3F)c3sc(N(C)C)nc3n2)C[C@@H](c2cnn(C3CC3)c2)O1] |
| I-26 | 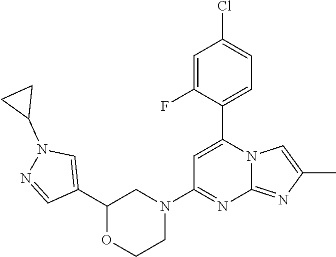 [SMILES: Cc1cn2c(-c3ccc(Cl)cc3F)cc(N3CCOC(c4cnn(C5CC5)c4)C3)nc2n1] |
| I-27 | 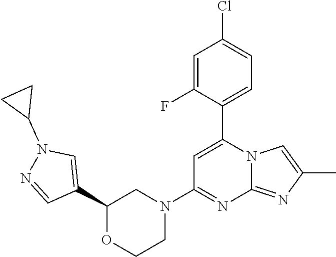 [SMILES: Cc1cn2c(-c3ccc(Cl)cc3F)cc(N3CCO[C@@H](c4cnn(C5CC5)c4)C3)nc2n1] |
| I-28 | 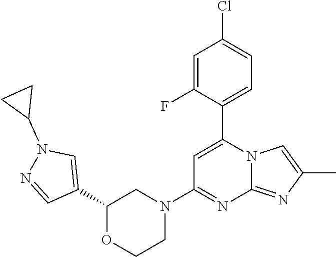 [SMILES: Cc1cn2c(-c3ccc(Cl)cc3F)cc(N3CCO[C@H](c4cnn(C5CC5)c4)C3)nc2n1] |
| I-29 | 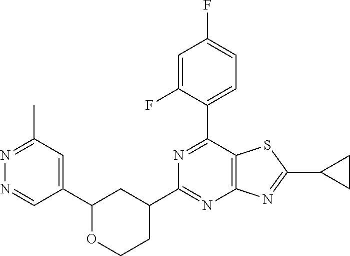 [SMILES: Cc1cc(C2CC(c3nc(-c4ccc(F)cc4F)c4sc(C5CC5)nc4n3)CCO2)cnn1] |
| I-30 | 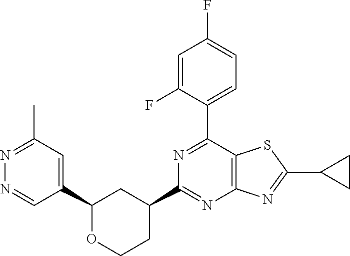 [SMILES: Cc1cc([C@H]2C[C@@H](c3nc(-c4ccc(F)cc4F)c4sc(C5CC5)nc4n3)CCO2)cnn1] |
| I-31 | 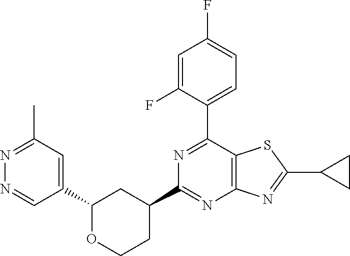 [SMILES: Cc1cc([C@@H]2C[C@@H](c3nc(-c4ccc(F)cc4F)c4sc(C5CC5)nc4n3)CCO2)cnn1] |
| I-32 | 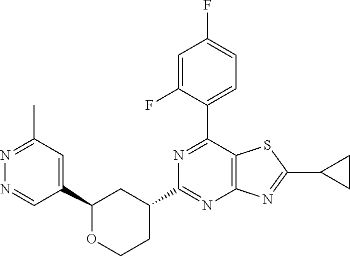 [SMILES: Cc1cc([C@H]2C[C@H](c3nc(-c4ccc(F)cc4F)c4sc(C5CC5)nc4n3)CCO2)cnn1] |
| I-33 | 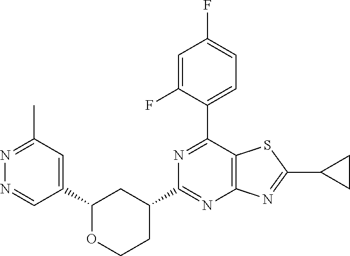 [SMILES: Cc1cc([C@@H]2C[C@H](c3nc(-c4ccc(F)cc4F)c4sc(C5CC5)nc4n3)CCO2)cnn1] |
| I-34 | 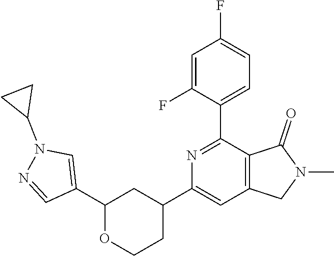 [SMILES: CN1Cc2cc(C3CCOC(c4cnn(C5CC5)c4)C3)nc(-c3ccc(F)cc3F)c2C1=O] |
| I-35 | 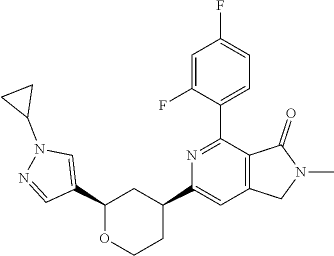 [SMILES: CN1Cc2cc([C@H]3CCO[C@@H](c4cnn(C5CC5)c4)C3)nc(-c3ccc(F)cc3F)c2C1=O] |
| I-36 | 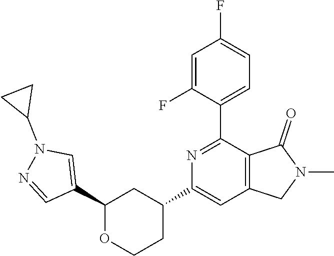 [SMILES: CN1Cc2cc([C@@H]3CCO[C@@H](c4cnn(C5CC5)c4)C3)nc(-c3ccc(F)cc3F)c2C1=O] |
| I-37 | 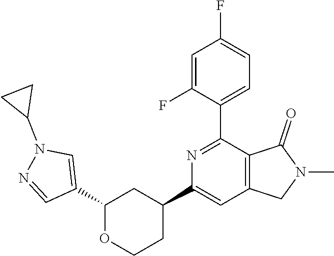 [SMILES: CN1Cc2cc([C@H]3CCO[C@H](c4cnn(C5CC5)c4)C3)nc(-c3ccc(F)cc3F)c2C1=O] |
| I-38 | 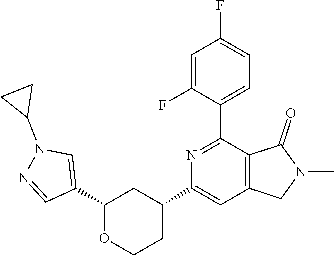 [SMILES: CN1Cc2cc([C@@H]3CCO[C@H](c4cnn(C5CC5)c4)C3)nc(-c3ccc(F)cc3F)c2C1=O] |
| I-39 | 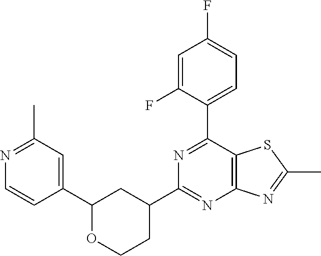 [SMILES: Cc1cc(C2CC(c3nc(-c4ccc(F)cc4F)c4sc(C)nc4n3)CCO2)ccn1] |
| I-40 | 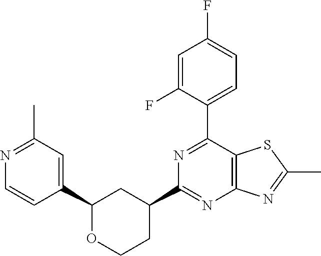 [SMILES: Cc1cc([C@H]2C[C@@H](c3nc(-c4ccc(F)cc4F)c4sc(C)nc4n3)CCO2)ccn1] |
| I-41 | 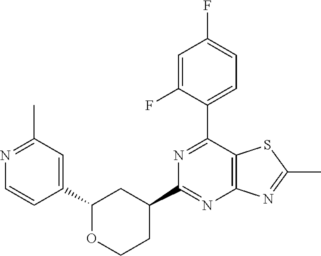 [SMILES: Cc1cc([C@@H]2C[C@@H](c3nc(-c4ccc(F)cc4F)c4sc(C)nc4n3)CCO2)ccn1] |
| I-42 | 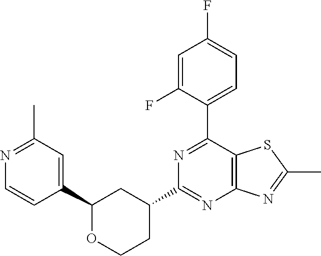 [SMILES: Cc1cc([C@H]2C[C@H](c3nc(-c4ccc(F)cc4F)c4sc(C)nc4n3)CCO2)ccn1] |
| I-43 | 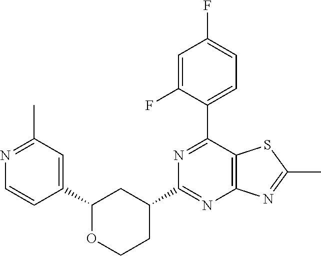 [SMILES: Cc1cc([C@@H]2C[C@H](c3nc(-c4ccc(F)cc4F)c4sc(C)nc4n3)CCO2)ccn1] |
| I-44 | 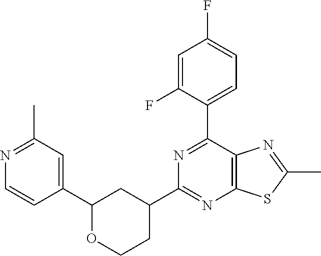 [SMILES: Cc1cc(C2CC(c3nc(-c4ccc(F)cc4F)c4nc(C)sc4n3)CCO2)ccn1] |
| I-45 | 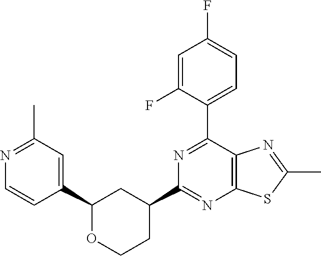 [SMILES: Cc1cc([C@H]2C[C@@H](c3nc(-c4ccc(F)cc4F)c4nc(C)sc4n3)CCO2)ccn1] |
| I-46 | 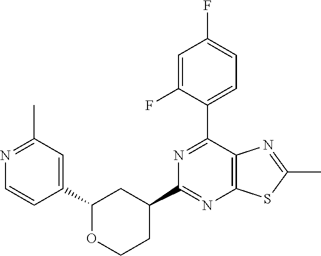 [SMILES: Cc1cc([C@@H]2C[C@@H](c3nc(-c4ccc(F)cc4F)c4nc(C)sc4n3)CCO2)ccn1] |
| I-47 | 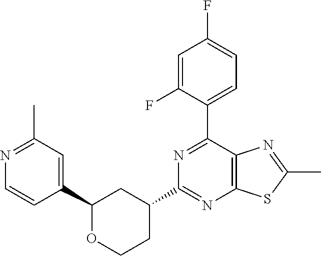 [SMILES: Cc1cc([C@H]2C[C@H](c3nc(-c4ccc(F)cc4F)c4nc(C)sc4n3)CCO2)ccn1] |
| I-48 | 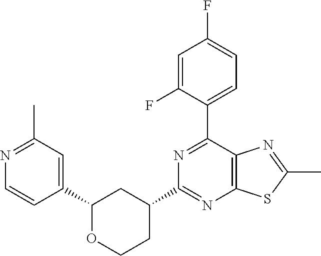 [SMILES: Cc1cc([C@@H]2C[C@H](c3nc(-c4ccc(F)cc4F)c4nc(C)sc4n3)CCO2)ccn1] |
| I-49 | 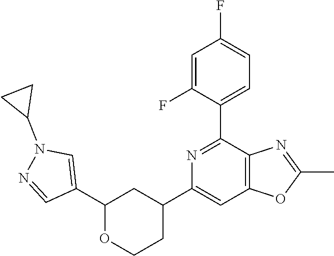 [SMILES: Cc1nc2c(-c3ccc(F)cc3F)nc(C3CCOC(c4cnn(C5CC5)c4)C3)cc2o1] |
| I-50 | 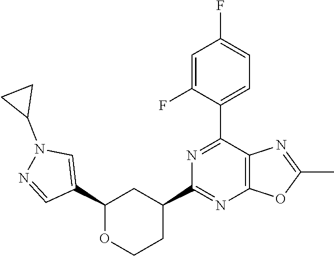 [SMILES: Cc1nc2c(-c3ccc(F)cc3F)nc([C@H]3CCO[C@@H](c4cnn(C5CC5)c4)C3)nc2o1] |
| I-51 | 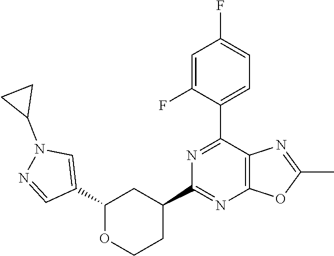 [SMILES: Cc1nc2c(-c3ccc(F)cc3F)nc([C@H]3CCO[C@H](c4cnn(C5CC5)c4)C3)nc2o1] |
| I-52 | 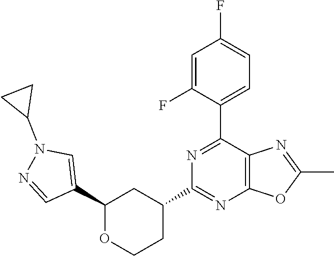 [SMILES: Cc1nc2c(-c3ccc(F)cc3F)nc([C@@H]3CCO[C@@H](c4cnn(C5CC5)c4)C3)nc2o1] |
| I-53 | 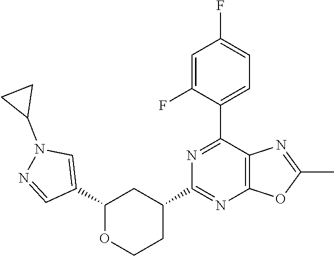 [SMILES: Cc1nc2c(-c3ccc(F)cc3F)nc([C@@H]3CCO[C@H](c4cnn(C5CC5)c4)C3)nc2o1] |
| I-54 | 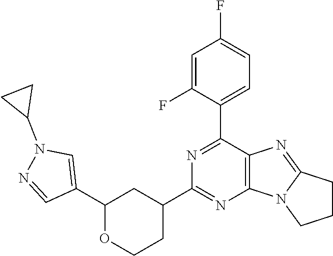 [SMILES: Fc1ccc(-c2nc(C3CCOC(c4cnn(C5CC5)c4)C3)nc3c2nc2n3CCC2)c(F)c1] |
| I-55 | 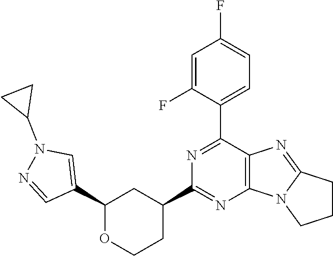 [SMILES: Fc1ccc(-c2nc([C@H]3CCO[C@@H](c4cnn(C5CC5)c4)C3)nc3c2nc2n3CCC2)c(F)c1] |
| I-56 | 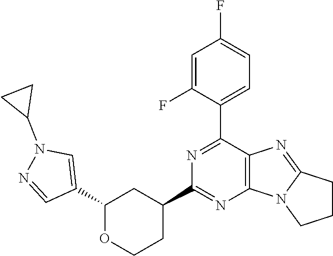 [SMILES: Fc1ccc(-c2nc([C@H]3CCO[C@H](c4cnn(C5CC5)c4)C3)nc3c2nc2n3CCC2)c(F)c1] |
| I-57 | 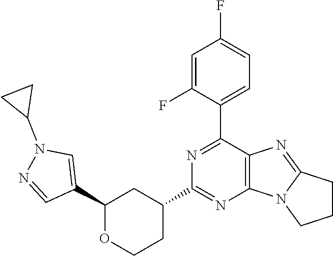 [SMILES: Fc1ccc(-c2nc([C@@H]3CCO[C@@H](c4cnn(C5CC5)c4)C3)nc3c2nc2n3CCC2)c(F)c1] |
| I-58 | 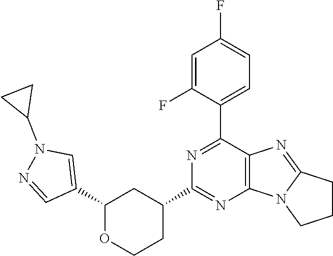 [SMILES: Fc1ccc(-c2nc([C@@H]3CCO[C@H](c4cnn(C5CC5)c4)C3)nc3c2nc2n3CCC2)c(F)c1] |
| I-59 | 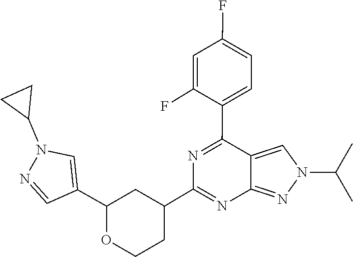 [SMILES: CC(C)n1cc2c(-c3ccc(F)cc3F)nc(C3CCOC(c4cnn(C5CC5)c4)C3)nc2n1] |
| I-60 | 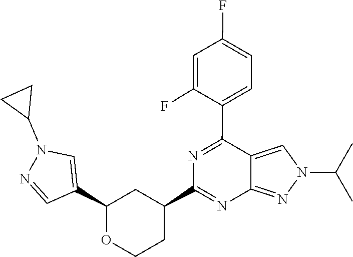 [SMILES: CC(C)n1cc2c(-c3ccc(F)cc3F)nc([C@H]3CCO[C@@H](c4cnn(C5CC5)c4)C3)nc2n1] |
| I-61 | 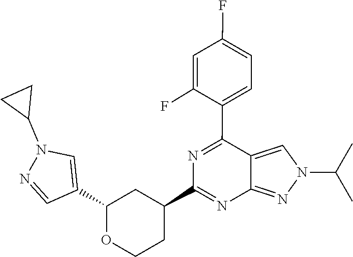 [SMILES: CC(C)n1cc2c(-c3ccc(F)cc3F)nc([C@H]3CCO[C@H](c4cnn(C5CC5)c4)C3)nc2n1] |
| I-62 | 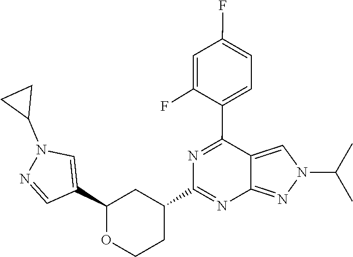 [SMILES: CC(C)n1cc2c(-c3ccc(F)cc3F)nc([C@@H]3CCO[C@@H](c4cnn(C5CC5)c4)C3)nc2n1] |
| I-63 | 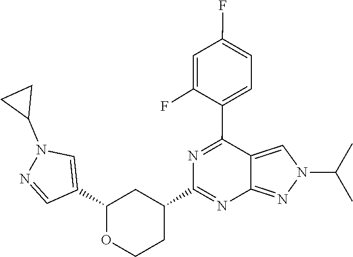 [SMILES: CC(C)n1cc2c(-c3ccc(F)cc3F)nc([C@@H]3CCO[C@H](c4cnn(C5CC5)c4)C3)nc2n1] |
| I-64 | 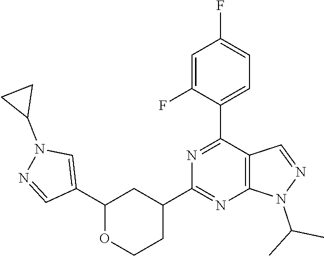 [SMILES: CC(C)n1ncc2c(-c3ccc(F)cc3F)nc(C3CCOC(c4cnn(C5CC5)c4)C3)nc21] |
| I-65 | 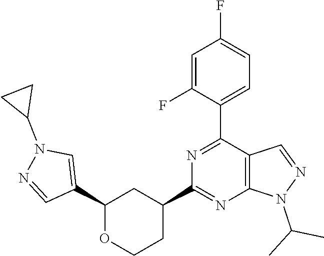 [SMILES: CC(C)n1ncc2c(-c3ccc(F)cc3F)nc([C@H]3CCO[C@@H](c4cnn(C5CC5)c4)C3)nc21] |
| I-66 | 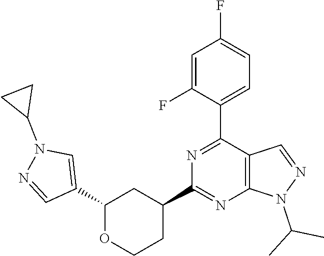 [SMILES: CC(C)n1ncc2c(-c3ccc(F)cc3F)nc([C@H]3CCO[C@H](c4cnn(C5CC5)c4)C3)nc21] |
| I-67 | 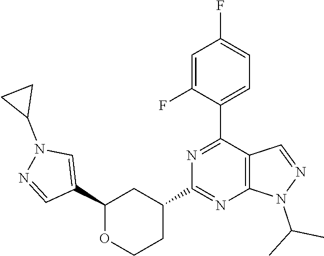 [SMILES: CC(C)n1ncc2c(-c3ccc(F)cc3F)nc([C@@H]3CCO[C@@H](c4cnn(C5CC5)c4)C3)nc21] |
| I-68 | 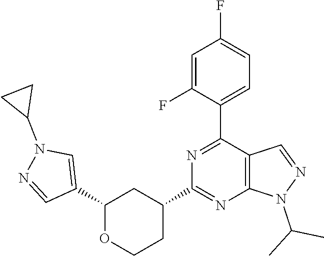 [SMILES: CC(C)n1ncc2c(-c3ccc(F)cc3F)nc([C@@H]3CCO[C@H](c4cnn(C5CC5)c4)C3)nc21] |
| I-69 | 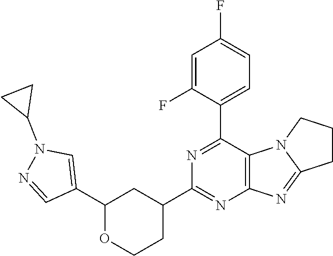 [SMILES: Fc1ccc(-c2nc(C3CCOC(c4cnn(C5CC5)c4)C3)nc3nc4n(c23)CCC4)c(F)c1] |
| I-70 | 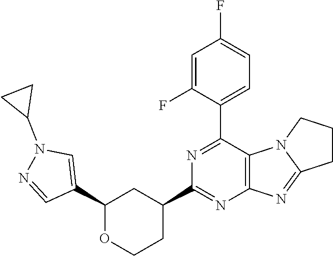 [SMILES: Fc1ccc(-c2nc([C@H]3CCO[C@@H](c4cnn(C5CC5)c4)C3)nc3nc4n(c23)CCC4)c(F)c1] |
| I-71 | 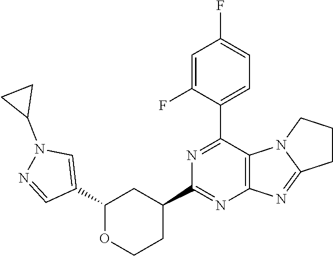 [SMILES: Fc1ccc(-c2nc([C@H]3CCO[C@H](c4cnn(C5CC5)c4)C3)nc3nc4n(c23)CCC4)c(F)c1] |
| I-72 | 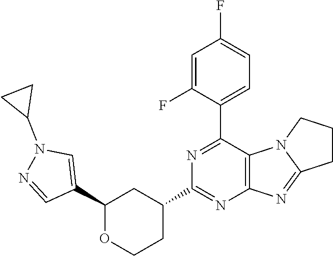 [SMILES: Fc1ccc(-c2nc([C@@H]3CCO[C@@H](c4cnn(C5CC5)c4)C3)nc3nc4n(c23)CCC4)c(F)c1] |
| I-73 | 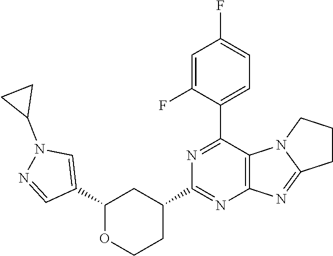 [SMILES: Fc1ccc(-c2nc([C@@H]3CCO[C@H](c4cnn(C5CC5)c4)C3)nc3nc4n(c23)CCC4)c(F)c1] |
| I-74 | 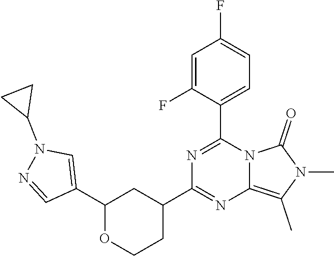 [SMILES: Cc1c2nc(C3CCOC(c4cnn(C5CC5)c4)C3)nc(-c3ccc(F)cc3F)n2c(=O)n1C] |
| I-75 | 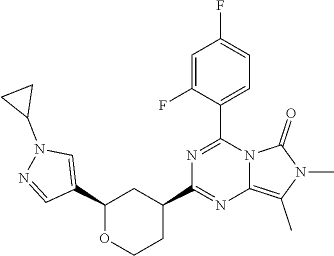 [SMILES: Cc1c2nc([C@H]3CCO[C@@H](c4cnn(C5CC5)c4)C3)nc(-c3ccc(F)cc3F)n2c(=O)n1C] |
| I-76 | 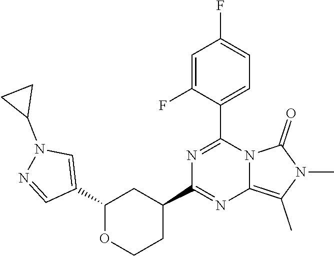 [SMILES: Cc1c2nc([C@H]3CCO[C@H](c4cnn(C5CC5)c4)C3)nc(-c3ccc(F)cc3F)n2c(=O)n1C] |
| I-77 | 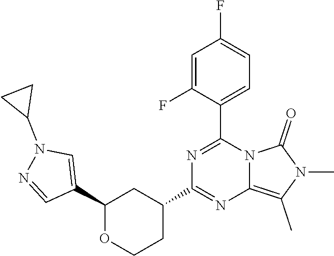 [SMILES: Cc1c2nc([C@@H]3CCO[C@@H](c4cnn(C5CC5)c4)C3)nc(-c3ccc(F)cc3F)n2c(=O)n1C] |
| I-78 | 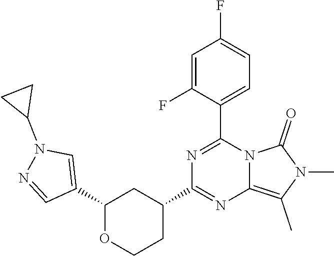 [SMILES: Cc1c2nc([C@@H]3CCO[C@H](c4cnn(C5CC5)c4)C3)nc(-c3ccc(F)cc3F)n2c(=O)n1C] |

Exemplary compounds of the invention are set forth in Table A2, below. In some embodiments, the compound is a compound set forth in Table A2, or a pharmaceutically acceptable salt thereof.

TABLE A2
Exemplary Compounds

| I-# | Structure |
|---|---|
| I-79 | 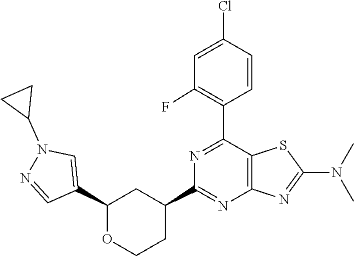 [SMILES: CN(C)c1nc2nc([C@H]3CCO[C@@H](c4cnn(C5CC5)c4)C3)nc(-c3ccc(Cl)cc3F)c2s1] |
| I-80 | 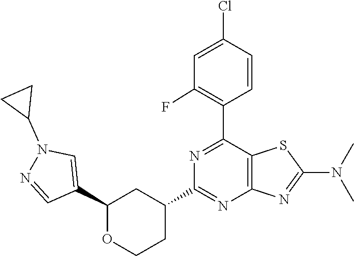 [SMILES: CN(C)c1nc2nc([C@@H]3CCO[C@@H](c4cnn(C5CC5)c4)C3)nc(-c3ccc(Cl)cc3F)c2s1] |
| I-81 | 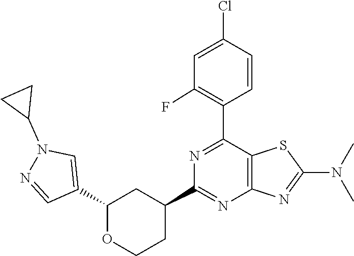 [SMILES: CN(C)c1nc2nc([C@H]3CCO[C@H](c4cnn(C5CC5)c4)C3)nc(-c3ccc(Cl)cc3F)c2s1] |
| I-82 | 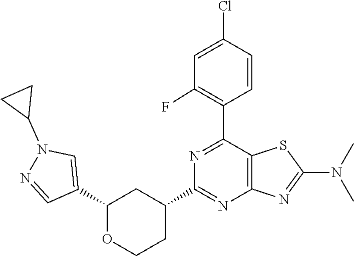 [SMILES: CN(C)c1nc2nc([C@@H]3CCO[C@H](c4cnn(C5CC5)c4)C3)nc(-c3ccc(Cl)cc3F)c2s1] |
| I-83 | 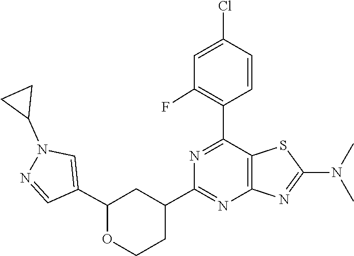 [SMILES: CN(C)c1nc2nc(C3CCOC(c4cnn(C5CC5)c4)C3)nc(-c3ccc(Cl)cc3F)c2s1] |
| I-84 | 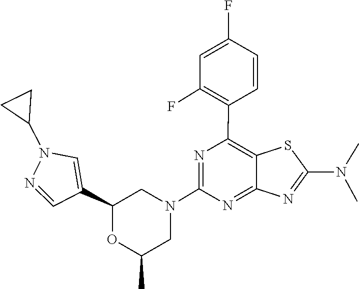 [SMILES: C[C@@H]1CN(c2nc(-c3ccc(F)cc3F)c3sc(N(C)C)nc3n2)C[C@H](c2cnn(C3CC3)c2)O1] |
| I-85 | 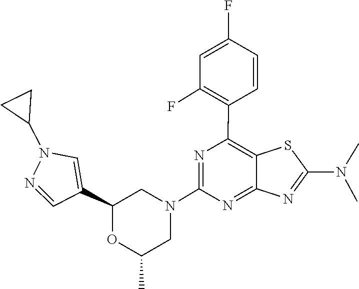 [SMILES: C[C@H]1CN(c2nc(-c3ccc(F)cc3F)c3sc(N(C)C)nc3n2)C[C@H](c2cnn(C3CC3)c2)O1] |
| I-86 | 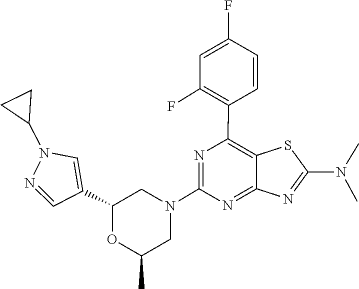 [SMILES: C[C@@H]1CN(c2nc(-c3ccc(F)cc3F)c3sc(N(C)C)nc3n2)C[C@@H](c2cnn(C3CC3)c2)O1] |
| I-87 | 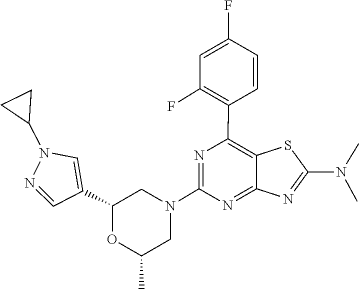 [SMILES: C[C@H]1CN(c2nc(-c3ccc(F)cc3F)c3sc(N(C)C)nc3n2)C[C@@H](c2cnn(C3CC3)c2)O1] |
| I-88 | 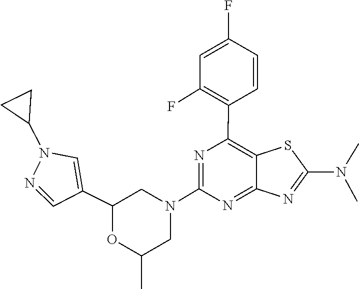 [SMILES: CC1CN(c2nc(-c3ccc(F)cc3F)c3sc(N(C)C)nc3n2)CC(c2cnn(C3CC3)c2)O1] |
| I-89 | 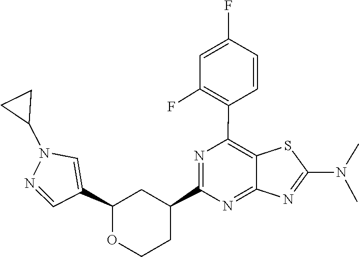 [SMILES: CN(C)c1nc2nc([C@H]3CCO[C@@H](c4cnn(C5CC5)c4)C3)nc(-c3ccc(F)cc3F)c2s1] |
| I-90 | 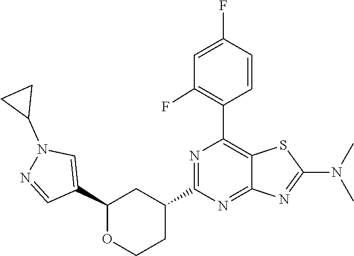 [SMILES: CN(C)c1nc2nc([C@@H]3CCO[C@@H](c4cnn(C5CC5)c4)C3)nc(-c3ccc(F)cc3F)c2s1] |
| I-91 | 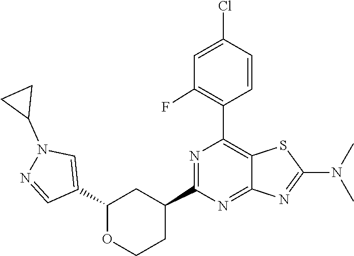 [SMILES: CN(C)c1nc2nc([C@H]3CCO[C@H](c4cnn(C5CC5)c4)C3)nc(-c3ccc(Cl)cc3F)c2s1] |
| I-92 | 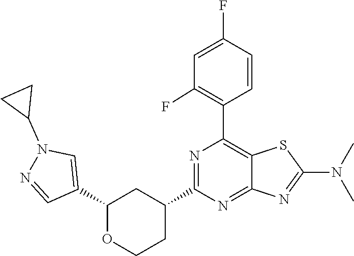 [SMILES: CN(C)c1nc2nc([C@@H]3CCO[C@H](c4cnn(C5CC5)c4)C3)nc(-c3ccc(F)cc3F)c2s1] |
| I-93 | 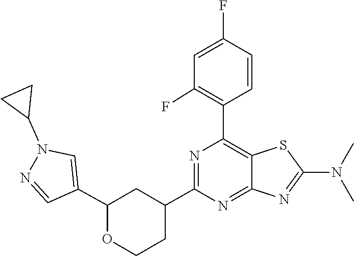 [SMILES: CN(C)c1nc2nc(C3CCOC(c4cnn(C5CC5)c4)C3)nc(-c3ccc(F)cc3F)c2s1] |
| I-94 | 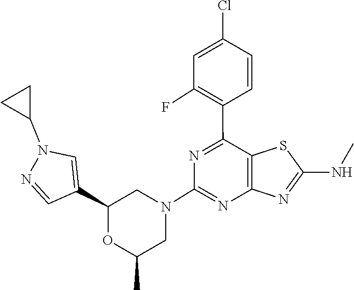 [SMILES: CNc1nc2nc(N3C[C@@H](C)O[C@@H](c4cnn(C5CC5)c4)C3)nc(-c3ccc(Cl)cc3F)c2s1] |
| I-95 | 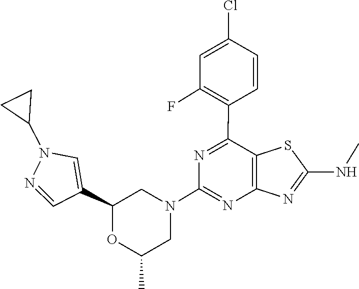 [SMILES: CNc1nc2nc(N3C[C@H](C)O[C@@H](c4cnn(C5CC5)c4)C3)nc(-c3ccc(Cl)cc3F)c2s1] |
| I-96 | 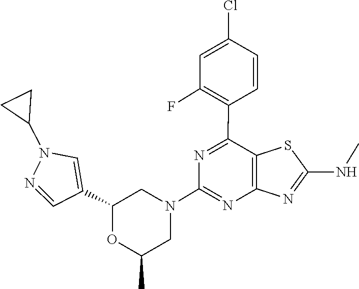 [SMILES: CNc1nc2nc(N3C[C@@H](C)O[C@H](c4cnn(C5CC5)c4)C3)nc(-c3ccc(Cl)cc3F)c2s1] |
| I-97 | 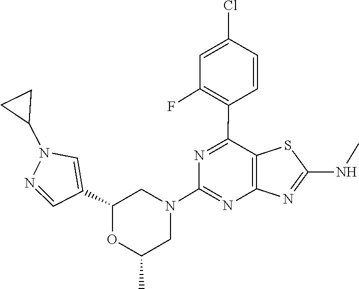 [SMILES: CNc1nc2nc(N3C[C@@H](c4cnn(C5CC5)c4)O[C@@H](C)C3)nc(-c3ccc(Cl)cc3F)c2s1] |
| I-98 | 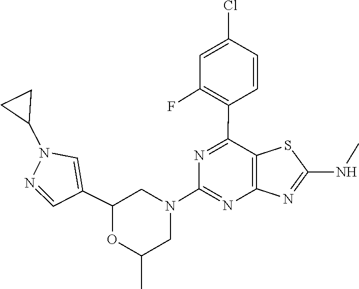 [SMILES: CNc1nc2nc(N3CC(C)OC(c4cnn(C5CC5)c4)C3)nc(-c3ccc(Cl)cc3F)c2s1] |
| I-99 | 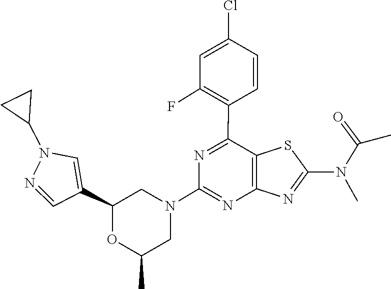 [SMILES: CC(=O)N(C)c1nc2nc(N3C[C@@H](C)O[C@@H](c4cnn(C5CC5)c4)C3)nc(-c3ccc(Cl)cc3F)c2s1] |
| I-100 | 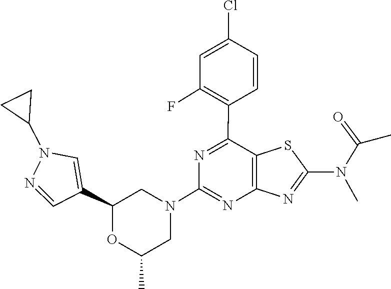 [SMILES: CC(=O)N(C)c1nc2nc(N3C[C@H](C)O[C@@H](c4cnn(C5CC5)c4)C3)nc(-c3ccc(Cl)cc3F)c2s1] |
| I-101 | 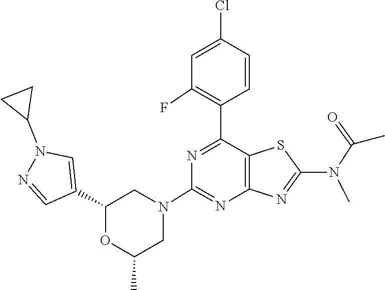 [SMILES: CC(=O)N(C)c1nc2nc(N3C[C@@H](c4cnn(C5CC5)c4)O[C@@H](C)C3)nc(-c3ccc(Cl)cc3F)c2s1] |
| I-102 | 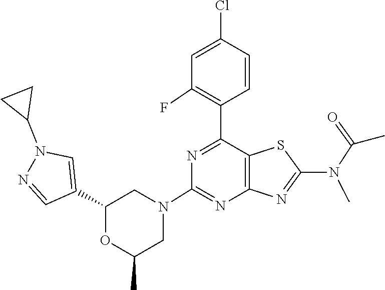 [SMILES: CC(=O)N(C)c1nc2nc(N3C[C@@H](C)O[C@H](c4cnn(C5CC5)c4)C3)nc(-c3ccc(Cl)cc3F)c2s1] |
| I-103 | 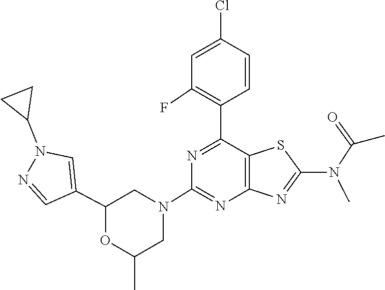 [SMILES: CC(=O)N(C)c1nc2nc(N3CC(C)OC(c4cnn(C5CC5)c4)C3)nc(-c3ccc(Cl)cc3F)c2s1] |
| I-104 | 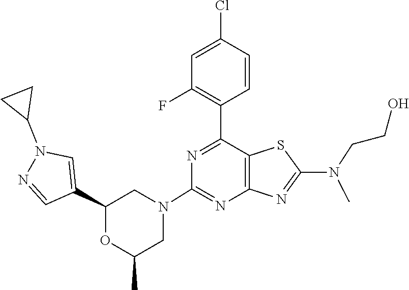 [SMILES: C[C@@H]1CN(c2nc(-c3ccc(Cl)cc3F)c3sc(N(C)CCO)nc3n2)C[C@H](c2cnn(C3CC3)c2)O1] |
| I-105 | 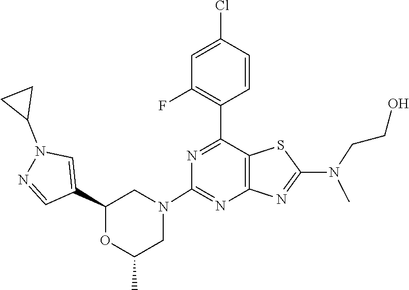 [SMILES: C[C@H]1CN(c2nc(-c3ccc(Cl)cc3F)c3sc(N(C)CCO)nc3n2)C[C@H](c2cnn(C3CC3)c2)O1] |
| I-106 | 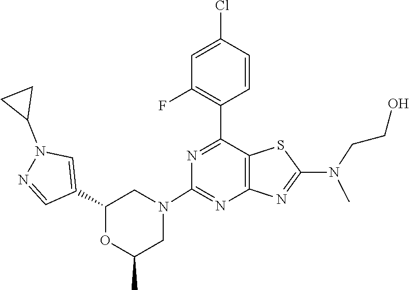 [SMILES: C[C@@H]1CN(c2nc(-c3ccc(Cl)cc3F)c3sc(N(C)CCO)nc3n2)C[C@@H](c2cnn(C3CC3)c2)O1] |
| I-107 | 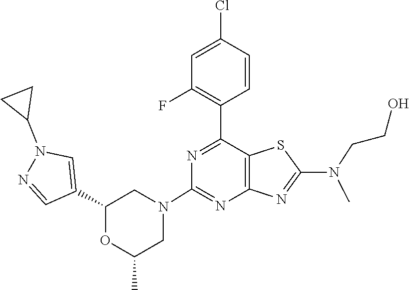 [SMILES: C[C@H]1CN(c2nc(-c3ccc(Cl)cc3F)c3sc(N(C)CCO)nc3n2)C[C@@H](c2cnn(C3CC3)c2)O1] |
| I-108 | 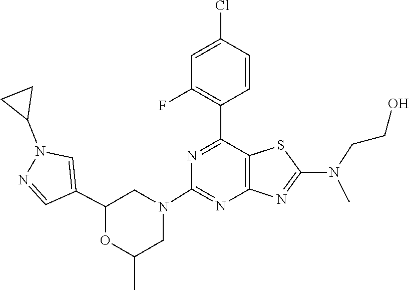 [SMILES: CC1CN(c2nc(-c3ccc(Cl)cc3F)c3sc(N(C)CCO)nc3n2)CC(c2cnn(C3CC3)c2)O1] |
| I-109 | 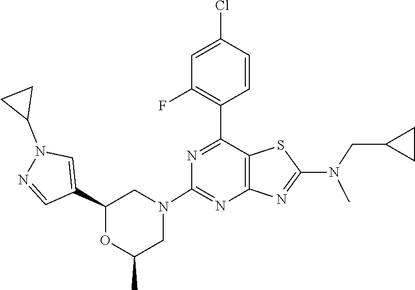 [SMILES: C[C@@H]1CN(c2nc(-c3ccc(Cl)cc3F)c3sc(N(C)CC4CC4)nc3n2)C[C@H](c2cnn(C3CC3)c2)O1] |
| I-110 | 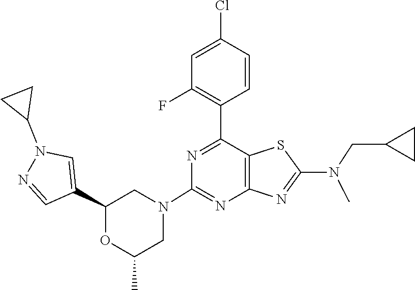 [SMILES: C[C@H]1CN(c2nc(-c3ccc(Cl)cc3F)c3sc(N(C)CC4CC4)nc3n2)C[C@H](c2cnn(C3CC3)c2)O1] |
| I-111 | 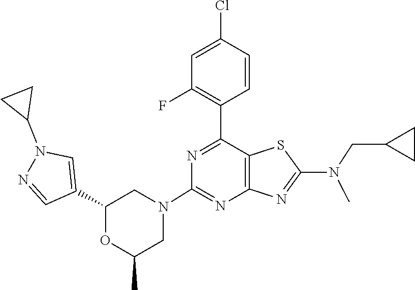 [SMILES: C[C@@H]1CN(c2nc(-c3ccc(Cl)cc3F)c3sc(N(C)CC4CC4)nc3n2)C[C@@H](c2cnn(C3CC3)c2)O1] |
| I-112 | 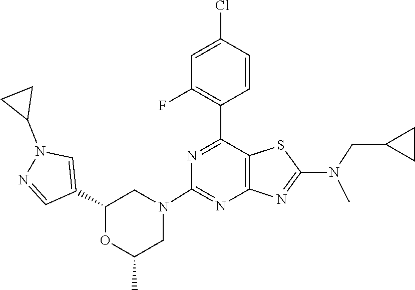 [SMILES: C[C@H]1CN(c2nc(-c3ccc(Cl)cc3F)c3sc(N(C)CC4CC4)nc3n2)C[C@@H](c2cnn(C3CC3)c2)O1] |
| I-113 | 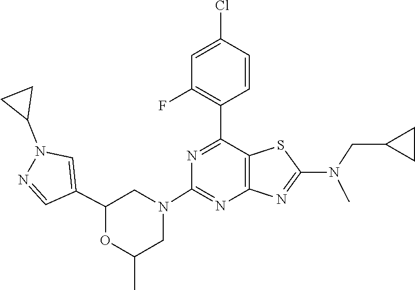 [SMILES: CC1CN(c2nc(-c3ccc(Cl)cc3F)c3sc(N(C)CC4CC4)nc3n2)CC(c2cnn(C3CC3)c2)O1] |
| I-114 | 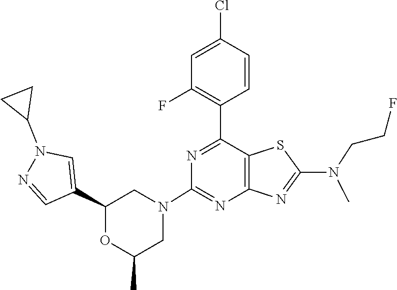 [SMILES: C[C@@H]1CN(c2nc(-c3ccc(Cl)cc3F)c3sc(N(C)CCF)nc3n2)C[C@H](c2cnn(C3CC3)c2)O1] |
| I-115 | 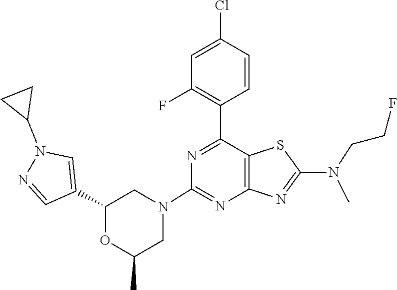 [SMILES: C[C@@H]1CN(c2nc(-c3ccc(Cl)cc3F)c3sc(N(C)CCF)nc3n2)C[C@@H](c2cnn(C3CC3)c2)O1] |
| I-116 | 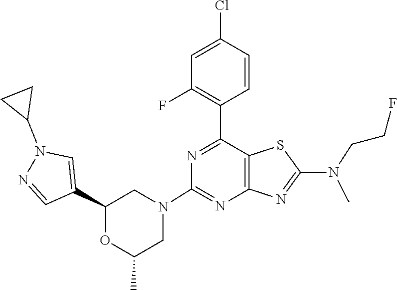 [SMILES: C[C@H]1CN(c2nc(-c3ccc(Cl)cc3F)c3sc(N(C)CCF)nc3n2)C[C@H](c2cnn(C3CC3)c2)O1] |
| I-117 | 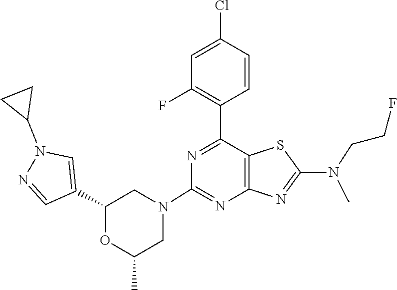 [SMILES: C[C@H]1CN(c2nc(-c3ccc(Cl)cc3F)c3sc(N(C)CCF)nc3n2)C[C@@H](c2cnn(C3CC3)c2)O1] |
| I-118 | 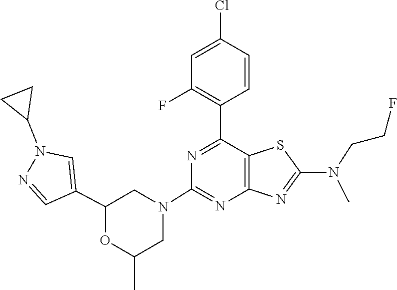 [SMILES: CC1CN(c2nc(-c3ccc(Cl)cc3F)c3sc(N(C)CCF)nc3n2)CC(c2cnn(C3CC3)c2)O1] |
| I-119 | 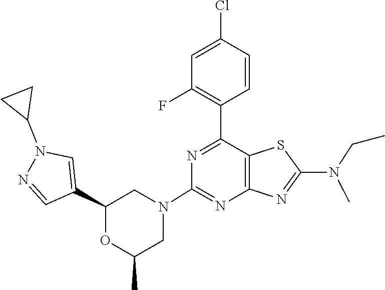 [SMILES: CCN(C)c1nc2nc(N3C[C@@H](C)O[C@@H](c4cnn(C5CC5)c4)C3)nc(-c3ccc(Cl)cc3F)c2s1] |
| I-120 | 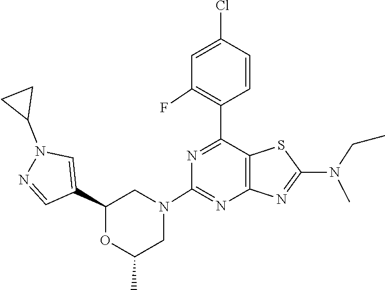 [SMILES: CCN(C)c1nc2nc(N3C[C@H](C)O[C@@H](c4cnn(C5CC5)c4)C3)nc(-c3ccc(Cl)cc3F)c2s1] |
| I-121 | 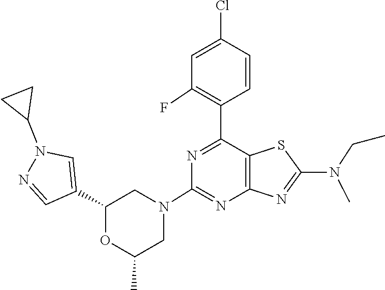 [SMILES: CCN(C)c1nc2nc(N3C[C@@H](c4cnn(C5CC5)c4)O[C@@H](C)C3)nc(-c3ccc(Cl)cc3F)c2s1] |
| I-122 | 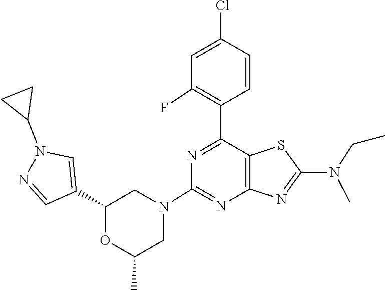 [SMILES: CCN(C)c1nc2nc(N3C[C@@H](c4cnn(C5CC5)c4)O[C@@H](C)C3)nc(-c3ccc(Cl)cc3F)c2s1] |
| I-123 | 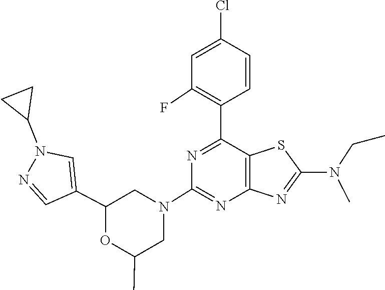 [SMILES: CCN(C)c1nc2nc(N3CC(C)OC(c4cnn(C5CC5)c4)C3)nc(-c3ccc(Cl)cc3F)c2s1] |
| I-124 | 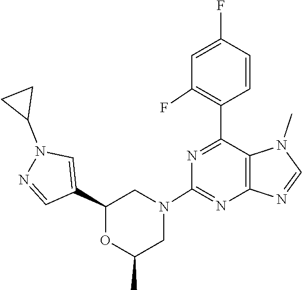 [SMILES: C[C@@H]1CN(c2nc(-c3ccc(F)cc3F)c3c(ncn3C)n2)C[C@H](c2cnn(C3CC3)c2)O1] |
| I-125 | 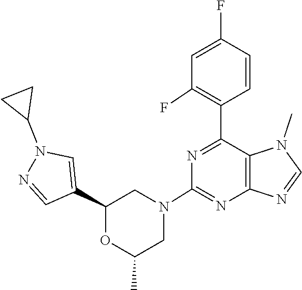 [SMILES: C[C@H]1CN(c2nc(-c3ccc(F)cc3F)c3c(ncn3C)n2)C[C@H](c2cnn(C3CC3)c2)O1] |
| I-126 | 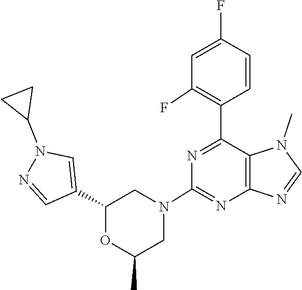 [SMILES: C[C@@H]1CN(c2nc(-c3ccc(F)cc3F)c3c(ncn3C)n2)C[C@@H](c2cnn(C3CC3)c2)O1] |
| I-127 | 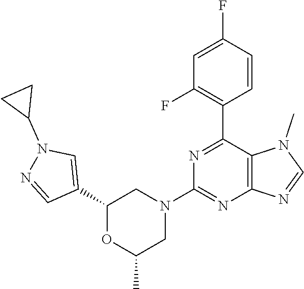 [SMILES: C[C@H]1CN(c2nc(-c3ccc(F)cc3F)c3c(ncn3C)n2)C[C@@H](c2cnn(C3CC3)c2)O1] |
| I-128 | 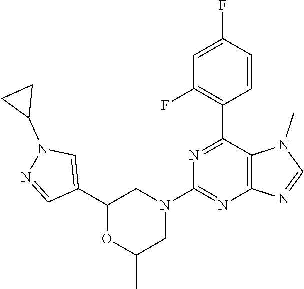 [SMILES: CC1CN(c2nc(-c3ccc(F)cc3F)c3c(ncn3C)n2)CC(c2cnn(C3CC3)c2)O1] |
| I-129 | 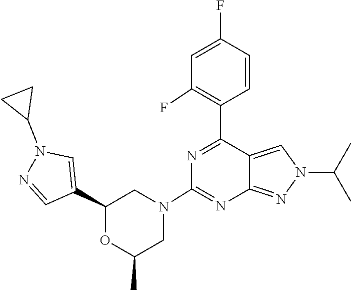 [SMILES: CC(C)n1cc2c(-c3ccc(F)cc3F)nc(N3C[C@@H](C)O[C@@H](c4cnn(C5CC5)c4)C3)nc2n1] |
| I-130 | 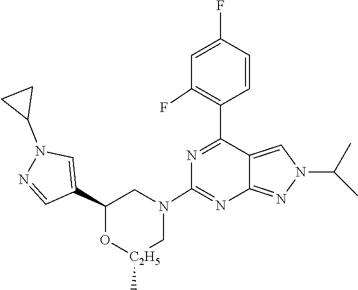 |
| I-131 | 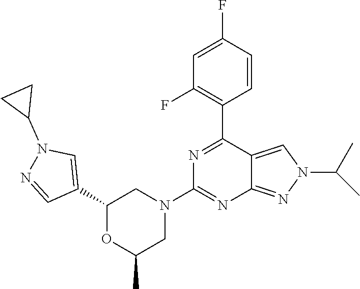 [SMILES: CC(C)n1cc2c(-c3ccc(F)cc3F)nc(N3C[C@@H](C)O[C@H](c4cnn(C5CC5)c4)C3)nc2n1] |
| I-132 | 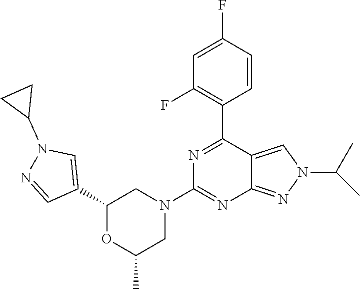 [SMILES: CC(C)n1cc2c(-c3ccc(F)cc3F)nc(N3C[C@@H](c4cnn(C5CC5)c4)O[C@@H](C)C3)nc2n1] |
| I-133 | 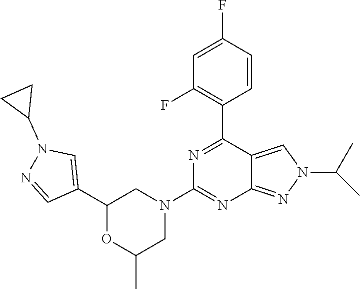 [SMILES: CC1CN(c2nc(-c3ccc(F)cc3F)c3cn(C(C)C)nc3n2)CC(c2cnn(C3CC3)c2)O1] |
| I-134 | 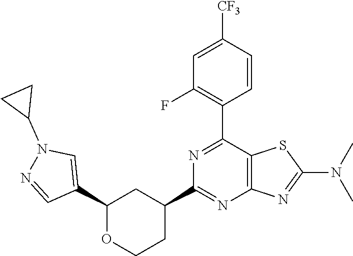 [SMILES: CN(C)c1nc2nc([C@H]3CCO[C@@H](c4cnn(C5CC5)c4)C3)nc(-c3ccc(C(F)(F)F)cc3F)c2s1] |
| I-135 | 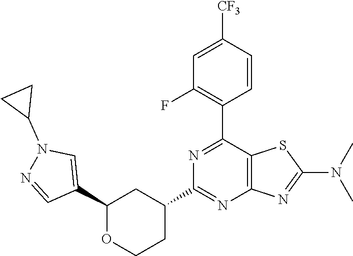 [SMILES: CN(C)c1nc2nc([C@@H]3CCO[C@@H](c4cnn(C5CC5)c4)C3)nc(-c3ccc(C(F)(F)F)cc3F)c2s1] |
| I-136 | 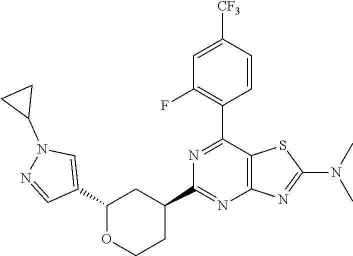 [SMILES: CN(C)c1nc2nc([C@H]3CCO[C@H](c4cnn(C5CC5)c4)C3)nc(-c3ccc(C(F)(F)F)cc3F)c2s1] |
| I-137 | 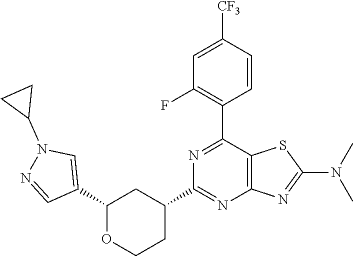 [SMILES: CN(C)c1nc2nc([C@@H]3CCO[C@H](c4cnn(C5CC5)c4)C3)nc(-c3ccc(C(F)(F)F)cc3F)c2s1] |
| I-138 | 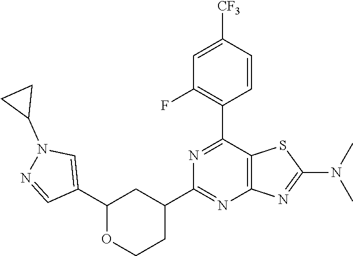 [SMILES: CN(C)c1nc2nc(C3CCOC(c4cnn(C5CC5)c4)C3)nc(-c3ccc(C(F)(F)F)cc3F)c2s1] |
| I-139 | 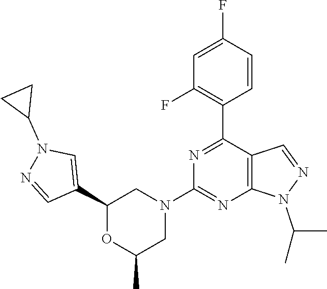 [SMILES: CC(C)n1ncc2c(-c3ccc(F)cc3F)nc(N3C[C@@H](C)O[C@@H](c4cnn(C5CC5)c4)C3)nc21] |
| I-140 | 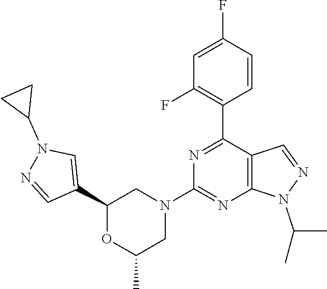 [SMILES: CC(C)n1ncc2c(-c3ccc(F)cc3F)nc(N3C[C@H](C)O[C@@H](c4cnn(C5CC5)c4)C3)nc21] |
| I-141 | 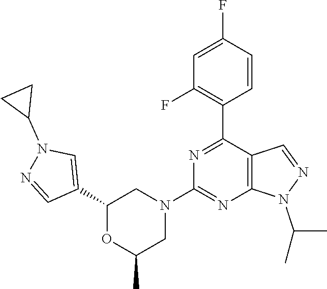 [SMILES: CC(C)n1ncc2c(-c3ccc(F)cc3F)nc(N3C[C@@H](C)O[C@H](c4cnn(C5CC5)c4)C3)nc21] |
| I-142 | 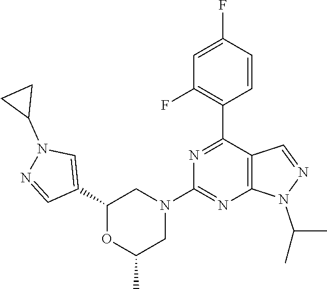 [SMILES: CC(C)n1ncc2c(-c3ccc(F)cc3F)nc(N3C[C@@H](c4cnn(C5CC5)c4)O[C@@H](C)C3)nc21] |
| I-143 | 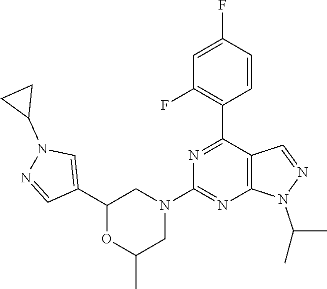 [SMILES: CC1CN(c2nc(-c3ccc(F)cc3F)c3cnn(C(C)C)c3n2)CC(c2cnn(C3CC3)c2)O1] |
| I-144 | 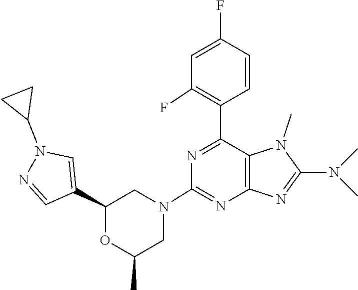 [SMILES: C[C@@H]1CN(c2nc(-c3ccc(F)cc3F)c3c(n2)nc(N(C)C)n3C)C[C@H](c2cnn(C3CC3)c2)O1] |
| I-145 | 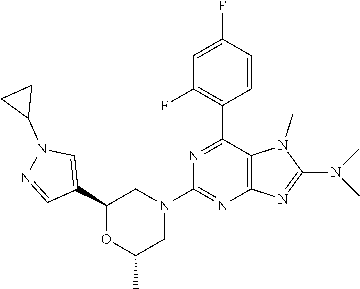 [SMILES: C[C@H]1CN(c2nc(-c3ccc(F)cc3F)c3c(n2)nc(N(C)C)n3C)C[C@H](c2cnn(C3CC3)c2)O1] |
| I-146 | 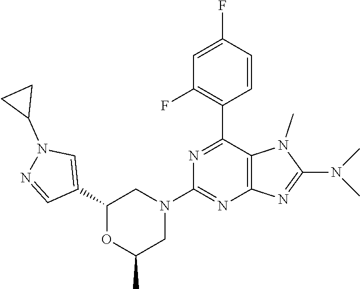 [SMILES: C[C@@H]1CN(c2nc(-c3ccc(F)cc3F)c3c(n2)nc(N(C)C)n3C)C[C@@H](c2cnn(C3CC3)c2)O1] |
| I-147 | 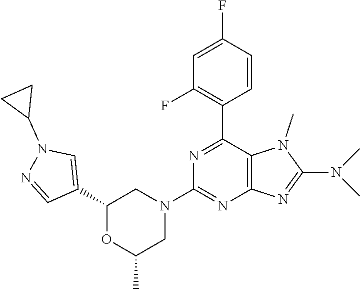 [SMILES: C[C@H]1CN(c2nc(-c3ccc(F)cc3F)c3c(n2)nc(N(C)C)n3C)C[C@@H](c2cnn(C3CC3)c2)O1] |
| I-148 | 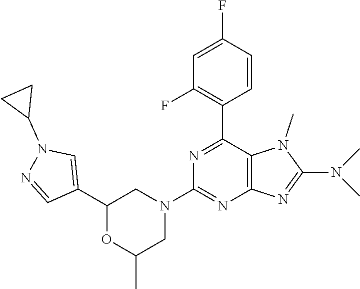 [SMILES: CC1CN(c2nc(-c3ccc(F)cc3F)c3c(n2)nc(N(C)C)n3C)CC(c2cnn(C3CC3)c2)O1] |
| I-149 | 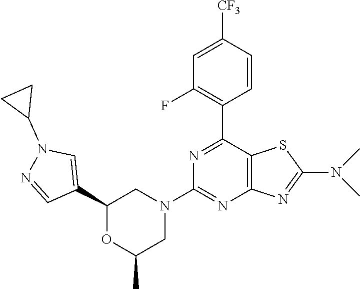 [SMILES: C[C@@H]1CN(c2nc(-c3ccc(C(F)(F)F)cc3F)c3sc(N(C)C)nc3n2)C[C@H](c2cnn(C3CC3)c2)O1] |
| I-150 | 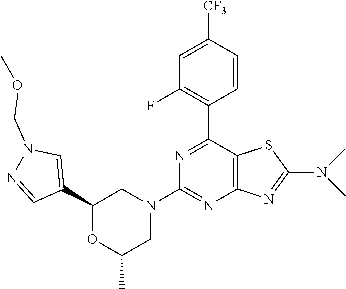 |
| I-151 | 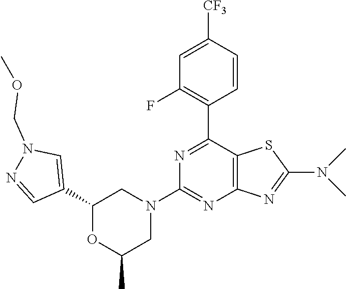 |
| I-152 | 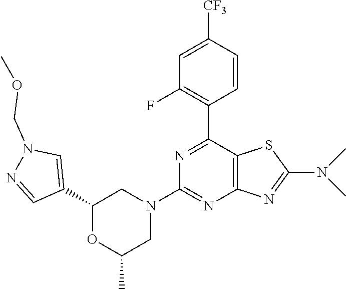 |
| I-153 | 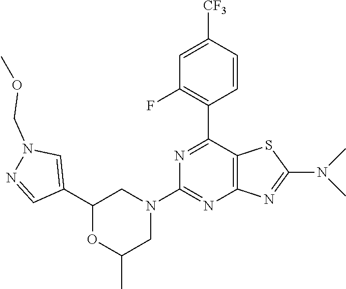 |
| I-154 | 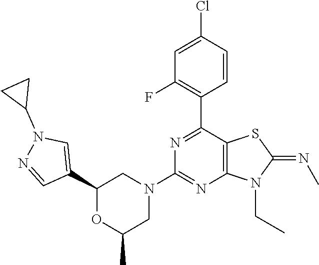 |
| I-155 | 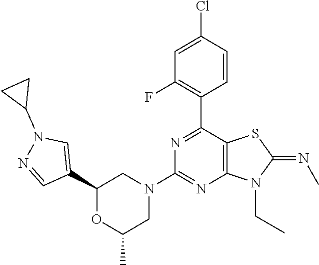 |
| I-156 | 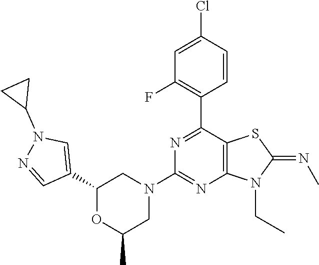 |
| I-157 | 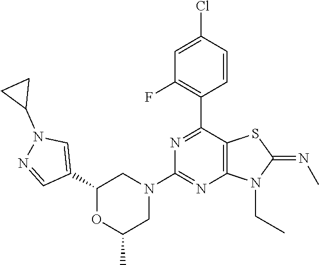 |
| I-158 | 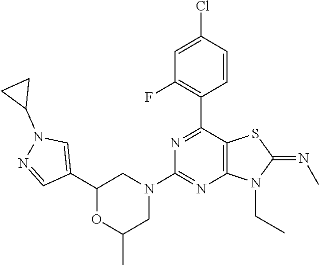 [SMILES: CCn1/c(=N\C)sc2c(-c3ccc(Cl)cc3F)nc(N3CC(C)OC(c4cnn(C5CC5)c4)C3)nc21] |
| I-159 | 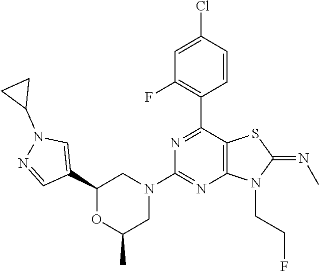 |
| I-160 | 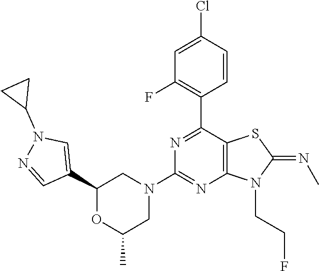 |
| I-161 | 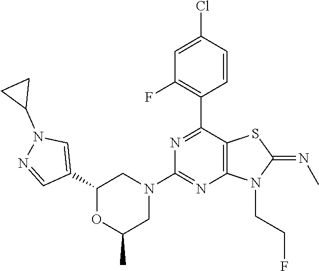 |
| I-162 | 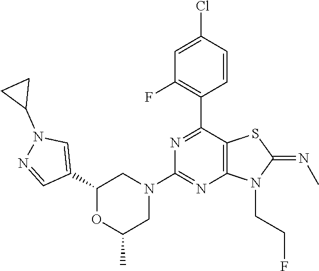 |
| I-163 | 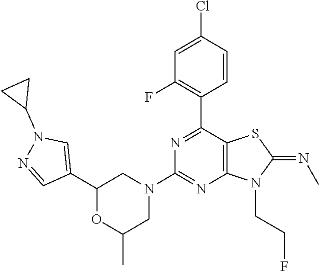 [SMILES: C/N=c1/sc2c(-c3ccc(Cl)cc3F)nc(N3CC(C)OC(c4cnn(C5CC5)c4)C3)nc2n1CCF] |
| I-164 | 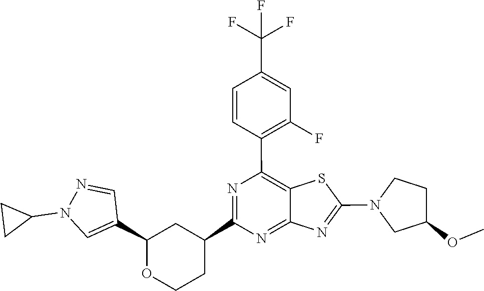 [SMILES: CO[C@@H]1CCN(c2nc3nc([C@H]4CCO[C@@H](c5cnn(C6CC6)c5)C4)nc(-c4ccc(C(F)(F)F)cc4F)c3s2)C1] |
| I-165 | 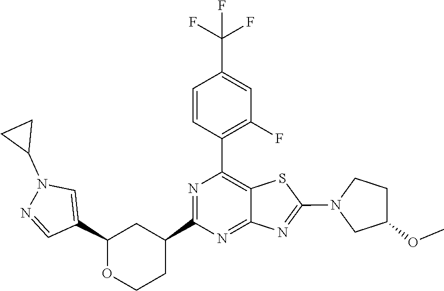 [SMILES: CO[C@H]1CCN(c2nc3nc([C@H]4CCO[C@@H](c5cnn(C6CC6)c5)C4)nc(-c4ccc(C(F)(F)F)cc4F)c3s2)C1] |
| I-166 | 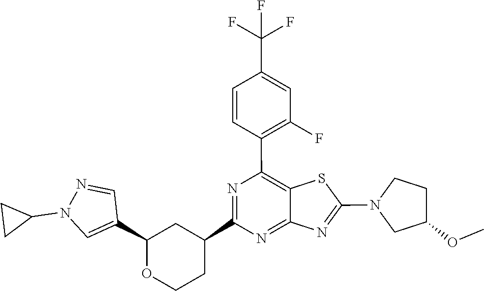 [SMILES: CO[C@H]1CCN(c2nc3nc([C@H]4CCO[C@@H](c5cnn(C6CC6)c5)C4)nc(-c4ccc(C(F)(F)F)cc4F)c3s2)C1] |
| I-167 | 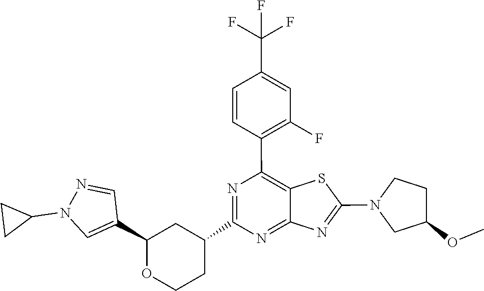 [SMILES: CO[C@@H]1CCN(c2nc3nc([C@@H]4CCO[C@@H](c5cnn(C6CC6)c5)C4)nc(-c4ccc(C(F)(F)F)cc4F)c3s2)C1] |
| I-168 | 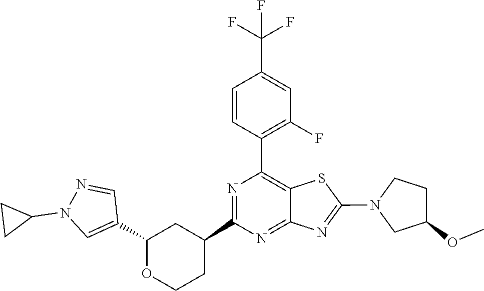 [SMILES: CO[C@@H]1CCN(c2nc3nc([C@H]4CCO[C@H](c5cnn(C6CC6)c5)C4)nc(-c4ccc(C(F)(F)F)cc4F)c3s2)C1] |
| I-169 | 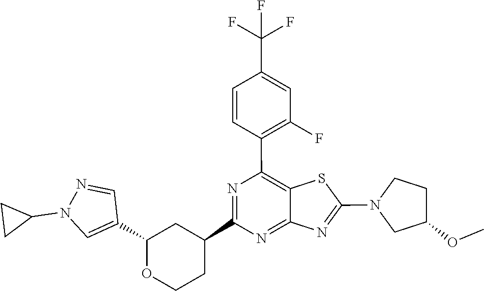 [SMILES: CO[C@H]1CCN(c2nc3nc([C@H]4CCO[C@H](c5cnn(C6CC6)c5)C4)nc(-c4ccc(C(F)(F)F)cc4F)c3s2)C1] |
| I-170 | 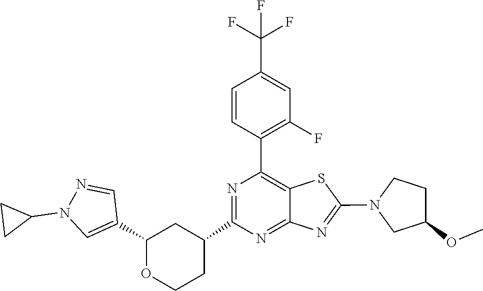 [SMILES: CO[C@@H]1CCN(c2nc3nc([C@@H]4CCO[C@H](c5cnn(C6CC6)c5)C4)nc(-c4ccc(C(F)(F)F)cc4F)c3s2)C1] |
| I-171 | 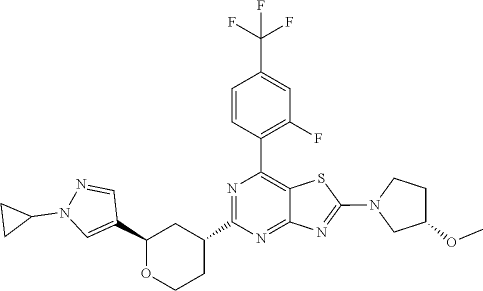 [SMILES: CO[C@H]1CCN(c2nc3nc([C@@H]4CCO[C@@H](c5cnn(C6CC6)c5)C4)nc(-c4ccc(C(F)(F)F)cc4F)c3s2)C1] |
| I-172 | 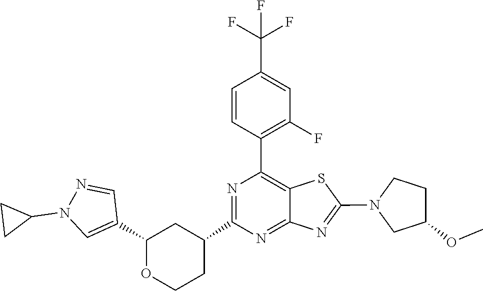 [SMILES: CO[C@H]1CCN(c2nc3nc([C@@H]4CCO[C@H](c5cnn(C6CC6)c5)C4)nc(-c4ccc(C(F)(F)F)cc4F)c3s2)C1] |
| I-173 | 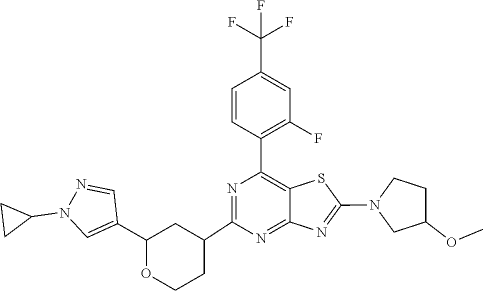 [SMILES: COC1CCN(c2nc3nc(C4CCOC(c5cnn(C6CC6)c5)C4)nc(-c4ccc(C(F)(F)F)cc4F)c3s2)C1] |
| I-174 | 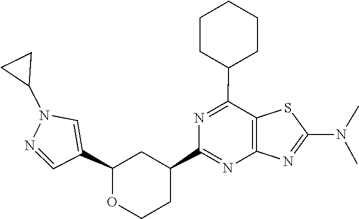 [SMILES: CN(C)c1nc2nc([C@H]3CCO[C@@H](c4cnn(C5CC5)c4)C3)nc(C3CCCCC3)c2s1] |
| I-175 | 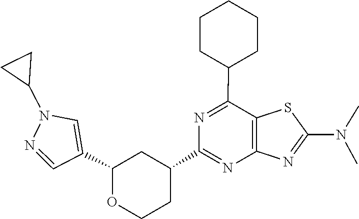 [SMILES: CN(C)c1nc2nc([C@@H]3CCO[C@H](c4cnn(C5CC5)c4)C3)nc(C3CCCCC3)c2s1] |
| I-176 | 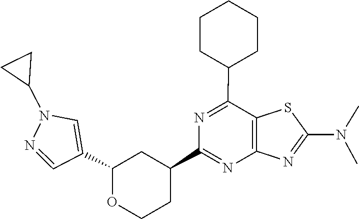 [SMILES: CN(C)c1nc2nc([C@H]3CCO[C@H](c4cnn(C5CC5)c4)C3)nc(C3CCCCC3)c2s1] |
| I-177 | 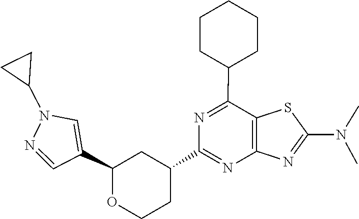 [SMILES: CN(C)c1nc2nc([C@@H]3CCO[C@@H](c4cnn(C5CC5)c4)C3)nc(C3CCCCC3)c2s1] |
| I-178 | 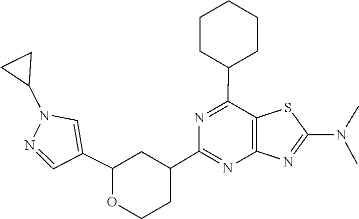 [SMILES: CN(C)c1nc2nc(C3CCOC(c4cnn(C5CC5)c4)C3)nc(C3CCCCC3)c2s1] |
| I-179 | 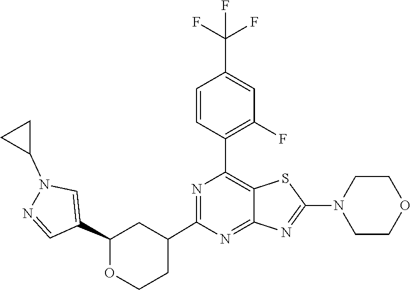 [SMILES: Fc1cc(C(F)(F)F)ccc1-c1nc(C2CCO[C@@H](c3cnn(C4CC4)c3)C2)nc2nc(N3CCOCC3)sc12] |
| I-180 | 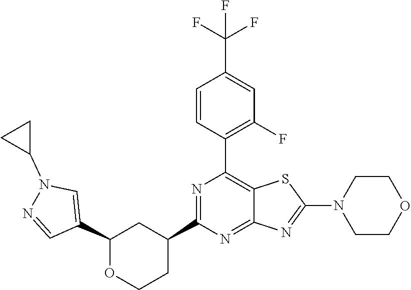 [SMILES: Fc1cc(C(F)(F)F)ccc1-c1nc([C@H]2CCO[C@@H](c3cnn(C4CC4)c3)C2)nc2nc(N3CCOCC3)sc12] |
| I-181 | 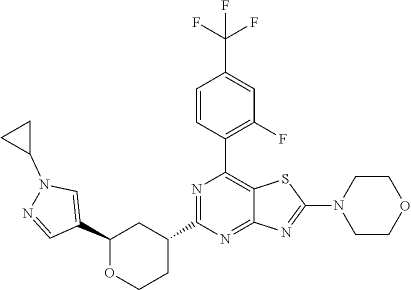 [SMILES: Fc1cc(C(F)(F)F)ccc1-c1nc([C@@H]2CCO[C@@H](c3cnn(C4CC4)c3)C2)nc2nc(N3CCOCC3)sc12] |
| I-182 | 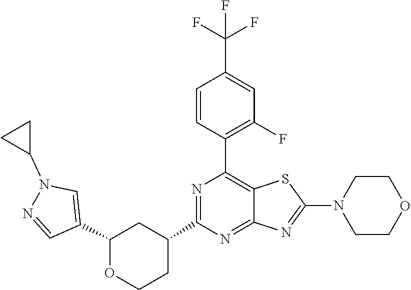 [SMILES: Fc1cc(C(F)(F)F)ccc1-c1nc([C@@H]2CCO[C@H](c3cnn(C4CC4)c3)C2)nc2nc(N3CCOCC3)sc12] |
| I-183 | 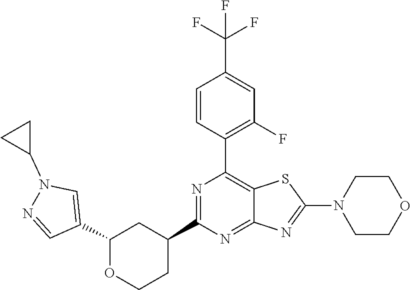 [SMILES: Fc1cc(C(F)(F)F)ccc1-c1nc([C@H]2CCO[C@H](c3cnn(C4CC4)c3)C2)nc2nc(N3CCOCC3)sc12] |
| I-184 | 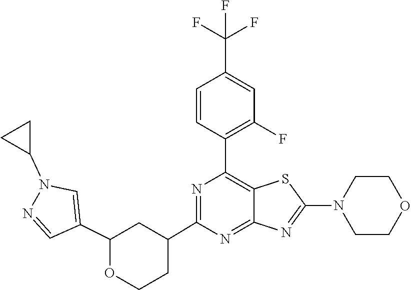 [SMILES: Fc1cc(C(F)(F)F)ccc1-c1nc(C2CCOC(c3cnn(C4CC4)c3)C2)nc2nc(N3CCOCC3)sc12] |
| I-185 | 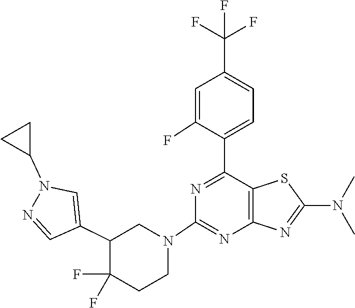 [SMILES: CN(C)c1nc2nc(N3CCC(F)(F)C(c4cnn(C5CC5)c4)C3)nc(-c3ccc(C(F)(F)F)cc3F)c2s1] |
| I-186 | 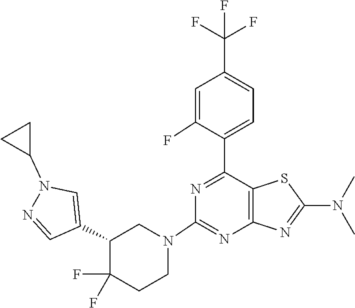 [SMILES: CN(C)c1nc2nc(N3CCC(F)(F)[C@H](c4cnn(C5CC5)c4)C3)nc(-c3ccc(C(F)(F)F)cc3F)c2s1] |
| I-187 | 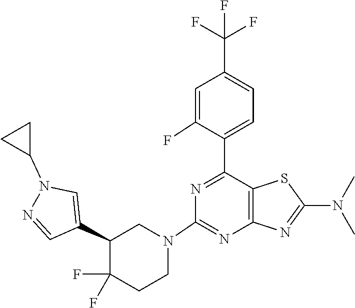 [SMILES: CN(C)c1nc2nc(N3CCC(F)(F)[C@@H](c4cnn(C5CC5)c4)C3)nc(-c3ccc(C(F)(F)F)cc3F)c2s1] |
| I-188 | 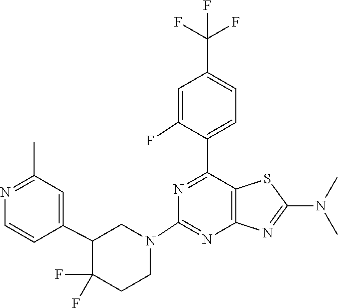 [SMILES: Cc1cc(C2CN(c3nc(-c4ccc(C(F)(F)F)cc4F)c4sc(N(C)C)nc4n3)CCC2(F)F)ccn1] |
| I-189 | 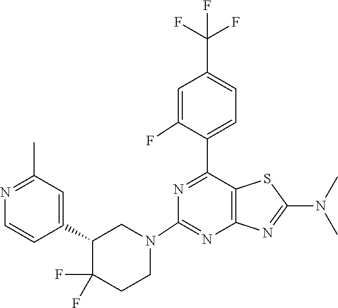 [SMILES: Cc1cc([C@@H]2CN(c3nc(-c4ccc(C(F)(F)F)cc4F)c4sc(N(C)C)nc4n3)CCC2(F)F)ccn1] |
| I-190 | 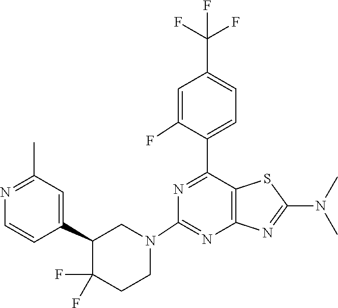 [SMILES: Cc1cc([C@H]2CN(c3nc(-c4ccc(C(F)(F)F)cc4F)c4sc(N(C)C)nc4n3)CCC2(F)F)ccn1] |
| I-191 | 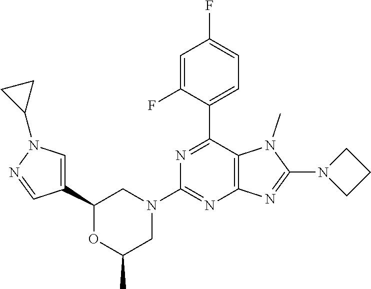 [SMILES: C[C@@H]1CN(c2nc(-c3ccc(F)cc3F)c3c(n2)nc(N2CCC2)n3C)C[C@H](c2cnn(C3CC3)c2)O1] |
| I-192 | 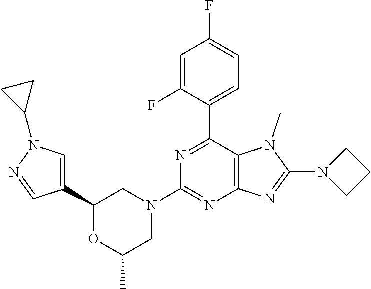 [SMILES: C[C@H]1CN(c2nc(-c3ccc(F)cc3F)c3c(n2)nc(N2CCC2)n3C)C[C@H](c2cnn(C3CC3)c2)O1] |
| I-193 | 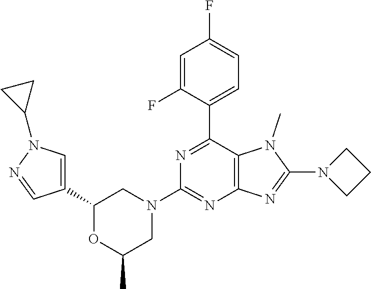 [SMILES: C[C@@H]1CN(c2nc(-c3ccc(F)cc3F)c3c(n2)nc(N2CCC2)n3C)C[C@@H](c2cnn(C3CC3)c2)O1] |
| I-194 | 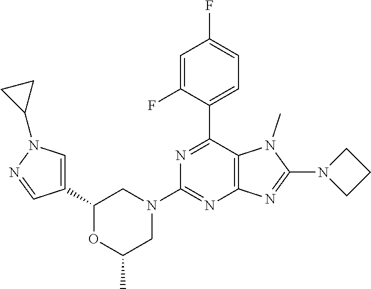 [SMILES: C[C@H]1CN(c2nc(-c3ccc(F)cc3F)c3c(n2)nc(N2CCC2)n3C)C[C@@H](c2cnn(C3CC3)c2)O1] |
| I-195 | 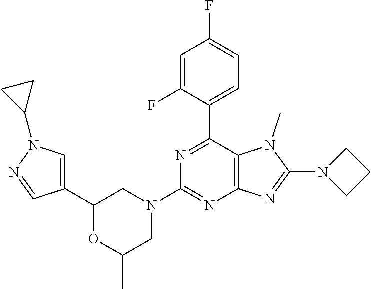 [SMILES: CC1CN(c2nc(-c3ccc(F)cc3F)c3c(n2)nc(N2CCC2)n3C)CC(c2cnn(C3CC3)c2)O1] |
| I-196 | 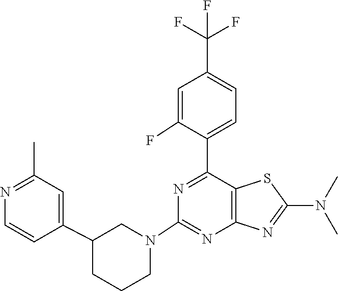 [SMILES: Cc1cc(C2CCCN(c3nc(-c4ccc(C(F)(F)F)cc4F)c4sc(N(C)C)nc4n3)C2)ccn1] |
| I-197 | 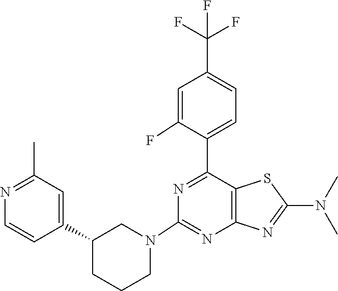 [SMILES: Cc1cc([C@H]2CCCN(c3nc(-c4ccc(C(F)(F)F)cc4F)c4sc(N(C)C)nc4n3)C2)ccn1] |
| I-198 | 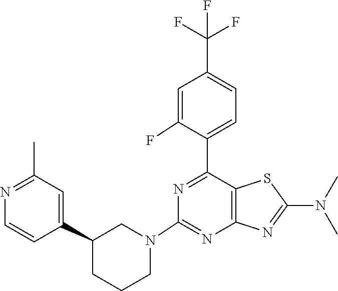 [SMILES: Cc1cc([C@@H]2CCCN(c3nc(-c4ccc(C(F)(F)F)cc4F)c4sc(N(C)C)nc4n3)C2)ccn1] |
| I-199 | 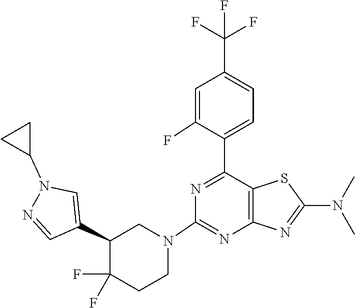 [SMILES: CN(C)c1nc2nc(N3CCC(F)(F)[C@@H](c4cnn(C5CC5)c4)C3)nc(-c3ccc(C(F)(F)F)cc3F)c2s1] |
| I-200 | 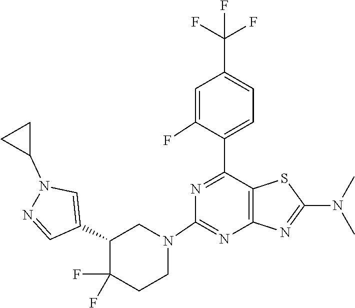 [SMILES: CN(C)c1nc2nc(N3CCC(F)(F)[C@H](c4cnn(C5CC5)c4)C3)nc(-c3ccc(C(F)(F)F)cc3F)c2s1] |
| I-201 | 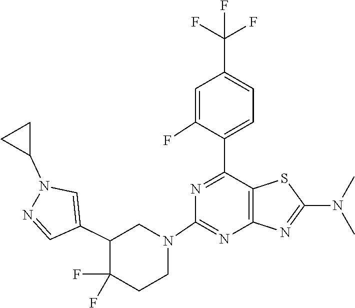 [SMILES: CN(C)c1nc2nc(N3CCC(F)(F)C(c4cnn(C5CC5)c4)C3)nc(-c3ccc(C(F)(F)F)cc3F)c2s1] |
| I-202 | 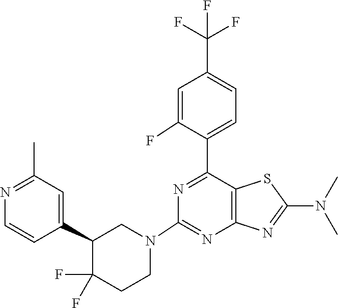 [SMILES: Cc1cc([C@H]2CN(c3nc(-c4ccc(C(F)(F)F)cc4F)c4sc(N(C)C)nc4n3)CCC2(F)F)ccn1] |
| I-203 | 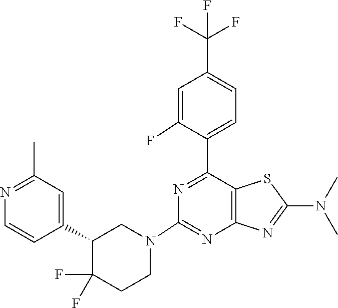 [SMILES: Cc1cc([C@@H]2CN(c3nc(-c4ccc(C(F)(F)F)cc4F)c4sc(N(C)C)nc4n3)CCC2(F)F)ccn1] |
| I-204 | 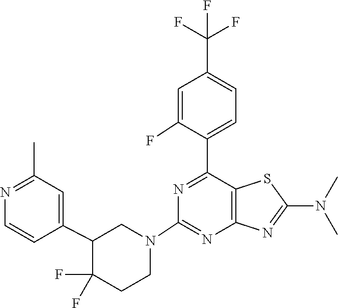 [SMILES: Cc1cc(C2CN(c3nc(-c4ccc(C(F)(F)F)cc4F)c4sc(N(C)C)nc4n3)CCC2(F)F)ccn1] |
| I-205 | 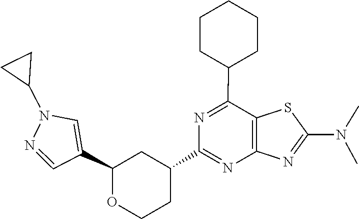 [SMILES: CN(C)c1nc2nc([C@@H]3CCO[C@@H](c4cnn(C5CC5)c4)C3)nc(C3CCCCC3)c2s1] |
| I-206 | 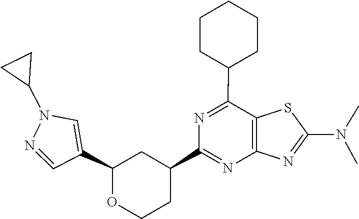 [SMILES: CN(C)c1nc2nc([C@H]3CCO[C@@H](c4cnn(C5CC5)c4)C3)nc(C3CCCCC3)c2s1] |
| I-207 | 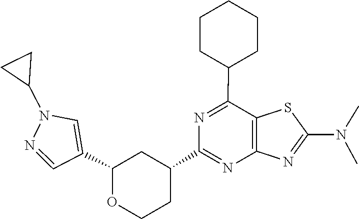 [SMILES: CN(C)c1nc2nc([C@@H]3CCO[C@H](c4cnn(C5CC5)c4)C3)nc(C3CCCCC3)c2s1] |
| I-208 | 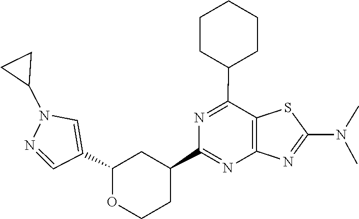 [SMILES: CN(C)c1nc2nc([C@H]3CCO[C@H](c4cnn(C5CC5)c4)C3)nc(C3CCCCC3)c2s1] |
| I-209 | 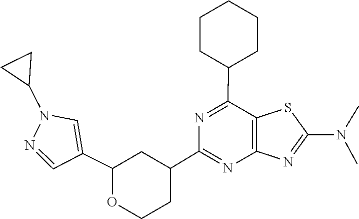 [SMILES: CN(C)c1nc2nc(C3CCOC(c4cnn(C5CC5)c4)C3)nc(C3CCCCC3)c2s1] |
| I-210 | 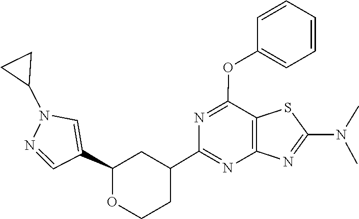 [SMILES: CN(C)c1nc2nc(C3CCO[C@@H](c4cnn(C5CC5)c4)C3)nc(Oc3ccccc3)c2s1] |
| I-211 | 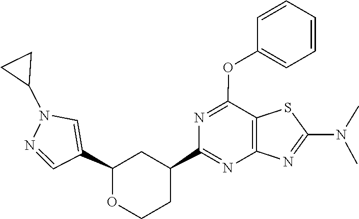 [SMILES: CN(C)c1nc2nc([C@H]3CCO[C@@H](c4cnn(C5CC5)c4)C3)nc(Oc3ccccc3)c2s1] |
| I-212 | 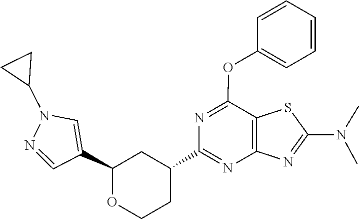 [SMILES: CN(C)c1nc2nc([C@@H]3CCO[C@@H](c4cnn(C5CC5)c4)C3)nc(Oc3ccccc3)c2s1] |
| I-213 | 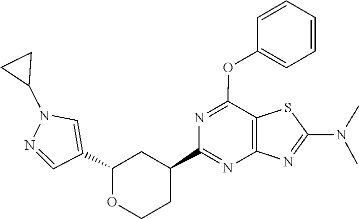 [SMILES: CN(C)c1nc2nc([C@H]3CCO[C@H](c4cnn(C5CC5)c4)C3)nc(Oc3ccccc3)c2s1] |
| I-214 | 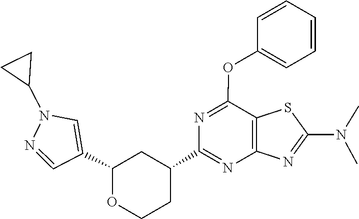 [SMILES: CN(C)c1nc2nc([C@@H]3CCO[C@H](c4cnn(C5CC5)c4)C3)nc(Oc3ccccc3)c2s1] |
| I-215 | 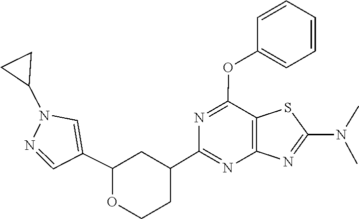 [SMILES: CN(C)c1nc2nc(C3CCOC(c4cnn(C5CC5)c4)C3)nc(Oc3ccccc3)c2s1] |
| I-216 | 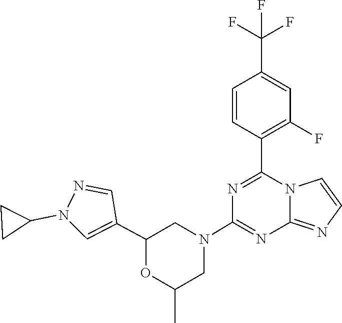 [SMILES: CC1CN(c2nc(-c3ccc(C(F)(F)F)cc3F)n3ccnc3n2)CC(c2cnn(C3CC3)c2)O1] |
| I-217 | 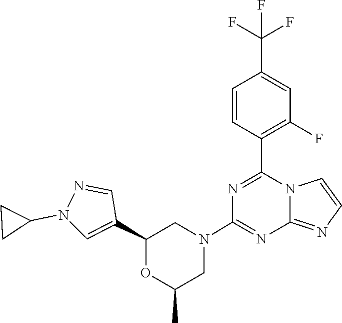 [SMILES: C[C@@H]1CN(c2nc(-c3ccc(C(F)(F)F)cc3F)n3ccnc3n2)C[C@H](c2cnn(C3CC3)c2)O1] |
| I-218 | 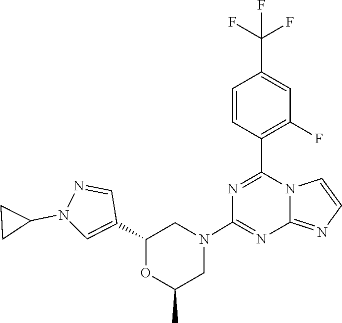 [SMILES: C[C@@H]1CN(c2nc(-c3ccc(C(F)(F)F)cc3F)n3ccnc3n2)C[C@@H](c2cnn(C3CC3)c2)O1] |
| I-219 | 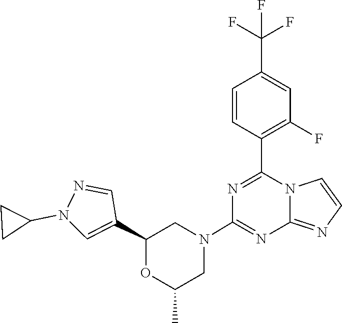 [SMILES: C[C@H]1CN(c2nc(-c3ccc(C(F)(F)F)cc3F)n3ccnc3n2)C[C@H](c2cnn(C3CC3)c2)O1] |
| I-220 | 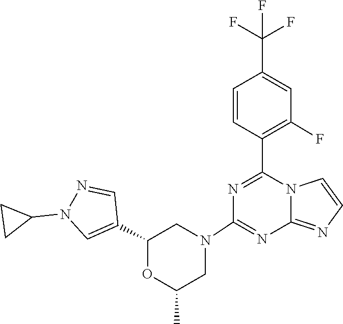 [SMILES: C[C@H]1CN(c2nc(-c3ccc(C(F)(F)F)cc3F)n3ccnc3n2)C[C@@H](c2cnn(C3CC3)c2)O1] |
| I-221 | 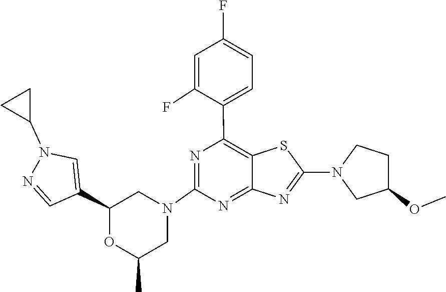 [SMILES: CO[C@@H]1CCN(c2nc3nc(N4C[C@@H](C)O[C@@H](c5cnn(C6CC6)c5)C4)nc(-c4ccc(F)cc4F)c3s2)C1] |
| I-222 | 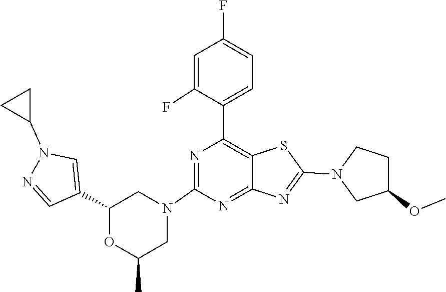 [SMILES: CO[C@@H]1CCN(c2nc3nc(N4C[C@@H](C)O[C@H](c5cnn(C6CC6)c5)C4)nc(-c4ccc(F)cc4F)c3s2)C1] |
| I-223 | 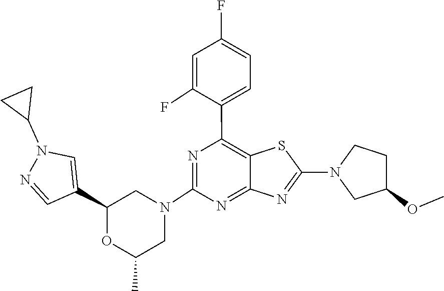 [SMILES: CO[C@@H]1CCN(c2nc3nc(N4C[C@H](C)O[C@@H](c5cnn(C6CC6)c5)C4)nc(-c4ccc(F)cc4F)c3s2)C1] |
| I-224 | 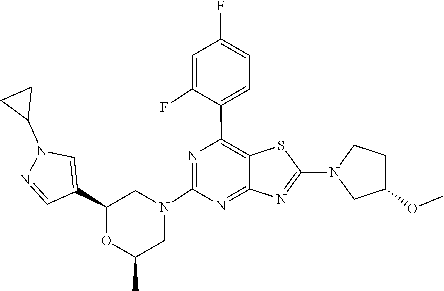 [SMILES: CO[C@H]1CCN(c2nc3nc(N4C[C@@H](C)O[C@@H](c5cnn(C6CC6)c5)C4)nc(-c4ccc(F)cc4F)c3s2)C1] |
| I-225 | 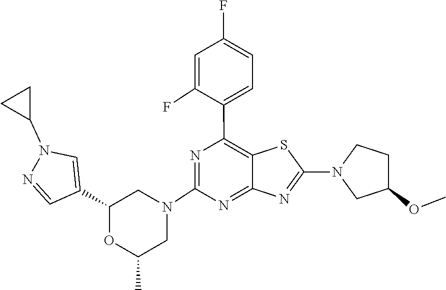 [SMILES: CO[C@@H]1CCN(c2nc3nc(N4C[C@@H](c5cnn(C6CC6)c5)O[C@@H](C)C4)nc(-c4ccc(F)cc4F)c3s2)C1] |
| I-226 | 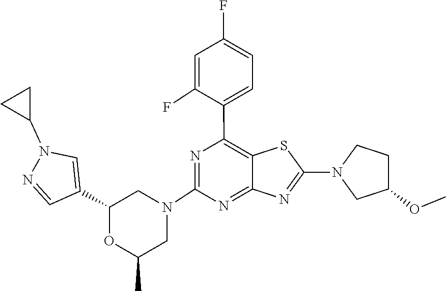 [SMILES: CO[C@H]1CCN(c2nc3nc(N4C[C@@H](C)O[C@H](c5cnn(C6CC6)c5)C4)nc(-c4ccc(F)cc4F)c3s2)C1] |
| I-227 | 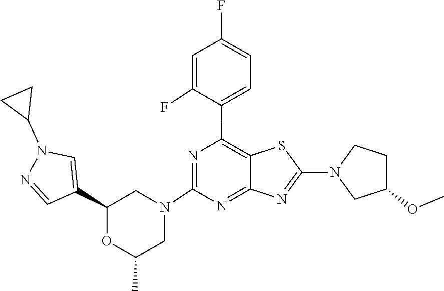 [SMILES: CO[C@H]1CCN(c2nc3nc(N4C[C@H](C)O[C@@H](c5cnn(C6CC6)c5)C4)nc(-c4ccc(F)cc4F)c3s2)C1] |
| I-228 | 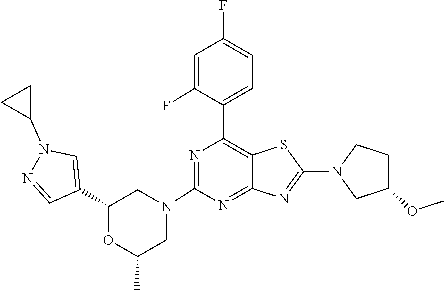 [SMILES: CO[C@H]1CCN(c2nc3nc(N4C[C@@H](c5cnn(C6CC6)c5)O[C@@H](C)C4)nc(-c4ccc(F)cc4F)c3s2)C1] |
| I-229 | 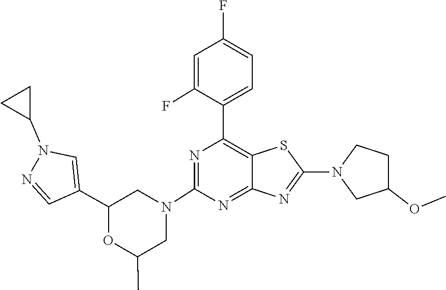 [SMILES: COC1CCN(c2nc3nc(N4CC(C)OC(c5cnn(C6CC6)c5)C4)nc(-c4ccc(F)cc4F)c3s2)C1] |
| I-230 | 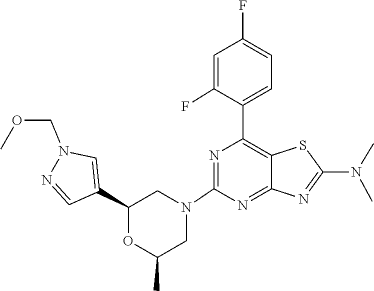 |
| I-231 | 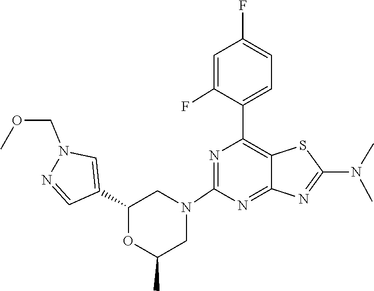 |
| I-232 | 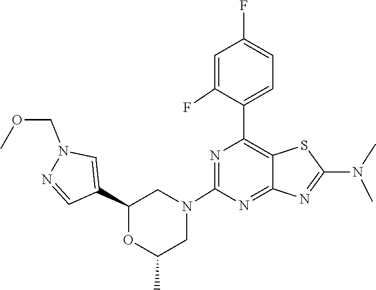 |
| I-233 | 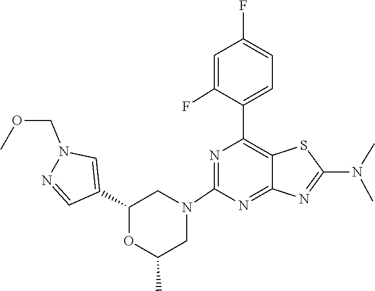 |
| I-234 | 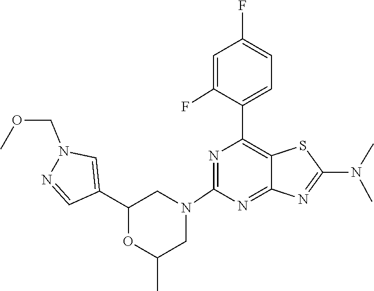 |
| I-235 | 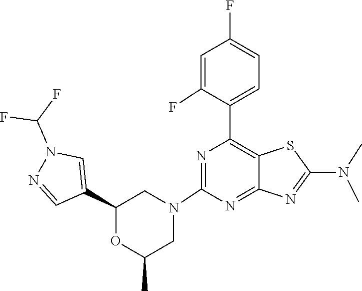 [SMILES: C[C@@H]1CN(c2nc(-c3ccc(F)cc3F)c3sc(N(C)C)nc3n2)C[C@H](c2cnn(C(F)F)c2)O1] |
| I-236 | 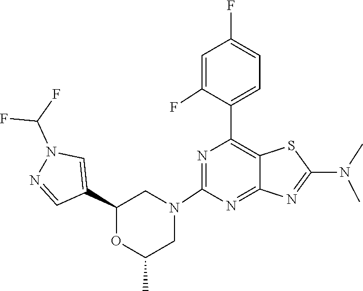 [SMILES: C[C@H]1CN(c2nc(-c3ccc(F)cc3F)c3sc(N(C)C)nc3n2)C[C@H](c2cnn(C(F)F)c2)O1] |
| I-237 | 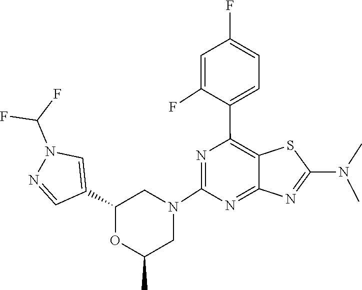 [SMILES: C[C@@H]1CN(c2nc(-c3ccc(F)cc3F)c3sc(N(C)C)nc3n2)C[C@@H](c2cnn(C(F)F)c2)O1] |
| I-238 | 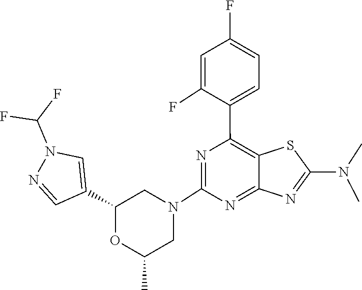 [SMILES: C[C@H]1CN(c2nc(-c3ccc(F)cc3F)c3sc(N(C)C)nc3n2)C[C@@H](c2cnn(C(F)F)c2)O1] |
| I-239 | 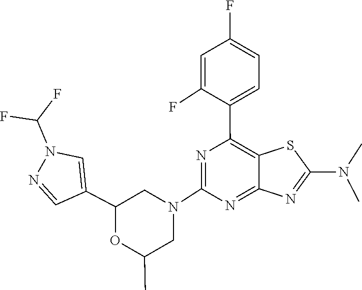 [SMILES: CC1CN(c2nc(-c3ccc(F)cc3F)c3sc(N(C)C)nc3n2)CC(c2cnn(C(F)F)c2)O1] |
| I-240 | 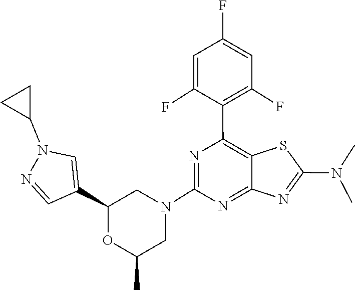 [SMILES: C[C@@H]1CN(c2nc(-c3c(F)cc(F)cc3F)c3sc(N(C)C)nc3n2)C[C@H](c2cnn(C3CC3)c2)O1] |
| I-241 | 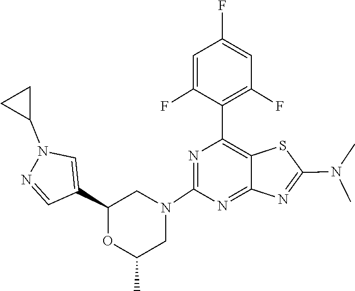 [SMILES: C[C@H]1CN(c2nc(-c3c(F)cc(F)cc3F)c3sc(N(C)C)nc3n2)C[C@H](c2cnn(C3CC3)c2)O1] |
| I-242 | 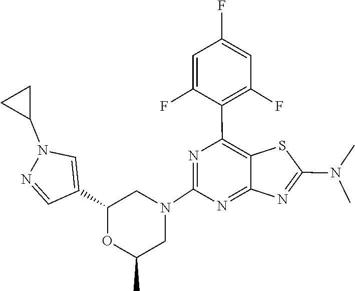 [SMILES: C[C@@H]1CN(c2nc(-c3c(F)cc(F)cc3F)c3sc(N(C)C)nc3n2)C[C@@H](c2cnn(C3CC3)c2)O1] |
| I-243 | 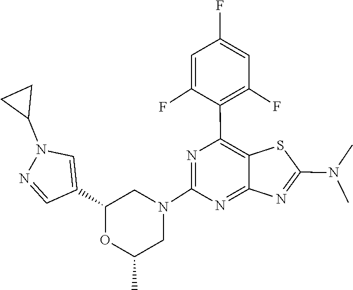 [SMILES: C[C@H]1CN(c2nc(-c3c(F)cc(F)cc3F)c3sc(N(C)C)nc3n2)C[C@@H](c2cnn(C3CC3)c2)O1] |
| I-244 | 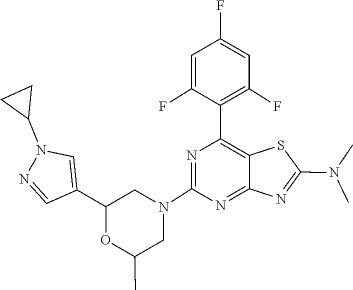 [SMILES: CC1CN(c2nc(-c3c(F)cc(F)cc3F)c3sc(N(C)C)nc3n2)CC(c2cnn(C3CC3)c2)O1] |
| I-245 | 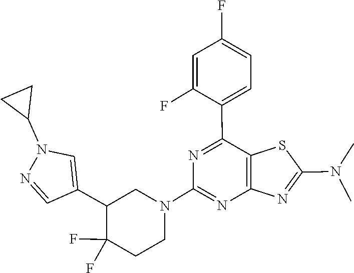 [SMILES: CN(C)c1nc2nc(N3CCC(F)(F)C(c4cnn(C5CC5)c4)C3)nc(-c3ccc(F)cc3F)c2s1] |
| I-246 | 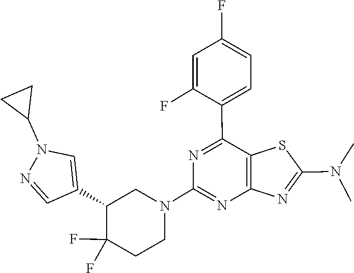 [SMILES: CN(C)c1nc2nc(N3CCC(F)(F)[C@H](c4cnn(C5CC5)c4)C3)nc(-c3ccc(F)cc3F)c2s1] |
| I-247 | 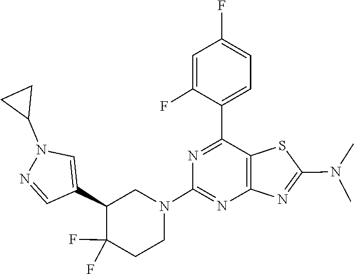 [SMILES: CN(C)c1nc2nc(N3CCC(F)(F)[C@@H](c4cnn(C5CC5)c4)C3)nc(-c3ccc(F)cc3F)c2s1] |
| I-248 | 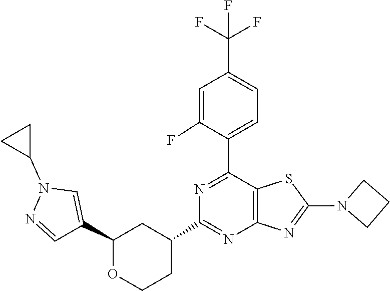 [SMILES: Fc1cc(C(F)(F)F)ccc1-c1nc([C@@H]2CCO[C@@H](c3cnn(C4CC4)c3)C2)nc2nc(N3CCC3)sc12] |
| I-249 | 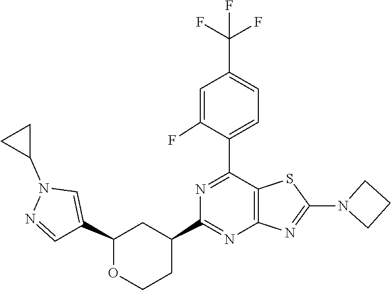 [SMILES: Fc1cc(C(F)(F)F)ccc1-c1nc([C@H]2CCO[C@@H](c3cnn(C4CC4)c3)C2)nc2nc(N3CCC3)sc12] |
| I-250 | 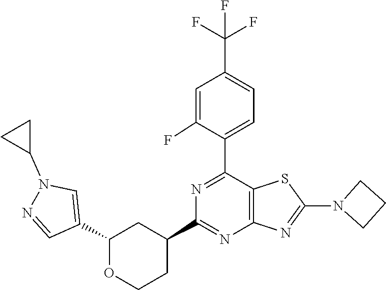 [SMILES: Fc1cc(C(F)(F)F)ccc1-c1nc([C@H]2CCO[C@H](c3cnn(C4CC4)c3)C2)nc2nc(N3CCC3)sc12] |
| I-251 | 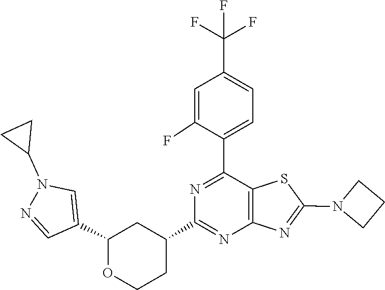 [SMILES: Fc1cc(C(F)(F)F)ccc1-c1nc([C@@H]2CCO[C@H](c3cnn(C4CC4)c3)C2)nc2nc(N3CCC3)sc12] |
| I-252 | 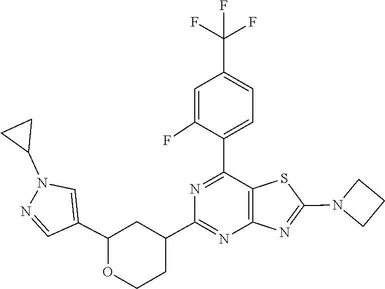 [SMILES: Fc1cc(C(F)(F)F)ccc1-c1nc(C2CCOC(c3cnn(C4CC4)c3)C2)nc2nc(N3CCC3)sc12] |
| I-253 | 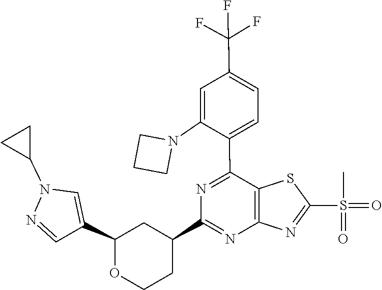 [SMILES: CS(=O)(=O)c1nc2nc([C@H]3CCO[C@@H](c4cnn(C5CC5)c4)C3)nc(-c3ccc(C(F)(F)F)cc3N3CCC3)c2s1] |
| I-254 | 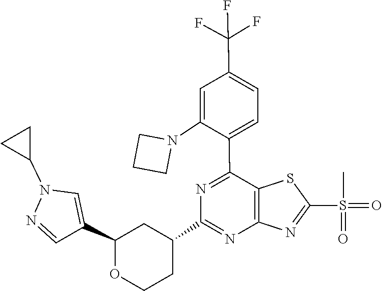 [SMILES: CS(=O)(=O)c1nc2nc([C@@H]3CCO[C@@H](c4cnn(C5CC5)c4)C3)nc(-c3ccc(C(F)(F)F)cc3N3CCC3)c2s1] |
| I-255 | 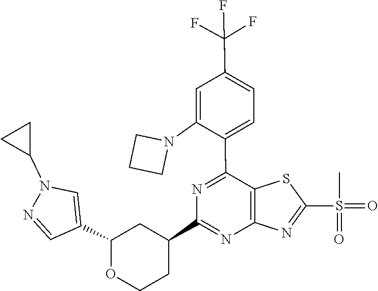 [SMILES: CS(=O)(=O)c1nc2nc([C@H]3CCO[C@H](c4cnn(C5CC5)c4)C3)nc(-c3ccc(C(F)(F)F)cc3N3CCC3)c2s1] |
| I-256 | 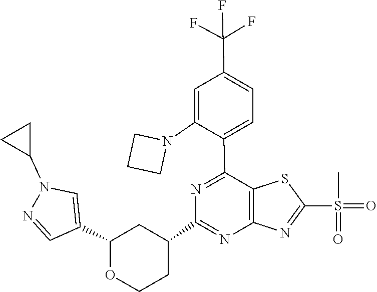 [SMILES: CS(=O)(=O)c1nc2nc([C@@H]3CCO[C@H](c4cnn(C5CC5)c4)C3)nc(-c3ccc(C(F)(F)F)cc3N3CCC3)c2s1] |
| I-257 | 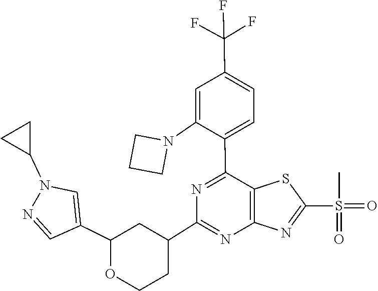 [SMILES: CS(=O)(=O)c1nc2nc(C3CCOC(c4cnn(C5CC5)c4)C3)nc(-c3ccc(C(F)(F)F)cc3N3CCC3)c2s1] |
| I-258 | 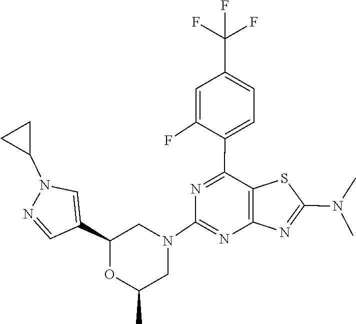 [SMILES: C[C@@H]1CN(c2nc(-c3ccc(C(F)(F)F)cc3F)c3sc(N(C)C)nc3n2)C[C@H](c2cnn(C3CC3)c2)O1] |
| I-259 | 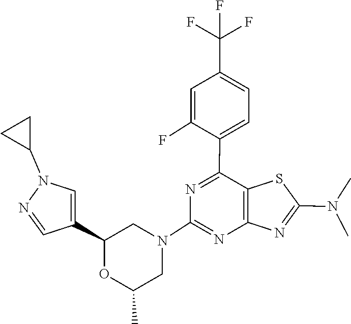 [SMILES: C[C@H]1CN(c2nc(-c3ccc(C(F)(F)F)cc3F)c3sc(N(C)C)nc3n2)C[C@H](c2cnn(C3CC3)c2)O1] |
| I-260 | 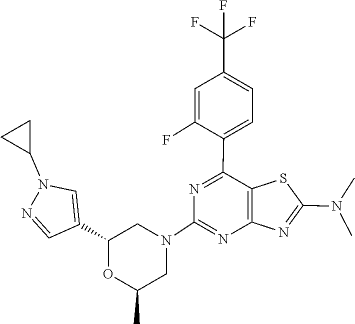 [SMILES: C[C@@H]1CN(c2nc(-c3ccc(C(F)(F)F)cc3F)c3sc(N(C)C)nc3n2)C[C@@H](c2cnn(C3CC3)c2)O1] |
| I-261 | 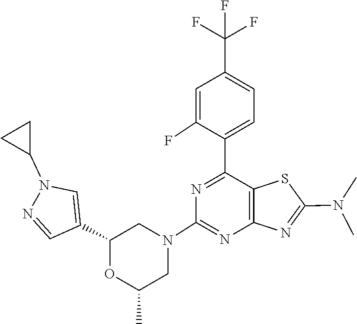 [SMILES: C[C@H]1CN(c2nc(-c3ccc(C(F)(F)F)cc3F)c3sc(N(C)C)nc3n2)C[C@@H](c2cnn(C3CC3)c2)O1] |
| I-262 | 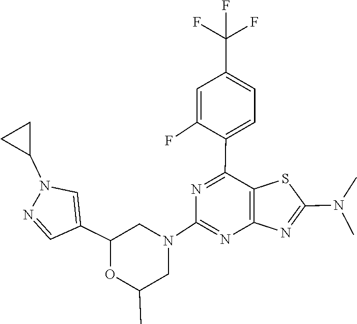 [SMILES: CC1CN(c2nc(-c3ccc(C(F)(F)F)cc3F)c3sc(N(C)C)nc3n2)CC(c2cnn(C3CC3)c2)O1] |
| I-263 | 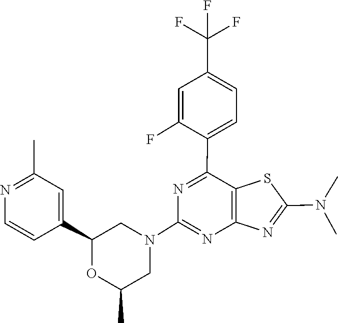 [SMILES: Cc1cc([C@H]2CN(c3nc(-c4ccc(C(F)(F)F)cc4F)c4sc(N(C)C)nc4n3)C[C@@H](C)O2)ccn1] |
| I-264 | 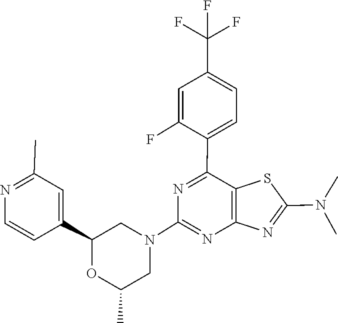 [SMILES: Cc1cc([C@H]2CN(c3nc(-c4ccc(C(F)(F)F)cc4F)c4sc(N(C)C)nc4n3)C[C@H](C)O2)ccn1] |
| I-265 | 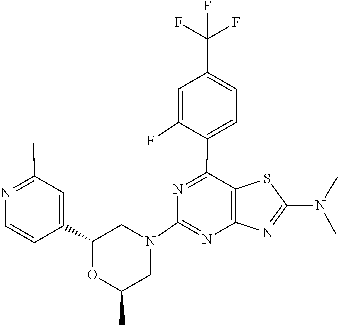 [SMILES: Cc1cc([C@@H]2CN(c3nc(-c4ccc(C(F)(F)F)cc4F)c4sc(N(C)C)nc4n3)C[C@@H](C)O2)ccn1] |
| I-266 | 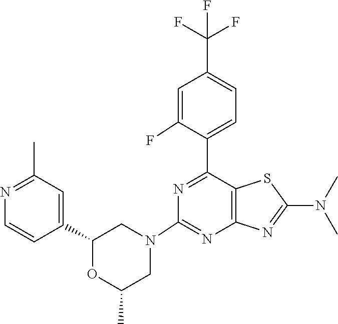 [SMILES: Cc1cc([C@@H]2CN(c3nc(-c4ccc(C(F)(F)F)cc4F)c4sc(N(C)C)nc4n3)C[C@H](C)O2)ccn1] |
| I-267 | 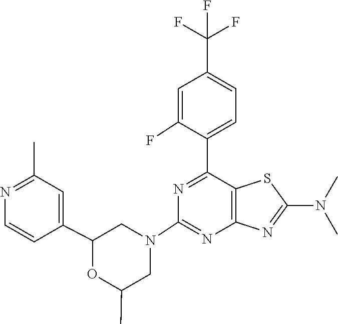 [SMILES: Cc1cc(C2CN(c3nc(-c4ccc(C(F)(F)F)cc4F)c4sc(N(C)C)nc4n3)CC(C)O2)ccn1] |
| I-268 | 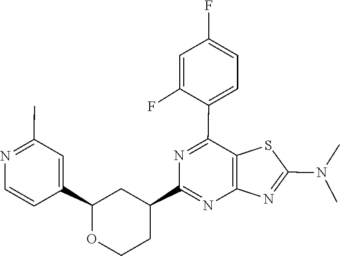 [SMILES: Cc1cc([C@H]2C[C@@H](c3nc(-c4ccc(F)cc4F)c4sc(N(C)C)nc4n3)CCO2)ccn1] |
| I-269 | 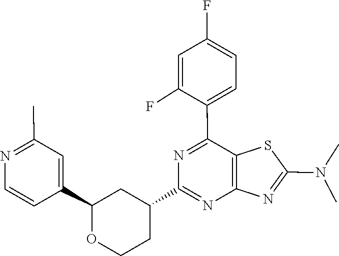 [SMILES: Cc1cc([C@H]2C[C@H](c3nc(-c4ccc(F)cc4F)c4sc(N(C)C)nc4n3)CCO2)ccn1] |
| I-270 | 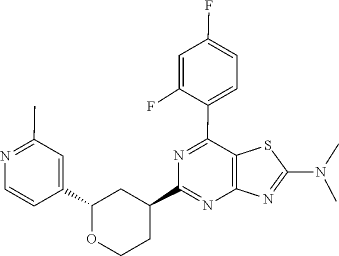 [SMILES: Cc1cc([C@@H]2C[C@@H](c3nc(-c4ccc(F)cc4F)c4sc(N(C)C)nc4n3)CCO2)ccn1] |
| I-271 | 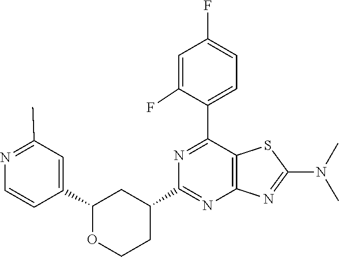 [SMILES: Cc1cc([C@@H]2C[C@H](c3nc(-c4ccc(F)cc4F)c4sc(N(C)C)nc4n3)CCO2)ccn1] |
| I-272 | 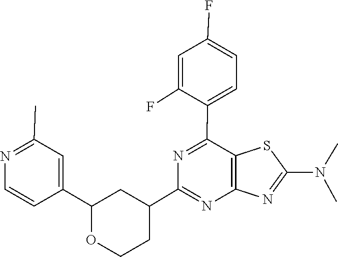 [SMILES: Cc1cc(C2CC(c3nc(-c4ccc(F)cc4F)c4sc(N(C)C)nc4n3)CCO2)ccn1] |
| I-273 | 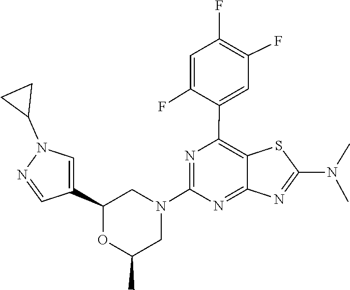 [SMILES: C[C@@H]1CN(c2nc(-c3cc(F)c(F)cc3F)c3sc(N(C)C)nc3n2)C[C@H](c2cnn(C3CC3)c2)O1] |
| I-274 | 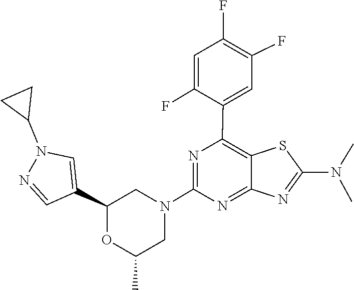 [SMILES: C[C@H]1CN(c2nc(-c3cc(F)c(F)cc3F)c3sc(N(C)C)nc3n2)C[C@H](c2cnn(C3CC3)c2)O1] |
| I-275 | 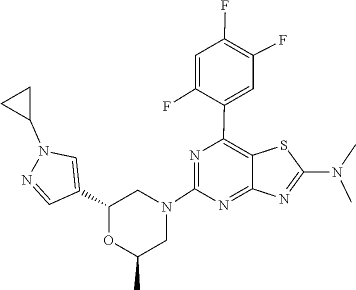 [SMILES: C[C@@H]1CN(c2nc(-c3cc(F)c(F)cc3F)c3sc(N(C)C)nc3n2)C[C@@H](c2cnn(C3CC3)c2)O1] |
| I-276 | 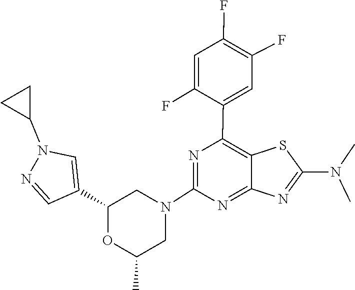 [SMILES: C[C@H]1CN(c2nc(-c3cc(F)c(F)cc3F)c3sc(N(C)C)nc3n2)C[C@@H](c2cnn(C3CC3)c2)O1] |
| I-277 | 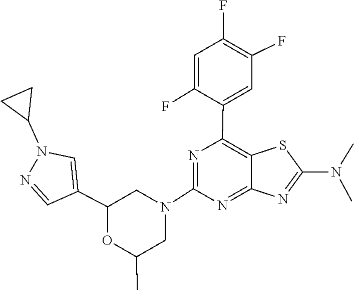 [SMILES: CC1CN(c2nc(-c3cc(F)c(F)cc3F)c3sc(N(C)C)nc3n2)CC(c2cnn(C3CC3)c2)O1] |
| I-278 | 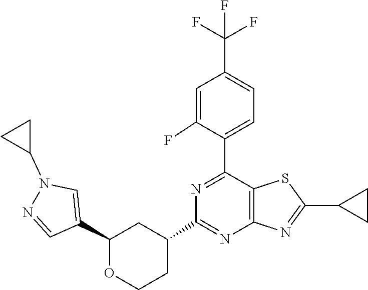 [SMILES: Fc1cc(C(F)(F)F)ccc1-c1nc([C@@H]2CCO[C@@H](c3cnn(C4CC4)c3)C2)nc2nc(C3CC3)sc12] |
| I-279 | 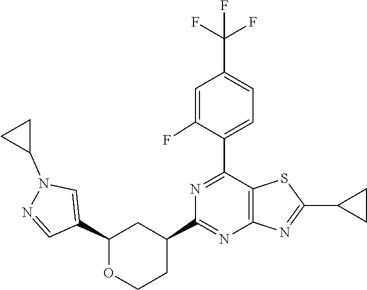 [SMILES: Fc1cc(C(F)(F)F)ccc1-c1nc([C@H]2CCO[C@@H](c3cnn(C4CC4)c3)C2)nc2nc(C3CC3)sc12] |
| I-280 | 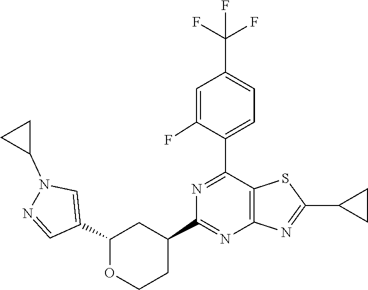 [SMILES: Fc1cc(C(F)(F)F)ccc1-c1nc([C@H]2CCO[C@H](c3cnn(C4CC4)c3)C2)nc2nc(C3CC3)sc12] |
| I-281 | 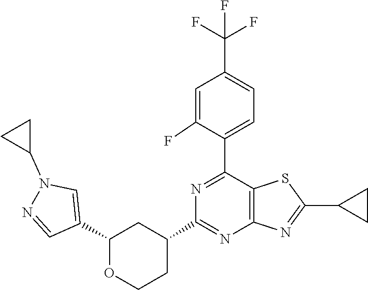 [SMILES: Fc1cc(C(F)(F)F)ccc1-c1nc([C@@H]2CCO[C@H](c3cnn(C4CC4)c3)C2)nc2nc(C3CC3)sc12] |
| I-282 | 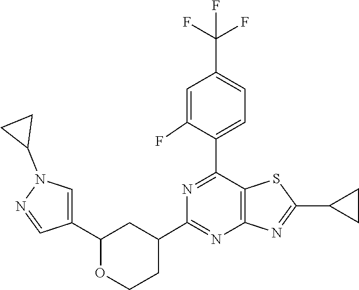 [SMILES: Fc1cc(C(F)(F)F)ccc1-c1nc(C2CCOC(c3cnn(C4CC4)c3)C2)nc2nc(C3CC3)sc12] |
| I-283 | 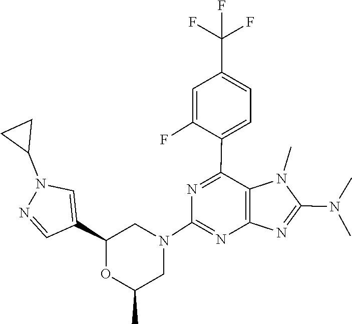 [SMILES: C[C@@H]1CN(c2nc(-c3ccc(C(F)(F)F)cc3F)c3c(n2)nc(N(C)C)n3C)C[C@H](c2cnn(C3CC3)c2)O1] |
| I-284 | 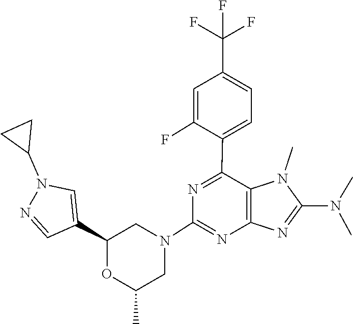 [SMILES: C[C@H]1CN(c2nc(-c3ccc(C(F)(F)F)cc3F)c3c(n2)nc(N(C)C)n3C)C[C@H](c2cnn(C3CC3)c2)O1] |
| I-285 | 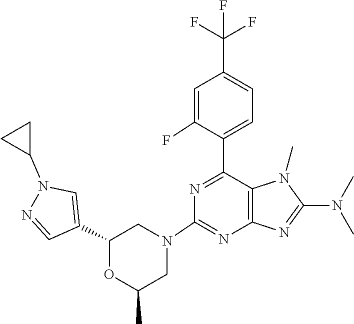 [SMILES: C[C@@H]1CN(c2nc(-c3ccc(C(F)(F)F)cc3F)c3c(n2)nc(N(C)C)n3C)C[C@@H](c2cnn(C3CC3)c2)O1] |
| I-286 | 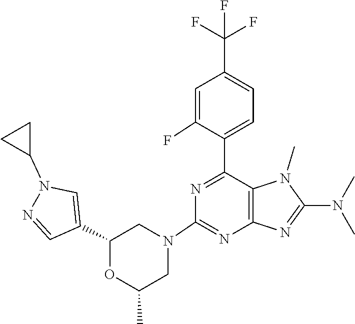 [SMILES: C[C@H]1CN(c2nc(-c3ccc(C(F)(F)F)cc3F)c3c(n2)nc(N(C)C)n3C)C[C@@H](c2cnn(C3CC3)c2)O1] |
| I-287 | 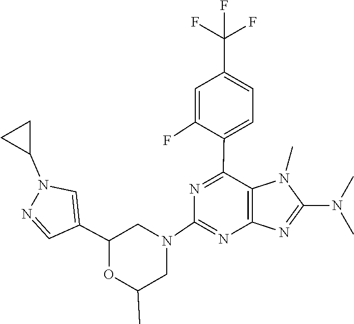 [SMILES: CC1CN(c2nc(-c3ccc(C(F)(F)F)cc3F)c3c(n2)nc(N(C)C)n3C)CC(c2cnn(C3CC3)c2)O1] |
| I-288 | 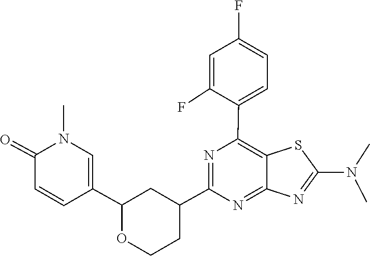 [SMILES: CN(C)c1nc2nc(C3CCOC(c4ccc(=O)n(C)c4)C3)nc(-c3ccc(F)cc3F)c2s1] |
| I-289 | 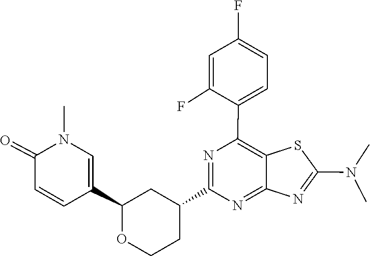 [SMILES: CN(C)c1nc2nc([C@@H]3CCO[C@@H](c4ccc(=O)n(C)c4)C3)nc(-c3ccc(F)cc3F)c2s1] |
| I-290 | 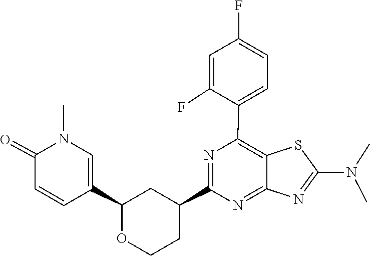 [SMILES: CN(C)c1nc2nc([C@H]3CCO[C@@H](c4ccc(=O)n(C)c4)C3)nc(-c3ccc(F)cc3F)c2s1] |
| I-291 | 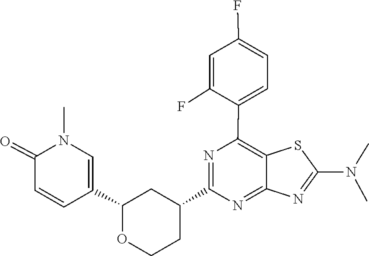 [SMILES: CN(C)c1nc2nc([C@@H]3CCO[C@H](c4ccc(=O)n(C)c4)C3)nc(-c3ccc(F)cc3F)c2s1] |
| I-292 | 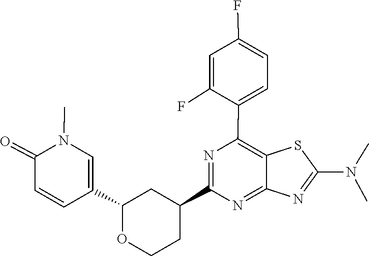 [SMILES: CN(C)c1nc2nc([C@H]3CCO[C@H](c4ccc(=O)n(C)c4)C3)nc(-c3ccc(F)cc3F)c2s1] |
| I-293 | 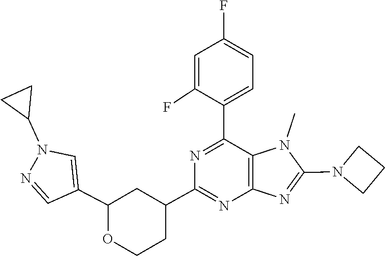 [SMILES: Cn1c(N2CCC2)nc2nc(C3CCOC(c4cnn(C5CC5)c4)C3)nc(-c3ccc(F)cc3F)c21] |
| I-294 | 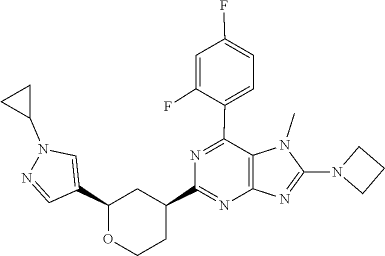 [SMILES: Cn1c(N2CCC2)nc2nc([C@H]3CCO[C@@H](c4cnn(C5CC5)c4)C3)nc(-c3ccc(F)cc3F)c21] |
| I-295 | 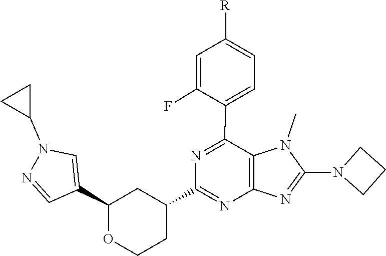 |
| I-296 | 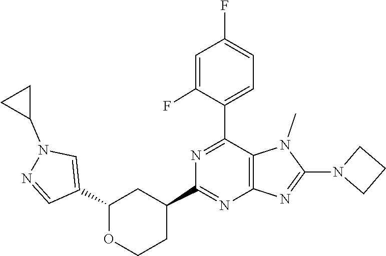 [SMILES: Cn1c(N2CCC2)nc2nc([C@H]3CCO[C@H](c4cnn(C5CC5)c4)C3)nc(-c3ccc(F)cc3F)c21] |
| I-297 | 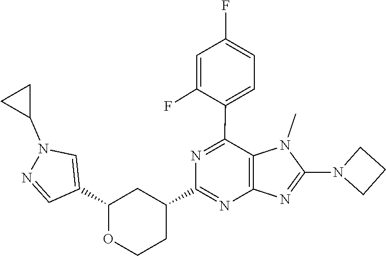 [SMILES: Cn1c(N2CCC2)nc2nc([C@@H]3CCO[C@H](c4cnn(C5CC5)c4)C3)nc(-c3ccc(F)cc3F)c21] |
| I-298 | 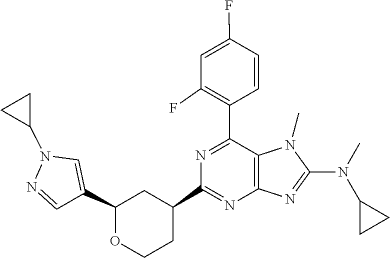 [SMILES: CN(c1nc2nc([C@H]3CCO[C@@H](c4cnn(C5CC5)c4)C3)nc(-c3ccc(F)cc3F)c2n1C)C1CC1] |
| I-299 | 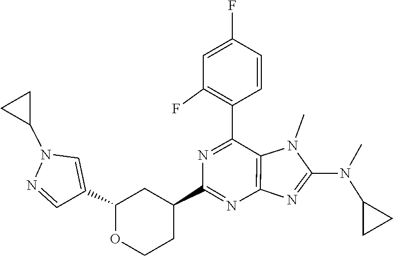 [SMILES: CN(c1nc2nc([C@H]3CCO[C@H](c4cnn(C5CC5)c4)C3)nc(-c3ccc(F)cc3F)c2n1C)C1CC1] |
| I-300 | 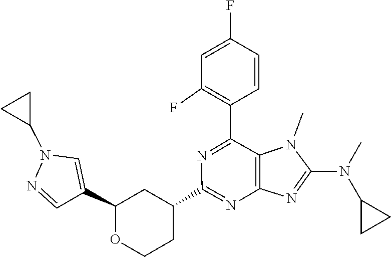 [SMILES: CN(c1nc2nc([C@@H]3CCO[C@@H](c4cnn(C5CC5)c4)C3)nc(-c3ccc(F)cc3F)c2n1C)C1CC1] |
| I-301 | 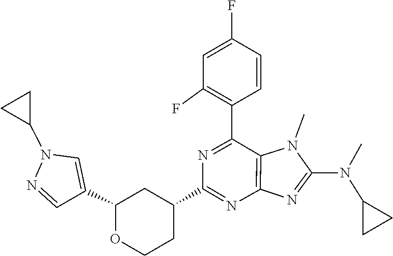 [SMILES: CN(c1nc2nc([C@@H]3CCO[C@H](c4cnn(C5CC5)c4)C3)nc(-c3ccc(F)cc3F)c2n1C)C1CC1] |
| I-302 | 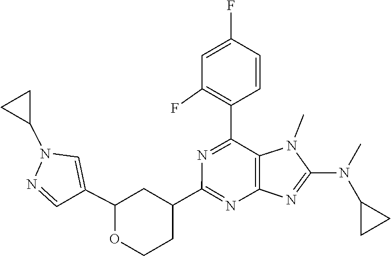 [SMILES: CN(c1nc2nc(C3CCOC(c4cnn(C5CC5)c4)C3)nc(-c3ccc(F)cc3F)c2n1C)C1CC1] |
| I-303 | 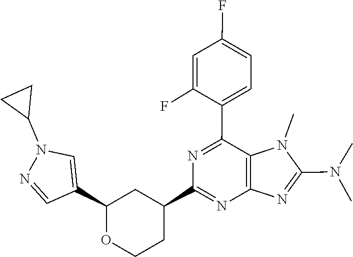 [SMILES: CN(C)c1nc2nc([C@H]3CCO[C@@H](c4cnn(C5CC5)c4)C3)nc(-c3ccc(F)cc3F)c2n1C] |
| I-304 | 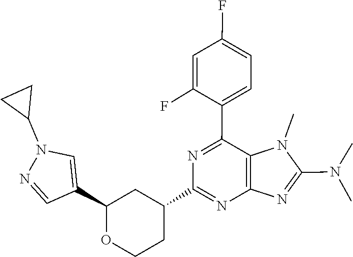 [SMILES: CN(C)c1nc2nc([C@@H]3CCO[C@@H](c4cnn(C5CC5)c4)C3)nc(-c3ccc(F)cc3F)c2n1C] |
| I-305 | 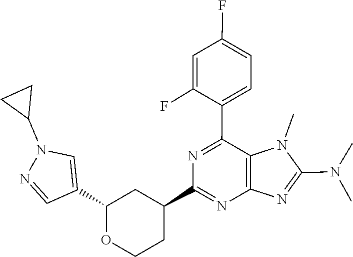 [SMILES: CN(C)c1nc2nc([C@H]3CCO[C@H](c4cnn(C5CC5)c4)C3)nc(-c3ccc(F)cc3F)c2n1C] |
| I-306 | 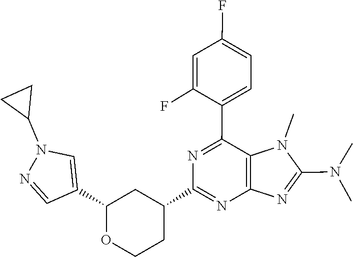 [SMILES: CN(C)c1nc2nc([C@@H]3CCO[C@H](c4cnn(C5CC5)c4)C3)nc(-c3ccc(F)cc3F)c2n1C] |
| I-307 | 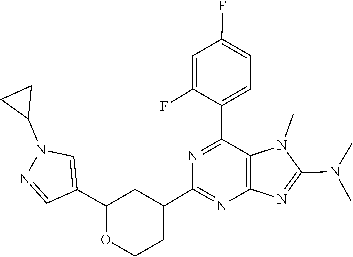 [SMILES: CN(C)c1nc2nc(C3CCOC(c4cnn(C5CC5)c4)C3)nc(-c3ccc(F)cc3F)c2n1C] |
| I-308 | 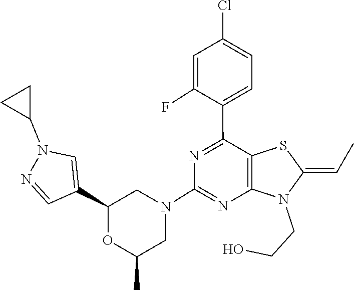 [SMILES: C/C=C1\Sc2c(-c3ccc(Cl)cc3F)nc(N3C[C@@H](C)O[C@@H](c4cnn(C5CC5)c4)C3)nc2N1CCO] |
| I-309 | 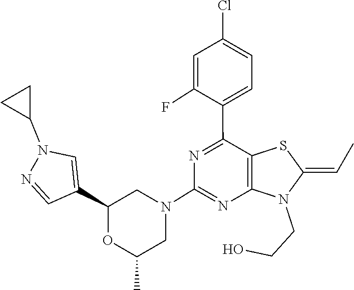 [SMILES: C/C=C1\Sc2c(-c3ccc(Cl)cc3F)nc(N3C[C@H](C)O[C@@H](c4cnn(C5CC5)c4)C3)nc2N1CCO] |
| I-310 | 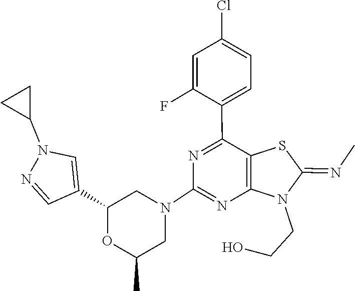 [SMILES: C/N=c1\sc2c(-c3ccc(Cl)cc3F)nc(N3C[C@@H](C)O[C@H](c4cnn(C5CC5)c4)C3)nc2n1CCO] |
| I-311 | 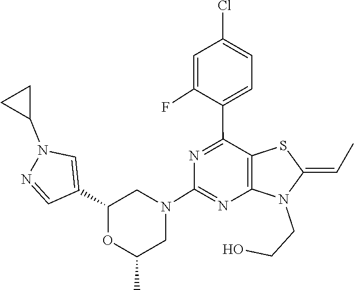 [SMILES: C/C=C1\Sc2c(-c3ccc(Cl)cc3F)nc(N3C[C@@H](c4cnn(C5CC5)c4)O[C@@H](C)C3)nc2N1CCO] |
| I-312 | 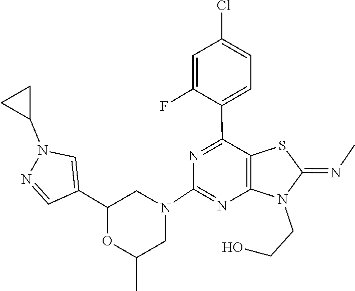 [SMILES: C/N=c1\sc2c(-c3ccc(Cl)cc3F)nc(N3CC(C)OC(c4cnn(C5CC5)c4)C3)nc2n1CCO] |

The foregoing merely summarizes certain aspects of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

In one aspect, the invention provides a pharmaceutical composition comprising a compound of the present disclosure, or a tautomer thereof, or a pharmaceutically acceptable salt of said compound or said tautomer, and a pharmaceutically acceptable excipient.

In another aspect, the invention provides a compound of the present disclosure, or a tautomer thereof, or a pharmaceutically acceptable salt of said compound or said tautomer, or a pharmaceutical composition comprising said compound, or said tautomer, or said salt, for use as a medicament.

### Pharmaceutically Acceptable Compositions

According to some embodiments, the present disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably activate a TREM2 protein, or a mutant thereof, in a biological sample or in a patient. In certain embodiments, the amount of compound in compositions of this disclosure is such that it is effective to measurably activate a TREM2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be affected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

Without wishing to be bound by any particular theory, the following is noted: TREM2 has been implicated in several myeloid cell processes, including phagocytosis, proliferation, survival, and regulation of inflammatory cytokine production. Ulrich and Holtzman 2016. In the last few years, TREM2 has been linked to several diseases. For instance, mutations in both TREM2 and DAP12 have been linked to the autosomal recessive disorder Nasu-Hakola Disease, which is characterized by bone cysts, muscle wasting and demyelination phenotypes. Guerreiro et al. 2013. More recently, variants in the TREM2 gene have been linked to increased risk for Alzheimer's disease (AD) and other forms of dementia including frontotemporal dementia. Jonsson et al. 2013, Guerreiro, Lohmann et al. 2013, and Jay, Miller et al. 2015. In particular, the R47H variant has been identified in genome-wide studies as being associated with increased risk for late-onset AD with an overall adjusted odds ratio (for populations of all ages) of 2.3, second only to the strong genetic association of ApoE to Alzheimer's. The R47H mutation resides on the extracellular lg V-set domain of the TREM2 protein and has been shown to impact lipid binding and uptake of apoptotic cells and Abeta (Wang et al. 2015; Yeh et al. 2016), suggestive of a loss-of-function linked to disease. Further, postmortem comparison of AD patients' brains with and without the R47H mutation are supportive of a novel loss-of-microglial barrier function for the carriers of the mutation, with the R47H carrier microglia putatively demonstrating a reduced ability to compact plaques and limit their spread. Yuan et al. 2016. Impairment in microgliosis has been reported in animal models of prion disease, multiple sclerosis, and stroke, suggesting that TREM2 may play an important role in supporting microgliosis in response to pathology or damage in the central nervous system. Ulrich and Holtzman 2016. In addition, knockdown of TREM2 has been shown to aggravate a-syn-induced inflammatory responses in vitro and exacerbate dopaminergic neuron loss in response to AAV-SYN in vivo (a model of Parkinson's disease), suggesting that impaired microglial TREM2 signaling exacerbates neurodegeneration by modulating microglial activation states. Guo et. al. 2019. A variety of animal models also suggest that Toll-Like Receptor (TLR) signaling is important in the pathogenesis of Rheumatoid Arthritis (RA) via persistent expression of pro-inflammatory cytokines by macrophages. Signaling through TREM2/DAP12 inhibits TLR responses by reducing MAPK (Erk1/2) activation, suggesting that TREM2 activation may act as a negative regulator of TLR driven RA pathogenesis. Huang and Pope 2009.

In view of the data indicating that deficits in TREM2 activity affect macrophage and microglia function, the compounds disclosed herein are of particular use in disorders, such as those described above and in the embodiments that follow and in neurodegenerative disorders more generally.

In one aspect, the invention provides a compound of the present disclosure, or a tautomer thereof, or a pharmaceutically acceptable salt of said compound or said tautomer, or a pharmaceutical composition thereof for use in treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in treating or preventing Parkinson's disease, rheumatoid arthritis, Alzheimer's disease, Nasu-Hakola disease, frontotemporal dementia, multiple sclerosis, prion disease, or stroke.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing Parkinson's disease, rheumatoid arthritis, Alzheimer's disease, Nasu-Hakola disease, frontotemporal dementia, multiple sclerosis, prion disease, or stroke.

In another aspect, the invention provides a method of treating or preventing a condition associated with a loss of function of human TREM2 in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

In another aspect, the invention provides a method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

### CSF1R

CSF1R is a cell-surface receptor primarily for the cytokine colony stimulating factor 1 (CSF-1), also known until recently as macrophage colony-stimulating factor (M-CSF), which regulates the survival, proliferation, differentiation and function of mononuclear phagocytic cells, including microglia of the central nervous system. CSF1R is composed of a highly glycosylated extracellular ligand-binding domain, a trans-membrane domain and an intracellular tyrosine-kinase domain. Binding of CSF-1 to CSF1R results in the formation of receptor homodimers and subsequent auto-phosphorylation of several tyrosine residues in the cytoplasmic domain, notably Syk. In the brain, CSF1R is predominantly expressed in microglial cells. It has been found that microglia in CSF1R+/−patients are depleted and show increased apoptosis (Oosterhof et al., 2018).

The present invention relates to the unexpected discovery that administration of a TREM2 agonist can rescue the loss of microglia in cells having mutations in CSF1R. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in CSF1R signaling caused by a decrease in the concentration of its ligand. In a 5×FAD murine Alzheimer's disease model of amyloid pathology, doses of a CSF1R inhibitor that almost completely eliminate microglia in the brains of wild-type animals show surviving microglia clustered around the amyloid plaques (Spangenberg et al, Nature Communications 2019). Plaque amyloid has been demonstrated in the past to be a ligand for TREM2, and it has been shown that microglial engagement with amyloid is dependent on TREM2 (Condello et al, Nat Comm., 2015). The present invention relates to the unexpected discovery that it is activation of TREM2 that rescued the microglia in the presence of the CSF1R inhibitor, and that this effect is also observed in patients suffering from loss of microglia due to CSF1R mutation. This discovery has not been previously taught or suggested in the available art.

Adult-onset leukoencephalopathy with axonal spheroids and pigmented glia (ALSP), previously recognized as hereditary diffuse leukoencephalopathy with axonal spheroids (HDLS) or pigmentary orthochromatic leukodystrophy (POLD), is an autosomal-dominant central nervous system disease that manifests in the form of variable behavioral, cognitive and motor function changes in patients suffering from the disease. ALSP is characterized by patchy cerebral white matter abnormalities visible by magnetic resonance imaging. However, the clinical symptoms and MRI changes are not specific to ALSP and are common for other neurological conditions, including Nasu-Hakola disease (NHD) and AD, making diagnosis and treatment of ALSP very difficult.

Recent studies have discovered that ALSP is a Mendelian disorder in which patients carry a heterozygous loss of function mutation in the kinase domain of CSF1R, suggesting a reduced level of signaling on the macrophage colony-stimulating factor (M-CSF)/CSF1R axis (Rademakers et al, Nat Genet 2012; Konno et al, Neurology 2018). In one aspect, the present invention relates to the surprising discovery that activation of the TREM2 pathway can rescue the loss of microglia in CSF1R+/−ALSP patients, preventing microglia apoptosis, thereby treating the ALSP condition.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of Colony stimulating factor 1 receptor (CSF1R, also known as macrophage colony-stimulating factor receptor/M-CSFR, or cluster of differentiation 115/CD115).

In one aspect, the invention provides a compound of the present disclosure, or a tautomer thereof, or a pharmaceutically acceptable salt of said compound or said tautomer, or a pharmaceutical composition thereof for use in treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of CSF1R.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In another aspect, the invention provides a method of treating or preventing a disease or disorder associated with dysfunction of CSF1R in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the subject is selected for treatment based on a diagnosis that includes the presence of a mutation in a CSF1R gene affecting the function of CSF1R. In some embodiments, the mutation in the CSF1R gene is a mutation that causes a decrease in CSF1R activity or a cessation of CSF1R activity. In some embodiments, the disease or disorder is caused by a heterozygous CSF1R mutation. In some embodiments, the disease or disorder is caused by a homozygous CSF1R mutation. In some embodiments, the disease or disorder is caused by a splice mutation in the csf1r gene. In some embodiments, the disease or disorder is caused by a missense mutation in the csf1r gene. In some embodiments, the disease or disorder is caused by a mutation in the catalytic kinase domain of CSF1R. In some embodiments, the disease or disorder is caused by a mutation in an immunoglobulin domain of CSF1R. In some embodiments, the disease or disorder is caused by a mutation in the ectodomain of CSF1R. In some embodiments, the disease or disorder is a disease or disorder resulting from a change (e.g. increase, decrease or cessation) in the activity of CSF1R. In some embodiments, the disease or disorder is a disease or disorder resulting from a decrease or cessation in the activity of CSF1R. CSF1R related activities that are changed in the disease or disorder include, but are not limited to: decrease or loss of microglia function; increased microglia apoptosis; decrease in Src signaling; decrease in Syk signaling; decreased microglial proliferation; decreased microglial response to cellular debris; decreased phagocytosis; and decreased release of cytokines in response to stimuli. In some embodiments, the disease or disorder is caused by a loss-of-function mutation in CSF1R. In some embodiments, the loss-of-function mutation results in a complete cessation of CSF1R function. In some embodiments, the loss-of-function mutation results in a partial loss of CSF1R function, or a decrease in CSF1R activity.

In another aspect, the invention provides a method of treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats or prevents ALSP, which is an encompassing and superseding name for both HDLS and POLD. In some embodiments, the disease or disorder is a homozygous mutation in CSF1R. In some embodiments, the method treats or prevents pediatric-onset leukoencephalopathy. In some embodiments, the method treats or prevents congenital absence of microglia. In some embodiments, the method treats or prevents brain abnormalities neurodegeneration and dysosteosclerosis (BANDDOS).

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting CSF1R dysfunction, or having a mutation in a gene affecting the function of CSF1R, the method comprising administering to the subject a therapeutically effective amount of a compound of the present disclosure, or a tautomer thereof, or a pharmaceutically acceptable salt of said compound or said tautomer, or a pharmaceutical composition thereof.

### ABCD1

The ABCD1 gene provides instructions for producing the adrenoleukodystrophy protein (ALDP). ABCD1 (ALDP) maps to Xq28. ABCD1 is a member of the ATP-binding cassette (ABC) transporter superfamily. The superfamily contains membrane proteins that translocate a wide variety of substrates across extra- and intracellular membranes, including metabolic products, lipids and sterols, and drugs. ALDP is located in the membranes of cell structures called peroxisomes. Peroxisomes are small sacs within cells that process many types of molecules. ALDP brings a group of fats called very long-chain fatty acids (VLCFAs) into peroxisomes, where they are broken down. As ABCD1 is highly expressed in microglia, it is possible that microglial dysfunction and their close interaction with other cell types actively participates in neurodegenerative processes (Gong et al., Annals of Neurology. 2017; 82(5):813-827.). It has been shown that severe microglia loss and damage is an early feature in patients with cerebral form of x-linked ALD (cALD) carrying ABCD1 mutations (Bergner et al., Glia. 2019; 67: 1196-1209). It has also been shown that ABCD1-deficiency leads to an impaired plasticity of myeloid lineage cells that is reflected in incomplete establishment of anti-inflammatory responses, thus possibly contributing to the devastating rapidly progressive demyelination in cerebral adrenoleukodystrophy (Weinhor et al., BRAIN 2018: 141; 2329-2342). These findings emphasize microglia/monocytes/macrophages as crucial therapeutic targets for preventing or stopping myelin destruction in patients with X-linked adrenoleukodystrophy.

The present invention relates to the unexpected discovery that administration of a TREM2 agonist can rescue the loss of microglia in cells having mutations in the ABCD1 gene. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in ABCD1 function leading to sustained activation, proliferation, chemotaxis of microglia, maintenance of anti-inflammatory environment and reduced astrocytosis caused by a decrease in ABCD1 and accumulation of VLCFAs. The present invention relates to the unexpected discovery that activation of TREM2 can rescue the microglia in the presence of the ABCD1 mutation and an increase in VLCFA, and that this effect may be also observed in patients suffering from loss of microglia due to ABCD1 mutation. This discovery has not been previously taught or suggested in the available art.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of ATP-binding cassette transporter 1 (ABCD1).

In one aspect, the invention provides a compound of the present disclosure, or a tautomer thereof, or a pharmaceutically acceptable salt of said compound or said tautomer, or a pharmaceutical composition thereof for use in treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of ABCD1.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In yet another aspect, the invention provides a method of treating or preventing a disease or disorder associated with dysfunction of ABCD1 in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the patient is selected for treatment based on a diagnosis that includes the presence of a mutation in an ABCD1 gene affecting the function of ABCD1. In some embodiments, the mutation in the ABCD1 gene is a mutation that causes a decrease in ABCD1 activity or a cessation of ABCD1 activity. In some embodiments, the disease or disorder is caused by a heterozygous ABCD1 mutation. In some embodiments, the disease or disorder is caused by a homozygous ABCD1 mutation. In some embodiments, the disease or disorder is caused by a splice mutation in the ABCD1 gene. In some embodiments, the disease or disorder is caused by a missense mutation in the ABCD1 gene. In some embodiments, the disease or disorder is a disease or disorder resulting from a change (e.g. increase, decrease or cessation) in the activity of ABCD1. In some embodiments, the disease or disorder is a disease or disorder resulting from a decrease or cessation in the activity of ABCD1. ABCD1 related activities that are changed in the disease or disorder include, but are not limited to peroxisomal import of fatty acids and/or fatty acyl-CoAs and production of adrenoleukodystrophy protein (ALDP). In some embodiments, the disease or disorder is caused by a loss-of-function mutation in ABCD1. In some embodiments, the loss-of-function mutation results in a complete cessation of ABCD1 function. In some embodiments, the loss-of-function mutation results in a partial loss of ABCD1 function, or a decrease in ABCD1 activity. In some embodiments, the disease or disorder is caused by a homozygous mutation in ABCD1. In some embodiments, the disease or disorder is a neurodegenerative disorder. In some embodiments, the disease or disorder is a neurodegenerative disorder caused by and/or associated with an ABCD1 dysfunction. In some embodiments, the disease or disorder is an immunological disorder. In some embodiments, the disease or disorder is an immunological disorder caused by and/or associated with an ABCD1 dysfunction.

In yet another aspect, the invention provides a method of treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, any of the aforementioned diseases are present in a patient exhibiting ABCD1 dysfunction or having a mutation in a gene affecting the function of ABCD1. In some embodiments, the method treats or prevents X-linked adrenoleukodystrophy (x-ALD). In some embodiments, the x-ALD is a cerebral form of x-linked ALD (cALD). In some embodiments, the method treats or prevents Addison disease wherein the patient has been found to have a mutation in one or more ABCD1 genes affecting ABCD1 function. In some embodiments, the method treats or prevents Addison disease, wherein the patient has a loss-of-function mutation in ABCD1.

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), or Parkinson's disease, wherein any of the aforementioned diseases or disorders are present in a patient exhibiting ABCD1 dysfunction, or having a mutation in a gene affecting the function of ABCD1, the method comprising administering to the subject a therapeutically effective amount of a compound of the present disclosure, or a tautomer thereof, or a pharmaceutically acceptable salt of said compound or said tautomer, or a pharmaceutical composition thereof.

### Autism Spectrum Disorders

It has been found that TREM2 deficient mice exhibit symptoms reminiscent of autism spectrum disorders (ASDs) (Filipello et al., Immunity, 2018, 48, 979-991). It has also been found that microglia depletion of the autophagy Aatg7 gene results in defective synaptic pruning and results in increased dendritic spine density, and abnormal social interaction and repetitive behaviors indicative of ASDs (Kim, et al., Molecular Psychiatry, 2017, 22, 1576-1584.). Further studies have shown that increased dendritic spin density detected in post-mortem ASD brains, likely caused by defective synaptic pruning, results in circuit hypoconnectivity and behavioral defects and are a potential origin of a number of neurodevelopmental diseases (Tang, et al., Neuron, 2014, 83, 1131-1143). Without intending to be limited to any particular theory, these findings suggest that TREM2 activation can reverse microglia depletion, and therefore correct the defective synaptic pruning that is central to neurodevelopmental diseases such as ASDs. The present invention relates to the unexpected discovery that activation of TREM2, using a compound of the present invention, can rescue microglia in subjects suffering from an ASD. This discovery has not been previously taught or suggested in the available art.

In another aspect, the present invention provides a compound of the present disclosure, or a tautomer thereof, or a pharmaceutically acceptable salt of said compound or said tautomer, or a pharmaceutical composition thereof for use in treating autism or autism spectrum disorders.

In yet another aspect, the present invention provides a compound of the present disclosure, or a tautomer thereof, or a pharmaceutically acceptable salt of said compound or said tautomer, or a pharmaceutical composition thereof for use in the preparation of a medicament for treating autism or autism spectrum disorders.

In yet another aspect, the present invention provides a method of treating autism or autism spectrum disorders in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats autism. In some embodiments, the method treats Asperger syndrome.

In some embodiments, the disclosure provides a method of increasing the activity of TREM2, the method comprising contacting a compound of the present disclosure, or a pharmaceutically acceptable salt thereof with the TREM2. In some embodiments, the contacting takes place in vitro. In some embodiments, the contacting takes place in vivo. In some embodiments, the TREM2 is human TREM2.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this disclosure may also be combined with include, without limitation: treatments for Parkinson's disease, rheumatoid arthritis, Alzheimer's disease, Nasu-Hakola disease, frontotemporal dementia, multiple sclerosis, prion disease, or stroke.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### Definitions

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. For example, (1R)-1-methyl-2-(trifluoromethyl)cyclohexane is meant to encompass (1R,2R)-1-methyl-2-(trifluoromethyl)cyclohexane and (1R,2S)-1-methyl-2-(trifluoromethyl)cyclohexane. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

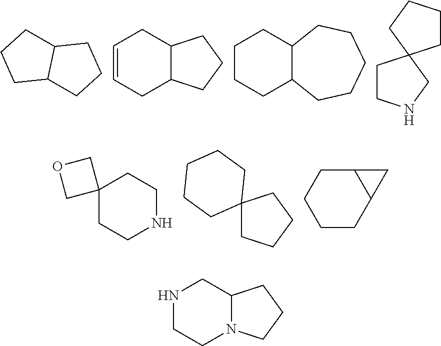

Exemplary bridged bicyclics include:

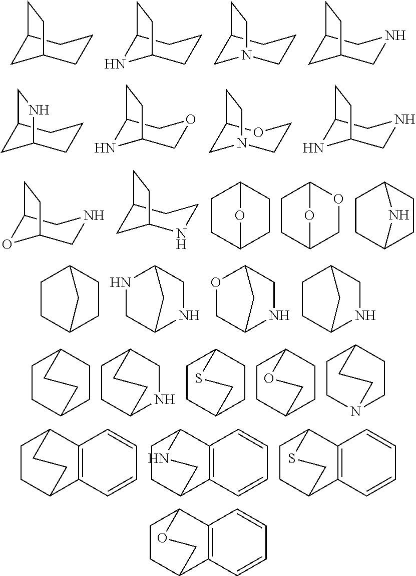

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋ₛ(or C₁-6) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14□ electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆R^{∘}; —(CH₂)₀₋₆OR^{∘}; —O(CH₂)₀₋₆R^{∘}, —O—(CH₂)₀₋₆C(O)OR^{∘}; —(CH₂)₀₋₆CH(OR^{∘})₂; —(CH₂)₀₋₆SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂; —CN; —N₃; —(CH₂)₀₋₆N(R^{∘})₂; —(CH₂)₀₋₆N(R^{∘})C(O)R^{∘}; —N(R^{∘})C(S)R^{∘}; —(CH₂)₀₋₆N(R^{∘})C(O)NR^{∘}₂; —N(R^{∘})C(S)NR^{∘}₂; —(CH₂)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆C(O)R^{∘}; —C(S)R^{∘}; —(CH₂)₀₋₆C(O)OR^{∘}; —(CH₂)₀₋₆C(O)SR^{∘}; —(CH₂)₀₋₆C(O)OSiR^{∘}₃; —(CH₂)₀₋₆OC(O)R^{∘}; —OC(O)(CH₂)₀₋₆SR^{∘}, —(CH₂)₀₋₆SC(O)R^{∘}; —(CH₂)₀₋₆C(O)NR^{∘}₂; —C(S)NR^{∘}₂; —C(S)SR^{∘}; —SC(S)SR^{∘}, —(CH₂)₀₋₆OC(O)NRR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₆SSR^{∘}; —(CH₂)₀₋₆S(O)₂R^{∘}; —(CH₂)₀₋₆S(O)₂OR^{∘}; —(CH₂)₀₋₆OS(O)₂R^{∘}; —S(O)₂NR^{∘}₂; —(CH₂)₀₋₆S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R¹⁰ may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*2)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of R^{†}, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

As used herein, the term “provided compound” or “compound of the present disclosure” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N*(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

The terms “patient” and “subject” as used herein refer to humans and mammals, including, but not limited to, primates, cows, sheep, goats, horses, dogs, cats, rabbits, rats, and mice. In one embodiment the subject is a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

The terms “C₁₋₃alkyl,” “C₁₋₅alkyl,” and “C₁₋₆alkyl” as used herein refer to a straight or branched chain hydrocarbon containing from 1 to 3, 1 to 5, and 1 to 6 carbon atoms, respectively. Representative examples of C₁₋₃alkyl, C₁₋₅alky, or C₁₋₆alkyl include, but are not limited to, methyl, ethyl, n-propyl, iso-propyl, n-butyl, sec-butyl, iso-butyl, tert-butyl, pentyl and hexyl.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₃₋₆cycloalkyl” as used herein refers to a saturated carbocyclic molecule wherein the cyclic framework has 3 to 6 carbon atoms. Representative examples of C₃₋₅cycloalkyl include, but are not limited to, cyclopropyl, cyclobutyl, cyclopentyl, and cyclohexyl.

The terms “diC₁₋₃alkylamino” as used herein refer to —NR*R**, wherein R* and R** independently represent a C₁₋₃alkyl as defined herein. Representative examples of diC₁₋₃alkylamino include, but are not limited to, —N(CH₃)₂, —N(CH₂CH₃)₂, —N(CH₃)(CH₂CH₃), —N(CH₂CH₂CH₃)₂, and —N(CH(CH₃)₂)₂.

The term “C₁₋₃alkoxy” and “C₁₋₆alkoxy” as used herein refer to —OR^{#}, wherein R^{#} represents a C₁₋₃alkyl and C₁₋₆alkyl group, respectively, as defined herein. Representative examples of C₁₋₃alkoxy or C₁₋₆alkoxy include, but are not limited to, methoxy, ethoxy, propoxy, iso-propoxy, and butoxy.

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆heterocycloalkyl” as used herein refers to a saturated carbocyclic molecule wherein the cyclic framework has 3 to 6 carbons and wherein one carbon atom is substituted with a heteroatom selected from N, O, and S. If the C₃₋₆heterocycloalkyl group is a C₆heterocycloalkyl, one or two carbon atoms are substituted with a heteroatom independently selected from N, O, and S. Representative examples of C₃₋₆heterocycloalkyl include, but are not limited to, aziridinyl, azetidinyl, oxetanyl, pyrrolidinyl, piperazinyl, morpholinyl, and thiomorpholinyl.

The term “C₅₋₈spiroalkyl” as used herein refers a bicyclic ring system, wherein the two rings are connected through a single common carbon atom. Representative examples of C₅₋₈spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈tricycloalkyl” as used herein refers a tricyclic ring system, wherein all three cycloalkyl rings share the same two ring atoms. Representative examples of C₅₋₈tricycloalkyl include, but are not limited to, tricyclo[1.1.1.0^{1,3}]pentanyl,

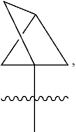 [SMILES: *C12CC(C1)C2]

tricyclo[2.1.1.0^{1,4}]hexanyl, tricyclo[3.1.1.0^{1,5}]hexanyl, and tricyclo[3.2.1.0^{1,5}]octanyl.

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following scheme are the variables as defined for Formula I, unless otherwise noted. All starting materials are either commercially available, for example, from Merck Sigma-Aldrich Inc. and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein. As used below, Z is a leaving group, which can include but is not limited to, halogens (e.g. fluoride, chloride, bromide, iodide), sulfonates (e.g. mesylate, tosylate, benzenesulfonate, brosylate, nosylate, triflate), diazonium, and the like. As used below, in certain embodiments Y is an organometal coupling reagent group, which can include but are not limited to, boronic acids and esters, organotin and organozinc reagents.

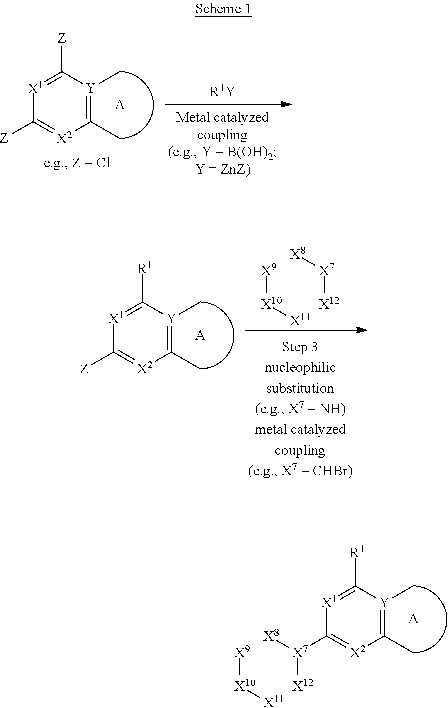

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid and gas phase), extraction, distillation, trituration, and reverse phase HPLC.

The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

### EXAMPLES

This section provides specific examples of compounds of Formula I and methods of making the same.

List of Abbreviations

| | |
|---|---|
| aq or aq. | aqueous |
| DCM | dichloromethane |
| DMAP | 4-dimethylaminopyridine |
| DMF | N,N-dimethylformamide |
| DMSO | dimethyl sulfoxide |
| Dppf, DPPF or dppf | 1,1′-bis(diphenylphosphino)ferrocene |
| eq or eq. or equiv. | equivalent |
| ESI or ES | electrospray ionization |
| Et | ethyl |
| EtOAc or EA | ethyl acetate |
| g | gram(s) |
| h or hr | hour(s) |
| HPLC | high pressure liquid chromatography |
| iPr | isopropyl |
| iPr₂NEt or DIPEA | N-ethyl diisopropylamine (Hunig's base) |
| LC MS, LCMS, LC-MS or | liquid chromatography mass spectroscopy |
| LC/MS | |
| m/z | mass divided by charge |
| Me | methyl |
| CH₃CN | acetonitrile |
| MeOH | methanol |
| mg | milligrams |
| min | minutes |
| mL | milliliters |
| MS | mass spectra |
| n-BuLi | n-butyllithium |
| NMR | nuclear magnetic resonance |
| PE | Petroleum ether |
| Ph | phenyl |
| RT or rt or r.t. | room temperature |
| RuPhos Pd G3 | (2-Dicyclohexylphosphino-2′,6′-diisopropoxy-1,1′- |
| | biphenyl)[2-(2′-amino-1,1′-biphenyl)]palladium(II) |
| | methanesulfonate |
| sat. | saturated |
| SFC | supercritical fluid chromatography |
| TEA or Et₃N | triethylamine |
| THF | tetrahydrofuran |
| Xantphos Pd G3 | [(4,5-Bis(diphenylphosphino)-9,9-dimethylxanthene)-2-(2′- |
| | amino-1,1′-biphenyl)]palladium(II) methanesulfonate |
| PE | Petroleum ether |

### General Analytical and Purification Methods

Provided in this section are descriptions of the general analytical and purification methods used to prepare the specific compounds provided herein.

### Chromatography:

Unless otherwise indicated, crude product-containing residues were purified by passing the crude material or concentrate through either a Biotage brand silica gel column pre-packed with flash silica (SiO₂) or reverse phase flash silica (C18) and eluting the product off the column with a solvent gradient as indicated. For example, a description of silica gel (0-40% EtOAc/hexane) means the product was obtained by elution from the column packed with silica using a solvent gradient of 0% to 40% EtOAc in hexanes.

### Preparative HPLC Method:

Where so indicated, the compounds described herein were purified via reverse phase HPLC using Waters Fractionlynx semi-preparative HPLC-MS system utilizing one of the following two HPLC columns: (a) Phenominex Gemini column (5 micron, C18, 150×30 mm) or (b) Waters X-select CSH column (5 micron, C18, 100×30 mm).

A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

### Analytical HPLC Method:

Where so indicated, the compounds described herein were analyzed using an Aglilent 1100 series instrument with DAD detector.

### Flash Chromatography Method:

Where so indicated, flash chromatography was performed on Teledyne Isco instruments using pre-packaged disposable SiO₂ stationary phase columns with eluent flow rate range of 15 to 200 mL/min, UV detection (254 and 220 nm).

### Preparative Chiral Supercritical Fluid Chromatography (SFC) Method:

Where so indicated, the compounds described herein were purified via chiral SFC using one of the two following chiral SFC columns: (a) Chiralpak IG 2×25 cm, 5 μm or (b) Chiralpak AD-H 2×15 cm, 5 μm.

Some CP Analytical-SFC experiments were run on SFC Method Station (Thar, Waters) with the following conditions: Column temperature: 40° C., Mobile phase: CO₂/Methanol (0.2% Methanol Ammonia)=Flow: 4.0 ml/min, Back Pressure: 120 Bar, Detection wavelength: 214 nm.

Some CP Analytical-SFC experiments were run on SFC-80 (Thar, Waters) with the following conditions: Column temperature: 35° C., Mobile phase (example): CO₂/Methanol (0.2% Methanol Ammonia)=Flow rate: 80 g/min, Back pressure: 100 bar, Detection wavelength: 214 nm.

Preparative CP Method: Acidic reversed phase MPLC: Instrument type: Reveleris™ prep MPLC; Column: Phenomenex LUNA C18(3) (150×25 mm, 10); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.

### Proton NMR Spectra:

Unless otherwise indicated, all ¹H NMR spectra were collected on a Bruker NMR Instrument at 300, 400 or 500 Mhz or a Varian NMR Instrument at 400 Mhz. Where so characterized, all observed protons are reported as parts-per-million (ppm) downfield from tetramethylsilane (TMS) using the internal solvent peak as reference. All NMR were collected at about 25° C.

### Mass Spectra (MS)

Unless otherwise indicated, all mass spectral data for starting materials, intermediates and/or exemplary compounds are reported as mass/charge (m/z), having an [M+H]⁺ molecular ion. The molecular ion reported was obtained by electrospray detection method (commonly referred to as an ESI MS) utilizing a Waters Acquity UPLC/MS system or a Gemini-NX UPLC/MS system. Compounds having an isotopic atom, such as bromine and the like, are generally reported according to the detected isotopic pattern, as appreciated by those skilled in the art.

### Compound Names

The compounds disclosed and described herein have been named using the IUPAC naming function of ChemDraw Professional 17.0.

### Specific Examples

Provided in this section are the procedures to synthesize specific examples of the compounds provided herein. All starting materials are either commercially available from Sigma-Aldrich Inc., unless otherwise noted, or known in the art and may be synthesized by employing known procedures using ordinary skill.

### Example 1—Synthesis of Compounds I-12 and I-14: 5-[(2R,4S)-2-(1-cyclopropylpyrazol-4-yl)tetrahydropyran-4-yl]-7-(2,4-difluorophenyl)-2-methyl-thiazolo[4,5-d]pyrimidine and 5-[(2R,4R)-2-(1-cyclopropylpyrazol-4-yl) tetrahydropyran-4-yl]-7-(2,4-difluorophenyl)-2-methyl-thiazolo[4,5-d]pyrimidine, respectively

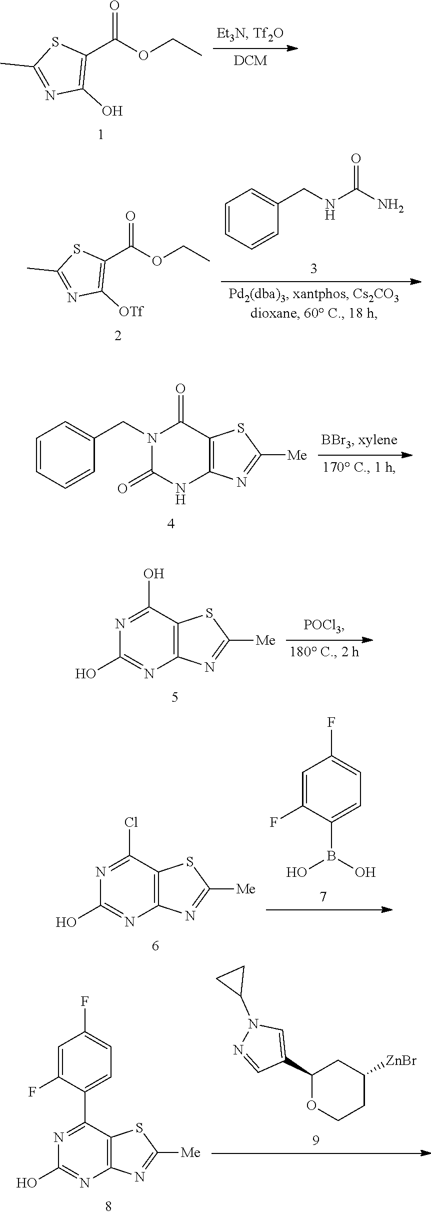

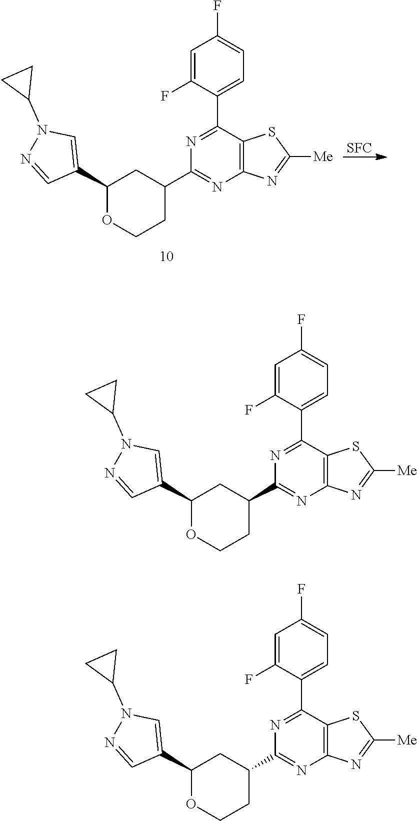

Step 1: To a solution of ethyl 4-hydroxy-2-methyl-thiazole-5-carboxylate (1.00 eq, 4.90 g, 26.2 mmol) in DCM (60 mL) was added TEA (1.50 eq, 5.4 mL, 39.3 mmol) and TRIFLUOROMETHANESULFONIC ANHYDRIDE (1.10 eq, 4.9 mL, 28.8 mmol) at −78° C., the mixture was stirred at −78° C. for 4 h. The mixture was concentrated to give a crude product. The crude product was purified by column chromatography on silica gel eluted with EA (0˜10%) in PE. ethyl 2-methyl-4-(trifluoromethylsulfonyloxy)thiazole-5-carboxylate (6.70 g, 21.0 mmol, 80.18% yield) was obtained as yellow oil. LC-MS: Rt: 0.860 min; m/z: 320.0 [M+H]+; 100% purity at 220 nm.

Step 2: To a solution of ethyl 2-methyl-4-(trifluoromethylsulfonyloxy)thiazole-5-carboxylate (1.00 eq, 6.70 g, 21.0 mmol) in 1,4-Dioxane (80 mL) was added benzylurea (1.10 eq, 3467 mg, 23.1 mmol), Cs₂CO₃ (2.00 eq, 13640 mg, 42.0 mmol), XantPhos (0.0500 eq, 607 mg, 1.05 mmol) and Pd₂(dba)₃ (0.0250 eq, 480 mg, 0.525 mmol), the mixture was stirred at 60° C. for 12 h. The mixture was poured into water and stirred for 15 min, then the mixture was filtered and the filtrate collected and reduced in volume in vacuo to approximately one-third. Then the solution was adjusted to pH=7 with HCl aq (1 mol/L). The resultant precipitate was collected by filtration, washed with EtOAc and dried in vacuo to give a crude product. The crude product used for next step without further purification. 6-benzyl-2-methyl-4H-thiazolo[4,5-d]pyrimidine-5,7-dione (2.40 g, 7.99 mmol, 38.08% yield) was obtained as yellow solid. LC-MS: Rt: 0.700 min; m/z: 274.1 [M+H]+; 90% purity at 220 nm.

Step 3: To a solution of 6-benzyl-2-methyl-4H-thiazolo[4,5-d]pyrimidine-5,7-dione (1.00 eq, 1.40 g, 5.12 mmol) in m-Xylene (20 mL) was added BBr3 (4.00 eq, 1.9 mL, 20.5 mmol) at 25° C., the mixture was stirred at 170° C. for 1 h. After the mixture was cooled to room temperature, the mixture was poured into MeOH (100 mL) at 0° C., The reaction mixture was filtered and the filter cake was washed with MeOH and H2O, dried in vacuum to give a crude product. The crude product used for next step without further purification. 2-methylthiazolo[4,5-d]pyrimidine-5,7-diol (780 mg, 4.26 mmol, 83.12% yield) was obtained as yellow solid. 1H NMR (400 MHz, DMSO-d6) δ=12.27 (s, 1H), 11.32 (s, 1H), 2.74 (s, 3H)

Step 4: To a solution of 2-methylthiazolo[4,5-d]pyrimidine-5,7-diol (1.00 eq, 1.00 g, 5.46 mmol) in POCl₃ (23.5 eq, 12 mL, 128 mmol) was added N,N-DIMETHYLANILINE (0.700 eq, 0.48 mL, 3.82 mmol), the mixture was stirred at 130° C. for 4 h. The reaction mixture was poured into water (1000 mL). The mixture was stirred at 30° C. for 30 min. The aqueous phase was extracted with DCM (1000 mL*3). The combined organic phase was washed with brine (1000 mL*3), dried with anhydrous Na2SO4, filtered and concentrated to give a crude product in vacuum. The crude product was purified by column chromatography use EA (0˜25%) in PE. 5,7-dichloro-2-methyl-thiazolo[4,5-d]pyrimidine (350 mg, 1.59 mmol, 29.13% yield) was obtained as red solid. 1H NMR (400 MHz, CHLOROFORM-d) δ=2.99 (s, 3H)

Step 5: To the mixture of 5,7-dichloro-2-methyl-thiazolo[4,5-d]pyrimidine (1.00 eq, 100 mg, 0.454 mmol), (2,4-difluorophenyl)boronic acid (1.05 eq, 75 mg, 0.477 mmol) and Pd(dppf)Cl2·DCM (0.200 eq, 74 mg, 0.0909 mmol) in 1,4-Dioxane (20 mL) was added a a solution of Cs₂CO₃ (1.05 eq, 155 mg, 0.477 mmol) in Water (2.5 mL). The resulting mixture was bubbled with N₂ for 1 min and stirred at 40° C. for 0.5 h. The reaction mixture was diluted with EtOAc (20 mL). brine (10 mL*2) was added to wash the system and the organic phase was dried over Na₂SO₄, concentrated to dryness to give a residue. The crude product was purified by prep-TLC (DCM). 5-chloro-7-(2,4-difluorophenyl)-2-methyl-thiazolo[4,5-d]pyrimidine (110 mg, 0.358 mmol, 78.88% yield) was obtained as yellow solid. LC-MS: Rt: 0.808 min; m/z: 298.0 [M+H]⁺, 97% purity at 220 nm. 1H NMR (400 MHz, CHLOROFORM-d) 6=7.83 (d, J=6.4 Hz, 1H), 7.10-7.01 (m, 1H), 6.95 (ddd, J=2.4, 8.5, 10.8 Hz, 1H), 2.90 (s, 3H)

Step 6: To a suspension of 5-chloro-7-(2,4-difluorophenyl)-2-methyl-thiazolo[4,5-d]pyrimidine (1.00 eq, 110 mg, 0.369 mmol) and C-phos (0.100 eq, 16 mg, 0.0369 mmol) in THF (3 mL) (99.5%, Extra Dry over Molecular Sieve, Stabilized, Acros) was added PALLADIUM(II) ACETATE (0.0500 eq, 4.1 mg, 0.0185 mmol) followed by bromo-[(2R,4R)-2-(1-cyclopropylpyrazol-4-yl)tetrahydropyran-4-yl]zinc (1.71 eq, 2.6 mL, 0.630 mmol) and the mixture was then stirred at 55° C. for 2 h. The mixture was cooled to rt, diluted with EtOAc (20 mL), sat. NaHCO₃ (20 mL) and sat. NaHSO₃ (2 mL). The phase were separated and extracted by EtOAc (20 mL*2). The organic phase was washed with brine and dried over Na₂SO₄, filtered and concentrated in vacuo. The residue was purified by flash silica gel chromatography (Silica Flash Column, Eluent of 0˜50% Ethyl acetate/Petroleum ether gradient) to give 5-[(2R)-2-(1-cyclopropylpyrazol-4-yl)tetrahydropyran-4-yl]-7-(2,4-difluorophenyl)-2-methyl-thiazolo[4,5-d]pyrimidine (67 mg, 0.148 mmol, 39.98% yield) as yellow gum. LC-MS: Rt: 0.858 min; m/z: 454.1 [M+H]+, 100% purity at 220 nm.

Step 7: The mixture of 5-[(2R)-2-(1-cyclopropylpyrazol-4-yl)tetrahydropyran-4-yl]-7-(2,4-difluorophenyl)-2-methyl-thiazolo[4,5-d]pyrimidine (1.00 eq, 67 mg, 0.148 mmol) were separated by chiral SFC eluting with CO₂/0.1% NH₃H₂O MeOH B %: 50%-50%, DAICEL CHIRALPAK AD (250 mm*30 mm, 10 um). 5-[(2R,4S)-2-(1-cyclopropylpyrazol-4-yl)tetrahydropyran-4-yl]-7-(2,4-difluorophenyl)-2-methyl-thiazolo[4,5-d]pyrimidine (47 mg, 0.100 mmol, 67.75% yield) was obtained as white solid, 5-[(2R,4R)-2-(1-cyclopropylpyrazol-4-yl) tetrahydropyran-4-yl]-7-(2,4-difluorophenyl)-2-methyl-thiazolo[4,5-d]pyrimidine (2.6 mg, 0.00549 mmol, 3.72% yield) was obtained as white solid. [00291]1-79: LC-MS: Rt: 0.857 min; m/z: 454.2 [M+H]+, 97.2% purity at 220 nm. 1H NMR (400 MHz, CHLOROFORM-d) 6=7.86 (dt, J=6.5, 8.4 Hz, 1H), 7.50 (s, 2H), 7.09 (dt, J=1.7, 8.3 Hz, 1H), 7.01 (ddd, J=2.4, 8.6, 10.8 Hz, 1H), 4.54 (dd, J=1.7, 11.4 Hz, 1H), 4.31-4.19 (m, 1H), 3.80 (dt, J=3.8, 11.2 Hz, 1H), 3.55 (tt, J=3.7, 7.2 Hz, 1H), 3.50-3.37 (m, 1H), 2.94 (s, 3H), 2.45-2.35 (m, 1H), 2.23-2.10 (m, 3H), 1.12-1.05 (m, 2H), 1.02-0.92 (m, 2H)

I-80: Rt: 0.857 min; m/z: 454.2 [M+H]+, 95.8% purity at 220 nm. 1H NMR (400 MHz, CHLOROFORM-d) 6=8.21-7.72 (m, 1H), 7.49 (d, J=7.0 Hz, 2H), 7.17-6.83 (m, 2H), 4.88 (d, J=6.0 Hz, 1H), 3.93 (d, J=3.8 Hz, 2H), 3.73-3.50 (m, 2H), 2.97 (s, 3H), 2.74 (d, J=13.3 Hz, 1H), 2.49-2.11 (m, 3H), 1.12-0.93 (m, 4H)

### Example 2—Synthesis of Compound I-17: 5-[(2R,4S)-2-(1-cyclopropylpyrazol-4-yl)tetrahydropyran-4-yl]-7-(2,4-difluorophenyl)-2-methyl-oxazolo[4,5-d]pyrimidine

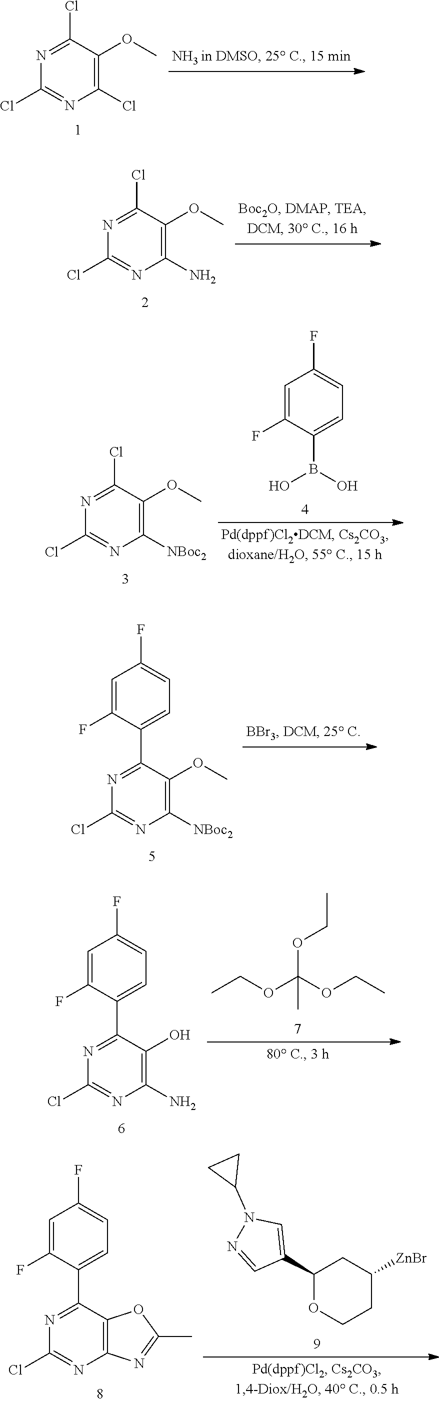

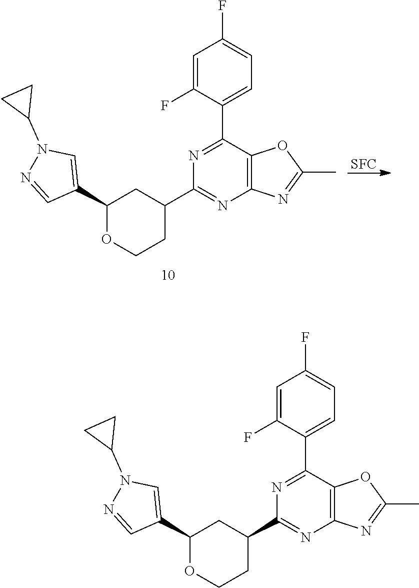

Step 1: A mixture of 2,4,6-trichloro-5-methoxy-pyrimidine (2.5 g, 11.71 mmol, 1 eq) in DMSO (15 mL) was treated with a stream of ammonia gas for 30 min at 25° C. LCMS showed the reaction was completed and a major peak with desired MS (81%, Rt: 0.439 min; [M+H]⁺=194.0 at 220 nm) was detected. The mixture was poured into 100 mL water and extracted with EtOAc (100 mL three times). The organic phase was concentrated under vacuum to give a crude. The crude was purified by flash silica gel chromatography (ISCO®; 20 g SepaFlash® Silica Flash Column, Eluent of 0˜50% Ethyl acetate/Petroleum ethergradient @35 mL/min) to give 2,6-dichloro-5-methoxy-pyrimidin-4-amine (1.8 g, 8.88 mmol, 75.80% yield, 95.7% purity) was obtained as white solid which confirmed with LCMS and 1H NMR. EW30176-52-1 (P1): [M+H]⁺=194.0; purity=95% (220 nm); Retention time=0.366 min ¹H NMR (400 MHz, DMSO-d6) δ=8.28-7.41 (m, 1H), 8.28-7.41 (m, 1H)

Step 2: A mixture of 2,6-dichloro-5-methoxy-pyrimidin-4-amine (1.00 eq, 600 mg, 3.09 mmol), TEA (3.00 eq, 1.3 mL, 9.28 mmol) and DMAP (0.10 eq, 38 mg, 0.309 mmol) in DCM (3 mL) was added di-tert-butyl dicarbonate (2.20 eq, 1.6 mL, 6.80 mmol) at 25° C. The mixture was stirred at 25° C. for 3 h. LCMS showed no starting material remained and a new major peak (94%, Rt: 0.952 min; [M+H]+=238.0 at 220 nm) found. The mixture was concentrated under vacuum to give a crude. The crude was purified by flash column (PE to PE:EtOAc=5:1, R_{f}=0.4) to give tert-butyl N-tert-butoxycarbonyl-N-(2,6-dichloro-5-methoxy-pyrimidin-4-yl)carbamate (1000 mg, 2.51 mmol, 81.12% yield) as colorless oil which confirmed by LCMS and ¹H NMR. SS-2021-01-65-1 (P1): [M+H]⁺=237.9; purity=98% (220 nm); Retention time=0.760 min ¹H NMR (400 MHz, CHLOROFORM-d) 6=3.96-3.86 (m, 3H), 1.50-1.42 (m, 19H).

Step 3: The 1000 mg was separated for two 500 mg reactions. A mixture of tert-butyl N-tert-butoxycarbonyl-N-(2,6-dichloro-5-methoxy-pyrimidin-4-yl)carbamate (1.00 eq, 1000 mg, 2.54 mmol) and (2,4-difluorophenyl)boronic acid (0.90 eq, 360 mg, 2.28 mmol) in 1,4-Dioxane (33 mL) and Water (6.6 mL) was added Cs₂CO₃ (3.00 eq, 2473 mg, 7.61 mmol) and Pd(dppf)Cl₂·DCM (0.10 eq, 206 mg, 0.25 mmol). The mixture was degassed with N₂ for 3 times and stirred at 55° C. for 12 h. LCMS showed starting material remained and desired MS found (39%, Rt: 0.804 min; [M+H]+=472.1 at 220 nm). The reaction was combined and final mixture was concentrated under vacuum to give a crude. The crude was purified by flash column (PE:EtOAc=5:1, UV, R_{f}=0.4) and concentrated under vacuum to give tert-butyl N-tert-butoxycarbonyl-N-[2-chloro-6-(2,4-difluorophenyl)-5-methoxy-pyrimidin-4-yl]carbamate (500 mg, 1.04 mmol, 40.90% yield) as colorless gum. SS-2021-01-66-1 (P1): [M+H]+=472.2; purity=97% (220 nm); Retention time=0.998 min H NMR: ¹H NMR (400 MHz, CHLOROFORM-d) δ=7.66-7.57 (m, 1H), 7.10-7.00 (m, 1H), 6.98-6.90 (m, 1H), 3.60 (s, 3H), 1.48-1.45 (m, 20H).

Step 4: To a mixture of tert-butyl N-tert-butoxycarbonyl-N-[2-chloro-6-(2,4-difluorophenyl)-5-methoxy-pyrimidin-4-yl]carbamate (1.00 eq, 400 mg, 0.84 mmol) in DCM (12 mL) was added BBr₃ (5.00 eq, 1.3 mL, 4.24 mmol). The mixture was stirred at 25° C. for 12 h. LCMS showed the starting material was consumed completely and a major peak with desired MS (98%, Rt: 0.488 min; [M+H]+=258.0 at 220 nm) was detected. The mixture was added to 100 mL water and extracted with EtOAc (100 mL three times). The organic phase was dried over anhydrous Na₂SO₄ and concentrated under vacuum to give 4-amino-2-chloro-6-(2,4-difluorophenyl)pyrimidin-5-ol (210 mg, 0.81 mmol, 96.16% yield) as light yellow solid and the crude was used for next step directly. SS-2021-01-71-1 (P1): [M+H]+=258.0; purity=97% (220 nm); Retention time=0.429 min ¹H NMR (400 MHz, CHLOROFORM-d) 6=7.80-7.64 (m, 1H), 7.14-7.03 (m, 1H), 7.02-6.89 (m, 1H), 5.69 (br s, 2H)

Step 5: A mixture of 4-amino-2-chloro-6-(2,4-difluorophenyl)pyrimidin-5-ol (1.00 eq, 210 mg, 0.81 mmol) in 1,1,1-triethoxyethane (34.9 eq, 5.2 mL, 28.5 mmol) was heated to 80° C. and stirred for 2 h. LCMS showed a major peak with desired MS (92%, Rt: 0.594 min; [M+H]+=282.0 at 220 nm). The mixture was concentrated under vacuum to give a crude. The crude was purified by flash column (PE EtOAc=3:1, UV) to give 5-chloro-7-(2,4-difluorophenyl)-2-methyl-oxazolo[4,5-d]pyrimidine (180 mg, 0.63 mmol, 78.40% yield) as light yellow solid. SS-2021-01-72-1 (P1): [M+H]+=281.7; purity=93% (220 nm); Retention time=0.895 min 1H NMR (400 MHz, CHLOROFORM-d) 6=8.10-8.00 (m, 1H), 7.16-7.07 (m, 1H), 7.06-6.97 (m, 1H), 2.81 (s, 3H).

Step 6: To a solution of 5-chloro-7-(2,4-difluorophenyl)-2-methyl-oxazolo[4,5-d]pyrimidine (1.00 eq, 150 mg, 0.53 mmol) and C-phos (0.10 eq, 23 mg, 0.05 mmol) in THF (3 mL) (99.5%, Extra Dry over Molecular Sieve, Stabilized, Acros) was added PALLADIUM(II) ACETATE (0.05 eq, 6.0 mg, 0.02 mmol) followed by bromo-[(2R,4R)-2-(1-cyclopropylpyrazol-4-yl)tetrahydropyran-4-yl]zinc (1.06 eq, 2.6 mL, 0.5 mmol) and the mixture was then stirred at 55° C. for 2 h. LCMS showed that desired MS (56%, Rt: 0.892 min; [M+H]⁺=437.0 at 220 nm) was detected. The mixture was cooled to rt, diluted with EtOAc (20 mL), sat. NaHCO₃ (20 mL) and sat. NaHSO₃ (2 mL). The phase were separated and extracted by EtOAc (20 mL twice). The organic phase was washed with brine and dried over Na₂SO₄, filtered and concentrated in vacuo. The residue was purified by flash silica gel chromatography (Silica Flash Column, Eluent of 0˜50% Ethyl acetate/Petroleum ether gradient) to give 5-[(2R)-2-(1-cyclopropylpyrazol-4-yl)tetrahydropyran-4-yl]-7-(2,4-difluorophenyl)-2-methyl-oxazolo[4,5-d]pyrimidine (64 mg, 0.14 mmol, 27.47% yield) as yellow gum. TH-2021-01-59-1 (P1): [M+H]⁺=437.0; Retention time=0.892 min

Step 7: The 5-[(2R)-2-(1-cyclopropylpyrazol-4-yl)tetrahydropyran-4-yl]-7-(2,4-difluorophenyl)-2-methyl-oxazolo[4,5-d]pyrimidine (1.00 eq, 62 mg, 0.14 mmol) was separated by SFC (Column: DAICEL CHIRALPAK AD (250 mm*30 mm, 10 um); Condition: 0.1% NH₃H₂O MEOH) and lyophilized to give a crude product. LCMS showed 62% was possible ring-open by-product and 35% was desired product (35%, Rt: 0.652 min; [M+H]⁺=438.1 at 220 nm). The crude product was purified by prep-TLC (EtOAc only, R_{f}=0.4) and lyophilized to give 5-[(2R,4S)-2-(1-cyclopropylpyrazol-4-yl)tetrahydropyran-4-yl]-7-(2,4-difluorophenyl)-2-methyl-oxazolo[4,5-d]pyrimidine (enantiopure, 7.3 mg, rt: 2.302 min) as light yellow solid. Absolute stereochemistry arbitrarily assigned. LC-MS: Rt: 0.647 min, m/z: 438.1 [M+H]⁺. 97.3% purity at 220 nm. ¹H NMR (400 MHz, CHLOROFORM-d) 6=8.05 (dt, J=6.6, 8.4 Hz, 1H), 7.51 (d, J=4.4 Hz, 2H), 7.11 (dt, J=1.9, 8.2 Hz, 1H), 7.02 (ddd, J=2.4, 8.5, 10.5 Hz, 1H), 4.55 (dd, J=1.7, 11.4 Hz, 1H), 4.30-4.21 (m, 1H), 3.80 (dt, J=3.2, 11.5 Hz, 1H), 3.58 (tt, J=3.9, 7.3 Hz, 1H), 3.50-3.38 (m, 1H), 2.79 (s, 3H), 2.41-2.33 (m, 1H), 2.23-2.05 (m, 3H), 1.15-1.08 (m, 2H), 1.06-0.97 (m, 2H).

### Example 3—Synthesis of I-26: 4-[5-(4-chloro-2-fluoro-phenyl)-2-methyl-imidazo[1,2-a]pyrimidin-7-yl]-2-(1-cyclopropylpyrazol-4-yl)morpholine

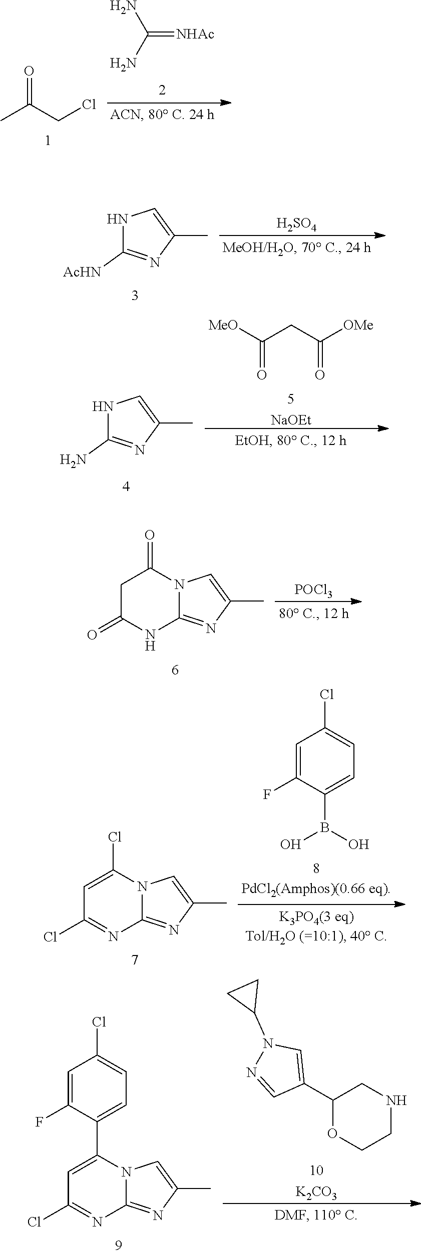

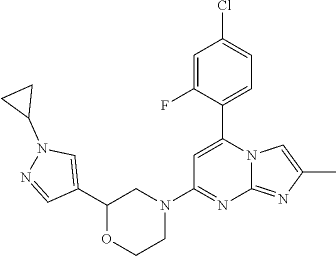 [SMILES: Cc1cn2c(-c3ccc(Cl)cc3F)cc(N3CCOC(c4cnn(C5CC5)c4)C3)nc2n1]

Step 1: To a solution of N-(diaminomethylene)acetamide (3.28 g, 32.4 mmol, 3.0 eq) in MeCN (20 mL) was added 1-chloropropan-2-one (1.0 g, 10.8 mmol, 1.0 eq). The resulting mixture was stirred at 82° C. for 24 h. The solvent was evaporated, and the residue was purified by column chromatography (DCM/MeOH=96/4) to obtain the desired product as a white solid (0.23 g, 15%). LCMS (M+H)⁺=140.20, Retention time=0.117 min. ¹H NMR (400 MHz, DMSO) δ 11.15 (s, 1H), 10.93 (s, 1H), 6.38 (s, 1H), 2.05 (s, 3H), 2.02 (s, 3H).

Step 2: To a solution of N-(4-methyl-1H-imidazol-2-yl) acetamide (100 mg, 0.72 mmol, 1.0 eq) in water (2.5 mL) and methanol (2.5 mL) was added H₂SO₄ (35 mg, 0.36 mmol, 0.5 eq). The resulting mixture was stirred at 80° C. for 24 h. Then the reaction mixture was basified to pH˜10 with 1% KOH in MeOH. The mixture was concentrated under reduced pressure to give the crude product. The product was purified by flash chromatography (MeOH/DCM=3-8%) to give the desired product as a light oil (59 mg, 80%). LCMS (M+H)=98.05, Retention time=0.143 min

Step 3: To a solution of 4-methyl-1H-imidazol-2-amine (881 mg, 9.07 mmol, 1.0 eq) and dimethyl malonate (1.43 g, 10.88 mmol, 1.20 eq) in ethanol (10 mL) was added sodium ethoxide (1.23 g, 18.14 mmol, 2.0 eq). The resulting mixture was stirred at 80° C. for 12 h. The mixture was acidified with 2N HCl (6.2 mL) till pH≈7. The mixture was subjected to lyophilization to give the crude product as a brown solid (1.03 g, 65%). LCMS (M+H)⁺=166.15, Retention time=0.177 min

Step 4: A mixture of 2-methyl-8H-imidazo[1,2-a]pyrimidine-5,7-dione (100 mg, 0.61 mmol, 1.0 eq) and POCl₃ (2 mL) was stirred at 80° C. for 12 h. The reaction was cooling down and POCl₃ was evaporated. The residue was quenched with H₂O (5 mL) and extracted with DCM (10 mL×5). The combined organic phase was washed with brine (10 mL×3), dried over anhydrous sodium sulfate, filtrated and concentrated under reduced pressure. The crude product was obtained as a brown solid (106 mg, 55%). LCMS (M+H)⁺=201.90, Retention time=0.619 min

Step 5: To a solution of 5,7-dichloro-2-methyl-imidazo[1,2-a]pyrimidine (130 mg, 0.64 mmol, 1.0 eq) and (4-chloro-2-fluoro-phenyl) boronic acid (112 mg, 0.64 mmol, 1.0 eq) in toluene (3.0 mL) and water (0.3 mL) were added K₃PO₄ (409 mg, 1.93 mmol, 3.0 eq) and PdCl₂(Amphos)₂ (27 mg, 0.039 mmol, 0.06 eq). The resulting mixture was stirred at 40° C. for 2 h. The reaction was diluted with H₂O (5 mL) and extracted with EtOAc (10 mL×3). The combined organic phase was washed with brine (5 mL×3), dried over anhydrous sodium sulfate, filtrated, and concentrated under reduced pressure. The residue was purified by flash chromatography with Petroleum ether/Ethyl acetate (=75/25) to give the product as a yellow solid (46 mg, 24%). ¹H NMR (400 MHz, CDCl₃) δ 7.52 (t, J=7.9 Hz, 1H), 7.46-7.35 (m, 2H), 7.09 (d, J=1.9 Hz, 1H), 6.86 (s, 1H), 2.46 (s, 3H).

Step 6: To a solution of 7-chloro-5-(4-chloro-2-fluoro-phenyl)-2-methyl-imidazo[1,2-a]pyrimidine (42 mg, 0.14 mmol, 1.0 eq) and 2-(1-cyclopropylpyrazol-4-yl)morpholine (27 mg, 0.14 mmol, 1.0 eq) in DMF (1 mL) was added K₂CO₃ (39 mg, 0.28 mmol, 2.0 eq). The resulting mixture was stirred at 110° C. for 12 h. Desired product was obtained by prep-HPLC as a yellow solid (4.3 mg, 6.3%). LCMS (M+H)⁺=453.30, Retention time=1.606 min. HPLC: purity=97.97% (254 nm); purity=95.40% (214 nm); Retention time=2.884 min. ¹H NMR (400 MHz, DMSO) δ 7.88-7.79 (m, 2H), 7.74 (t, J=7.1 Hz, 1H), 7.63 (d, J=8.3 Hz, 1H), 7.48 (s, 2H), 7.32 (s, 1H), 4.79-4.58 (m, 1H), 4.53 (d, J=9.6 Hz, 1H), 4.49-4.14 (m, 1H), 4.07-4.03 (m, 1H), 3.68-3.65 (m, 2H), 3.41-3.14 (m, 2H), 2.28 (s, 3H), 0.96-0.94 (m, 4H).

### Example 4—Synthesis of Compounds I-22: 7-(4-chloro-2-fluoro-phenyl)-N,N-dimethyl-5-[rac-(2S,6R)-2-(1-cyclopropylpyrazol-4-yl)-6-methyl-morpholin-4-yl]thiazolo[4,5-d]pyrimidin-2-amine

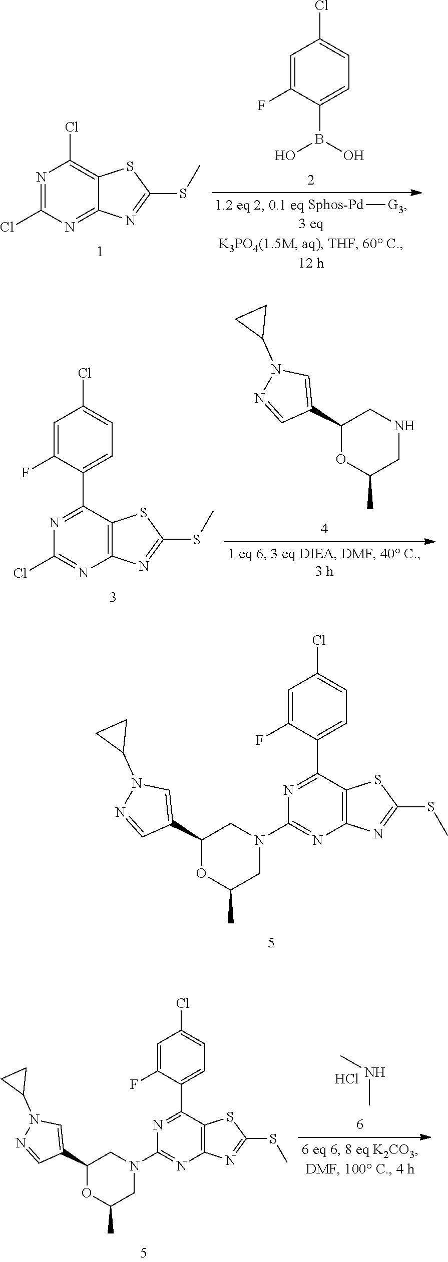

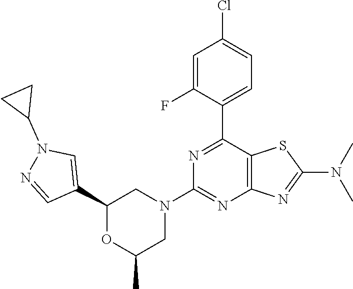 [SMILES: C[C@@H]1CN(c2nc(-c3ccc(Cl)cc3F)c3sc(N(C)C)nc3n2)C[C@H](c2cnn(C3CC3)c2)O1]

Step 1: To a solution of 5,7-dichloro-2-methylsulfanyl-thiazolo[4,5-d]pyrimidine (1.00 eq, 200 mg, 0.793 mmol) in THF (10 mL) and was added (4-chloro-2-fluoro-phenyl)boronic acid (1.20 eq, 166 mg, 0.952 mmol), K₃PO₄ (aq) (3.00 eq, 1.6 mL, 2.38 mmol) and Sphos-Pd-G3 (0.1000 eq, 69 mg, 0.0793 mmol). The reaction mixture was replaced with nitrogen 3 times. Then the reaction mixture was stirred at 60° C. for 16 hours under N₂ atmosphere. LCMS (5-95AB/1.5 min): RT=1.022 min, 346.0=[M+H]⁺, ESI+ showed 45.7% of desired product. The reaction was diluted with water (50 mL) and then extracted with ethyl acetate (50 mL*3). The combined organic layers were washed with brine, dried over Na₂SO₄, filtered and concentrated under reduced pressure to give a residue. The residue was purified by flash chromatography on silica gel eluting with PE/EtOAc (2:1) (TLC, PE:EtOAc=1:1, R_{f}=0.70) to afford 5-chloro-7-(4-chloro-2-fluoro-phenyl)-2-methylsulfanyl-thiazolo[4,5-d]pyrimidine (193 mg, 0.407 mmol, 51.30% yield) as yellow solid. LCMS: Rt: 0.907 min; [M+H]+=346.0; 73% purity at 220 nm.

Step 2: To a solution of 5-chloro-7-(4-chloro-2-fluoro-phenyl)-2-methylsulfanyl-thiazolo[4,5-d]pyrimidine (1.00 eq, 270 mg, 0.585 mmol) and (2S,6R)-2-(1-cyclopropylpyrazol-4-yl)-6-methyl-morpholine (1.00 eq, 303 mg, 0.585 mmol) in DMF (8 mL) was added DIEA (4.00 eq, 302 mg, 2.34 mmol). Then the reaction mixture was stirred at 50° C. for 12 hours. (5-95AB/1.5 min): RT=1.106 min, 517.2=[M+H]⁺, ESI+ showed 38% of desired product and RT=1.012 min, 505.1=[M+H]⁺+, ESI+ showed 30.6% of by-product. TLC (PE:EtOAc=5:1) showed most of starting material was consumed (R_{f}=0.50) and TLC (PE:EtOAc=1:2) showed two new spots was formed (R_{f}=0.55, R_{f}=0.6). The reaction was diluted with water (50 mL) and then extracted with ethyl acetate (50 mL*3). The combined organic layers were washed with brine, dried over Na₂SO₄, filtered and concentrated under reduced pressure to give a residue. The residue was purified by flash chromatography on silica gel eluting. (2S,6R)-4-[7-(4-chloro-2-fluoro-phenyl)-2-methylsulfanyl-thiazolo[4,5-d]pyrimidin-5-yl]-2-(1-cyclopropylpyrazol-4-yl)-6-methyl-morpholine (208 mg, 0.402 mmol, 68.78% yield) ([petroleum ether]/[ethyl acetate](1:1)) was afford as yellow solid. LCMS: Rt: 1.101 min; [M+H]⁺=517.1; 84% purity at 220 nm. 5-chloro-7-(4-chloro-2-fluoro-phenyl)-2-methylsulfanyl-thiazolo[4,5-d]pyrimidine (37 mg, 0.107 mmol, 18.27% yield) ([petroleum ether]/[ethyl acetate](9:1)) was recycled as withe solid. LCMS: Rt: 1.021 min; [M+H]⁺=345.9; 98% purity at 220 nm.

Step 3: To a solution of rac-(2S,6R)-4-[7-(4-chloro-2-fluoro-phenyl)-2-methylsulfanyl-thiazolo[4,5-d]pyrimidin-5-yl]-2-(1-cyclopropylpyrazol-4-yl)-6-methyl-morpholine (1.00 eq, 200 mg, 0.387 mmol) and dimethylamine hydrochloride (6.00 eq, 189 mg, 2.32 mmol) in DMF (10 mL) was added K₂CO₃ (8.00 eq, 428 mg, 3.09 mmol). Then the reaction mixture was stirred at 100° C. for 2 hours. LCMS (5-95AB/1.5 min): RT=1.039 min, 514.2=[M+H]⁺, ESI+ showed 40% of desired product. The reaction was diluted with water (50 mL) and then extracted with ethyl acetate (50 mL*3). The combined organic layers were washed with brine, dried over Na₂SO₄, filtered and concentrated under reduced pressure to give a residue. The residue was purified by prep-HPLC (Column, [Phenomenex Synergi C18 150*25 mm*10 um]; mobile phase: [ACN] and [H₂O](conditions: [water (0.225% FA)-ACN], B %: 67%-87%; Detector, UV 254 nm. RT: [10 min]) to afford crude product (45 mg) as yellow solid, which confirmed by LCMS (5-95AB/1.5 min): RT=1.027 min, 514.2=[M+H]+ showed 68% of crude product. The crude product was separated by SFC (Column: REGIS(S,S)WHELK-O1 (250 mm*25 mm, 10 um); Mobile phase: 0.1% NH₃·H₂O MeOH in CO₂ from 50% to 50%; Flow rate: 80 mL/min Wavelength: 220 nm) to give 7-(4-chloro-2-fluoro-phenyl)-N,N-dimethyl-5-[rac-(2S,6R)-2-(1-cyclopropylpyrazol-4-yl)-6-methyl-morpholin-4-yl]thiazolo[4,5-d]pyrimidin-2-amine (17 mg, 0.0339 mmol, 8.75% yield) as white solid.

I-92: LCMS: Rt: 1.028 min; [M+H]⁺=514.2; 100% purity at 220 nm and ¹H NMR (400 MHz, CHLOROFORM-d) δ ppm 0.97-1.03 (m, 2H) 1.08-1.15 (m, 2H) 1.30 (d, J=6.13 Hz, 3H) 2.72 (dd, J=12.88, 10.88 Hz, 1H) 2.90-3.01 (m, 1H) 3.27 (br s, 6H) 3.57 (tt, J=7.13, 3.69 Hz, 1H) 3.80 (td, J=6.41, 3.06 Hz, 1H) 4.58 (dd, J=10.76, 2.13 Hz, 1H) 4.79 (br d, J=13.01 Hz, 1H) 4.90 (br d, J=13.13 Hz, 1H) 7.22 (br d, J=10.63 Hz, 1H) 7.28 (s, 1H) 7.53 (d, J=5.38 Hz, 2H) 7.71 (t, J=8.07 Hz, 1H) and ¹⁹F NMR and HPLC.

### Example 5—Synthesis of Compound I-79: 7-(4-chloro-2-fluoro-phenyl)-5-[(2R,4S)-2-(1-cyclopropylpyrazol-4-yl)tetrahydropyran-4-yl]-N,N-dimethyl-thiazolo[4,5-d]pyrimidin-2-amine

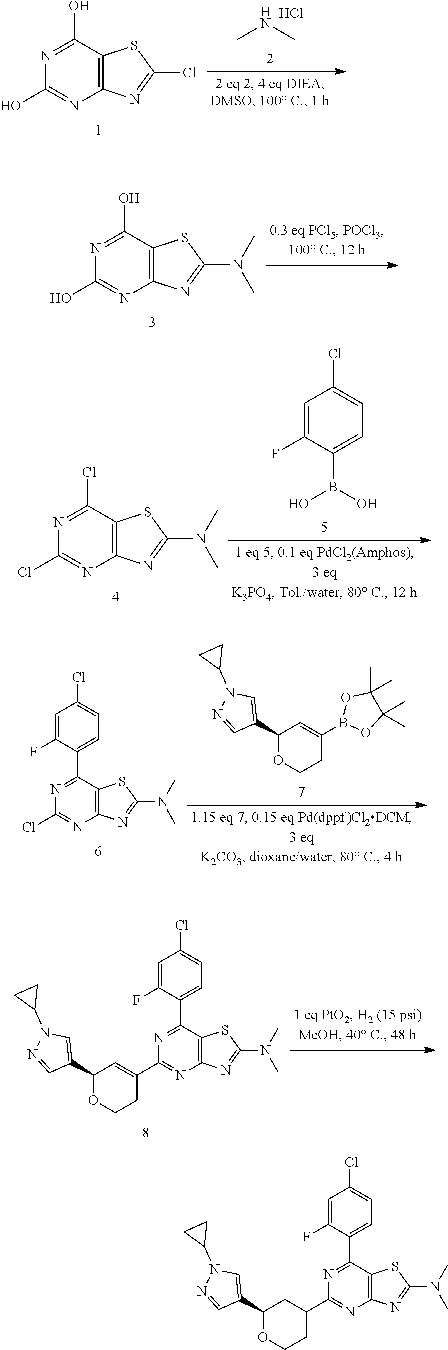

Step 1: To a solution of 2-chlorothiazolo[4,5-d]pyrimidine-5,7-diol (1.00 eq, 680 mg, 3.34 mmol) and Me₂NH HCl (2.00 eq, 545 mg, 6.68 mmol) in DMSO (10 mL) was added DIEA (4.00 eq, 2.3 mL, 13.4 mmol). Then the reaction mixture was stirred at 80° C. for 1 hour. After completion, a large amount of precipitation were formed. The precipitate was collected by filtration. The filter cake was washed with PE (80 mL) and water (40 mL) and then dried under high vacuum. 2-(dimethylamino)thiazolo[4,5-d]pyrimidine-5,7-diol (640 mg, 3.02 mmol, 90.29% yield) was given as pink solid. The crude product was used to next step without further purification.

Step 2: To a suspension of 2-(dimethylamino)thiazolo[4,5-d]pyrimidine-5,7-diol (1.00 eq, 640 mg, 3.02 mmol) in POCl₃ (53.2 eq, 15 mL, 160 mmol) was added PCl₅ (0.271 eq, 170 mg, 0.816 mmol) at 25° C., then stirred at 100° C. for 16 hours. LCMS (5-95AB/1.5 min): RT=0.787 min, 248.9=[M+H]⁺, ESI+ showed 99.8% of desired product. The mixture was concentrated under reduced pressure to give a residue. The residue was quenched with saturated NaHCO₃ aqueous solution (80 ml) and then extracted with DCM (100 mL*3). The combined organic layers were dried over Na₂SO₄, filtered and concentrated under reduced pressure to give a residue. The residue was purified by flash chromatography on silica gel eluting with DCM/EtOAc (10:1) (TLC, DCM:EtOAc=0:1, Rf=0.70) to afford 5,7-dichloro-N,N-dimethyl-thiazolo[4,5-d]pyrimidin-2-amine (620 mg, 2.35 mmol, 77.83% yield) as white solid. (P1): [M+H]⁺=249.0; purity=94.3% (220 nm); Retention time=0.633 min;

Step 3: To a solution of (4-chloro-2-fluoro-phenyl)boronic acid (1.10 eq, 477 mg, 2.74 mmol) in Toluene (10 mL) was added 5,7-dichloro-N,N-dimethyl-thiazolo[4,5-d]pyrimidin-2-amine (1.00 eq, 620 mg, 2.49 mmol), PdCl₂(Amphos) (0.1000 eq, 176 mg, 0.249 mmol) and K₃PO₄ (3.00 eq, 1583 mg, 7.47 mmol). The reaction mixture was replaced with nitrogen 3 times. Then the reaction mixture was stirred at 80° C. for 16 hours under N₂ atmosphere. LCMS (5-95AB/1.5 min): RT=0.771 min, 343.0=[M+H]⁺, ESI+ showed 35.8% of desired product. The reaction was diluted with water (80 mL) and then extracted with DCM (80 mL*3). The combined organic layers were washed with brine, dried over Na₂SO₄, filtered and concentrated under reduced pressure to give a residue. The residue was purified by silica gel chromatography using 10% ethyl acetate in DCM to afford 5-chloro-7-(4-chloro-2-fluoro-phenyl)-N,N-dimethyl-thiazolo[4,5-d]pyrimidin-2-amine (540 mg, 1.57 mmol, 63.22% yield) as white solid. (P1): [M+H]⁺=342.9; purity=33.1% (220 nm); Retention time=0.920 min

Step 4: To a solution of 5-chloro-7-(4-chloro-2-fluoro-phenyl)-N,N-dimethyl-thiazolo[4,5-d]pyrimidin-2-amine (1.00 eq, 540 mg, 0.944 mmol) 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 328 mg, 1.04 mmol) and K₂CO₃ (4.00 eq, 522 mg, 3.78 mmol) in 1,4-Dioxane (15 mL) and Water (1.5 mL) was added Pa(dppf)Cl₂·DCM (0.150 eq, 104 mg, 0.142 mmol). The reaction mixture was stirred at 80° C. for 2 hours under N₂ atmosphere. LCMS (5-95AB/1.5 min): RT=0.978 min, 497.1=[M+H]⁺, ESI+ showed 44% of desired product. The reaction solution was concentrated under reduced pressure to afford a residue. The reaction was diluted with water (100 mL) and then extracted with DCM (100 mL*3). The combined organic layers were washed with brine, dried over Na₂SO₄, filtered and concentrated under reduced pressure to give a residue. The residue was purified by flash chromatography on silica gel eluting with DCM/EtOAc (1:1) (TLC, PE:EtOAc=0:1, Rf=0.45) to afford 7-(4-chloro-2-fluoro-phenyl)-N,N-dimethyl-5-[(6R)-6-(1-cyclopropylpyrazol-4-yl)-3,6-dihydro-2H-pyran-4-yl]thiazolo[4,5-d]pyrimidin-2-amine (340 mg, 0.635 mmol, 67.25% yield) as yellow oil. (P1): [M+H]⁺=497.1; purity=92.8% (220 nm); Retention time=0.978 min;

Step 5: To a solution of 7-(4-chloro-2-fluoro-phenyl)-5-[(6R)-6-(1-cyclopropylpyrazol-4-yl)-3,6-dihydro-2H-pyran-4-yl]-N,N-dimethyl-thiazolo[4,5-d]pyrimidin-2-amine (1.00 eq, 340 mg, 0.684 mmol) in methanol (20 mL) was added PtO₂ (1.00 eq, 155 mg, 0.684 mmol) under N₂ atmosphere. The mixture was purged with H₂ (15 psi) 3 times, then the mixture was stirred at 30° C. for 24 hours under H₂ (15 psi) atmosphere. LCMS (5-95AB/1.5 min): RT=0.944 min, 499.1=[M+H]⁺, ESI+ showed 43% of desired product and RT=0.991 min, 497.1=[M+H]⁺, ESI+ showed 40% of starting material. Then the reaction mixture was filtered through a pad of celite. The filter cake was washed with MeOH (80 mL) and DCM (30 mL). The filtrate was concentrated under reduced pressure to afford a residue. The residue was dissolved in methanol (30 mL) and PtO₂ (1.00 eq, 155 mg, 0.684 mmol) was added. The mixture was purged with H₂ (15 psi) 3 times, then the mixture was stirred at 40° C. for 48 hours under H₂ (15 psi) atmosphere. LCMS (5-95AB/1.5 min): RT=0.947 min, 499.1=[M+H]⁺, ESI+ showed 61.1% of desired product and RT=0.992 min, 497.1=[M+H]⁺, ESI+ showed 14.3% of starting material. The reaction mixture was filtered through a pad of celite. The filter cake was washed with MeOH (60 mL). The filtrate was concentrated under reduced pressure to afford a residue. The residue was purified by prep-HPLC (Column, [Unisil 3-100 C18 Ultra 150*50 mm*3 um]; mobile phase: [ACN] and [H₂O](conditions: [water (0.225% FA)-ACN], B %: 51%-81%; Detector, UV 254 nm. RT: [10 min]) to afford 7-(4-chloro-2-fluoro-phenyl)-5-[(2R,4S)-2-(1-cyclopropylpyrazol-4-yl)tetrahydropyran-4-yl]-N,N-dimethyl-thiazolo[4,5-d]pyrimidin-2-amine (67 mg, 0.134 mmol, 19.52% yield) as white solid. (P1, single enantiomer of known absolute configuration): [M+H]⁺=499.1; purity=100% (220 nm); Retention time=0.925 min; ¹H NMR (400 MHz, CHLOROFORM-d) δ ppm 0.92-1.01 (m, 2H) 1.05-1.14 (m, 2H) 2.01-2.22 (m, 3H) 2.33 (dt, J=13.17, 1.67 Hz, 1H) 3.32 (ddt, J=15.67, 7.93, 3.79, 3.79 Hz, 7H) 3.55 (tt, J=7.27, 3.73 Hz, 1H) 3.76 (td, J=11.86, 2.32 Hz, 1H) 4.19-4.26 (m, 1H) 4.51 (dd, J=11.37, 1.96 Hz, 1H) 7.25 (d, J=1.96 Hz, 1H) 7.32 (dd, J=8.38, 1.77 Hz, 1H) 7.48 (s, 2H) 7.75 (t, J=8.13 Hz, 1H).

### Example 6—Synthesis of Compound I-84: 7-(2,4-difluorophenyl)-N,N-dimethyl-5-[(2S,6R)-2-(1-cyclopropylpyrazol-4-yl)-6-methyl-morpholin-4-yl]thiazolo[4,5-d]pyrimidin-2-amine

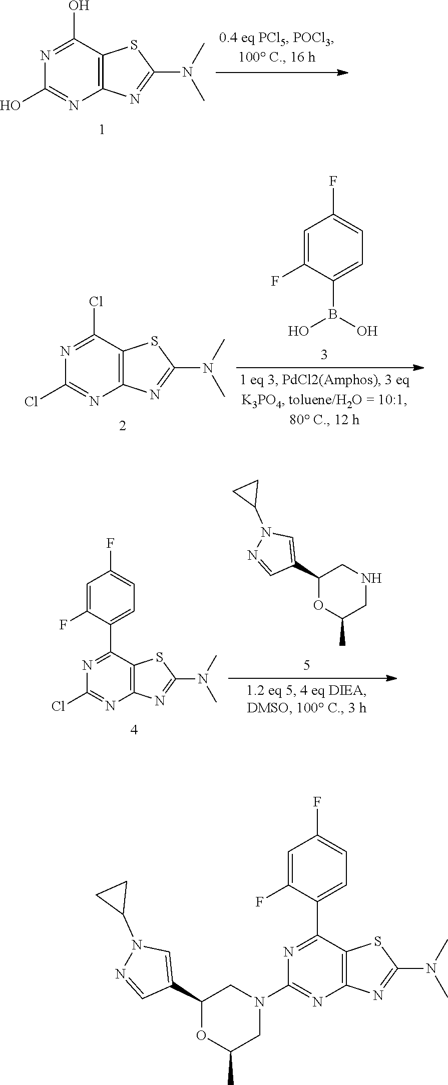 [SMILES: CN(C)c1nc2nc(O)nc(O)c2s1 ; CN(C)c1nc2nc(Cl)nc(Cl)c2s1 ; OB(O)c1ccc(F)cc1F ; CN(C)c1nc2nc(Cl)nc(-c3ccc(F)cc3F)c2s1 ; C[C@@H]1CNC[C@H](c2cnn(C3CC3)c2)O1 ; C[C@@H]1CN(c2nc(-c3ccc(F)cc3F)c3sc(N(C)C)nc3n2)C[C@H](c2cnn(C3CC3)c2)O1]

### Step 1: 5,7-dichloro-N,N-dimethyl-thiazolo[4,5-d]pyrimidin-2-amine

To a suspension of 2-(dimethylamino)thiazolo[4,5-d]pyrimidine-5,7-diol (1.00 eq, 1600 mg, 7.54 mmol) in POCl₃ (63.8 eq, 45 mL, 481 mmol) was added PCl₅ (0.437 eq, 686 mg, 3.29 mmol) at 25° C., then stirred at 100° C. for 16 hours. LCMS (5-95AB/1.5 min): RT=0.593 min, 249.0=[M+H]⁺, ESI+ showed 94.8% of desired product. The mixture was concentrated under reduced pressure to give a residue. The residue was quenched with saturated NaHCO₃ aqueous solution (120 ml) and then extracted with DCM (300 mL*3). The combined organic layers were dried over Na₂SO₄, filtered and concentrated under reduced pressure to give 5,7-dichloro-N,N-dimethyl-thiazolo[4,5-d]pyrimidin-2-amine (1.76 g, 7.06 mmol, 93.71% yield) as brown solid. (P1): [M+H]⁺=249.0; purity=94.8% (220 nm); Retention time=0.593 min

Step 2: Charge 5,7-dichloro-N,N-dimethyl-thiazolo[4,5-d]pyrimidin-2-amine (1.00 eq, 100 mg, 0.401 mmol), (2,4-difluorophenyl)boronic acid (1.00 eq, 63 mg, 0.401 mmol), K₃PO₄ (3.00 eq, 256 mg, 1.20 mmol) and PdCl₂(Amphos) (0.100 eq, 28 mg, 0.0401 mmol) into sealed bottle under N₂ atmosphere and purged with N₂ for three times, then Toluene (5 mL) and Water (0.5000 mL) was added in one portion at 15° C., then the mixture was stirred at 15° C. for 1 h and 80° C. for 12 h. LCMS (5-95AB/1.5 min): RT=0.680 min, 327.0=[M+H]⁺, ESI+ showed 46.5% of desired product. The reaction was diluted with water (20 mL) and then extracted with DCM (20 mL*3). The combined organic layers were washed with brine, dried over Na₂SO₄, filtered and concentrated under reduced pressure to give a residue. The residue was purified by prep-TLC (PE:EtOAc=1:1, Rf=0.25) to afford 5-chloro-7-(2,4-difluorophenyl)-N,N-dimethyl-thiazolo[4,5-d]pyrimidin-2-amine (78 mg, 0.112 mmol, 27.95% yield) as off-white solid, which confirmed by LCMS (0-60AB/1.5 min): RT=0.891 min, 327.0=[M+H]⁺, ESI+ showed 49.3% of desired product. The crude (P1): [M+H]⁺=327.0; purity=49.3% (220 nm); Retention time=0.891 min

Step 3: To a solution of (2S,6R)-2-(1-cyclopropylpyrazol-4-yl)-6-methyl-morpholine (1.50 eq, 91 mg, 0.175 mmol) and 5-chloro-7-(2,4-difluorophenyl)-N,N-dimethyl-thiazolo[4,5-d]pyrimidin-2-amine (1.00 eq, 78 mg, 0.117 mmol) in DMSO (2 mL) was added DIEA (4.00 eq, 60 mg, 0.468 mmol) at 25° C. Then the reaction mixture was stirred at 100° C. for 6 hours. LCMS (5-95AB/1.5 min): RT=0.768 min, 498.2=[M+H]⁺, ESI+ showed 20.7% of desired product. The reaction was diluted with water (40 mL) and then extracted with DCM (40 mL*3). The combined organic layers were washed with brine, dried over Na₂SO₄, filtered and concentrated under reduced pressure to give a residue. The residue was purified by prep-HPLC (Column, [Unisil 3-100 C18 Ultra 150*50 mm*3 um]; mobile phase: [ACN] and [H₂O](conditions: [water (0.225% FA)-ACN], B %: 54%-84%; Detector, UV 254 nm. RT: [7 min]) to afford 7-(2,4-difluorophenyl)-N,N-dimethyl-5-[(2S,6R)-2-(1-cyclopropylpyrazol-4-yl)-6-methyl-morpholin-4-yl]thiazolo[4,5-d]pyrimidin-2-amine (28 mg, 0.0559 mmol, 47.77% yield) as white solid. (P1, single enantiomer of known absolute configuration): [M+H]+=498.2; purity=100% (220 nm); Retention time=0.770 min; ¹H NMR (400 MHz, CHLOROFORM-d) δ ppm 0.96-1.04 (m, 2H) 1.08-1.15 (m, 2H) 1.30 (d, J=6.24 Hz, 3H) 2.73 (dd, J=13.08, 10.76 Hz, 1H) 2.96 (dd, J=13.08, 11.00 Hz, 1H) 3.28 (br s, 6H) 3.57 (tt, J=7.27, 3.73 Hz, 1H) 3.76-3.85 (m, 1H) 4.58 (dd, J=10.94, 2.51 Hz, 1H) 4.80 (br d, J=13.08 Hz, 1H) 4.91 (br d, J=12.84 Hz, 1H) 6.89-6.97 (m, 1H) 6.99-7.05 (m, 1H) 7.53 (d, J=4.52 Hz, 2H) 7.71-7.81 (m, 1H).

### Example 7—Synthesis of Compound I-89: Synthesis of Compound 5-[(2R,4S)-2-(1-cyclopropylpyrazol-4-yl)tetrahydropyran-4-yl]-7-(2,4-difluorophenyl)-N,N-dimethyl-thiazolo[4,5-d]pyrimidin-2-amine

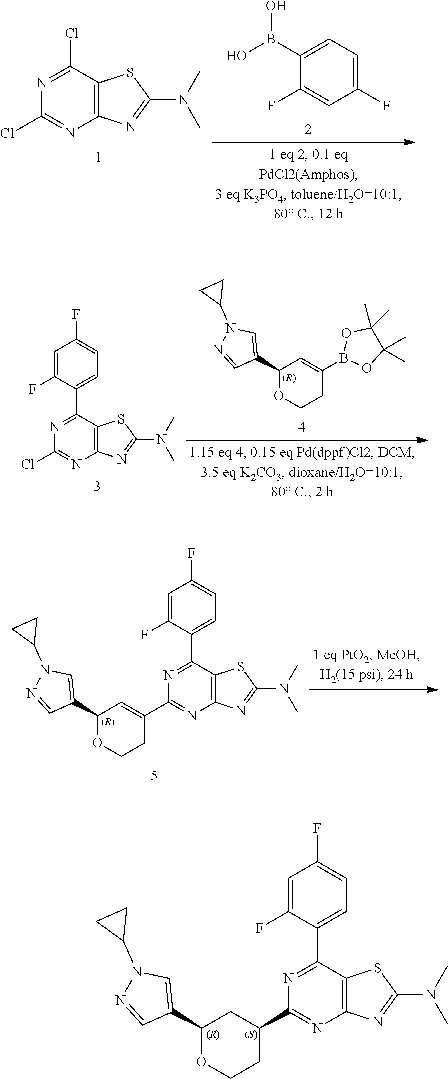

Step 1: Charge 5,7-dichloro-NN-dimethyl-thiazolo[4,5-d]pyrimidin-2-amine (1.00 eq, 400 mg, 1.61 mmol), (2,4-difluorophenyl)boronic acid (1.00 eq, 254 mg, 1.61 mmol), K₃PO₄ (3.00 eq, 1022 mg, 4.82 mmol) and PdCl₂(Amphos) (0.100 eq, 114 mg, 0.161 mmol) into sealed bottle under N₂ atmosphere and purged with N₂ for three times, then Toluene (10 mL) and Water (1 mL) was added in one portion at 15° C., then the mixture was stirred at 15° C. for 1 h and 80° C. for 12 h. LCMS (5-95AB/1.5 min): RT=0.687 min, 327.0=[M+H]+, ESI+ showed 52.1% of desired product. The reaction was diluted with water (50 mL) and then extracted with DCM (50 mL*3). The combined organic layers were washed with brine, dried over Na₂SO₄, filtered and concentrated under reduced pressure to give a residue. The residue was purified by flash chromatography on silica gel eluting with PE/EtOAc (1:1) (TLC, PE:EtOAc=1: 2, Rf=0.45) to afford crude product 5-chloro-7-(2,4-difluorophenyl)-N,N-dimethyl-thiazolo[4,5-d]pyrimidin-2-amine (280 mg, 0.360 mmol, 22.42% yield) as white solid. (P1): [M+H]⁺=327.0; purity=41% (220 nm); Retention time=0.683 min

Step 2: To a solution of 5-chloro-7-(2,4-difluorophenyl)-N,N-dimethyl-thiazolo[4,5-d]pyrimidin-2-amine (1.00 eq, 150 mg, 0.193 mmol), Pd(dppf)Cl₂·DCM (0.150 eq, 21 mg, 0.0289 mmol) and K₂CO₃ (3.50 eq, 93 mg, 0.675 mmol) in 1,4-Dioxane (10 mL) and Water (1 mL) was added Pd(dppf)Cl₂·DCM (0.150 eq, 21 mg, 0.0289 mmol). The reaction mixture was stirred at 80° C. for 3 hours under N₂ atmosphere. LCMS (5-95AB/1.5 min): RT=0.743 min, 481.1=[M+H]⁺, ESI+ showed 38% of desired product. The reaction was diluted with water (40 mL) and then extracted with ethyl acetate (40 mL*3). The combined organic layers were washed with brine, dried over Na₂SO₄, filtered and concentrated under reduced pressure to give a residue. The residue was purified by flash chromatography on silica gel eluting with PE/EtOAc (1:1) (TLC, PE:EtOAc=0:1, Rf=0.2) to afford 7-(2,4-difluorophenyl)-N,N-dimethyl-5-[(6R)-6-(1-cyclopropylpyrazol-4-yl)-3,6-dihydro-2H-pyran-4-yl]thiazolo[4,5-d]pyrimidin-2-amine (100 mg, 0.179 mmol, 92.82% yield) as yellow gum. (P1): [M+H]⁺=481.1; purity=86% (220 nm); Retention time=0.739 min

Step 3: To a solution of 5-[(6R)-6-(1-cyclopropylpyrazol-4-yl)-3,6-dihydro-2H-pyran-4-yl]-7-(2,4-difluorophenyl)-N,N-dimethyl-thiazolo[4,5-d]pyrimidin-2-amine (1.00 eq, 100 mg, 0.179 mmol) in Ethanol (20 mL) was added PtO₂ (1.00 eq, 41 mg, 0.179 mmol) under N₂ atmosphere. The mixture was purged with H₂ (15 psi) 3 times, then the mixture was stirred at 30° C. for 24 hours under H₂ (15 psi) atmosphere. LCMS (5-95AB/1.5 min): RT=0.704 min, 483.1=[M+H]⁺, ESI+ showed 10% of desired product and RT=0.737 min, 481.1=[M+H]⁺, ESI+ showed 62.2% of starting material. Then the reaction was purged with H₂ (15 psi) 3 times and stirred at 40° C. for 48 hours. LCMS (5-95AB/1.0 min): RT=0.591 min, 483.1=[M+H]⁺, ESI+ showed 24.7% of desired product and RT=0.637 min, 481.1=[M+H]⁺, ESI+ showed 15.1% of starting material. Then the reaction mixture was filtered through a pad of diatomite. The filter cake was washed with EtOH (40 ml). The filtrate was concentrated under reduced pressure to afford a residue. The residue was dissolved in Methanol (20 mL) and PtO₂ (1.00 eq, 41 mg, 0.179 mmol) was added. The mixture was purged with H₂ (15 psi) 3 times, then the mixture was stirred at 30° C. for 24 hours under H₂ (15 psi) atmosphere. LCMS (5-95AB/1.0 min): RT=0.587 min, 483.1=[M+H]⁺, ESI+ showed 50.9% of desired product and RT=0.633 min, 481.1=[M+H]⁺, ESI+ showed 19.4% of starting material. Then the reaction mixture was filtered through a pad of diatomite. The filter cake was washed with MeOH (40 ml). The filtrate was concentrated under reduced pressure to afford a residue. The residue was purified by prep-HPLC (Column, [Unisil 3-100 C18 Ultra 150*50 mm*3 um]; mobile phase: [ACN] and [H₂O](conditions: [water (0.225% FA)-ACN], B %: 47%-77%; Detector, UV 254 nm. RT: [7 min]) to afford 5-[(2R,4S)-2-(1-cyclopropylpyrazol-4-yl)tetrahydropyran-4-yl]-7-(2,4-difluorophenyl)-N,N-dimethyl-thiazolo[4,5-d]pyrimidin-2-amine (15 mg, 0.0306 mmol, 17.09% yield) as white solid. (P1, single enantiomer of known absolute configuration): [M+H]⁺=483.2; purity=98.6% (220 nm); Retention time=0.721 min. ¹H NMR (400 MHz, CHLOROFORM-d) δ ppm 0.94-1.03 (m, 2H) 1.04-1.13 (m, 2H) 2.03-2.22 (m, 3H) 2.33 (br d, J=13.20 Hz, 1H) 3.08-3.51 (m, 7H) 3.55 (tt, J=7.23, 3.71 Hz, 1H) 3.76 (td, J=11.83, 2.38 Hz, 1H) 4.19-4.26 (m, 1H) 4.51 (dd, J=11.31, 1.77 Hz, 1H) 6.97 (ddd, J=10.70, 8.62, 2.45 Hz, 1H) 7.06 (td, J=8.16, 2.14 Hz, 1H) 7.49 (s, 2H) 7.80 (td, J=8.44, 6.48 Hz, 1H).

### Example 8—Synthesis of Compound I-94: 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine

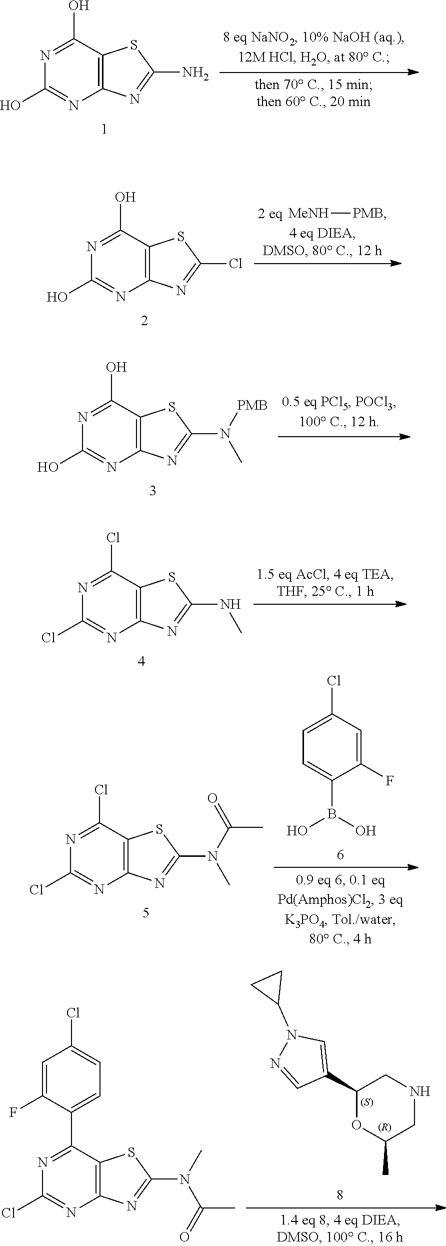

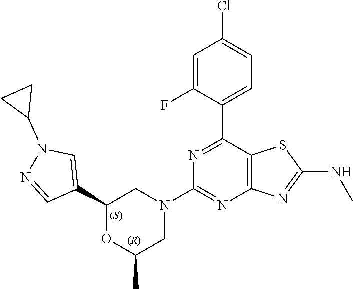

Step 1: A solution of 2-aminothiazolo[4,5-d]pyrimidine-5,7-diol (1.00 eq, 2000 mg, 10.9 mmol) and NaNO₂ (8.00 eq, 5.99 g, 86.9 mmol) in 10% aqueous NaOH (1.00 eq, 20 mL, 10.9 mmol) was added gradually to a stirred solution of concentrated HCl (1.00 eq, 64 mL, 10.9 mmol) and water (16 mL) at 80° C. Then the reaction mixture was stirred at 70° C. for 35 min. Then the mixture was stirred for a further 30 min at 60° C. Then the reaction mixture chilled in ice, and the precipitate was collected, washed with water, and dried under reduced pressure to afford 2-chlorothiazolo[4,5-d]pyrimidine-5,7-diol (1.71 g, 8.40 mmol, 77.34% yield) as orange solid, which confirmed by ¹H NMR. ¹H NMR (400 MHz, DMSO-d6) δ ppm 11.54 (s, 1H) 12.52 (s, 1H).

Step 2: To a solution of 2-chlorothiazolo[4,5-d]pyrimidine-5,7-diol (1.00 eq, 1500 mg, 7.37 mmol) and 1-(4-methoxyphenyl)-N-methyl-methanamine (2.00 eq, 2228 mg, 14.7 mmol) in DMSO (15 mL) was added DIEA (4.00 eq, 5.1 mL, 29.5 mmol). Then the reaction mixture was stirred at 80° C. for 12 hours. LCMS (5-95AB/1.5 min): RT=0.777 min, 319.0=[M+H]⁺, ESI+ showed 97.3% of desired product. The reaction was diluted with water (10 mL). During this period, red precipitate was formed. It was collected by filtration and washed with PE (30 mL*3) and dried under high vacuum. Crude product 2-[(4-methoxyphenyl)methyl-methyl-amino]thiazolo[4,5-d]pyrimidine-5,7-diol (2.62 g, 8.23 mmol, 111.71% yield) was afford as red solid, which confirmed by LCMS (5-95AB/1.5 min): RT=0.583 min, 319.1=[M+H]⁺, ESI+ showed 99.2% of desired product. The crude product was used to next step without further purification. (P1): [M+H]⁺=319.1; purity=99.2% (220 nm); Retention time=0.583 min

Step 3: To a suspension of 2-[(4-methoxyphenyl)methyl-methyl-amino]thiazolo[4,5-d]pyrimidine-5,7-diol (1.00 eq, 2.62 g, 8.23 mmol) in POCl₃ (52.0 eq, 40 mL, 428 mmol) was added PCl₅ (0.500 eq, 857 mg, 4.11 mmol) at 25° C., then stirred at 100° C. for 16 h. The reaction was concentrated under reduced pressure to give a residue. The residue was quenched with saturated NaHCO₃ aqueous solution (120 mL) and then extracted with DCM (300 mL*3). The combined organic layers were dried over Na₂SO₄, filtered and concentrated under reduced pressure to give a residue. The residue was purified by flash chromatography on silica gel eluting with PE/EtOAc (3:2) (TLC, PE:EtOAc=1:1, Rf=0.65) to afford 5,7-dichloro-N-[(4-methoxyphenyl)methyl]-N-methyl-thiazolo[4,5-d]pyrimidin-2-amine (460 mg, 1.29 mmol, 15.73% yield) as yellow gum. 5,7-dichloro-N-methyl-thiazolo[4,5-d]pyrimidin-2-amine (1000 mg, 4.25 mmol, 51.69% yield) was purified by flash chromatography on silica gel eluting with PE/EtOAc (1:1) (TLC, PE:EtOAc=1:1, Rf=0.35) as off-white solid. (P1): [M+H]⁺=243.9; purity=42% (220 nm); Retention time=0.753 min

Step 4: To a solution of 5,7-dichloro-N-methyl-thiazolo[4,5-d]pyrimidin-2-amine (1.00 eq, 950 mg, 4.04 mmol) and TEA (4.00 eq, 2.2 mL, 16.2 mmol) in THF (15 mL) was added AcCl (1.50 eq, 0.43 mL, 6.06 mmol) at 30° C. Then the reaction was stirred at 30° C. for 2 hours. LCMS (5-95AB/1.5 min): RT=0.826 min, 276.9=[M+H]⁺, ESI+ showed 86.5% of desired product. The reaction mixture was adjusted to pH=6-7 with saturated NaHCO₃ aqueous solution, then diluted with water (50 mL) and then extracted with ethyl acetate (50 mL*3). The combined organic layers were washed with brine, dried over Na₂SO₄, filtered and concentrated under reduced pressure to give crude product N-(5,7-dichlorothiazolo[4,5-d]pyrimidin-2-yl)-N-methyl-acetamide (1.15 g, 3.59 mmol, 88.83% yield) as yellow solid. The crude product was used to next step without further purification. (P1): [M+H]+=276.9; purity=86.5% (220 nm); Retention time=0.826 min

Step 5: Charge N-(5,7-dichlorothiazolo[4,5-d]pyrimidin-2-yl)-N-methyl-acetamide (1.00 eq, 1150 mg, 3.53 mmol), (4-chloro-2-fluoro-phenyl)boronic acid (0.900 eq, 554 mg, 3.17 mmol), K₃PO₄ (3.00 eq, 2243 mg, 10.6 mmol) and PdCl₂(Amphos) (0.100 eq, 250 mg, 0.353 mmol) into sealed bottle under N₂ atmosphere and purged with N₂ for three times, then Toluene (15 mL) and Water (1.5 mL) was added in one portion at 15° C., then the mixture was stirred at 90° C. for 6 hours. LCMS (5-95AB/1.5 min): RT=0.784 min, 371.0=[M+H]⁺, ESI+ showed 44.4% of desired product. The reaction mixture was concentrated under reduced pressure to afford a residue. The residue was diluted with water (80 mL) and then extracted with DCM (80 mL*3). The combined organic layers were washed with brine, dried over Na₂SO₄, filtered and concentrated under reduced pressure to give a residue. The residue was purified by flash chromatography on silica gel eluting with DCM/EtOAc (5:1) (TLC, DCM:EtOAc=10:1, Rf=0.60) to afford N-[7-(4-chloro-2-fluoro-phenyl)-5-fluoro-thiazolo[4,5-d]pyrimidin-2-yl]-N-methyl-acetamide (790 mg, 1.74 mmol, 49.24% yield) as yellow solid. (P1): [M+H]⁺=370.9; purity=78% (220 nm); Retention time=0.942 min

Step 6: To a solution of (2S,6R)-2-(1-cyclopropylpyrazol-4-yl)-6-methyl-morpholine (1.04 eq, 1115 mg, 2.15 mmol), N-[5-chloro-7-(4-chloro-2-fluoro-phenyl)thiazolo[4,5-d]pyrimidin-2-yl]-N-methyl-acetamide (1.00 eq, 770 mg, 2.07 mmol) and (2S,6R)-2-(1-cyclopropylpyrazol-4-yl)-6-methyl-morpholine (0.326 eq, 140 mg, 0.675 mmol) in DMSO (15 mL) was added DIEA (5.00 eq, 1340 mg, 10.4 mmol) at 25° C. Then the reaction mixture was stirred at 100° C. for 16 hours. (5-95AB/1.5 min): RT=0.958 min, 500.1=[M+H]⁺, ESI+ showed 56.7% of desired product. The reaction was diluted with water (100 mL) and then extracted with ethyl acetate (100 mL*3). The combined organic layers were washed with brine, dried over Na₂SO₄, filtered and concentrated under reduced pressure to give a residue. The residue was purified by flash chromatography on silica gel eluting with PE/EtOAc (1:4) (TLC, PE:EtOAc=0:1, Rf=0.55) to afford 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine (650 mg, 1.26 mmol, 60.79% yield) as yellow solid. (P1, single enantiomer of known absolute configuration): [M+H]⁺=500.1; purity=97.0% (220 nm); Retention time=0.955 min; ¹H NMR (400 MHz, CHLOROFORM-d) δ ppm 0.97-1.06 (m, 2H) 1.06-1.15 (m, 2H) 1.30 (d, J=6.25 Hz, 3H) 2.73 (dd, J=13.26, 10.63 Hz, 1H) 2.96 (dd, J=13.20, 10.94 Hz, 1H) 3.17 (s, 3H) 3.57 (tt, J=7.30, 3.71 Hz, 1H) 3.75-3.87 (m, 1H) 4.58 (dd, J=10.88, 2.63 Hz, 1H) 4.78 (br d, J=12.88 Hz, 1H) 4.89 (br d, J=12.88 Hz, 1H) 5.99-6.49 (m, 1H) 7.20-7.25 (m, 1H) 7.27-7.31 (m, 1H) 7.53 (d, J=3.50 Hz, 2H) 7.71 (t, J=8.13 Hz, 1H).

### Example 9—Synthesis of Compound I-99: N-[7-(4-chloro-2-fluoro-phenyl)-5-[(2S,6R)-2-(1-cyclopropylpyrazol-4-yl)-6-methyl-morpholin-4-yl]thiazolo[4,5-d]pyrimidin-2-yl]-N-methyl-acetamide

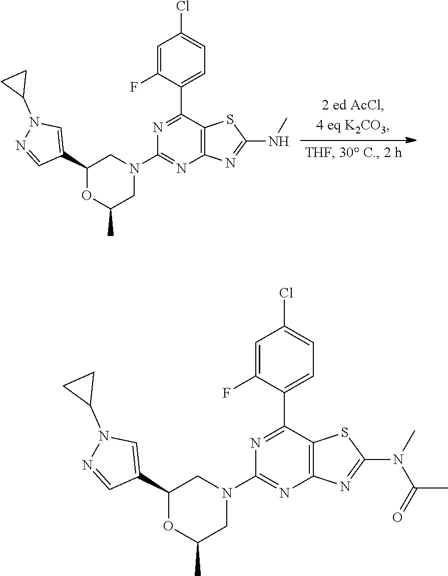 [SMILES: CNc1nc2nc(N3C[C@@H](C)O[C@@H](c4cnn(C5CC5)c4)C3)nc(-c3ccc(Cl)cc3F)c2s1 ; CC(=O)N(C)c1nc2nc(N3C[C@@H](C)O[C@@H](c4cnn(C5CC5)c4)C3)nc(-c3ccc(Cl)cc3F)c2s1]

Step 1: To a solution of 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine (1.00 eq, 80 mg, 0.160 mmol) and TEA (4.00 eq, 0.089 mL, 0.640 mmol) in THF (1.5 mL) was added AcCl (2.50 eq, 0.028 mL, 0.400 mmol) at 30° C. Then the reaction was stirred at 30° C. for 5 hours. LCMS (5-95AB/1.5 min): RT=1.029 min, 542.1=[M+H]⁺, ESI+ showed 89.7% of desired product. The combined reaction mixture was adjusted to pH=7-8 with saturated NaHCO₃ aqueous solution. The reaction was diluted with water (10 mL) and then extracted with ethyl acetate (10 mL*3). The combined organic layers were washed with brine, dried over Na₂SO₄, filtered and concentrated under reduced pressure to give a residue. The residue was purified by prep-HPLC (Column, [Unisil 3-100 C18 Ultra 150*50 mm*3 um]; mobile phase: [ACN] and [H₂O](conditions: [water (0.225% FA)-ACN], B %: 60%-90%; Detector, UV 254 nm. RT: [7 min]) to afford N-[7-(4-chloro-2-fluoro-phenyl)-5-[(2S,6R)-2-(1-cyclopropylpyrazol-4-yl)-6-methyl-morpholin-4-yl]thiazolo[4,5-d]pyrimidin-2-yl]-N-methyl-acetamide (49 mg, 0.0896 mmol, 55.98% yield) as yellow solid. (P1, single enantiomer of known absolute configuration): [M+H]+=542.2; purity=100% (220 nm); Retention time=1.011 min; ¹H NMR (400 MHz, CHLOROFORM-d) δ ppm 0.98-1.04 (m, 2H) 1.09-1.15 (m, 2H) 1.32 (d, J=6.24 Hz, 3H) 2.49 (s, 3H) 2.78 (dd, J=13.14, 10.70 Hz, 1H) 3.01 (dd, J=13.20, 10.88 Hz, 1H) 3.58 (tt, J=7.29, 3.71 Hz, 1H) 3.78-3.85 (m, 1H) 3.86 (s, 3H) 4.60 (dd, J=10.88, 2.57 Hz, 1H) 4.82 (br d, J=13.08 Hz, 1H) 4.94 (br d, J=12.35 Hz, 1H) 7.24-7.27 (m, 1H) 7.29-7.32 (m, 1H) 7.54 (d, J=3.79 Hz, 2H) 7.73 (t, J=8.01 Hz, 1H).

### Example 10—Synthesis of Compound I-104 and I-308: 2-[[7-(4-chloro-2-fluoro-phenyl)-5-[(2S,6R)-2-(1-cyclopropylpyrazol-4-yl)-6-methyl-morpholin-4-yl]thiazolo[4,5-d]pyrimidin-2-yl]-methyl-amino]ethanol and 2-((Z)-7-(4-chloro-2-fluorophenyl)-5-((2S,6R)-2-(1-cyclopropyl-1H-pyrazol-4-yl)-6-methylmorpholino)-2-(methylimino)thiazolo[4,5-d]pyrimidin-3(2H)-yl)ethan-1-ol

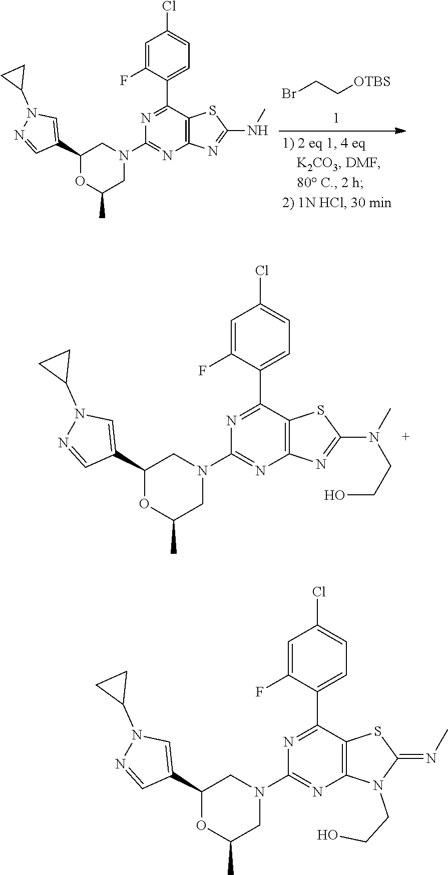

Step 1: To a mixture of 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine (1.00 eq, 90 mg, 0.180 mmol) and K₂CO₃ (4.00 eq, 100 mg, 0.720 mmol) in DMF (2 mL) was added (2-bromoethoxy)(tert-butyl)dimethylsilane (2.00 eq, 0.078 mL, 0.360 mmol) at 25° C. Then the reaction mixture was stirred at 80° C. for 5 hours. TLC (PE:EtOAc=2:1) showed the starting material was consumed (Rf=0.60) and new spot was formed (Rf=0.35). The reaction mixture was adjusted to pH=2-3 with 1 M HCl aqueous solution and stirred at 30° C. for 30 min. LCMS (5-95AB/1.5 min): RT=0.953 min, 544.2=[M+H]⁺, ESI+ showed 79.8% of desired product and RT=0.860 min, 544.2=[M+H]⁺, ESI+ showed 18.4% of isomer. The reaction was diluted with water (15 mL) and then extracted with ethyl acetate (20 mL*3). The combined organic layers were washed with brine, dried over Na₂SO₄, filtered and concentrated under reduced pressure to give a residue. The residue was purified by prep-HPLC (Column, [Phenomenex luna C18 250*50 mm*10 um]; mobile phase: [ACN] and [H₂O](conditions: [water (0.225% FA)-ACN], B %: 53%-83%; Detector, UV 254 nm. RT: [7 min]) to afford 2-[[7-(4-chloro-2-fluoro-phenyl)-5-[(2S,6R)-2-(1-cyclopropylpyrazol-4-yl)-6-methyl-morpholin-4-yl]thiazolo[4,5-d]pyrimidin-2-yl]-methyl-amino]ethanol (40 mg, 0.0736 mmol, 40.87% yield) as white solid. (P1, single enantiomer of known absolute configuration): [M+H]⁺=544.1; purity=99.2% (220 nm); Retention time=0.958 min; ¹H NMR (400 MHz, CHLOROFORM-d) δ ppm 0.97-1.03 (m, 2H) 1.07-1.14 (m, 2H) 1.30 (d, J=6.24 Hz, 3H) 2.72 (dd, J=13.14, 10.58 Hz, 1H) 2.95 (dd, J=13.20, 11.00 Hz, 1H) 3.28 (br s, 3H) 3.57 (tt, J=7.27, 3.73 Hz, 1H) 3.72-4.00 (m, 5H) 4.57 (dd, J=10.94, 2.63 Hz, 1H) 4.76 (br d, J=13.08 Hz, 1H) 4.87 (br d, J=13.20 Hz, 1H) 7.20-7.26 (m, 1H) 7.29 (d, J=1.96 Hz, 1H) 7.53 (d, J=3.91 Hz, 2H) 7.70 (t, J=8.07 Hz, 1H).

Another peak (RT=0.860 min) was given as white solid, but HPLC showed it was impure. Then it was purified by prep-TLC (PE:EtOAc=0:1, Rf=0.25) to afford isomer 2-[(2E)-7-(4-chloro-2-fluoro-phenyl)-2-methylimino-5-[rac-(2S,6R)-2-(1-cyclopropylpyrazol-4-yl)-6-methyl-morpholin-4-yl]thiazolo[4,5-d]pyrimidin-3-yl]ethanol (1.7 mg, 0.00312 mmol, 1.74% yield) as white solid. LCMS and HPLC showed 100% purity of isomer, but ¹H NMR showed the purity is unqualified. NOTE 2: The absolute configuration of isomer was confirmed by 2D NMR.

### Example 11—Synthesis of Compound I-109: 7-(4-chloro-2-fluoro-phenyl)-N-(cyclopropylmethyl)-N-methyl-5-[(2S,6R)-2-(1-cyclopropylpyrazol-4-yl)-6-methyl-morpholin-4-yl]thiazolo[4,5-d]pyrimidin-2-amine

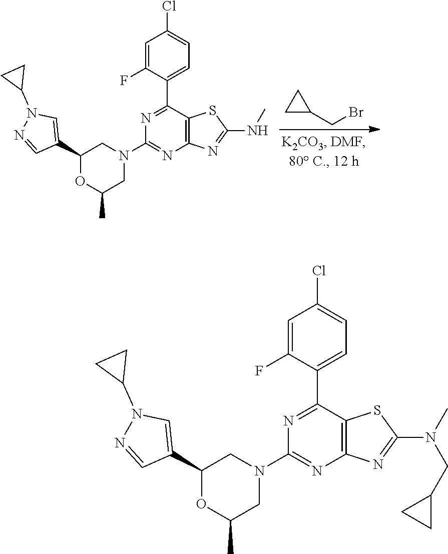

Step 1: To a mixture of 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine (1.00 eq, 90 mg, 0.180 mmol) and K₂CO₃ (4.00 eq, 100 mg, 0.720 mmol) in DMF (1.5 mL) was added bromomethylcyclopropane (4.00 eq, 97 mg, 0.720 mmol) at 25° C. Then the reaction mixture was stirred at 80° C. for 16 hours. LCMS (5-95AB/1.5 min): RT=1.092 min, 554.1=[M+H]⁺, ESI+ showed 53.1% of desired product and RT=0.925 min, 554.1=[M+H]⁺, ESI+ showed 18.0% of isomer and RT=0.974 min, 500.1=[M+H]⁺, ESI+ showed 25.5% of starting material. The reaction was filtered by a filter. The filtrate was purified by prep-HPLC (Column, [Waters Xbridge 150*25 mm*5 um]; mobile phase: [ACN] and [H₂O](conditions: [water (NH₄HCO₃)-ACN], B %: 69%-99%; Detector, UV 254 nm. RT: [8 min]) to afford 7-(4-chloro-2-fluoro-phenyl)-N-(cyclopropylmethyl)-N-methyl-5-[(2S,6R)-2-(1-cyclopropylpyrazol-4-yl)-6-methyl-morpholin-4-yl]thiazolo [4,5-d]pyrimidin-2-amine (34 mg, 0.0613 mmol, 34.08% yield) as white solid (P1, single enantiomer of known absolute configuration): [M+H]⁺=554.1; purity=100% (220 nm); Retention time=1.084 min; ¹H NMR (400 MHz, CHLOROFORM-d) δ ppm 0.33 (q, J=4.93 Hz, 2H) 0.55-0.65 (m, 2H) 0.97-1.03 (m, 2H) 1.07-1.16 (m, 3H) 1.30 (d, J=6.24 Hz, 3H) 2.66-2.79 (m, 1H) 2.95 (dd, J=13.20, 10.88 Hz, 1H) 3.09-3.71 (m, 6H) 3.80 (ddd, J=10.45, 6.30, 2.57 Hz, 1H) 4.58 (dd, J=10.88, 2.69 Hz, 1H) 4.79 (br d, J=12.96 Hz, 1H) 4.90 (br d, J=12.72 Hz, 1H) 7.22 (dd, J=10.21, 1.90 Hz, 1H) 7.26 (d, J=2.32 Hz, 1H) 7.53 (d, J=5.14 Hz, 2H) 7.70 (t, J=8.07 Hz, 1H).

The starting material 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine (20 mg, 0.0404 mmol, 22.44% yield) was recycled as white solid, which confirmed by LCMS (5-95AB/1.5 min): RT=1.012 min, 500.2=[M+H]⁺, ESI+ showed 100% of starting material. NOTE: Absolute configuration of isomer was confirmed by 2D NMR. This product should have same regio-selectivity for final step.

### Example 12—Synthesis of Compound: 7-(4-chloro-2-fluoro-phenyl)-N-(2-fluoroethyl)-N-methyl-5-[(2S,6R)-2-(1-cyclopropylpyrazol-4-yl)-6-methyl-morpholin-4-yl]thiazolo[4,5-d]pyrimidin-2-amine (I-114) and (E)-7-(4-chloro-2-fluoro-phenyl)-3-(2-fluoroethyl)-N-methyl-5-[(2S,6R)-2-(1-cyclopropylpyrazol-4-yl)-6-methyl-morpholin-4-yl]thiazolo[4,5-d]pyrimidin-2-imine (I-159)

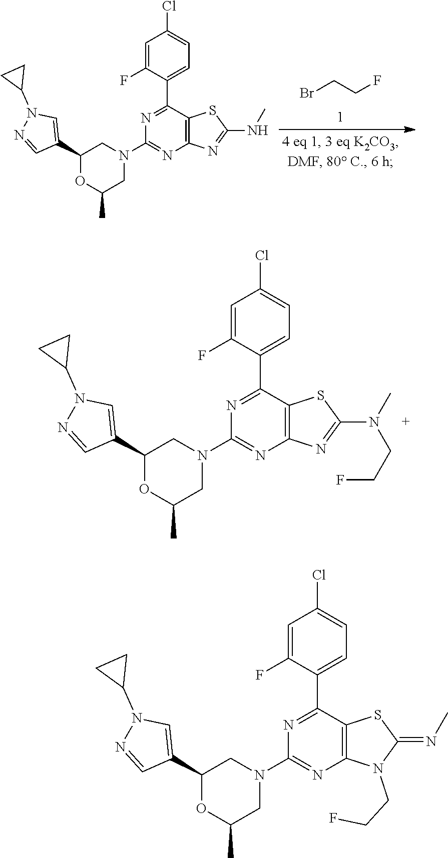

Step 1: To a mixture of 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine (1.00 eq, 90 mg, 0.180 mmol) and K₂CO₃ (4.00 eq, 100 mg, 0.720 mmol) in DMF (1.5 mL) was added 1-bromo-2-fluoroethane (2.50 eq, 0.034 mL, 0.450 mmol) at 25° C. Then the reaction mixture was stirred at 80° C. for 6 hours. LCMS (5-95AB/1.5 min): RT=1.023 min, 546.1=[M+H]⁺, ESI+ showed 69.4% of desired product and RT=0.901 min, 546.1=[M+H]⁺, ESI+ showed 22.7% of isomer. The combined reaction was filtered by a filter. The filtrate was purified by prep-HPLC (Column, [Waters Xbridge 150*25 mm*5 um]; mobile phase: [ACN] and [H₂O](conditions: [water (NH₄HCO₃)-ACN], B %: 60%-90%; Detector, UV 254 nm. RT: [8 min]) to afford 7-(4-chloro-2-fluoro-phenyl)-N-(2-fluoroethyl)-N-methyl-5-[(2S,6R)-2-(1-cyclopropylpyrazol-4-yl)-6-methyl-morpholin-4-yl]thiazolo[4,5-d]pyrimidin-2-amine (38 mg, 0.0683 mmol, 37.93% yield) as white solid.

(P1, single enantiomer of known absolute configuration): [M+H]⁺=546.1; purity=98.8% (220 nm); Retention time=1.026 min; ¹H NMR (400 MHz, CHLOROFORM-d) δ ppm 0.97-1.06 (m, 2H) 1.06-1.14 (m, 2H) 1.30 (d, J=6.24 Hz, 3H) 2.73 (dd, J=13.20, 10.64 Hz, 1H) 2.96 (dd, J=13.20, 10.88 Hz, 1H) 3.29 (br s, 3H) 3.57 (tt, J=7.31, 3.82 Hz, 1H) 3.76-3.86 (m, 1H) 3.92-4.18 (m, 2H) 4.58 (dd, J=10.82, 2.63 Hz, 1H) 4.68 (t, J=4.46 Hz, 1H) 4.74-4.83 (m, 2H) 4.89 (br d, J=13.08 Hz, 1H) 7.23 (dd, J=10.21, 1.90 Hz, 1H) 7.29 (br d, J=1.71 Hz, 1H) 7.53 (d, J=4.03 Hz, 2H) 7.71 (t, J=8.07 Hz, 1H).

(E)-7-(4-chloro-2-fluoro-phenyl)-3-(2-fluoroethyl)-N-methyl-5-[(2S,6R)-2-(1-cyclopropylpyrazol-4-yl)-6-methyl-morpholin-4-yl]thiazolo[4,5-d]pyrimidin-2-imine (5.6 mg, 0.00993 mmol, 5.51% yield) as white solid. (P2, single enantiomer of known absolute configuration): [M+H]⁺=546.1; purity=96.45% (220 nm); Retention time=0.905 min; ¹H NMR (400 MHz, CHLOROFORM-d) δ ppm 0.98-1.05 (m, 2H) 1.13 (br d, J=2.50 Hz, 2H) 1.31 (br d, J=6.00 Hz, 3H) 2.67-2.77 (m, 1H) 2.95 (br t, J=12.01 Hz, 1H) 3.09 (s, 3H) 3.54-3.63 (m, 1H) 3.73-3.84 (m, 1H) 4.30-4.43 (m, 2H) 4.56 (br d, J=11.01 Hz, 1H) 4.64 (br d, J=13.01 Hz, 1H) 4.67-4.77 (m, 2H) 4.81 (br t, J=5.00 Hz, 1H) 7.22 (br d, J=10.51 Hz, 1H) 7.25-7.27 (m, 1H) 7.54 (s, 2H) 7.61 (t, J=8.00 Hz, 1H).

### Example 13—Synthesis of Compounds: Synthesis of Compounds 7-(4-chloro-2-fluoro-phenyl)-N-ethyl-N-methyl-5-[(2S,6R)-2-(1-cyclopropylpyrazol-4-yl)-6-methyl-morpholin-4-yl]thiazolo[4,5-d]pyrimidin-2-amine (I-119) and (E)-7-(4-chloro-2-fluoro-phenyl)-3-ethyl-N-methyl-5-[(2S,6R)-2-(1-cyclopropylpyrazol-4-yl)-6-methyl-morpholin-4-yl]thiazolo[4,5-d]pyrimidin-2-imine (I-154)

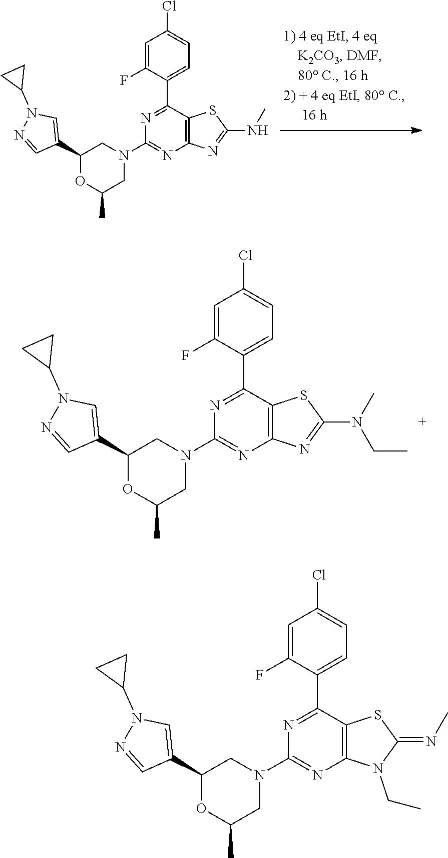

Step 1: To a mixture of 7-(4-chloro-2-fluoro-phenyl)-N-methyl-5-[(2S,6R)-2-(1-cyclopropylpyrazol-4-yl)-6-methyl-morpholin-4-yl]thiazolo[4,5-d]pyrimidin-2-amine (1.00 eq, 90 mg, 0.180 mmol) and K₂CO₃ (4.00 eq, 100 mg, 0.720 mmol) in DMF (1.5 mL) was added EtI (2.50 eq, 0.036 mL, 0.450 mmol) at 25° C. Then the reaction mixture was stirred at 80° C. for 24 hours. LCMS (5-95AB/1.5 min): RT=1.041 min, 528.1=[M+H]⁺, ESI+ showed 33% of desired product and RT=0.966 min, 500.1=[M+H]⁺, ESI+ showed 33% of starting material and RT=0.890 min, 528.1=[M+H]⁺, ESI+ showed 27.2% of isomer. Then IODOETHANE (3.00 eq, 0.044 mL, 0.540 mmol) was added and the reaction was stirred at 80° C. for 24 h. LCMS (5-95AB/1.5 min): RT=1.036 min, 528.1=[M+H]⁺, ESI+ showed 38% of desired product and RT=0.961 min, 500.1=[M+H]⁺, ESI+ showed 16.8% of starting material and RT=0.885 min, 528.1=[M+H]⁺, ESI+ showed 36% of isomer. The reaction was filtered by a filter. The filtrate was purified by prep-HPLC (Column, [Waters Xbridge 150*25 mm*5 um]; mobile phase: [ACN] and [H₂O] (conditions: [water (NH₄HCO₃)-ACN], B %: 62%-92%; Detector, UV 254 nm. RT: [8 min]) to afford 7-(4-chloro-2-fluoro-phenyl)-N-ethyl-N-methyl-5-[(2S,6R)-2-(1-cyclopropylpyrazol-4-yl)-6-methyl-morpholin-4-yl]thiazolo[4,5-d]pyrimidin-2-amine (25 mg, 0.0481 mmol, 26.72% yield) as white solid. (P1, single enantiomer of known absolute configuration): [M+H]⁺=528.1; purity=100% (220 nm); Retention time=1.037 min. ¹H NMR (400 MHz, CHLOROFORM-d) δ ppm 0.98-1.03 (m, 2H) 1.08-1.14 (m, 2H) 1.26-1.31 (m, 6H) 2.72 (dd, J=13.14, 10.70 Hz, 1H) 2.96 (dd, J=13.14, 10.94 Hz, 1H) 3.08-3.40 (m, 3H) 3.44-3.94 (m, 4H) 4.58 (dd, J=10.88, 2.57 Hz, 1H) 4.79 (br d, J=13.20 Hz, 1H) 4.90 (br d, J=13.08 Hz, 1H) 7.22 (dd, J=10.15, 1.83 Hz, 1H) 7.28 (d, J=1.83 Hz, 1H) 7.53 (d, J=4.77 Hz, 2H) 7.70 (t, J=8.07 Hz, 1H).

(E)-7-(4-chloro-2-fluoro-phenyl)-3-ethyl-N-methyl-5-[(2S,6R)-2-(1-cyclopropylpyrazol-4-yl)-6-methyl-morpholin-4-yl]thiazolo[4,5-d]pyrimidin-2-imine (13 mg, 0.0242 mmol, 13.47% yield) as white solid. (P2, single enantiomer of known absolute configuration): [M+H]⁺=528.1; purity=100% (220 nm); Retention time=0.886 min. ¹H NMR (400 MHz, CHLOROFORM-d) δ ppm 0.98-1.05 (m, 2H) 1.09-1.17 (m, 2H) 1.29-1.34 (m, 6H) 2.71 (dd, J=13.14, 10.70 Hz, 1H) 2.95 (dd, J=13.20, 11.00 Hz, 1H) 3.10 (s, 3H) 3.58 (tt, J=7.29, 3.77 Hz, 1H) 3.74-3.85 (m, 1H) 4.09 (br d, J=7.09 Hz, 2H) 4.57 (dd, J=10.82, 2.51 Hz, 1H) 4.65 (br d, J=12.96 Hz, 1H) 4.76 (br d, J=13.45 Hz, 1H) 7.22 (dd, J=10.15, 1.83 Hz, 1H) 7.26 (d, J=1.96 Hz, 1H) 7.54 (s, 2H) 7.62 (t, J=8.07 Hz, 1H).

### Example 14—Synthesis of Compound: (2S,6R)-2-(1-cyclopropyl-1H-pyrazol-4-yl)-4-(4-(2,4-difluorophenyl)-2-isopropyl-2H-pyrazolo[3,4-d]pyrimidin-6-yl)-6-methylmorpholine (I-129) and 2S,6R)-2-(1-cyclopropyl-1H-pyrazol-4-yl)-4-(4-(2,4-difluorophenyl)-1-isopropyl-1H-pyrazolo[3,4-d]pyrimidin-6-yl)-6-methylmorpholine (I-139)

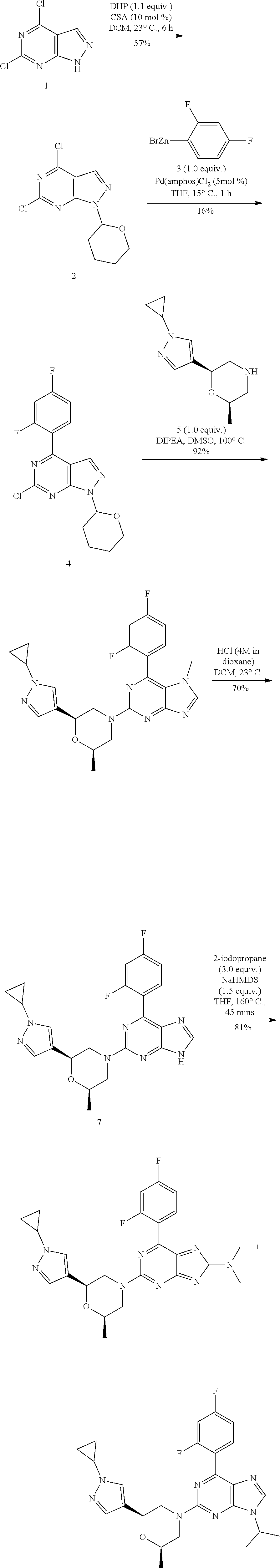

Step 1: To a solution of 4,6-dichloro-1H-pyrazolo[3,4-d]pyrimidine (1.0 equiv., 2.0 g, 10.6 mmol) and (1S)-(+)-10-camphorsulphonic acid (0.1 equiv., 246 mg, 1.1 mmol) in DCM (105 mL) at 0° C. was added 3,4-dihydro-2H-pyran (1.1 eq, 1.1 mL, 11.6 mmol) and the resulting solution was stirred overnight at room temperature. When the reaction was judged complete by TLC analysis, the reaction mixture was washed with sat. Na₂CO₃ (aq.), water, brine, dried over MgSO₄ filtrated and the solvent was removed under reduced pressure to give the 4,6-dichloro-1-(tetrahydro-2H-pyran-2-yl)-1H-pyrazolo[3,4-d]pyrimidine 2 (1.67 g, 6.11 mmol, 57%). ¹H NMR (400 MHz, CDCl₃): OH 8.25 (s, 1H), 6.04-6.00 (d, 1H), 4.18-4.13 (m, 1H), 3.85-3.80 (t, 1H), 2.60-2.58 (m, 1H), 2.17-2.15 (m, 1H), 1.99-1.96 (d, 1H), 1.84-1.82 (t, 2H), 1.77-1.76 (d, 1H).

Step 2: A flame dried 100 mL round-bottom flask under argon was charged with Pd(amphos)Cl₂ (0.05 equiv., 110 mg, 0.16 mmol), 4,6-dichloro-1-(tetrahydro-2H-pyran-2-yl)-1H-pyrazolo[3,4-d]pyrimidine (1.0 eq, 850 mg, 3.1 mmol) and THF (35 mL). Bromo-(2,4-difluorophenyl)zinc (1.0 equiv., 24 mL, 3.1 mmol) was added dropwise over 30 min and the reaction mixture was stirred for an additional 1.5 h at room temperature. At this point, the reaction mixture was filtrated over a pad of celite and the solvent was removed under reduced pressure. The crude material was purified by flash chromatography (Biotage® Sfar column 50 g, using a gradient from 100% hexanes to 30% EtOAc). The selected fractions were evaporated to yield the desired (4,6-dichloro-1-(tetrahydro-2H-pyran-2-yl)-1H-pyrazolo[3,4-d]pyrimidine 4 (180 mg, 0.51 mmol, 16%). ¹H NMR (DMSO-d6, 400 MHz): δ_{H} 8.46 (1H, d, J=4.2 Hz), 8.03 (1H, td, J=8.6, 6.5 Hz), 7.57 (1H, ddd, J=11.2, 9.3, 2.5 Hz), 7.36 (1H, td, J=8.5, 2.5 Hz), 5.97 (1H, dd, J=10.2, 2.5 Hz), 3.95 (1H, d, J=11.5 Hz), 3.71-3.77 (1H, m), 2.38-2.44 (1H, m), 2.02 (1H, br s), 1.93 (1H, dd, J=13.1, 3.6 Hz), 1.78 (1H, br s), 1.58 (2H, s).

Step 3: DIPEA (3.0 eq, 0.27 mL, 1.5 mmol) was added to a solution of 4,6-dichloro-1-(tetrahydro-2H-pyran-2-yl)-1H-pyrazolo[3,4-d]pyrimidine (1.00 eq, 180 mg, 0.513 mmol) in DMSO (2.5 mL) and the resulting solution was heated to 100° C. for 30 min. The reaction mixture was cooled to room temperature and the reaction mixture was directly purified by C18 chromatography (Biotage® column 24 g, using a gradient from 5% CH₃CN in water (0.1% FA) to 85% CH₃CN in water (0.1% FA)). The selected fractions were evaporated to yield the desired (2S,6R)-2-(1-cyclopropyl-1H-pyrazol-4-yl)-4-(4-(2,4-difluorophenyl)-1-(tetrahydro-2H-pyran-2-yl)-1H-pyrazolo[3,4-d]pyrimidin-6-yl)-6-methylmorpholine 6 (240 mg, 0.46 mmol, 89%). ESI-MS (m/z+): 522.3 [M+H]+, LC-RT: 1.86 min.

Step 4: HCl (20.0 eq, 2.3 mL, 9.20 mmol) was added to a solution ofrac-(2S,6R)-2-(1-cyclopropylpyrazol-4-yl)-4-[4-(2,4-difluorophenyl)-1-tetrahydropyran-2-yl-pyrazolo[3,4-d]pyrimidin-6-yl]-6-methyl-morpholine (1.00 eq, 240 mg, 0.460 mmol) in DCM (2 mL)/1,4-Dioxane (2 mL). The reaction mixture was stirred at RT for 1 h. At this point the reaction mixture was dilluted with EtOAc (20 mL) and washed with sat. Na₂CO₃ (aq.), water, brine, dried over MgSO₄, filtrated and the solvents were removed under reduced pressure. The crude material was purified by C18 chromatography (Biotage column 24 g, using a gradient from 5% CH₃CN in water (0.1% FA) to 85% CH₃CN in water (0.1% FA). The selected fractions were evaporated to yield the desired product (2S,6R)-2-(1-cyclopropyl-1H-pyrazol-4-yl)-4-(4-(2,4-difluorophenyl)-1H-pyrazolo[3,4-d]pyrimidin-6-yl)-6-methylmorpholine 7 (140 mg, 0.32 mmol, 70%). ESI-MS (m/z+): 438.3 [M+1]+, LC-RT: 1.52 min.

Step 5: To a solution of (2S,6R)-2-(1-cyclopropyl-1H-pyrazol-4-yl)-4-(4-(2,4-difluorophenyl)-1H-pyrazolo[3,4-d]pyrimidin-6-yl)-6-methylmorpholine (1.0 equiv., 80 mg, 0.18 mmol) in THF (230 μL) at 0° C. was added NaHMDS (1.5 equiv., 0.27 mL, 0.27 mmol) followed by 2-iodopropane (3.0 equiv., 0.06 mL, 0.55 mmol) and the mixture stirred at 160° C. for 1 h under controlled microwave irradiation. When the reaction was judge complete by LCMS (1 h). The reaction mixture was cooled down to room temperature and the solvent was removed under reduced pressure. The crude material was purified by C18 chromatography (Biotage® C18 column 40 g, using a gradient from 30% ACN in water (0.1% FA) to 85% ACN in water (0.1% FA). The selected fractions were evaporated to yield the desired products (2S,6R)-2-(1-cyclopropyl-1H-pyrazol-4-yl)-4-(4-(2,4-difluorophenyl)-2-isopropyl-2H-pyrazolo[3,4-d]pyrimidin-6-yl)-6-methylmorpholine (57 mg, 0.12 mmol, 65%) and 2S,6R)-2-(1-cyclopropyl-1H-pyrazol-4-yl)-4-(4-(2,4-difluorophenyl)-1-isopropyl-1H-pyrazolo[3,4-d]pyrimidin-6-yl)-6-methylmorpholine (14 mg, 0.03 mmol, 14%).

I-129: ¹H NMR (CHCl₃-d, 400 MHz): SH 7.93 (1H, td, J=8.5, 6.5 Hz), 7.86 (1H, d, J=4.6 Hz), 7.51 (2H, d, J=4.0 Hz), 7.03 (1H, td, J=8.3, 2.4 Hz), 6.95 (1H, ddd, J=10.9, 8.7, 2.5 Hz), 5.00 (1H, dd, J=13.3, 2.3 Hz), 4.87 (1H, dd, J=13.2, 2.1 Hz), 4.55-4.63 (2H, m), 3.76-3.84 (1H, m), 3.53-3.57 (1H, m), 2.99 (1H, dd, J=13.3, 10.9 Hz), 2.75 (1H, dd, J=13.3, 10.6 Hz), 1.28 (3H, d, J=6.2 Hz), 1.07-1.11 (2H, m), 0.95-1.00 (2H, m). ¹⁹F NMR (CHCl₃-d, 376 MHz): δ_{F} −106.1, −108.8.

I-139: ¹H NMR (DMSO-d₆, 400 MHz): δ_{H} 7.91-7.95 (2H, m), 7.82 (1H, s), 7.43-7.49 (2H, m), 7.27 (1H, td, J=8.5, 2.4 Hz), 4.92-4.99 (1H, m), 4.67-4.75 (2H, m), 4.49 (1H, dd, J=10.9, 2.6 Hz), 3.65-3.73 (2H, m), 3.00 (1H, dd, J=13.2, 11.0 Hz), 2.70 (1H, dd, J=13.2, 10.6 Hz), 1.41 (6H, d, J=6.7 Hz), 1.18 (3H, d, J=6.2 Hz), 0.97-1.02 (2H, m), 0.87-0.94 (2H, m). ¹⁹F NMR (DMSO-d₆, 376 MHz): δ_{F} −106.4, −109.6.

### Example 15—Synthesis of Compounds: 5-[(2R,4R)-2-(1-cyclopropylpyrazol-4-yl)tetrahydropyran-4-yl]-7-[2-fluoro-4-(trifluoromethyl)phenyl]-N,N-dimethyl-thiazolo[4,5-d]pyrimidin-2-amine (I-135) and 5-[(2R,4S)-2-(1-cyclopropylpyrazol-4-yl)tetrahydropyran-4-yl]-7-[2-fluoro-4-(trifluoromethyl)phenyl]-N,N-dimethyl-thiazolo[4,5-d]pyrimidin-2-amine (I-134)

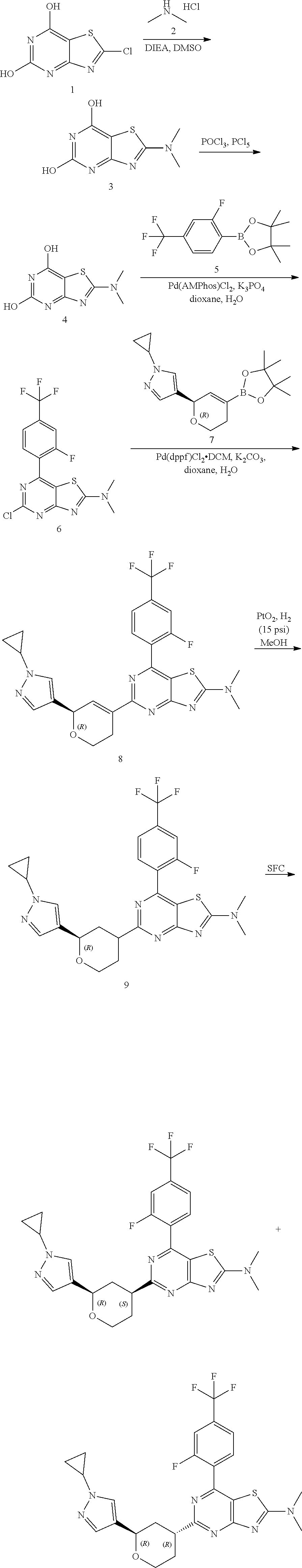

Step 1: To a solution of 2-chlorothiazolo[4,5-d]pyrimidine-5,7-diol (1.00 eq, 680 mg, 3.34 mmol) and DIMETHYLAMINE HYDROCHLORIDE (2.00 eq, 545 mg, 6.68 mmol) in DMSO (10 mL) was added DIEA (4.00 eq, 2.3 mL, 13.4 mmol). The reaction mixture was stirred at 80° C. for 1 hour. After completion, a large amount of precipitation were formed. The precipitate was collected by filtration. The filter cake was washed with PE (80 mL) and water (40 mL), then dried under high vacuum to give the 2-(dimethylamino)thiazolo[4,5-d]pyrimidine-5,7-diol (720 mg, 3.39 mmol, 69.08% yield) as pink solid. The crude product was used to next step without further purification.

Step 2: To a suspension of 2-(dimethylamino)thiazolo[4,5-d]pyrimidine-5,7-diol (1.00 eq, 720 mg, 3.39 mmol) in POCl₃ (53.2 eq, 17 mL, 180 mmol) was added PCl₅ (0.271 eq, 191 mg, 0.918 mmol) at 25° C. The mixture was stirred at 100° C. for 16 hours. LCMS showed 99.8% of desired product was detected (99.8%, Rt=0.768 min; [M+H]⁺=249.1 at 220 nm). The mixture was concentrated under reduced pressure to give a residue. The residue was quenched with saturation NaHCO₃ aqueous solution (80 mL) and then extracted with DCM (100 mL*3). The combined organic layers were dried over Na₂SO₄, filtered and concentrated under reduced pressure to give a residue. The residue was purified by flash chromatography on silica gel eluting with DCM/EtOAc (10:1) (TLC, DCM/EtOAc=0:1/V:V, Rf=0.7) to afford 5,7-dichloro-N,N-dimethyl-thiazolo[4,5-d]pyrimidin-2-amine (730 mg, 2.76 mmol, 81.45% yield) as white solid, LCMS confirmed the structure. [M+H]⁺=249.1; purity=94.6% (220 nm); Retention time=0.766 min

Step 3: Charge 2-[2-fluoro-4-(trifluoromethyl)phenyl]-4,4,5,5-tetramethyl-1,3,2-dioxaborolane (1.10 eq, 871 mg, 3.00 mmol), 5,7-dichloro-N,N-dimethyl-thiazolo[4,5-d]pyrimidin-2-amine (1.00 eq, 680 mg, 2.73 mmol), K₃PO₄ (3.00 eq, 1738 mg, 8.19 mmol) and Pd(Amphos)Cl₂ (0.130 eq, 251 mg, 0.355 mmol) into sealed bottle under N₂ atmosphere and purged with N₂ for three times, then 1,4-Dioxane (15 mL) and Water (1.5 mL) was added in one portion at 15° C., then the mixture was stirred at 60° C. for 24 hours. LCMS showed ˜47.4% of desired product was detected (47.4%, Rt=0.906 min, [M+H]+=377.1 at 220 nm). The mixture was quenched by H₂O (50 mL), extracted with EA (100 mL*3), the combined organic layers was dried over anhydrous Na₂SO₄, filtered and concentrated under reduced pressure to give the residue. The residue was purified by silical gel chromatography (PE:EA=0:1 to 1:0, PE:EA=1:1, the desired product Rf=0.3) to give the 5-chloro-7-[2-fluoro-4-(trifluoromethyl)phenyl]-N,N-dimethyl-thiazolo[4,5-d]pyrimidin-2-amine (600 mg, 1.59 mmol, 58.34% yield) as white solid, the LCMS confirmed the structure. (P1): [M+H]⁺=377.0; purity=98% (220 nm); Retention time=0.906 min

Step 4: To a solution of 5-chloro-7-[2-fluoro-4-(trifluoromethyl)phenyl]-N,N-dimethyl-thiazolo[4,5-d]pyrimidin-2-amine (1.00 eq, 550 mg, 1.46 mmol) 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 508 mg, 1.61 mmol) and K₂CO₃ (4.00 eq, 807 mg, 5.84 mmol) in 1,4-Dioxane (22 mL) and Water (2.2 mL) was added Pd(dppf)Cl₂·DCM (0.150 eq, 179 mg, 0.219 mmol) under N₂ atmosphere. The reaction mixture purged with N₂ for three times then the mixture was stirred at 80° C. for 2 hours under N₂ atmosphere. LCMS showed ˜64% of desired product was detected (64%, Rt=0.997 min, [M+H]⁺=531.2). The mixture was quenched by 100 mL H₂O, extracted with EA (200 mL*3), the combine organic layers was dried over anhydrous Na₂SO₄, filtered and concentrated under reduced pressure to give the residue. The residue was purified by silical gel chromatography (PE:EA=1:0 to 0:1, PE:EA=0:1, the desired product Rf=0.3) to give the 5-[(6R)-6-(1-cyclopropylpyrazol-4-yl)-3,6-dihydro-2H-pyran-4-yl]-7-[2-fluoro-4-(trifluoromethyl)phenyl]-N,N-dimethyl-thiazolo[4,5-d]pyrimidin-2-amine (450 mg, 0.848 mmol, 58.10% yield) as white solid, the LCMS, 1H NMR and 19F NMR confirmed the structure. [M+H]⁺=531.3; purity=100% (220 nm); Retention time=0.998 min. ¹H NMR (400 MHz, DMSO-d6) δ=8.03 (t, J=7.8 Hz, 1H), 7.97 (d, J=11.2 Hz, 1H), 7.85-7.78 (m, 2H), 7.48-7.44 (m, 1H), 7.28 (br d, J=1.2 Hz, 1H), 4.61 (dd, J=3.4, 9.9 Hz, 1H), 4.46 (br d, J=2.0 Hz, 2H), 3.69 (tt, J=3.9, 7.4 Hz, 1H), 3.30 (s, 6H), 3.00 (br d, J=16.9 Hz, 1H), 2.66-2.60 (m, 1H), 1.06-0.97 (m, 2H), 0.96-0.88 (m, 2H)

Step 5: To a solution of 5-[(6R)-6-(1-cyclopropylpyrazol-4-yl)-3,6-dihydro-2H-pyran-4-yl]-7-[2-fluoro-4-(trifluoromethyl)phenyl]-N,N-dimethyl-thiazolo[4,5-d]pyrimidin-2-amine (1.00 eq, 430 mg, 0.810 mmol) in Methanol (10 mL) was added PtO₂ (1.16 eq, 100 mg, 0.943 mmol) under N₂ atmosphere. The mixture was purged with H₂ (15 psi) 3 times, then the mixture was stirred at 30° C. for 12 h under H₂ (15 psi) atmosphere. LCMS showed ˜52% of desired product was detected (52%, Rt=0.923 min, [M+H]⁺=533.1 at 220 nm) and ˜44% of starting material was remained. The reaction mixture was stirred at 30° C. for another 24 h. LCMS showed ˜20% of starting material was remained and ˜70% of desired product was detected (70%, Rt=0.923 min, [M+H]⁺=533.1 at 220 nm). The mixture was filtered and concentrated. The residue was dissolved in Methanol (20 mL), PtO₂ (0.544 eq, 100 mg, 0.441 mmol) was added to the reaction mixture and then purged with H₂ three times. The mixture was stirred at 30° C. for another 12 h under H₂ (15 psi) atmosphere. LCMS showed ˜15% of starting material was remained and ˜76% of desired product was detected (76%, Rt=0.923 min, [M+H]⁺=533.1 at 220 nm). The mixture was filtered and the filter cake was washed with MeOH (20 mL*3), the combined organic layers was concentrated under reduced pressure to give the residue. The residue was purified by silical gel chromatography (DCE:MeOH=10:1, the desired product R_{f}=0.5) to give the residue. The residue was purified again by Prep-HPLC (Phenomenex luna C18 150*25 mm*10 um, water (FA)-ACN) and lyophilized to give the 5-[(2R)-2-(1-cyclopropylpyrazol-4-yl)tetrahydropyran-4-yl]-7-[2-fluoro-4-(trifluoromethyl)phenyl]-N,N-dimethyl-thiazolo[4,5-d]pyrimidin-2-amine (100 mg, 0.188 mmol, 23.17% yield) as white solid and then the residue was purified again by SFC (Column: Chiralpak AD-3 50×4.6 mm I.D., 3 um; Mobile phase: Phase A for CO₂, and Phase B for IPA (0.05% DEA); Gradient elution:IPA (0.05% DEA) in CO₂ from 5% to 40%; Flow rate: 3 mL/min; Detector: PDA; Column Temp: 35 C; Back Pressure: 100Ba) to give 5-[(2R,4R)-2-(1-cyclopropylpyrazol-4-yl)tetrahydropyran-4-yl]-7-[2-fluoro-4-(trifluoromethyl)phenyl]-N,N-dimethyl-thiazolo[4,5-d]pyrimidin-2-amine I-153 (P1, 18 mg, 0.0329 mmol, 17.51% yield) as white solid, which was confirmed by LCMS, HPLC, H NMR, F NMR, SFC and 5-[(2R,4S)-2-(1-cyclopropylpyrazol-4-yl)tetrahydropyran-4-yl]-7-[2-fluoro-4-(trifluoromethyl)phenyl]-N,N-dimethyl-thiazolo[4,5-d]pyrimidin-2-amine I-152 (P2, 63 mg, 0.118 mmol, 62.75% yield) as white solid, which was confirmed by LCMS, HPLC, H NMR, F NMR, SFC.

I-135: (P1, single enantiomer of known absolute configuration), LCMS: (M+H)+=533.2; purity=98.6% (220 nm); Retention time=0.908 min. ¹H NMR (400 MHz, CHLOROFORM-d) δ ppm 0.93-1.02 (m, 2H) 1.06-1.14 (m, 2H) 2.06-2.17 (m, 1H) 2.25 (ddd, J=13.29, 8.10, 4.75 Hz, 1H) 2.33-2.45 (m, 1H) 2.64-2.76 (m, 1H) 3.34 (br d, J=2.25 Hz, 6H) 3.49-3.62 (m, 2H) 3.83-3.98 (m, 2H) 4.89 (dd, J=7.82, 3.19 Hz, 1H) 7.43-7.54 (m, 3H) 7.60 (br d, J=7.88 Hz, 1H) 7.94 (t, J=7.44 Hz, 1H).

I-134: (P2, single enantiomer of known absolute configuration), LCMS: (M+H)+=533.2; purity=100% (220 nm); Retention time=0.907 min. ¹H NMR (400 MHz, CHLOROFORM-d) δ ppm 0.93-1.02 (m, 2H) 1.04-1.12 (m, 2H) 2.04-2.22 (m, 3H) 2.33 (br d, J=13.13 Hz, 1H) 3.33 (ddd, J=11.76, 8.00, 3.88 Hz, 7H) 3.55 (tt, J=7.25, 3.69 Hz, 1H) 3.76 (td, J=11.82, 2.25 Hz, 1H) 4.23 (br dd, J=11.44, 2.81 Hz, 1H) 4.51 (dd, J=11.32, 1.69 Hz, 1H) 7.45-7.54 (m, 3H) 7.59 (br d, J=8.13 Hz, 1H) 7.93 (t, J=7.44 Hz, 1H).

### Example 16—Synthesis of Compound: (2S,6R)-2-(1-cyclopropyl-1H-pyrazol-4-yl)-4-(6-(2,4-difluorophenyl)-7-methyl-7H-purin-2-yl)-6-methylmorpholine (I-124) and (2-((2S,6R)-2-(1-cyclopropyl-1H-pyrazol-4-yl)-6-methylmorpholino)-6-(2,4-difluorophenyl)-N,N,7-trimethyl-7H-purin-8-amine (I-144)

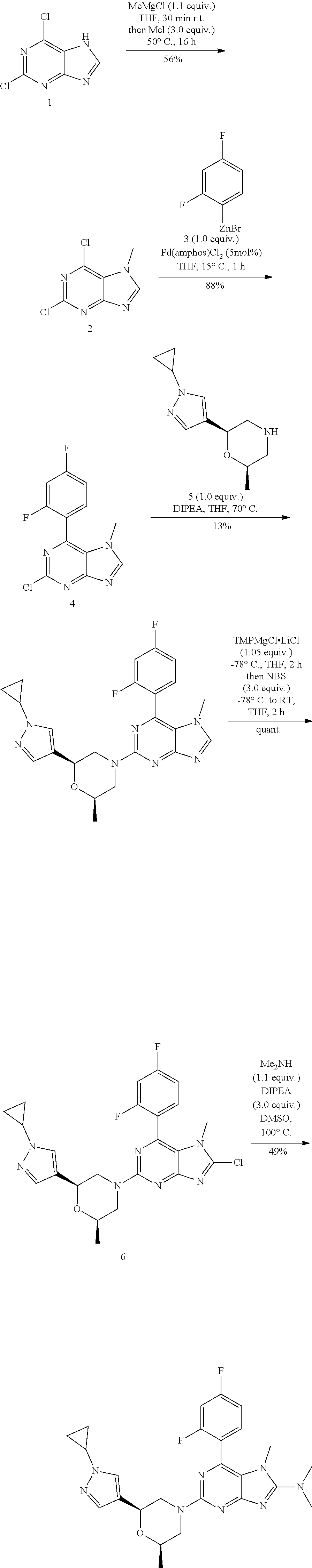

Step 1: MeMgCl (3.0 M in THF) (1.1 equiv., 3.9 mL, 11.6 mmol) was added dropwise to a solution of 2,6-dichloro-9H-purine (1.0 equiv., 2.0 g, 10.6 mmol) in THF (40 mL) at room temperature. The reaction mixture was stirred for 30 min and MeI (3.0 eq, 2.0 mL, 31.7 mmol) was added in one portion. The reaction was then stirred at 50° C. for 16 h. When the reaction was judged complete by LCMS, solution was cooled to room temperature and MeOH (2 mL) was added to neutralize the unreacted base. The solvents were removed under reduced pressure and the residue was directly purified by flash chromatography (Biotage® Sfar column 100 g, using a gradient from 2% MeOH in DCM to 10% MeOH in DCM). The selected fractions were evaporated to yield the desired 2,6-dichloro-7-methyl-purine 2 (1.20 g, 5.9 mmol, 56%). ¹H NMR (CHCl₃-d, 400 MHz): δ_{H} 8.19 (1H, s), 4.16 (3H, s). Note: the other regioisomer is observed at 8.08 ppm and 3.9 ppm (˜4%).

Step 2: A flame dried round-bottom flask under argon was charged with 2,6-dichloro-7-methyl-purine (1.0 equiv., 1.45 g, 7.1 mmol), Pd(amPhos)Cl₂ (0.05 equiv., 253 mg, 0.36 mmol) and THF (70 mL). The solution was cooled down to −10° C. and chloro-(2,4-difluorophenyl)zinc (1.05 eq, 30 mL, 7.5 mmol) solution in THF was added dropwise. The reaction was stirred at 0° C. for 2 h. When the reaction was judged complete by LCMS, the reaction mixture was filtered over a pad of Celite and the solvent was removed under reduced pressure. The crude material was purified by flash chromatography (Biotage Sfar® column 100 g, using a gradient from 100% CH₂Cl₂ to 100% EtOAc). The selected fractions were evaporated to yield the desired 2-chloro-6-(2,4-difluorophenyl)-7-methyl-7H-purine 4 (1.08 g, 3.8 mmol, 54%). ¹H NMR (CHCl₃-d, 400 MHz): δ_{H} 7.65 (1H, td, J=8.4, 6.1 Hz), 7.13 (1H, td, J=8.3, 2.4 Hz), 6.99-7.04 (1H, m), 4.20 (1H, s), 3.79 (3H, s).

Step 3: To a solution of 2-chloro-6-(2,4-difluorophenyl)-7-methyl-7H-purine (1.0 equiv., 0.5 g, 1.8 mmol) in THF (15 ml) was added DIPEA (5.0 eq, 1.6 mL, 8.9 mmol) followed by (2S,6R)-2-(1-cyclopropylpyrazol-4-yl)-6-methyl-morpholine (1.0 eq, 0.37 g, 1.8 mmol). The mixture was then stirred at 70° C. for 16 h. The reaction was cooled down to room temperature, diluted with EtOAc and washed with a sat. NH₄Cl (sat.), brine and dried over Na₂SO₄, and the solvent was removed under reduced pressure. The crude material was purified by flash chromatography (Biotage Sfar® column 50 g, using a gradient from 100% DCM to 100% EtOAc). The selected fractions were evaporated to yield the desired (2S,6R)-2-(1-cyclopropyl-1H-pyrazol-4-yl)-4-(6-(2,4-difluorophenyl)-7-methyl-7H-purin-2-yl)-6-methylmorpholine I-124 (110 mg, 0.24 mmol, 13%). ¹H NMR (CHCl₃-d, 400 MHz): SH 7.90 (1H, s), 7.55 (1H, td, J=8.4, 6.3 Hz), 7.51 (2H, d, J=3.1 Hz), 7.06 (1H, td, J=8.3, 2.5 Hz), 6.96 (1H, ddd, J=9.8, 8.7, 2.5 Hz), 4.83-4.87 (1H, m), 4.71-4.75 (1H, m), 4.57 (1H, dd, J=10.9, 2.7 Hz), 3.75-3.82 (1H, m), 3.51-3.56 (4H, m), 2.93 (1H, dd, J=13.2, 10.9 Hz), 2.70 (1H, dd, J=13.2, 10.6 Hz), 1.28 (3H, d, J=6.2 Hz), 0.94-1.01 (2H, m). ¹⁹F NMR (CHCl₃-d, 376 MHz): δ_{F} −110.3, −106.6. ESI-MS (m/z+): 452.3 [M+1]+, LC-RT: 1.36 min. Note: 10% of the other regioisomer is observed in 1H NMR.

Step 4: (2S,6R)-2-(1-cyclopropylpyrazol-4-yl)-4-[6-(2,4-difluorophenyl)-7-methyl-purin-2-yl]-6-methyl-morpholine (1.0 eq, 121 mg, 0.27 mmol) was dissolved in anhydrous THF (2.5 mL) and cooled to −78° C. TMPMgCl·LiCl (1 M in THF) (1.05 eq, 281 μL, 0.281 mmol) was added dropwise at −78° C. and the reaction mixture was stirred at −78° C. for 2 hours. A solution of NBS (3.0 eq, 143 mg, 0.8 mmol) in THF (1 mL) was added into the reaction mixture at −78° C. The reaction was allowed to warm to room temperature and stirred for 16 hours. The reaction was quenched with saturated NH₄Cl solution, extracted with CH₂Cl₂ (2×20 mL). The combined organic extracts were washed with brine, dried over anhydrous Na₂SO₄, filtrated and concentrated under reduced pressure. The crude material was purified by flash chromatography (Biotage Sfar® column 50 g, using a gradient from 100% hexanes to 10% EtOAc in hexanes) to afford (2S,6R)-4-(8-chloro-6-(2,4-difluorophenyl)-7-methyl-7H-purin-2-yl)-2-(1-cyclopropyl-1H-pyrazol-4-yl)-6-methylmorpholine 6 (110 mg, 0.23 mmol, 85% yield). ESI-MS (m/z+): 486.3 [M+H]+, LC-RT: 1.55 min. No NMR data available.

Step 5: A flame dried microwave vial under N₂ was charged with (2S,6R)-4-(8-chloro-6-(2,4-difluorophenyl)-7-methyl-7H-purin-2-yl)-2-(1-cyclopropyl-1H-pyrazol-4-yl)-6-methylmorpholine (1.0 eq, 40 mg, 0.08 mmol), dimethylamine (1.1 eq, 4.6 μL, 0.1 mmol), DIPEA (3.0 eq, 43 μL, 0.3 mmol) and DMSO (1.0 mL) and the reaction mixture was heated to 115° C. for 2 h. The reaction mixture was cooled down to room temperature and directly loaded onto a Biotage 12 g C18 column. The desired product was eluted using a gradient from 30% CH₃CN in water (0.1% FA) to 95% CH₃CN in water (0.1% FA). The desired fractions were lyophilized to afford (2-((2S,6R)-2-(1-cyclopropyl-1H-pyrazol-4-yl)-6-methylmorpholino)-6-(2,4-difluorophenyl)-N,N,7-trimethyl-7H-purin-8-amine (20 mg, 0.04 mmol, 49%) as a white solid. ¹H NMR (DMSO-d6, 400 MHz): SH 7.80 (1H, s), 7.70-7.76 (1H, m), 7.40-7.44 (2H, m), 7.26 (1H, td, J=8.5, 2.5 Hz), 4.51-4.56 (2H, m), 4.45-4.49 (2H, m), 3.65-3.71 (2H, m), 3.13 (4H, s), 3.07 (7H, s), 2.80 (2H, t, J=11.9 Hz), 2.55 (1H, d, J=11.7 Hz), 1.18 (4H, d, J=6.2 Hz), 1.00-1.03 (3H, m), 0.92-0.95 (2H, m). ¹⁹F NMR (DMSO-d6, 376 MHz): δ_{F} −110.7, −108.3. ESI-MS (m/z+): 495.3 [M+1]+, LC-RT: 1.27 min.

I-191 and I-283 are synthesized following the procedure for I-144, with the following starting materials.

| I # | Structure | Name | Starting Material 1 | Starting Material 2 |
|---|---|---|---|---|
| I-191 | 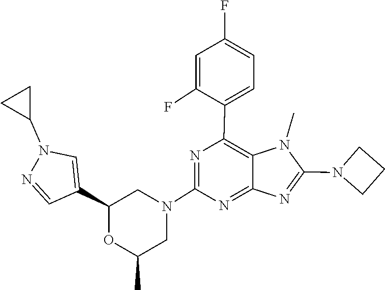 [SMILES: C[C@@H]1CN(c2nc(-c3ccc(F)cc3F)c3c(n2)nc(N2CCC2)n3C)C[C@H](c2cnn(C3CC3)c2)O1] | (2S,6R)-4-(8-(azetidin-1-yl)- 6-(2,4-difluorophenyl)-7- methyl-7H-purin-2-yl)-2-(1- cyclopropyl-1H-pyrazol-4- yl)-6-methylmorpholine | (2S,6R)-4-(8-chloro-6- (2,4-difluorophenyl)-7- methyl-7H-purin-2-yl)- 2-(1-cyclopropyl-1H- pyrazol-4-yl)-6- methylmorpholine 6 | Azetidine hydrochloride |
| I-283 | 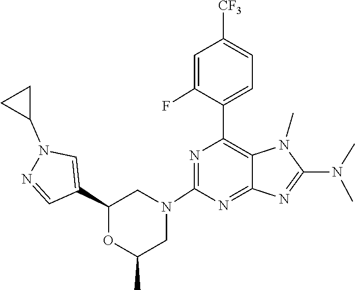 [SMILES: C[C@@H]1CN(c2nc(-c3ccc(C(F)(F)F)cc3F)c3c(n2)nc(N(C)C)n3C)C[C@H](c2cnn(C3CC3)c2)O1] | 2-((2S,6R)-2-(1- cyclopropyl-1H-pyrazol-4- yl)-6-methylmorpholino)-6- (2-fluoro-4- (trifluoromethyl)phenyl)- N,N,7-trimethyl-7H-purin-8- amine | 2,6-dichloro-9H-purine 1 | (2-fluoro-4- (trifluoromethyl) phenyl)zinc(II) bromide |

### Analytical Data:

| Ex # | H¹ NMR | F¹⁹ NMR | M + H |
|---|---|---|---|
| I-191 | ¹H NMR (DMSO-d₆, 400 MHz): δ_{H} 7.76 (1H, s), 7.63 (1H, td, J = | ¹⁹F NMR (DMSO- | 507.4 |
| | 8.5, 6.6 Hz), 7.35-7.41 (2H, m), 4.40-4.50 (3H, m), 4.23 (4H, | d₆, 376 MHz): δ_{F} −110.9 | |
| | t, J = 7.7 Hz), 3.61-3.67 (2H, m), 3.03 (3H, d, J = 1.2 Hz), 2.75 | (1F, m), −108.3 | |
| | (1H, dd, J = 12.9, 10.8 Hz), 2.36 (2H, t, J = 7.7 Hz), 1.14 (3H, | (1F, m). | |
| | d, J = 6.2 Hz), 0.96-0.99 (2H, m), 0.87-0.91 (2H, m). One peak | | |
| | of the morpholine is masked by the water peak. | | |
| I-283 | ¹H NMR (DMSO-d₆, 400 MHz): δ_{H} 7.93 (1H, t, J = 7.5 Hz), | ¹⁹F NMR (DMSO- | 545.4 |
| | 7.87 (1H, d, J = 9.9 Hz), 7.80 (1H, s), 7.76 (1H, d, J = 8.1 Hz), | d₆, 376 MHz): δF −112.3 | |
| | 7.43 (1H, s), 4.44-4.56 (3H, m), 3.65-3.71 (2H, m), 3.13 (3H, s), | (1F, t, 7 Hz), −61.2 | |
| | 3.08 (6H, s), 2.82 (1H, dd, J = 13.0, 10.8 Hz), 1.18 (3H, d, J = | (3F, s). | |
| | 6.2 Hz), 1.00-1.03 (2H, m), 0.92-0.95 (2H, m). | | |

### Example 17—Synthesis of Compound I-149: 7-[2-fluoro-4-(trifluoromethyl)phenyl]-5-[(2S,6R)-2-[1-(methoxymethyl)pyrazol-4-yl]-6-methyl-morpholin-4-yl]-N,N-dimethyl-thiazolo[4,5-d]pyrimidin-2-amine

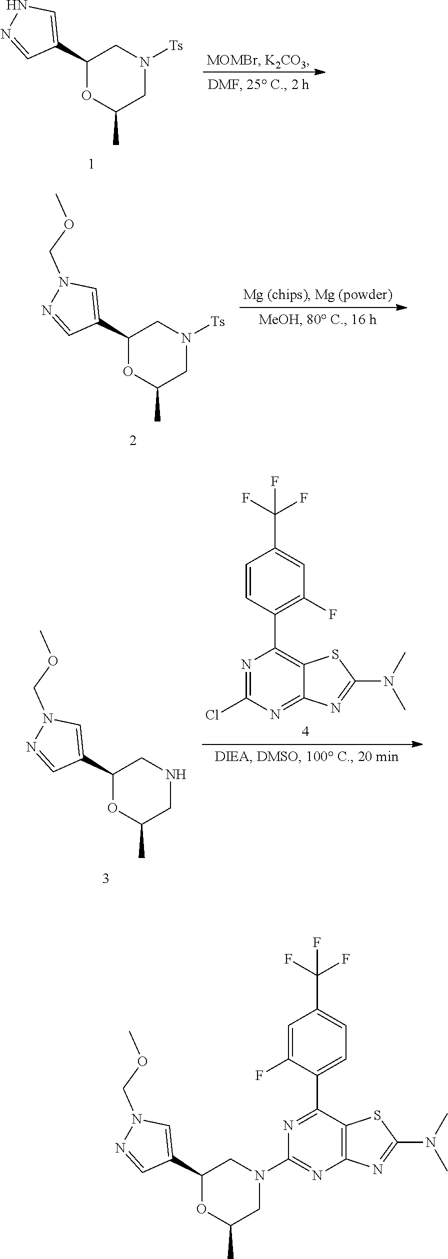

Step 1: To a solution of (2R,6S)-2-methyl-4-(p-tolylsulfonyl)-6-(1H-pyrazol-4-yl)morpholine (1.00 eq, 800 mg, 2.49 mmol) and K₂CO₃ (2.00 eq, 688 mg, 4.98 mmol) in DMF (10 mL) was added bromo(methoxy)methane (1.70 eq, 0.35 mL, 4.23 mmol). The mixture solution was stirred at 25° C. for 2 h. LCMS (5-95AB/1.5 min): RT=0.820 min, 366.1=[M+H]+, ESI+ showed ˜85% of desired product was detected. The mixture was quenched by 20 mL saturation NaHCO₃ solution, extracted with EA (3*100 mL). The combined organic layer was dried over anhydrous Na₂SO₄, filtered and concentrated under reduced pressure to get the crude residue. The residue was purified by column chromatography on silica gel (eluted with petroleum ether/ethyl acetate=100:1 to 1:1, PE:EA=1:1, the desired product R_{f}=0.4) to afford (2S,6R)-2-[1-(methoxymethyl)pyrazol-4-yl]-6-methyl-4-(p-tolylsulfonyl)morpholine (600 mg, 1.64 mmol, 65.96% yield) as colourless oil, LCMS and H NMR confirmed the structure. (P1): (M+H)+=366.2; purity=99.3% (220 nm); Retention time=0.824 min. ¹H NMR (400 MHz, CHLOROFORM-d) 6=7.64 (d, J=8.3 Hz, 2H), 7.51 (d, J=8.1 Hz, 2H), 7.36 (d, J=8.0 Hz, 2H), 5.34 (s, 2H), 4.75-4.57 (m, 1H), 3.91-3.82 (m, 1H), 3.76 (td, J=2.1, 11.4 Hz, 1H), 3.64 (td, J=2.1, 11.3 Hz, 1H), 3.33 (s, 3H), 2.46 (s, 3H), 2.29-2.19 (m, 1H), 2.09-2.06 (m, 1H), 2.03 (s, 1H), 1.21 (d, J=6.4 Hz, 3H)

Step 2: To a solution of (2S,6R)-2-[1-(methoxymethyl)pyrazol-4-yl]-6-methyl-4-(p-tolylsulfonyl)morpholine (1.00 eq, 600 mg, 1.64 mmol) in Methanol (30 mL) was added Mg (powder) (15.2 eq, 600 mg, 25.0 mmol) and Mg (chips) (15.2 eq, 600 mg, 25.0 mmol) at 25° C. and then the mixture was stirred for 16 h at 80° C. LCMS showed ˜32% of starting material was remained and one new peak was detected (no desired mass signal). Mg (chips) (15.2 eq, 600 mg, 25.0 mmol) was added to the reaction mixture and then the mixture was stirred at 80° C. for another 16 h. LCMS showed the starting material was consumed completely and one new peak was detected (no desired mass signal). The mixture was filtered and the filter cake was washed with MeOH (20 mL*3). The combine organic layers was concentrated under reduced pressure to give crude (2S, 6R)-2-[1-(methoxymethyl) pyrazol-4-yl]-6-methyl-morpholine (840 mg, 3.98 mmol, 242.18% yield) as a white solid and the residue was used to the next step directly.

Step 3: To a solution of 5-chloro-7-[2-fluoro-4-(trifluoromethyl)phenyl]-N,N-dimethyl-thiazolo[4,5-d]pyrimidin-2-amine (1.00 eq, 100 mg, 0.265 mmol) and (2S,6R)-2-[1-(methoxymethyl)pyrazol-4-yl]-6-methyl-morpholine (2.50 eq, 140 mg, 0.664 mmol) in DMSO (2 mL) was added DIEA (5.00 eq, 0.54 mL, 3.26 mmol), then the mixture was stirred at 100° C. for 1.5 h. LCMS (5-95AB/1.5 min): RT=0.830 min, 552.1=[M+H]+, ESI+ showed starting material was consumed completely and desired mass was detected. The reaction mixture was diluted with water 5 mL and extracted with EA (10 mL*3). The combined organic layers were dried over [Na₂SO₄], filtered and concentrated under reduced pressure to give a residue. The residue was purified by prep-TLC (SiO₂, EA) (TLC: EA, R_{f}=0.4) to give the crude product. The residue was purified by prep-HPLC (Phenomenex C18 75*30 mm*3 um, water (FA)-ACN) to give 7-[2-fluoro-4-(trifluoromethyl)phenyl]-5-[(2S,6R)-2-[1-(methoxymethyl)pyrazol-4-yl]-6-methyl-morpholin-4-yl]-N,N-dimethyl-thiazolo [4,5-d]pyrimidin-2-amine (103 mg, 0.186 mmol, 70.03% yield) as off-white solid, which was confirmed by LCMS, HPLC, H NMR, F NMR and SFC.

I-149 (P1, single enantiomer of known absolute configuration), LCMS: (M+H)+=552.0; purity=100% (220 nm); Retention time=1.013 min. ¹H NMR (400 MHz, CHLOROFORM-d) δ ppm 1.31 (d, J=6.25 Hz, 3H) 2.75 (dd, J=13.26, 10.76 Hz, 1H) 2.98 (dd, J=13.20, 10.94 Hz, 1H) 3.15-3.42 (m, 9H) 3.77-3.87 (m, 1H) 4.58-4.67 (m, 1H) 4.80 (br d, J=13.13 Hz, 1H) 4.90-4.99 (m, 1H) 5.37 (s, 2H) 7.47 (d, J=10.26 Hz, 1H) 7.56 (d, J=7.75 Hz, 1H) 7.64 (s, 2H) 7.89 (t, J=7.50 Hz, 1H).

### Example 18—Synthesis of Compound I-179: N-(4-chloro-2-fluoro-phenyl)-2-[[(2R,4S)-2-(1-cyclopropylpyrazol-4-yl)tetrahydropyran-4-yl]oxymethyl]-6-methyl-5-propyl-pyridin-3-amine

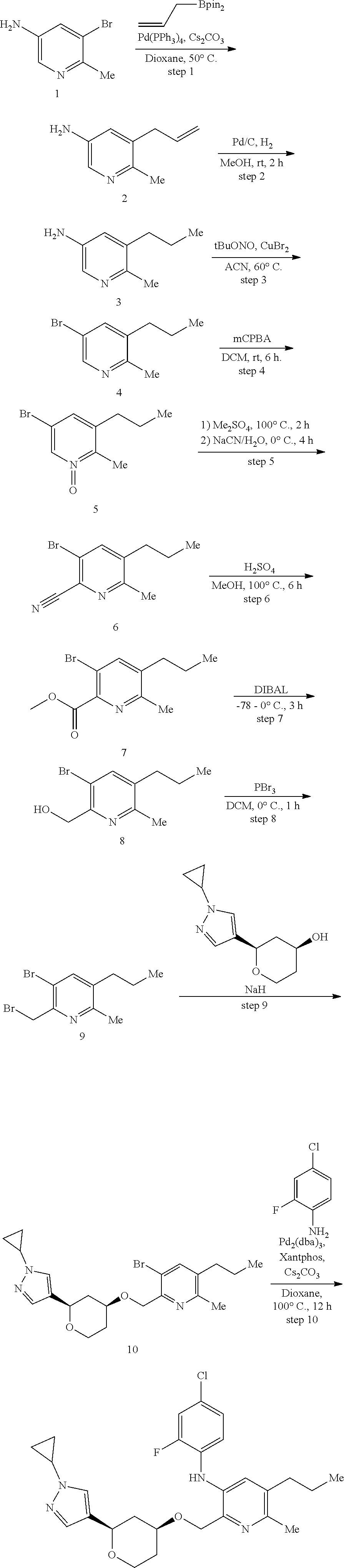

Step 1: A mixture of 5-bromo-6-methylpyridin-3-amine (8.90 g, 59.2 mmol, 1.0 eq), 2-allyl-4,4,5,5-tetramethyl-1,3,2-dioxaborolane (11.9 g, 71.1 mmol, 1.2 eq), Pd(PPh₃)₄ (1.37 g, 1.18 mmol, 0.02 eq) and Cs₂CO₃ (57.8 g, 177.7 mmol, 3.0 eq) was prepared in a round bottom flask under nitrogen. Then 1,4-dioxane (150 mL) was added and the suspension was stirred at 100° C. for 8 hours. LCMS indicated the starting material was consumed completely and 80% of the desired compound was detected. The reaction mixture was cooled to room temperature, filtered through a plug of silica gel and concentrated under reduced pressure. The crude product was purified by column chromatography (SiO₂, EtOAc/Et₃N=5:1) to give the product 5-allyl-6-methylpyridin-3-amine (6.43 g, 30.0 mmol, 50% yield) as a yellow solid. ¹H NMR (400 MHz, DMSO) δ 7.67 (d, J=2.4 Hz, 1H), 6.68 (d, J=2.4 Hz, 1H), 5.94-5.83 (m, 1H), 5.07-5.00 (m, 4H), 3.21 (d, J=6.8 Hz, 2H), 2.24 (s, 3H).

Step 2: To a solution of 5-allyl-6-methylpyridin-3-amine (6.43 g, 43.4 mmol, 1.0 eq) in MeOH (30 mL) was added 10% Pd/C (1.84 g, 1.74 mmol, 0.04 eq). Then the mixture was stirred at room temperature under H₂ for 4 hours. LCMS indicated the starting material was consumed and the desired compound was detected. The suspension was filtered through a plug of silica gel and concentrated under reduced pressure. The crude product was used for the next step without further purification.

Step 3: To a solution of 6-methyl-5-propyl-pyridin-3-amine (6.0 g, 39.9 mmol, 1.0 eq) and CuBr₂ (11.6 g, 51.9 mmol, 1.3 eq) in 45% HBr (50 mL) was added a solution of NaNO₂ (4.7 g, 67.9 mmol, 1.7 eq) in water (20 mL) at 0° C. dropwise under nitrogen. The mixture was stirred at 0° C. for an additional 2 hours. LCMS indicated that the starting material was consumed, and the desired compound was detected. The resulting solution was basified by aq. NaOH solution to pH 7-8 and extracted with ethyl acetate (50 mL×3). The organic phase was dried over Na₂SO₄ and concentrated under reduced pressure. The crude product was purified by chromatography (SiO₂, Petroleum ether/Ethyl acetate=5:1) to give the product 5-bromo-2-methyl-3-propyl-pyridine (5.16 g, 60.3 mmol, 60% yield) as a yellow solid. ¹H NMR (400 MHz, DMSO) δ 8.39 (s, 1H), 7.77 (d, J=2.0 Hz, 1H), 2.56 (t, J=7.6 Hz, 2H), 2.42 (s, 3H), 1.60-1.51 (m, 2H), 1.08 (s, 1H), 0.93 (t, J=7.4 Hz, 3H).

Step 4: To a solution of 5-bromo-2-methyl-3-propyl-pyridine (5.0 g, 23.4 mmol, 1.0 eq) in DCM (30 mL) was added m-CPBA (6.04 g, 35.0 mmol, 1.5 eq) under nitrogen. The mixture was stirred at room temperature for 3 hours. LCMS indicated that the starting material was consumed, and desired product was detected. The reaction was quenched with aq. NaS₂O₃ solution, extracted with ethyl acetate (20 mL×3) and washed with water. The organic phase was dried over Na₂SO₄ and concentrated under reduced pressure. The crude product was purified by column chromatography (SiO₂, Petroleum ether/Ethyl acetate=1:1) to give 5-bromo-2-methyl-3-propyl-pyridine 1-oxide (5.0 g, 21.7 mmol, 93% yield) as a yellow oil. LC-MS: Rt: 0.907 min, m/z: 229.9 [M+H]⁺. 87% purity at 254 nm.

Step 5: A mixture of 5-bromo-2-methyl-3-propyl-pyridine-1-oxide (4.4 g, 19.1 mmol, 1.0 eq) and Me₂SO₄ (12.0 g, 95.3 mmol, 5.0 eq) was stirred at 100° C. for 2 hours. Then the mixture was cooled to room temperature and a solution of NaCN (3.73 g, 76.2 mmol, 4.0 eq) in water (30 mL) was added. The resulting solution was stirred at room temperature for 12 hours. LCMS indicated that the starting material was consumed, and the desired product was detected. The reaction was quenched with aq. Na₂S₂O₃, washed with water and extracted with EtOAc. The organic phase was dried over Na₂SO₄ and concentrated under reduced pressure. The crude product was purified by column chromatography (silica, Petroleum ether/Ethyl acetate=10:1) to give the product 3-bromo-6-methyl-5-propyl-pyridine-2-carbonitrile as a yellow crystalline solid (1.30 g, 5.44 mmol, 29% yield). ¹H NMR (400 MHz, DMSO) δ 8.11 (s, 1H), 3.44 (s, 3H), 2.65 (t, J=7.8 Hz, 2H), 1.63-1.54 (m, 2H), 0.94 (t, J=7.4 Hz, 3H).

Step 6: To a solution of 3-bromo-6-methyl-5-propyl-pyridine-2-carbonitrile (1.65 g, 6.90 mmol, 1.0 eq) in MeOH (35 mL) was added H₂SO₄ (5 mL) at room temperature. Then the solution was stirred at 100° C. for 12 hours. LCMS indicated that the starting material was consumed, and the desired product was detected. The solution was cooled to room temperature and basified by aq. NaOH to pH-7. Then the solution was washed with water and extracted with EtOAc. The organic phase was dried over Na₂SO₄ and concentrated under reduced pressure. The crude product was purified by column chromatography (silica, Petroleum ether/Ethyl acetate=5:1) to give the desired product methyl 3-bromo-6-methyl-5-propyl-pyridine-2-carboxylate (1.10 g, 4.04 mmol, 59% yield) as a light yellow solid.

¹H NMR (400 MHz, DMSO) δ 7.95 (s, 1H), 3.87 (s, 3H), 2.61 (t, J=7.6 Hz, 2H), 2.44 (s, 3H), 1.63-1.52 (m, 2H), 0.93 (t, J=7.2 Hz, 3H).

Step 7: To a solution of methyl 3-bromo-6-methyl-5-propyl-pyridine-2-carboxylate (1.0 g, 3.7 mmol, 1.0 eq) in THF (10 mL) under nitrogen was added DIBAL-H (15 mL, 14.7 mmol, 4.0 equiv) dropwise at −78° C. The reaction mixture was slowly warmed to room temperature and stirred for 3 hours. LCMS indicated that the starting material was consumed, and the desired product was detected. The reaction was quenched with water and extracted with EtOAc. The organic phase was dried over Na₂SO₄ and concentrated under reduced pressure. The crude product was used for the next step without further purification. LC-MS: Rt: 0.774 min, m/z: 243.9 [M+H]*. 30% purity at 214 nm.

Step 8: To a solution of (3-bromo-6-methyl-5-propyl-2-pyridyl)methanol (900 mg, 3.69 mmol, 1.0 eq) in DCM (15 mL) was added PBr₃ (924 mg, 3.69 mmol, 1.0 eq) at 0° C. under N₂. Then the mixture was stirred at 0° C. for 2 hours. LCMS indicated that the starting material was consumed, and the desired product was detected. The mixture was poured into ice water, and then extracted with ethyl acetate. The organic phase was concentrated under reduced pressure and purified by column chromatography (SiO₂, Petroleum ether/Ethyl acetate=5:1) to give the desired product 3-bromo-2-(bromomethyl)-6-methyl-5-propyl-pyridine (550 mg, 1.79 mmol, 49% yield) as a white solid. ¹H NMR (400 MHz, DMSO) δ 7.83 (s, 1H), 4.68 (s, 3H), 2.57 (t, J=7.6 Hz, 2H), 2.42 (s, 3H), 1.59-1.52 (m, 2H), 0.93 (t, J=7.4 Hz, 3H).

Step 9: To a solution of (2R,4S)-2-(1-cyclopropylpyrazol-4-yl)tetrahydropyran-4-ol (68 mg, 0.33 mmol, 1.0 eq) in THF (5 mL) was added 60% NaH (26 mg, 0.65 mmol, 2.0 eq) at 0° C. under N₂, and the mixture was stirred at 0° C. for 30 minutes. Then a solution of 3-bromo-2-(bromomethyl)-6-methyl-5-propyl-pyridine (100 mg, 0.33 mmol, 1.0 eq) in THF (5 mL) was added, and the mixture was stirred at 60° C. for an additional 8 hours. LCMS indicated that the starting material was consumed, and the desired product was detected. The mixture was poured into ice water, and then extracted with ethyl acetate. The organic phase was concentrated under reduced pressure and purified by column chromatography (SiO₂, Petroleum ether/Ethyl acetate=5:1) to give the desired product 3-bromo-2-[[(2R,4S)-2-(1-cyclopropylpyrazol-4-yl)tetrahydropyran-4-yl]oxymethyl]-6-methyl-5-propyl-pyridine (72 mg, 0.17 mmol, 51% yield) as a colorless oil. ¹H NMR (400 MHz, DMSO) δ 7.81 (s, 1H), 7.70 (s, 1H), 7.35 (s, 1H), 4.61 (s, 3H), 4.27 (t, J=12.8 Hz, 1H), 3.93 (dd, J=11.6, 4.8 Hz, 1H), 3.70-3.61 (m, 2H), 3.44 (t, J=12.8 Hz, 3H), 2.56 (t, J=7.6 Hz, 2H), 2.42 (s, 3H), 2.24 (d, J=14.4 Hz, 1H), 2.03-2.00 (m, 1 h), 1.60-1.50 (m, 2H), 1.48-1.34 (m, 2H), 1.01-0.84 (m, 7H).

Step 10: A mixture of 3-bromo-2-[[(2R,4S)-2-(1-cyclopropylpyrazol-4-yl)tetrahydropyran-4-yl](65 mg, 0.15 mmol, 1.0 eq), 4-chloro-2-fluoro-aniline (22 mg, 0.15 mmol, 1.0 eq), Pd₂(dba)₃ (9 mg, 0.02 mmol, 0.1 eq), Cs₂CO₃ (97 mg, 0.30 mmol, 2.0 eq) and XantPhos (17 mg, 0.03 mmol, 0.2 eq) was prepared under nitrogen in a flask. Then dioxane (5 mL) was added and the mixture was stirred at 100° C. for 3 hours. LCMS indicated that the starting material was consumed, and the desired product was detected. The mixture was filtered through a plug of silica gel and concentrated under reduced pressure. The crude product was purified by column chromatography (SiO₂, Petroleum ether/Ethyl acetate=5:1) to give the desired product N-(4-chloro-2-fluoro-phenyl)-2-[[(2R,4S)-2-(1-cyclopropylpyrazol-4-yl)tetrahydropyran-4-yl]oxymethyl]-6-methyl-5-propyl-pyridin-3-amine (60 mg, 0.12 mmol, 80% yield) as a yellow solid. LC-MS: Rt: 1.11 min, m/z: 499.2 [M+H]⁺. 97% purity at 214 nm. ¹H NMR (400 MHz, DMSO) δ 7.66 (s, 1H), 7.44-7.41 (m, 2H), 7.32 (s, 2H), 7.14-7.12 (m, 1H), 7.05 (t, J=8.8 Hz, 1H), 4.65 (s, 2H), 4.21 (d, J=11.2 Hz, 1H), 3.92 (dd, J=11.2, 3.2 Hz, 1H), 3.65-3.62 (m, 2H), 3.40 (t, J=12.0 Hz, 1H), 2.55-2.52 (m, 1H), 2.40 (s, 3H), 2.21-2.17 (m, 1H), 1.94 (d, J=12.8 Hz, 2H), 1.54-1.48 (m, 2H), 1.42-1.33 (m, 2H), 0.98-0.90 (m, 7H). LC-MS [M+H]⁺=499.2 R.T=1.109 min. HPLC: Rt: 3.03 min, 95% purity at 214 nm

### Example 19—Synthesis of Compound I-164: Synthesis of Compound 5-((2R,4S)-2-(1-cyclopropyl-1H-pyrazol-4-yl)tetrahydro-2H-pyran-4-yl)-7-(2-fluoro-4-(trifluoromethyl)phenyl)-2-((R)-3-methoxypyrrolidin-1-yl)thiazolo[4,5-d]pyrimidine

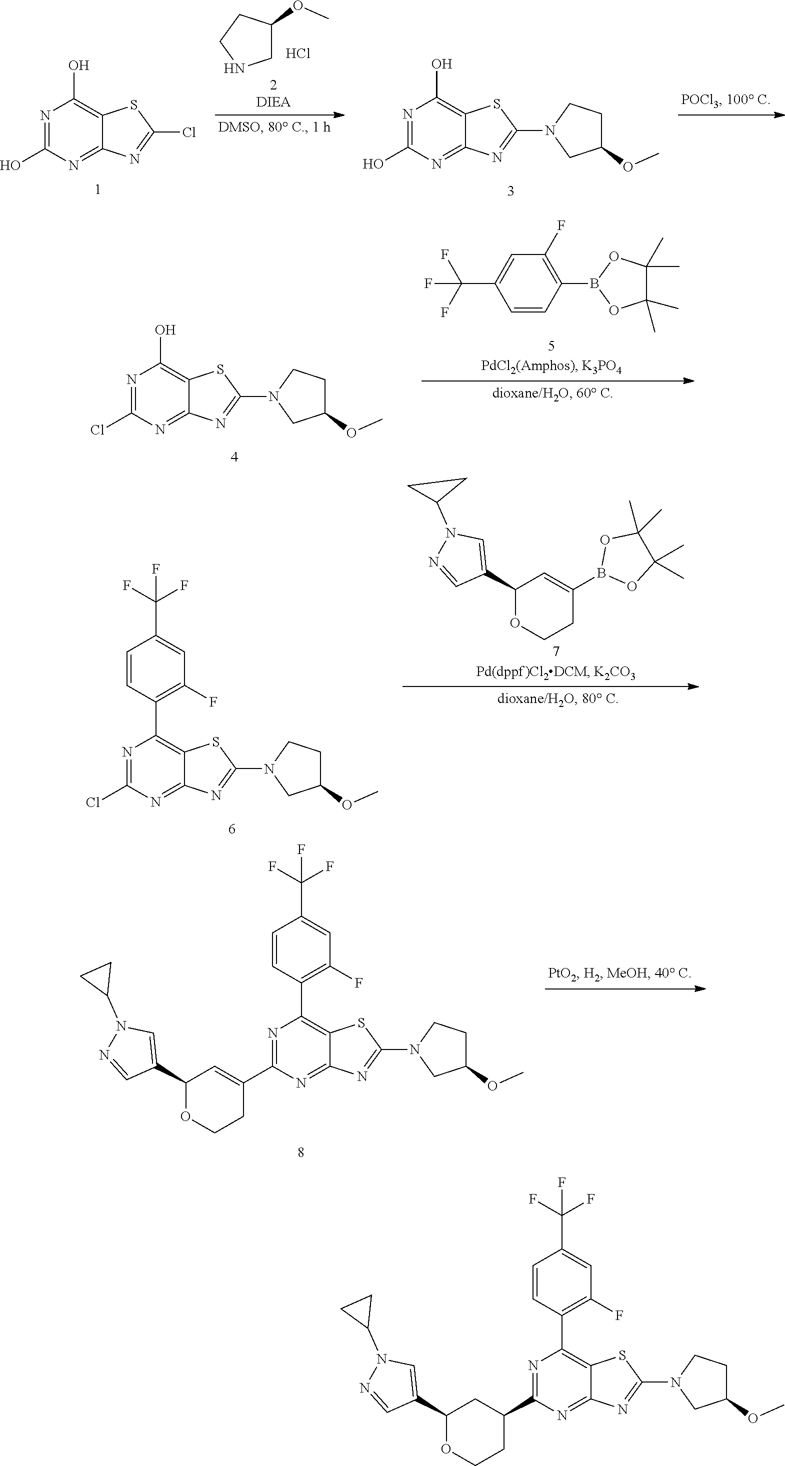

Step 1: To a solution of 2-chlorothiazolo[4,5-d]pyrimidine-5,7-diol (1.00 eq, 500 mg, 2.46 mmol) and (3R)-3-methoxypyrrolidine;hydrochloride (2.00 eq, 676 mg, 4.91 mmol) in DMSO (5 mL) was added DIEA (4.00 eq, 1.6 mL, 9.82 mmol). Then the reaction mixture was stirred at 80° C. for 1 hours. LCMS (5-95AB/1.5 min): RT=0.625 min, 269.1=[M+H]⁺, ESI+ showed 100% of desired product. The reaction mixture was adjusted to PH<7 with FA, purified by reversed-phase HPLC (0.1% FA condition) and lyophilized to give (R)-2-(3-methoxypyrrolidin-1-yl)thiazolo[4,5-d]pyrimidine-5,7-diol (500 mg, 1.86 mmol, 75.89% yield) as off-white solid, checked by LCMS and H NMR. (M+H)=269.1; purity=100% (220 nm); Retention time=0.486 min. ¹H NMR (400 MHz, DMSO) δ=11.87-11.65 (m, 1H), 10.86-10.81 (m, 1H), 3.30 (s, 1H), 3.26 (s, 3H), 2.54-2.51 (m, 4H), 2.14 (br s, 2H).

Step 2: A mixture of 2-[(3R)-3-methoxypyrrolidin-1-yl]thiazolo[4,5-d]pyrimidine-5,7-diol (1.00 eq, 500 mg, 1.86 mmol) in POCl₃ (1.00 eq, 5.0 mL, 1.86 mmol) was stirred at 100° C. for 2 hours. LCMS (5-95AB/1.5 min): RT=0.810 min, 305.0=[M+H]⁺, ESI+ showed 60% of desired product. The reaction was stirred at 100° C. for 4 hours. LCMS (5-95AB/1.5 min): RT=0.592 min, 305.0=[M+H]+, ESI+ showed 86% of desired product. The reaction mixture was concentrated under reduced pressure to give a residue, the residue was partitioned between DCM (100 mL*2) and NaHCO₃ (aq., 100 mL). The combined organic layers were dried over Na₂SO₄, filtered and concentrated under reduced pressure to give a residue, and purified by reversed-phase HPLC (0.1% FA condition) and lyophilized to give (R)-5,7-dichloro-2-(3-methoxypyrrolidin-1-yl)thiazolo[4,5-d]pyrimidine (530 mg, 1.74 mmol, 93.19% yield) as yellow brown solid, checked by LCMS and H NMR. (M+H)+=304.9; purity=96% (220 nm); Retention time=0.833 min. ¹H NMR (400 MHz, DMSO) δ=4.21-4.13 (m, 1H), 3.92-3.71 (m, 2H), 3.59-3.50 (m, 2H), 3.29 (d, J=4.8 Hz, 3H), 2.25-2.11 (m, 2H)

Step 3: Charge 5,7-dichloro-2-[(3R)-3-methoxypyrrolidin-1-yl]thiazolo[4,5-d]pyrimidine (1.10 eq, 579 mg, 1.90 mmol), 2-[2-fluoro-4-(trifluoromethyl)phenyl]-4,4,5,5-tetramethyl-1,3,2-dioxaborolane (1.00 eq, 500 mg, 1.72 mmol), K₃PO₄ (3.50 eq, 1281 mg, 6.03 mmol) and PdCl₂(Amphos) (0.1000 eq, 122 mg, 0.172 mmol) into sealed bottle under N₂ atmosphere and purged with N₂ for three times, then 1,4-Dioxane (5 mL) and Water (0.5 mL) was added in one portion at 15° C., then the mixture was stirred at 60° C. for 16 hours. LCMS (5-95AB/1.5 min): RT=0.938 min, 433.0=[M+H]⁺, ESI+ showed 42% of desired product. The reaction mixture was partitioned between ethyl acetate (50 mL*2) and water (80 mL). The combined organic layers were dried over Na₂SO₄, filtered and concentrated under reduced pressure to give a residue. The crude product was purified by reversed-phase HPLC (0.1% FA condition) and lyophilized to give (R)-5-chloro-7-(2-fluoro-4-(trifluoromethyl)phenyl)-2-(3-methoxypyrrolidin-1-yl)thiazolo[4,5-d]pyrimidine (250 mg, 0.578 mmol, 33.51% yield) as yellow brown solid, checked by LCMS and H NMR. (M+H)+=433.0; purity=100% (220 nm); Retention time=0.948 min. ¹H NMR (400 MHz, CDCl₃) δ=7.96 (t, J=7.7 Hz, 1H), 7.62-7.58 (m, 1H), 7.53-7.48 (m, 1H), 4.23-4.04 (m, 2H), 3.97-3.78 (m, 1H), 3.68-3.46 (m, 2H), 3.38 (s, 3H), 2.31 (br s, 2H).

Step 4: Charge 5-chloro-7-[2-fluoro-4-(trifluoromethyl)phenyl]-2-[(3R)-3-methoxypyrrolidin-1-yl]thiazolo[4,5-d]pyrimidine (1.00 eq, 250 mg, 0.578 mmol), 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 201 mg, 0.635 mmol), K₂CO₃ (3.50 eq, 279 mg, 2.02 mmol) and Pd(dppf)Cl₂·DCM (0.100 eq, 41 mg, 0.0578 mmol) into sealed bottle under N₂ atmosphere and purged with N₂ for three times, then 1,4-Dioxane (2 mL) and Water (0.2 mL) was added in one portion at 15° C., then the mixture was stirred at 80° C. for 16 hours. LCMS (5-95AB/1.5 min): RT=1.061 min, 587.2=[M+H]⁺, ESI+ showed 82% of desired product. The reaction mixture was partitioned between DCM (50 mL*2) and water (80 mL). The combined organic layers were dried over Na₂SO₄, filtered and concentrated under reduced pressure to give a residue. The crude product was purified by column chromatography on silica gel chromatography (PE/EtOAc=0˜100%, PE/EtOAc=0/1, the desired product R_{f}=0.1) to give 5-((R)-6-(1-cyclopropyl-1H-pyrazol-4-yl)-3,6-dihydro-2H-pyran-4-yl)-7-(2-fluoro-4-(trifluoromethyl)phenyl)-2-((R)-3-methoxypyrrolidin-1-yl)thiazolo[4,5-d]pyrimidine (270 mg, 0.460 mmol, 79.69% yield) as light yellow oil, checked by LCMS and H NMR. (M+H)=587.3; purity=96% (220 nm); Retention time=0.703 min. ¹H NMR (400 MHz, CDCl₃) δ=7.99-7.94 (m, 1H), 7.61-7.58 (m, 1H), 7.57-7.50 (m, 3H), 4.72-4.67 (m, 1H), 4.59-4.54 (m, 2H), 4.21-4.16 (m, 1H), 4.08 (br d, J=6.2 Hz, 2H), 3.62-3.55 (m, 2H), 3.38 (s, 3H), 3.25-3.18 (m, 1H), 2.91-2.83 (m, 1H), 2.38-2.06 (m, 3H), 1.13-1.10 (m, 2H), 1.02-0.98 (m, 2H)

Step 5: A mixture of 5-[(6R)-6-(1-cyclopropylpyrazol-4-yl)-3,6-dihydro-2H-pyran-4-yl]-7-[2-fluoro-4-(trifluoromethyl)phenyl]-2-[(3R)-3-methoxypyrrolidin-1-yl]thiazolo[4,5-d]pyrimidine (1.00 eq, 270 mg, 0.460 mmol) in Methanol (5 mL) was added PtO₂ (2.00 eq, 209 mg, 0.921 mmol) under N₂ atmosphere. The mixture was purged with H₂ (50 psi) 3 times, then the mixture was stirred at 40° C. for 16 h under H₂ (50 psi). LCMS (5-95AB/1.5 min): RT=0.949 min, 589.2=[M+H]⁺, ESI+ showed 49.6% of desired product. The reaction mixture was added PtO₂ (1.00 eq, 104 mg, 0.460 mmol) under N₂ atmosphere. The mixture was purged with H₂ (50 psi) 3 times, then the mixture was stirred at 40° C. for 16 h under H₂ (50 psi). LCMS (5-95AB/1.5 min): RT=0.920 min, 589.3=[M+H]⁺, ESI+ showed 80% of desired product, but include the starting material's MS. The reaction mixture was filtered and concentrated under reduced pressure to give a residue. The residue in Methanol (5 mL) was added PtO₂ (2.00 eq, 209 mg, 0.921 mmol) under N₂ atmosphere. The mixture was purged with H₂ (15 psi) 3 times, then the mixture was stirred at 40° C. for 16 h under H₂ (15 psi). LCMS (5-95AB/1.5 min): RT=1.031 min, 589.2=[M+H]⁺, ESI+ showed 72% of desired product, but include the starting material's MS. The reaction mixture was filtered and concentrated under reduced pressure to give a residue. The residue was purified by prep-HPLC (Unisil 3-100 C18 Ultra 150*50 mm*3 um; mobile phase: [water (0.1% FA)-ACN]; B %: 56%-86%, 9 min) and lyophilized to give 48 mg and also have some mixture kept in hands. 5-((2R,4S)-2-(1-cyclopropyl-1H-pyrazol-4-yl)tetrahydro-2H-pyran-4-yl)-7-(2-fluoro-4-(trifluoromethyl)phenyl)-2-((R)-3-methoxypyrrolidin-1-yl)thiazolo[4,5-d]pyrimidine (2.4 mg, 0.00408 mmol, 0.8900% yield) as white solid, checked by LCMS, HNMR, FNMR. (M+H)⁺=589.2; purity=100% (220 nm); Retention time=0.971 min. ¹H NMR (400 MHz, CDCl₃) δ (ppm)=7.93 (t, J=7.5 Hz, 1H), 7.60 (d, J=8.1 Hz, 1H), 7.54-7.44 (m, 3H), 4.51 (dd, J=1.5, 11.2 Hz, 1H), 4.23 (br dd, J=3.6, 11.3 Hz, 1H), 4.20-4.00 (m, 2H), 3.99-3.81 (m, 1H), 3.77 (dt, J=2.4, 11.7 Hz, 1H), 3.70-3.44 (m, 3H), 3.38 (s, 3H), 3.36-3.27 (m, 1H), 2.39-2.27 (m, 2H), 2.24-2.04 (m, 4H), 1.09 (br d, J=3.1 Hz, 2H), 1.04-0.95 (m, 2H).

### Example 20—Synthesis of Compound I-165: 5-[(2R)-2-(1-cyclopropylpyrazol-4-yl) tetrahydropyran-4-yl]-7-[2-fluoro-4-(trifluoromethyl) phenyl]-2-[(3S)-3-methoxypyrrolidin-1-yl]thiazolo [4,5-d]pyrimidine

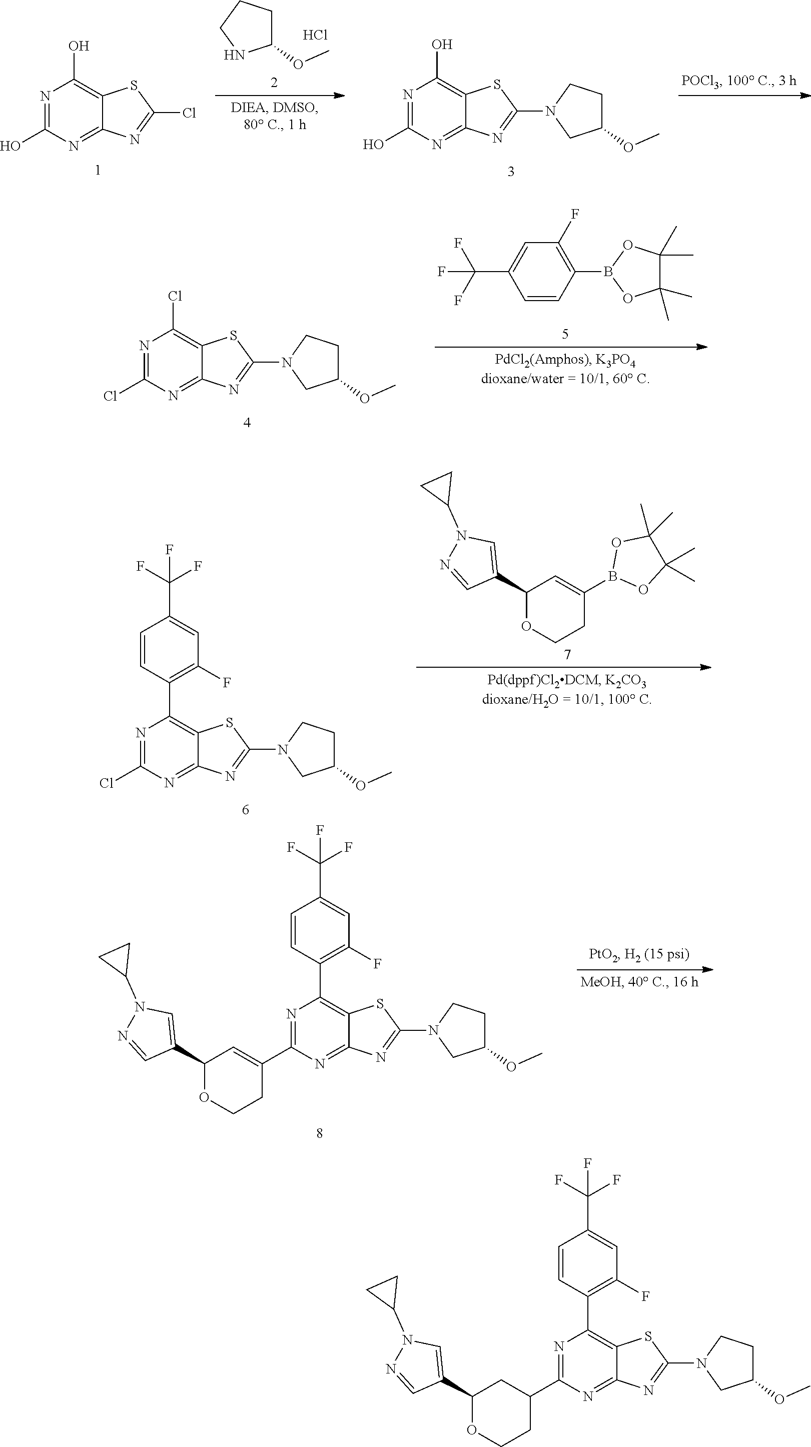

Step 1: To a solution of 2-chlorothiazolo [4, 5-d]pyrimidine-5, 7-diol (1.00 eq, 600 mg, 2.95 mmol) and (3S)-3-methoxypyrrolidine; hydrochloride (1.50 eq, 608 mg, 4.42 mmol) in DMSO (10 mL) was added DIEA (4.00 eq, 1.9 mL, 11.8 mmol). Then the reaction mixture was stirred at 80° C. for 1 hour. TLC indicated Reactant 1 was consumed completely and one new spot formed. (PE/EA=1/1, starting material Rf=0.4, new spot Rf=0.5). The mixture was purified by reversed-phase chromatography (H₂O (FA)-MeCN)). After reverse phase purification, the eluent was concentrated to remove organic solvents. The residual aqueous solution was lyophilized to give 2-[(3S)-3-methoxypyrrolidin-1-yl]thiazolo [4,5-d]pyrimidine-5,7-diol (700 mg, 2.61 mmol, 88.54% yield) as yellow solid, checked by LCMS. [M+H]+=269.0; purity=100% (220 nm); Retention time=0.300 min.

Step 2: A mixture of 2-[(3S)-3-methoxypyrrolidin-1-yl]thiazolo [4,5-d]pyrimidine-5,7-diol (1.00 eq, 600 mg, 2.24 mmol) in POCl₃ (1.00 eq, 8.0 mL, 2.24 mmol) was stirred at 100° C. for 3 hours. LCMS showed raw material was consumed completely and the major peak showed MS (78%, Rt: 0.829 min; [M+H]+=304.9 at 220 nm). The reaction mixture was concentrated under reduced pressure to give a residue, the residue was partitioned between DCM (50 mL*2) and NaHCO₃ (aq, 40 mL). The combined organic layers were dried over Na₂SO₄, filtered and concentrated under reduced pressure to give 5,7-dichloro-2-[(3S)-3-methoxypyrrolidin-1-yl]thiazolo[4,5-d]pyrimidine (650 mg, 1.77 mmol, 79.05% yield) as black solid, checked by LCMS RT=0.830 min, 304.9=[M+H]+, ESI+. [M+H]+=304.9; purity=81.5% (220 nm); Retention time=0.830 min.

Step 3: Charge 5,7-dichloro-2-[(3S)-3-methoxypyrrolidin-1-yl]thiazolo[4,5-d]pyrimidine (1.10 eq, 579 mg, 1.90 mmol), 2-[2-fluoro-4-(trifluoromethyl)phenyl]-4,4,5,5-tetramethyl-1,3,2-dioxaborolane (1.00 eq, 500 mg, 1.72 mmol), K₃PO₄ (3.50 eq, 1.28 g, 6.03 mmol) and PdCl₂(Amphos) (0.130 eq, 159 mg, 0.224 mmol) into sealed bottle under N₂ atmosphere and purged with N₂ for three times, then 1,4-Dioxane (10 mL) and Water (1 mL) was added in one portion at 15° C., then the mixture was stirred at 60° C. for 4 hours. LCMS: showed raw material was consumed completely and the major peak showed MS (48%, Rt: 0.958 min; [M+H]+=433.0 at 220 nm). The reaction was purified by reversed-phase chromatography (water(FA)-MeCN). After reversed-phase purification, the eluent was concentrated or evaporated to remove organic solvents. The residual aqueous solution was lyophilized to give 5-chloro-7-[2-fluoro-4-(trifluoromethyl)phenyl]-2-[(3S)-3-methoxypyrrolidin-1-yl]thiazolo[4,5-d]pyrimidine (250 mg, 0.566 mmol, 32.84%% yield) as white solid, checked by LCMS.=433.0; purity=98% (220 nm); Retention time=0.956 min.

Step 4: To a solution of 5-chloro-7-[2-fluoro-4-(trifluoromethyl)phenyl]-2-[(3S)-3-methoxypyrrolidin-1-yl]thiazolo[4,5-d]pyrimidine (1.00 eq, 50 mg, 0.116 mmol) and 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 40 mg, 0.127 mmol) in 1,4-Dioxane (2 mL) and Water (0.2000 mL) was added K₂CO₃ (3.00 eq, 48 mg, 0.347 mmol) and Pd(dppf)Cl₂·CH₂Cl₂ (0.200 eq, 19 mg, 0.0231 mmol). The mixture was stirred at 100° C. for 12 hours. LCMS (1B) showed 9% of reactant was remained and ˜43% of desired mass was detected, the reaction solution was poured into H₂O (5 mL), extracted with EtOAc (5 mL*3), and evaporated under reduced pressure to give the crude, which was then purified with flash column (80% EA, Rf=0.4) and evaporated under reduced pressure to give 5-[(6R)-6-(1-cyclopropylpyrazol-4-yl)-3,6-dihydro-2H-pyran-4-yl]-7-[2-fluoro-4-(trifluoromethyl)phenyl]-2-[(3S)-3-methoxypyrrolidin-1-yl]thiazolo[4,5-d]pyrimidine (40 mg, 0.0682 mmol, 59.03% yield) as yellow solid. (P1): [M+H]+=587.1; Retention time=0.956 min.

Step 5: A solution of 5-[(6R)-6-(1-cyclopropylpyrazol-4-yl)-3,6-dihydro-2H-pyran-4-yl]-7-[2-fluoro-4-(trifluoromethyl)phenyl]-2-[(3S)-3-methoxypyrrolidin-1-yl]thiazolo[4,5-d]pyrimidine (1.00 eq, 40 mg, 0.0682 mmol) in Methanol (10 mL) was added PtO₂ (1.29 eq, 20 mg, 0.0881 mmol) at N₂ (15 PSI) atmosphere, then stirred at 40° C. for 16 hours under H2 (15 PSI). LCMS showed 40% of desired product (5-95AB/1 min): RT=0.675 min, 589.2=[M+H]+, ESI+). The reaction mixture was filtered through a pad of celite. The filter cake was washed with MeOH (20 mL). The filtrate was concentrated under reduced pressure. The residue was purified by prep-HPLC (Column, [Phenomenex luna C18 250*50 mm*10 um]; mobile phase: [ACN] and [H2O](conditions: [water (0.225% FA)-ACN], B %: 65%-90%; Detector, UV 254 nm. RT: [22 min]) to afford 5-[(2R)-2-(1-cyclopropylpyrazol-4-yl)tetrahydropyran-4-yl]-7-[2-fluoro-4-(trifluoromethyl)phenyl]-2-[(3S)-3-methoxypyrrolidin-1-yl]thiazolo[4,5-d]pyrimidine (3.7 mg, 0.00618 mmol, 9.07%% yield) as white solid, which confirmed by LCMS (1B1) (5-95AB/1.5 min): RT=0.972 min, 589.2=[M+H]+ and HNMR (1A1), HPLC (1B2), FNMR (1A1). [M+H]+=589.2; purity=99.69% (220 nm); Retention time=0.972 min. 1H NMR (400 MHz, CDCl₃) δ=7.93 (br t, J=7.6 Hz, 1H), 7.60 (br d, J=8.3 Hz, 1H), 7.53-7.45 (m, 3H), 4.88 (dd, J=3.2, 8.2 Hz, 1H), 4.51 (br d, J=11.4 Hz, 1H), 4.26-4.14 (m, 2H), 3.91 (br d, J=5.5 Hz, 1H), 3.82-3.72 (m, 1H), 3.64-3.46 (m, 3H), 3.38 (s, 3H), 3.34-3.28 (m, 1H), 2.37-2.31 (m, 1H), 2.30-2.24 (m, 1H), 2.16 (br dd, J=6.8, 12.8 Hz, 2H), 2.09 (br s, 1H), 1.33-1.23 (m, 2H), 1.10 (br s, 2H), 1.02-0.95 (m, 2H).

### Example 21—Synthesis of Compound: 7-cyclohexyl-5-[(2R,4R)-2-(1-cyclopropylpyrazol-4-yl)tetrahydropyran-4-yl]-N,N-dimethyl-thiazolo[4,5-d]pyrimidin-2-amine (I-205) & 7-cyclohexyl-5-[(2R,4S)-2-(1-cyclopropylpyrazol-4-yl)tetrahydropyran-4-yl]-N,N-dimethyl-thiazolo[4,5-d]pyrimidin-2-amine (I-174)

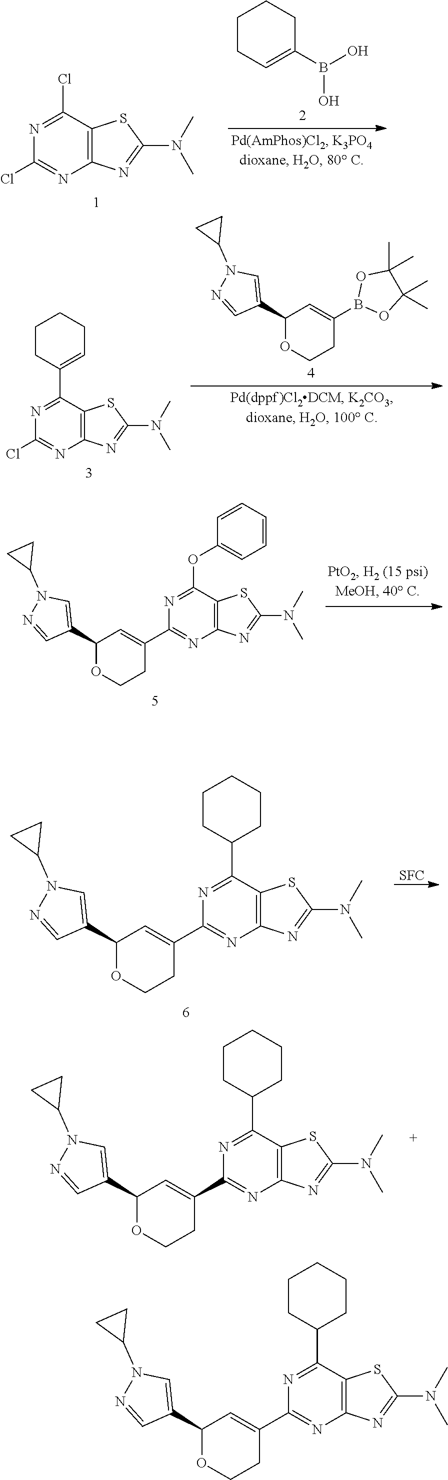

Step 1: To a solution of cyclohexen-1-ylboronic acid (1.00 eq, 394 mg, 3.13 mmol) and 5,7-dichloro-N,N-dimethyl-thiazolo[4,5-d]pyrimidin-2-amine (1.00 eq, 780 mg, 3.13 mmol), K₃PO₄ (3.00 eq, 1994 mg, 9.39 mmol) in 1,4-Dioxane (10 mL) and Water (1 mL), then Pd(Amphos)Cl₂ (0.0500 eq, 111 mg, 0.157 mmol) was added to the mixture under N₂, the mixture solution was stirred for 16 h at 80° C. LCMS (1A) showed raw material consumed and the major peak showed desired MS (M+H)+=295.0 and BP-MS (M+H)+=341.1, purity=59.73%, uv=220 nm, Retention time=0.642 min. The reaction solution was added water (30 mL) and then extracted with ethyl acetate (10 mL*2), the organics washed with 10 mL saturated brine. The organics were then separated and dried (Na₂SO₄) before concentration to dryness. The crude was then purified by silica gel column (PE/EA=1/1, Rf=0.4) to give 5-chloro-7-(cyclohexen-1-yl)-N,N-dimethyl-thiazolo[4,5-d]pyrimidin-2-amine (300 mg, 1.02 mmol, 32.50% yield) as light brown oil. (P1): ¹H NMR (400 MHz, CHLOROFORM-d) δ ppm 1.65-1.86 (m, 4H) 2.26-2.38 (m, 2H) 2.50-2.68 (m, 2H) 3.05-3.52 (m, 6H) 6.78 (tt, J=3.85, 1.78 Hz, 1H)

Step 2: To a solution of 5-chloro-7-(cyclohexen-1-yl)-N,N-dimethyl-thiazolo[4,5-d]pyrimidin-2-amine (1.00 eq, 240 mg, 0.814 mmol) in 1,4-Dioxane (10 mL) and water (1 mL) was added K₂CO₃ (3.00 eq, 338 mg, 2.44 mmol) and 1-cyclopropyl-4-[(6R)-4-(4,4,5,5-tetramethyl-1,3,2-dioxaborolan-2-yl)-3,6-dihydro-2H-pyran-6-yl]pyrazole (1.50 eq, 386 mg, 1.22 mmol), then Pd(dppf)Cl₂·DCM (0.200 eq, 119 mg, 0.163 mmol) was added to the mixture under N₂ and then stirred for 16 h at 100° C. LCMS (1A) showed raw material consumed and the major peak showed desired MS (M+H)+=449.2, purity=55.71%, uv=220 nm, Ret. Time=0.955 min. The reaction solution added water (30 mL) and then extracted with ethyl acetate (10 mL*2) and then the organics washed with 10 mL saturated brine solution. The organics were then separated and dried (Na₂SO₄) before concentration to dryness. The crude was then purified by silica gel column (PE/EA=0/1, Rf=0.5) to give 7-(cyclohexen-1-yl)-5-[(6R)-6-(1-cyclopropylpyrazol-4-yl)-3,6-dihydro-2H-pyran-4-yl]-N,N-dimethyl-thiazolo[4,5-d]pyrimidin-2-amine (180 mg, 0.401 mmol, 49.29% yield) as yellow oil. (P1): MS (M+H)+=449.2, purity=62.39%, uv=220 nm, Ret. Time=0.642 min.

Step 3: To a solution of 7-(cyclohexen-1-yl)-5-[(6R)-6-(1-cyclopropylpyrazol-4-yl)-3,6-dihydro-2H-pyran-4-yl]-N,N-dimethyl-thiazolo[4,5-d]pyrimidin-2-amine (1.00 eq, 180 mg, 0.401 mmol) in Methanol (20 mL) was added PtO₂ (1.00 eq, 91 mg, 0.401 mmol) under N₂ atmosphere. The mixture was purged with H₂ three times, then the mixture was stirred at 40° C. for 16 hours under H₂ (15 psi) atmosphere. LCMS (1A) showed 40.47% raw material remained. The reaction solution was added PtO₂ (1.00 eq, 91 mg, 0.401 mmol) under N₂ atmosphere. The mixture was purged with H₂ three times, then the mixture was stirred at 40° C. for 16 hours under H₂ (15 psi) atmosphere. LCMS (1A1) showed 15.01% raw material remained. The reaction solution filter and filtrate was concentration in vacuo and then dissolve in Methanol (15 mL) and added PtO₂ (1.00 eq, 91 mg, 0.401 mmol) under N₂ atmosphere. The mixture was purged with H₂ three times, then the mixture was stirred at 40° C. for 16 hours under H₂ (15 psi) atmosphere. LCMS (1B3) showed the major peak showed desired MS (M+H)+=453.2, purity=30.29% and 18% intermediate remained. The reaction solution was added PtO₂ (1.00 eq, 91 mg, 0.401 mmol) under N₂ atmosphere. The mixture was purged with H₂ three times, then the mixture was stirred at 40° C. for 16 hours under H₂ (15 psi) atmosphere. LCMS (1C1) showed the major peak include raw material MS and intermediate MS remained. The reaction solution was added PtO₂ (1.50 eq, 137 mg, 0.602 mmol) under N₂ atmosphere. The mixture was purged with H₂ three times, then the mixture was stirred at 40° C. for 16 hours under H₂ (15 psi) atmosphere. LCMS (1C2) showed 29.93% raw material remained and the major peak showed desired MS (M+H)+=453.2, purity=48.7%, uv=220 nm, Retention time=0.544 min. The reaction was filter and the filtrate was concentrated in vacuo and the crude purified by silica gel column (PE/EA=1/1, Rf=0.4) to give 130 mg (86.99% purity) which was confirmed by LCMS (1E2). The crude product was purified by prep-HPLC (FA) and freeze-drying to give 7-cyclohexyl-5-[(2R)-2-(1-cyclopropylpyrazol-4-yl)tetrahydropyran-4-yl]-N,N-dimethyl-thiazolo[4,5-d]pyrimidin-2-amine (50 mg, 0.110 mmol, 27.53% yield) as white solid which was confirmed by LCMS (1G1): (P1): MS (M+H)+=453.2, purity=100%, uv=220 nm, Retention time=0.848 min. HPLC (1G2) showed the product were a mixture (the ratio was ˜2/1).

Step 4: The racemate product was purified by SFC (DAICEL CHIRALPAK AD (250 mm*30 mm, 10 um), 0.1% NH₃H₂O ETOH) to give 7-cyclohexyl-5-[(2R,4S)-2-(1-cyclopropylpyrazol-4-yl)tetrahydropyran-4-yl]-N,N-dimethyl-thiazolo[4,5-d]pyrimidin-2-amine (I-174) (35 mg, 0.0771 mmol, 58.13% yield) as white solid and 7-cyclohexyl-5-[(2R,4R)-2-(1-cyclopropylpyrazol-4-yl)tetrahydropyran-4-yl]-N,N-dimethyl-thiazolo[4,5-d]pyrimidin-2-amine (I-205) (13 mg, 0.0287 mmol, 21.68% yield) as white solid.

(P1): peak 2 in SFC, Ret. Time=2.834 min. MS (M+H)+=453.2, purity=100%, uv=220 nm, Ret. Time=0.847 min. 1H NMR (400 MHz, CHLOROFORM-d) δ ppm 0.91-1.01 (m, 2H) 1.04-1.13 (m, 2H) 1.28-1.49 (m, 3H) 1.73-1.79 (m, 2H) 1.84-1.95 (m, 4H) 1.96-2.16 (m, 3H) 2.21-2.32 (m, 1H) 2.64-2.74 (m, 1H) 3.15-3.41 (m, 7H) 3.55 (tt, J=7.29, 3.77 Hz, 1H) 3.75 (td, J=11.89, 2.26 Hz, 1H) 4.20 (dd, J=11.37, 3.18 Hz, 1H) 4.49 (dd, J=11.37, 1.83 Hz, 1H) 7.47 (d, J=0.98 Hz, 2H)

(P2): peak 1 in SFC, Ret. Time=1.738 min. MS (M+H)+=453.2, purity=100%, uv=220 nm, Ret.Time=0.865 min. 1H NMR (400 MHz, CHLOROFORM-d) δ ppm 0.94-1.01 (m, 2H) 1.07-1.14 (m, 2H) 1.31-1.46 (m, 4H) 1.71-1.81 (m, 4H) 1.83-1.98 (m, 4H) 1.99-2.22 (m, 2H) 2.30-2.41 (m, 1H) 2.60-2.74 (m, 2H) 3.30 (br s, 6H) 3.39-3.49 (m, 1H) 3.51-3.60 (m, 1H) 3.83-3.92 (m, 2H) 4.85 (dd, J=8.44, 3.06 Hz, 1H) 7.46 (d, J=8.93 Hz, 2H)

### Example 22—Synthesis of Compound I-179: N-(4-chloro-2-fluoro-phenyl)-2-[[(2R,4S)-2-(1-cyclopropylpyrazol-4-yl)tetrahydropyran-4-yl]oxymethyl]-6-methyl-5-propyl-pyridin-3-amine

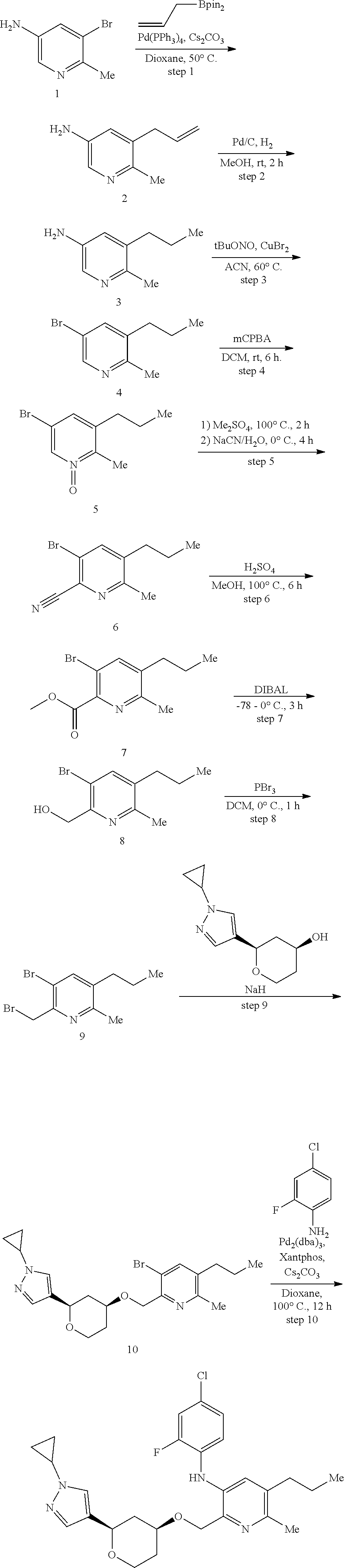

Step 1: A mixture of 5-bromo-6-methylpyridin-3-amine (8.90 g, 59.2 mmol, 1.0 eq), 2-allyl-4,4,5,5-tetramethyl-1,3,2-dioxaborolane (11.9 g, 71.1 mmol, 1.2 eq), Pd(PPh₃)₄ (1.37 g, 1.18 mmol, 0.02 eq) and Cs₂CO₃ (57.8 g, 177.7 mmol, 3.0 eq) was prepared in a round bottom flask under nitrogen. Then 1,4-dioxane (150 mL) was added and the suspension was stirred at 100° C. for 8 hours. LCMS indicated the starting material was consumed completely and 80% of the desired compound was detected. The reaction mixture was cooled to room temperature, filtered through a plug of silica gel and concentrated under reduced pressure. The crude product was purified by column chromatography (SiO₂, EtOAc/Et₃N=5:1) to give the product 5-allyl-6-methylpyridin-3-amine (6.43 g, 30.0 mmol, 50% yield) as a yellow solid. ¹H NMR (400 MHz, DMSO) δ 7.67 (d, J=2.4 Hz, 1H), 6.68 (d, J=2.4 Hz, 1H), 5.94-5.83 (m, 1H), 5.07-5.00 (m, 4H), 3.21 (d, J=6.8 Hz, 2H), 2.24 (s, 3H).

Step 2: To a solution of 5-allyl-6-methylpyridin-3-amine (6.43 g, 43.4 mmol, 1.0 eq) in MeOH (30 mL) was added 10% Pd/C (1.84 g, 1.74 mmol, 0.04 eq). Then the mixture was stirred at room temperature under H₂ for 4 hours. LCMS indicated the starting material was consumed and the desired compound was detected. The suspension was filtered through a plug of silica gel and concentrated under reduced pressure. The crude product was used for the next step without further purification.

Step 3: To a solution of 6-methyl-5-propyl-pyridin-3-amine (6.0 g, 39.9 mmol, 1.0 eq) and CuBr₂ (11.6 g, 51.9 mmol, 1.3 eq) in 45% HBr (50 mL) was added a solution of NaNO₂ (4.7 g, 67.9 mmol, 1.7 eq) in water (20 mL) at 0° C. dropwise under nitrogen. The mixture was stirred at 0° C. for an additional 2 hours. LCMS indicated that the starting material was consumed, and the desired compound was detected. The resulting solution was basified by aq. NaOH solution to pH 7-8 and extracted with ethyl acetate (50 mL×3). The organic phase was dried over Na₂SO₄ and concentrated under reduced pressure. The crude product was purified by chromatography (SiO₂, Petroleum ether/Ethyl acetate=5:1) to give the product 5-bromo-2-methyl-3-propyl-pyridine (5.16 g, 60.3 mmol, 60% yield) as a yellow solid. ¹H NMR (400 MHz, DMSO) δ 8.39 (s, 1H), 7.77 (d, J=2.0 Hz, 1H), 2.56 (t, J=7.6 Hz, 2H), 2.42 (s, 3H), 1.60-1.51 (m, 2H), 1.08 (s, 1H), 0.93 (t, J=7.4 Hz, 3H).

Step 4: To a solution of 5-bromo-2-methyl-3-propyl-pyridine (5.0 g, 23.4 mmol, 1.0 eq) in DCM (30 mL) was added m-CPBA (6.04 g, 35.0 mmol, 1.5 eq) under nitrogen. The mixture was stirred at room temperature for 3 hours. LCMS indicated that the starting material was consumed, and desired product was detected. The reaction was quenched with aq. NaS₂O₃ solution, extracted with ethyl acetate (20 mL×3) and washed with water. The organic phase was dried over Na₂SO₄ and concentrated under reduced pressure. The crude product was purified by column chromatography (SiO₂, Petroleum ether/Ethyl acetate=1:1) to give 5-bromo-2-methyl-3-propyl-pyridine 1-oxide (5.0 g, 21.7 mmol, 93% yield) as a yellow oil. LC-MS: Rt: 0.907 min, m/z: 229.9 [M+H]+. 87% purity at 254 nm.

Step 5: A mixture of 5-bromo-2-methyl-3-propyl-pyridine-1-oxide (4.4 g, 19.1 mmol, 1.0 eq) and Me₂SO₄ (12.0 g, 95.3 mmol, 5.0 eq) was stirred at 100° C. for 2 hours. Then the mixture was cooled to room temperature and a solution of NaCN (3.73 g, 76.2 mmol, 4.0 eq) in water (30 mL) was added. The resulting solution was stirred at room temperature for 12 hours. LCMS indicated that the starting material was consumed, and the desired product was detected. The reaction was quenched with aq. Na₂S₂O₃, washed with water and extracted with EtOAc. The organic phase was dried over Na₂SO₄ and concentrated under reduced pressure. The crude product was purified by column chromatography (silica, Petroleum ether/Ethyl acetate=10:1) to give the product 3-bromo-6-methyl-5-propyl-pyridine-2-carbonitrile as a yellow crystalline solid (1.30 g, 5.44 mmol, 29% yield). ¹H NMR (400 MHz, DMSO) δ 8.11 (s, 1H), 3.44 (s, 3H), 2.65 (t, J=7.8 Hz, 2H), 1.63-1.54 (m, 2H), 0.94 (t, J=7.4 Hz, 3H).

Step 6: To a solution of 3-bromo-6-methyl-5-propyl-pyridine-2-carbonitrile (1.65 g, 6.90 mmol, 1.0 eq) in MeOH (35 mL) was added H₂SO₄ (5 mL) at room temperature. Then the solution was stirred at 100° C. for 12 hours. LCMS indicated that the starting material was consumed, and the desired product was detected. The solution was cooled to room temperature and basified by aq. NaOH to pH-7. Then the solution was washed with water and extracted with EtOAc. The organic phase was dried over Na₂SO₄ and concentrated under reduced pressure. The crude product was purified by column chromatography (silica, Petroleum ether/Ethyl acetate=5:1) to give the desired product methyl 3-bromo-6-methyl-5-propyl-pyridine-2-carboxylate (1.10 g, 4.04 mmol, 59% yield) as a light yellow solid. ¹H NMR (400 MHz, DMSO) δ 7.95 (s, 1H), 3.87 (s, 3H), 2.61 (t, J=7.6 Hz, 2H), 2.44 (s, 3H), 1.63-1.52 (m, 2H), 0.93 (t, J=7.2 Hz, 3H).

Step 7: To a solution of methyl 3-bromo-6-methyl-5-propyl-pyridine-2-carboxylate (1.0 g, 3.7 mmol, 1.0 eq) in THF (10 mL) under nitrogen was added DIBAL-H (15 mL, 14.7 mmol, 4.0 equiv) dropwise at −78° C. The reaction mixture was slowly warmed to room temperature and stirred for 3 hours. LCMS indicated that the starting material was consumed, and the desired product was detected. The reaction was quenched with water and extracted with EtOAc. The organic phase was dried over Na₂SO₄ and concentrated under reduced pressure. The crude product was used for the next step without further purification. LC-MS: Rt: 0.774 min, m/z: 243.9 [M+H]⁺. 30% purity at 214 nm.

Step 8: To a solution of (3-bromo-6-methyl-5-propyl-2-pyridyl)methanol (900 mg, 3.69 mmol, 1.0 eq) in DCM (15 mL) was added PBr₃ (924 mg, 3.69 mmol, 1.0 eq) at 0° C. under N₂. Then the mixture was stirred at 0° C. for 2 hours. LCMS indicated that the starting material was consumed, and the desired product was detected. The mixture was poured into ice water, and then extracted with ethyl acetate. The organic phase was concentrated under reduced pressure and purified by column chromatography (SiO₂, Petroleum ether/Ethyl acetate=5:1) to give the desired product 3-bromo-2-(bromomethyl)-6-methyl-5-propyl-pyridine (550 mg, 1.79 mmol, 49% yield) as a white solid. ¹H NMR (400 MHz, DMSO) δ 7.83 (s, 1H), 4.68 (s, 3H), 2.57 (t, J=7.6 Hz, 2H), 2.42 (s, 3H), 1.59-1.52 (m, 2H), 0.93 (t, J=7.4 Hz, 3H).

Step 9: To a solution of (2R,4S)-2-(1-cyclopropylpyrazol-4-yl)tetrahydropyran-4-ol (68 mg, 0.33 mmol, 1.0 eq) in THF (5 mL) was added 60% NaH (26 mg, 0.65 mmol, 2.0 eq) at 0° C. under N₂, and the mixture was stirred at 0° C. for 30 minutes. Then a solution of 3-bromo-2-(bromomethyl)-6-methyl-5-propyl-pyridine (100 mg, 0.33 mmol, 1.0 eq) in THF (5 mL) was added, and the mixture was stirred at 60° C. for an additional 8 hours. LCMS indicated that the starting material was consumed, and the desired product was detected. The mixture was poured into ice water, and then extracted with ethyl acetate. The organic phase was concentrated under reduced pressure and purified by column chromatography (SiO₂, Petroleum ether/Ethyl acetate=5:1) to give the desired product 3-bromo-2-[[(2R,4S)-2-(1-cyclopropylpyrazol-4-yl)tetrahydropyran-4-yl]oxymethyl]-6-methyl-5-propyl-pyridine (72 mg, 0.17 mmol, 51% yield) as a colorless oil. ¹H NMR (400 MHz, DMSO) δ 7.81 (s, 1H), 7.70 (s, 1H), 7.35 (s, 1H), 4.61 (s, 3H), 4.27 (t, J=12.8 Hz, 1H), 3.93 (dd, J=11.6, 4.8 Hz, 1H), 3.70-3.61 (m, 2H), 3.44 (t, J=12.8 Hz, 3H), 2.56 (t, J=7.6 Hz, 2H), 2.42 (s, 3H), 2.24 (d, J=14.4 Hz, 1H), 2.03-2.00 (m, 1 h), 1.60-1.50 (m, 2H), 1.48-1.34 (m, 2H), 1.01-0.84 (m, 7H).

Step 10: A mixture of 3-bromo-2-[[(2R,4S)-2-(1-cyclopropylpyrazol-4-yl)tetrahydropyran-4-yl](65 mg, 0.15 mmol, 1.0 eq), 4-chloro-2-fluoro-aniline (22 mg, 0.15 mmol, 1.0 eq), Pd₂(dba)₃ (9 mg, 0.02 mmol, 0.1 eq), Cs₂CO₃ (97 mg, 0.30 mmol, 2.0 eq) and XantPhos (17 mg, 0.03 mmol, 0.2 eq) was prepared under nitrogen in a flask. Then dioxane (5 mL) was added and the mixture was stirred at 100° C. for 3 hours. LCMS indicated that the starting material was consumed, and the desired product was detected. The mixture was filtered through a plug of silica gel and concentrated under reduced pressure. The crude product was purified by column chromatography (SiO₂, Petroleum ether/Ethyl acetate=5:1) to give the desired product N-(4-chloro-2-fluoro-phenyl)-2-[[(2R,4S)-2-(1-cyclopropylpyrazol-4-yl)tetrahydropyran-4-yl]oxymethyl]-6-methyl-5-propyl-pyridin-3-amine (60 mg, 0.12 mmol, 80% yield) as a yellow solid. LC-MS: Rt: 1.11 min, m/z: 499.2 [M+H]+. 97% purity at 214 nm. ¹H NMR (400 MHz, DMSO) δ 7.66 (s, 1H), 7.44-7.41 (m, 2H), 7.32 (s, 2H), 7.14-7.12 (m, 1H), 7.05 (t, J=8.8 Hz, 1H), 4.65 (s, 2H), 4.21 (d, J=11.2 Hz, 1H), 3.92 (dd, J=11.2, 3.2 Hz, 1H), 3.65-3.62 (m, 2H), 3.40 (t, J=12.0 Hz, 1H), 2.55-2.52 (m, 1H), 2.40 (s, 3H), 2.21-2.17 (m, 1H), 1.94 (d, J=12.8 Hz, 2H), 1.54-1.48 (m, 2H), 1.42-1.33 (m, 2H), 0.98-0.90 (m, 7H). LC-MS [M+H]⁺=499.2 R.T=1.109 min. HPLC: Rt: 3.03 min, 95% purity at 214 nm

### Example 23—Synthesis of Compounds: 5-(4,4-difluoro-3-(2-methylpyridin-4-yl)piperidin-1-yl)-7-(2-fluoro-4-(trifluoromethyl)phenyl)-N,N-dimethylthiazolo[4,5-d]pyrimidin-2-amine (1-188), (S)-5-(4,4-difluoro-3-(2-methylpyridin-4-yl)piperidin-1-yl)-7-(2-fluoro-4-(trifluoromethyl)phenyl)-N,N-dimethylthiazolo[4,5-d]pyrimidin-2-amine (I-202) and (R)-5-(4,4-difluoro-3-(2-methylpyridin-4-yl)piperidin-1-yl)-7-(2-fluoro-4-(trifluoromethyl)phenyl)-N,N-dimethylthiazolo[4,5-d]pyrimidin-2-amine (I-203)

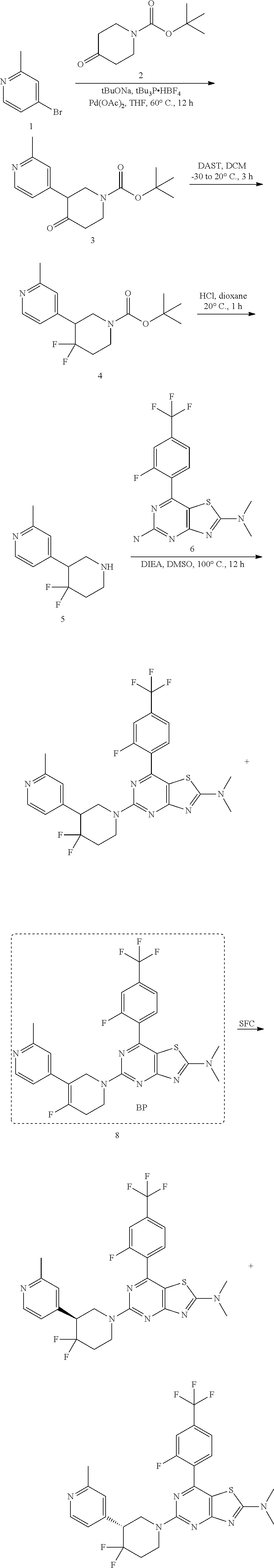

Step 1: To a mixture of tert-butyl 4-oxopiperidine-1-carboxylate (1.00 eq, 5000 mg, 25.1 mmol), 4-bromo-2-methylpyridine (1.00 eq, 4317 mg, 25.1 mmol), tBuONa (1.09 eq, 2628 mg, 27.3 mmol) and tBu₃P·HBF₄ (0.100 eq, 728 mg, 2.51 mmol) in THF (100 mL) was added Pd(OAc)₂ (0.100 eq, 563 mg, 2.51 mmol) at 25° C. The mixture was heated to 60° C. and stirred for 12 h under N₂. LCMS (XM-2021-03-041-069-P1A) showed 24% of the starting material remained and 33.4% of the desired mass was detected (33.4%, Rt: 0.462 min; [M+H]+=291.4 at 220 nm). The reaction mixture was concentrated under reduced pressure. The crude product was purified by column chromatography on silica gel eluted (PE/EtOAc=1/0 to 1/2; PE/EtOAc=1/1, the desired product Rf=0.25 showed by 254 nm) to give tert-butyl 3-(2-methylpyridin-4-yl)-4-oxopiperidine-1-carboxylate (1800 mg, 6.20 mmol, 24.70% yield) as yellow oil, checked by LCMS and H NMR. [M+H]⁺=291.2; purity=95% (220 nm); Retention time=0.487 min. ¹H NMR (400 MHz, CDCl₃) δ=8.51-8.44 (m, 1H), 7.00 (s, 1H), 6.94 (d, J=5.1 Hz, 1H), 4.34-4.12 (m, 2H), 3.72-3.47 (m, 3H), 2.64-2.53 (m, 5H), 1.51 (s, 9H)

Step 2: To a solution of tert-butyl 3-(2-methylpyridin-4-yl)-4-oxopiperidine-1-carboxylate (1.00 eq, 800 mg, 2.76 mmol) in DCM (30 mL) was added DAST (10.0 eq, 4441 mg, 27.6 mmol) at −30° C. under N₂, the mixture was stirred at 20° C. under N₂ for 3 h. LCMS 5-P1A showed that the starting material was consumed completely and 25% of the desired mass was detected (25%, Rt: 0.493 min; [M+H]+=313.4 at 220 nm). The reaction mixture was quenched by addition sat. NaHCO₃ (50 mL) at 0° C., and then extracted with DCM (40 mL×2). The combined organic layers were washed with brine (10 mL), dried over Na₂SO₄, filtered and concentrated under reduced pressure to give a residue. The residue was purified by flash silica gel chromatography (ISCO; 40 g SepaFlash Silica Flash Column, Eluent of 40˜60% EtOAc/PE; gradient: 60 mL/min; PE/EtOAc=1/1, the desired product R_{f}=0.3) to give tert-butyl 4,4-difluoro-3-(2-methylpyridin-4-yl)piperidine-1-carboxylate (250 mg, 0.800 mmol, 29.05% yield) as yellow solid, checked by LCMS 5-P1B and HNMR 5-P1A. (P1): [M+H]⁺=313.1; purity=42% (220 nm); Retention time=0.388 min. ¹H NMR (400 MHz, CDCl₃) δ=8.47 (d, J=5.1 Hz, 1H), 7.14-7.02 (m, 2H), 4.31-4.14 (m, 2H), 3.41-2.91 (m, 3H), 2.58-2.57 (m, 3H), 2.22-2.12 (m, 1H), 2.01-1.89 (m, 1H), 1.48 (s, 9H)

Step 3: To a solution of tert-butyl 4,4-difluoro-3-(2-methylpyridin-4-yl)piperidine-1-carboxylate (200 mg, 1.00 eq, 0.0029 mL, 0.640 mmol) in DCM (5 mL) was adde HCl/dioxane (4M, 2 mL) and stirred at 20° C. under N₂ for 1 h. LCMS 3-P1A showed that the starting material was consumed completely and the desired mass was major (94%, Rt: 0.720 min; [M+H]+=213.2 at 220 nm). The reaction mixture was concentrated under reduced pressure to give 4-(4,4-difluoropiperidin-3-yl)-2-methylpyridine;hydrochloride (160 mg, 0.450 mmol, crude) as white solid, which was used directly for the next step. (P1): purity=94%, Rt: 0.720 min; [M+H]+=213.2 at 220 nm

Step 4: To a solution of 4-(4,4-difluoropiperidin-3-yl)-2-methylpyridine;hydrochloride (1.54 eq, 160 mg, 0.450 mmol) and 5-chloro-7-(2-fluoro-4-(trifluoromethyl)phenyl)-N,N-dimethylthiazolo[4,5-d]pyrimidin-2-amine (1.00 eq, 110 mg, 0.292 mmol) in DMSO (4 mL) was added DIEA (5.00 eq, 0.24 mL, 1.46 mmol). Then the mixture was stirred at 100° C. for 12 h. LCMS 1A showed that 41% of the starting material remained and 47% of the desired mass was detected (contained the by-product, 47%, Rt: 0.581 min; [M+H]⁺=533.0, 553.0 at 220 nm). The mixture was diluted with water (50 mL) and extracted with EtOAc (40 mL) twice. The combined organic layers were washed with an aqueous solution with brine (30 mL) three times and dried over Na₂SO₄. The solvent was filtered and concentrated under reduced pressure. The residue was purified by prep-TLC (SiO₂, DCM/EtOAc=3/1, the desired product R_{f}=0.4, the by-product R_{f}=0.35, showed by 254 nm) to give 5-(4,4-difluoro-3-(2-methylpyridin-4-yl)piperidin-1-yl)-7-(2-fluoro-4-(trifluoromethyl)phenyl)-N,N-dimethylthiazolo[4,5-d]pyrimidin-2-amine (45 mg, 0.0814 mmol, 27.89% yield) as white solid, checked by LCMS, HPLC, HNMR 4-P1C, FNMR. (P1): [M+H]⁺=553.2; purity=100% (220 nm); Retention time=0.890 min. ¹H NMR (400 MHz, CDCl₃) δ=8.47 (d, J=5.3 Hz, 1H), 7.85 (t, J=7.4 Hz, 1H), 7.55 (d, J=7.9 Hz, 1H), 7.48 (d, J=10.0 Hz, 1H), 7.17 (s, 1H), 7.13 (br d, J=5.3 Hz, 1H), 5.16-5.00 (m, 2H), 3.54-3.46 (m, 1H), 3.45-3.04 (m, 8H), 2.59 (s, 3H), 2.30-2.18 (m, 1H), 2.15-1.99 (m, 1H). SFC showed two peaks ˜1:1. And by-product 5-(4-fluoro-2′-methyl-5,6-dihydro-[3,4′-bipyridin]-1(2H)-yl)-7-(2-fluoro-4-(trifluoromethyl)phenyl)-N,N-dimethylthiazolo[4,5-d]pyrimidin-2-amine (50 mg, 0.0939 mmol, 32.16% yield) as yellow solid, checked by LCMS, HNMR, FNMR 4-P2A. (P2): [M+H]+=533.2; purity=99% (220 nm); Retention time=0.584 min. ¹H NMR (400 MHz, CDCl₃) δ=8.48 (d, J=5.3 Hz, 1H), 7.89 (t, J=7.8 Hz, 1H), 7.57 (d, J=8.6 Hz, 1H), 7.49 (d, J=10.1 Hz, 1H), 7.28 (br s, 1H), 7.25 (br d, J=5.7 Hz, 1H), 4.71 (br d, J=6.2 Hz, 2H), 4.27-4.20 (m, 2H), 3.44-3.15 (m, 6H), 2.59 (s, 5H).

Step 5: The 5-(4,4-difluoro-3-(2-methylpyridin-4-yl)piperidin-1-yl)-7-(2-fluoro-4-(trifluoromethyl)phenyl)-N,N-dimethylthiazolo[4,5-d]pyrimidin-2-amine (1.00 eq, 41 mg, 0.0742 mmol) was purified by SFC (Flow: 70 min/mL; column: DAICEL CHIRALPAK IC (250 mm×30 mm, 10 um); Mobile phase: Phase A for CO₂, and Phase B for 0.1% NH₃H₂O EtOH; Gradient elution: 0.1% NH₃H₂O EtOH in CO₂ in 40%, 10 min) to give (S)-5-(4,4-difluoro-3-(2-methylpyridin-4-yl)piperidin-1-yl)-7-(2-fluoro-4-(trifluoromethyl)phenyl)-N,N-dimethylthiazolo[4,5-d]pyrimidin-2-amine (17 mg, 0.0307 mmol, 41.42% yield) as white solid. (P1, peak 1 in SFC): [M+H]⁺=553.1; purity=98.9% (220 nm); Retention time=0.700 min. ¹H NMR (400 MHz, CDCl₃) δ=8.47 (br d, J=5.0 Hz, 1H), 7.85 (t, J=7.6 Hz, 1H), 7.55 (d, J=8.4 Hz, 1H), 7.48 (d, J=10.0 Hz, 1H), 7.17 (s, 1H), 7.13 (br d, J=4.2 Hz, 1H), 5.08 (br d, J=13.3 Hz, 2H), 3.50 (br t, J=12.6 Hz, 1H), 3.46-3.00 (m, 8H), 2.59 (s, 3H), 2.32-2.18 (m, 1H), 2.17-1.99 (m, 1H). (R)-5-(4,4-difluoro-3-(2-methylpyridin-4-yl)piperidin-1-yl)-7-(2-fluoro-4-(trifluoromethyl)phenyl)-N,N-dimethylthiazolo[4,5-d]pyrimidin-2-amine (17 mg, 0.0313 mmol, 42.15% yield)) as white solid. (P2, peak 2 in SFC): [M+H]⁺=553.2; purity=100% (220 nm); Retention time=0.695 min. ¹H NMR (400 MHz, CDCl₃) δ=8.47 (br d, J=5.1 Hz, 1H), 7.85 (t, J=7.6 Hz, 1H), 7.55 (d, J=8.2 Hz, 1H), 7.48 (d, J=10.0 Hz, 1H), 7.17 (s, 1H), 7.13 (br d, J=4.4 Hz, 1H), 5.08 (br d, J=13.1 Hz, 2H), 4.81 (br d, J=6.4 Hz, 1H), 3.53-3.46 (m, 1H), 3.46-2.98 (m, 8H), 2.59 (s, 3H), 2.31-2.18 (m, 1H), 2.17-1.98 (m, 1H). Absolute configuration of P1 and P2 were not confirmed. P1 was peak 1 in SFC; P2 was peak 2 in SFC.

### Example 24—Synthesis of Compound I-196: 7-(2-fluoro-4-(trifluoromethyl)phenyl)-N,N-dimethyl-5-(3-(2-methylpyridin-4-yl)piperidin-1-yl)thiazolo[4,5-d]pyrimidin-2-amine

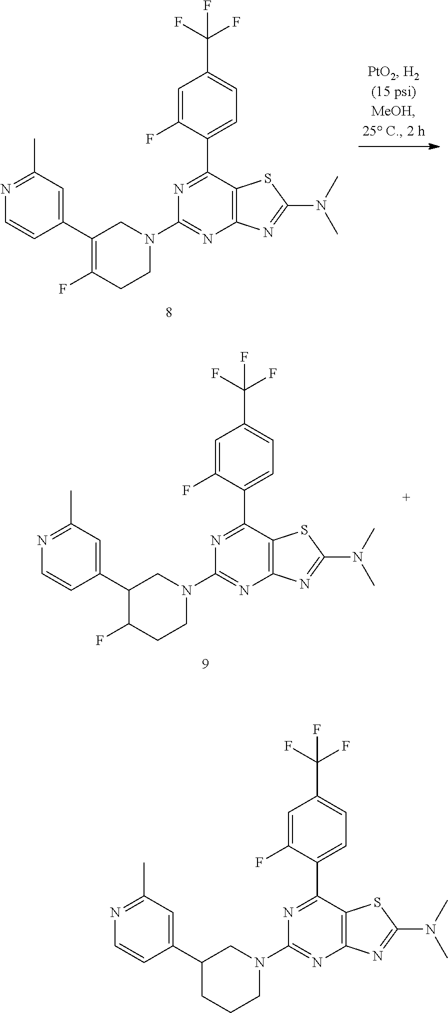 [SMILES: Cc1cc(C2=C(F)CCN(c3nc(-c4ccc(C(F)(F)F)cc4F)c4sc(N(C)C)nc4n3)C2)ccn1 ; Cc1cc(C2CN(c3nc(-c4ccc(C(F)(F)F)cc4F)c4sc(N(C)C)nc4n3)CCC2F)ccn1 ; Cc1cc(C2CCCN(c3nc(-c4ccc(C(F)(F)F)cc4F)c4sc(N(C)C)nc4n3)C2)ccn1]

Step 1: To a solution of 5-(4-fluoro-2′-methyl-5,6-dihydro-[3,4′-bipyridin]-1(2H)-yl)-7-(2-fluoro-4-(trifluoromethyl)phenyl)-N,N-dimethylthiazolo[4,5-d]pyrimidin-2-amine (1.00 eq, 10 mg, 0.0188 mmol) in MeOH (2 mL) was added PtO₂ (1.00 eq, 4.3 mg, 0.0188 mmol) under N₂ atmosphere. The mixture was purged with H₂ three times, then the mixture was stirred at 25° C. for 2 hours under H₂ (15 psi) atmosphere. LCMS showed that the starting material was consumed completely but no desired mass was detected and the by-product mass was major (92%, Rt: 0.570 min; [M+H]⁺=517.2 at 220 nm). The mixture was filtered and concentrated under reduced pressure. The residue was purified by prep-TLC (SiO₂, DCM/EtOAc=3/1, the by-product Rf=0.3, showed by 254 nm) to give 7-(2-fluoro-4-(trifluoromethyl)phenyl)-N,N-dimethyl-5-(3-(2-methylpyridin-4-yl)piperidin-1-yl)thiazolo[4,5-d]pyrimidin-2-amine (4.0 mg, 0.00732 mmol, 38.98% yield) as yellow solid, checked by LCMS, H NMR, F NMR, HPLC. (P2, racemic): [M+H]⁺=517.3; purity=95% (220 nm); Retention time=0.529 min. ¹H NMR (400 MHz, CDCl₃) δ=8.50-8.39 (m, 1H), 7.93-7.85 (m, 1H), 7.55 (d, J=8.2 Hz, 1H), 7.47 (d, J=10.3 Hz, 1H), 7.21-7.04 (m, 2H), 5.08-4.86 (m, 2H), 3.46-3.12 (m, 6H), 3.02 (q, J=11.7 Hz, 2H), 2.85-2.74 (m, 1H), 2.63-2.55 (m, 3H), 2.10-2.01 (m, 1H), 1.90-1.82 (m, 1H), 1.79-1.71 (m, 2H).

### Example 25—Synthesis of Compounds: 5-(3-(1-cyclopropyl-1H-pyrazol-4-yl)-4,4-difluoropiperidin-1-yl)-7-(2-fluoro-4-(trifluoromethyl)phenyl)-N,N-dimethylthiazolo[4,5-d]pyrimidin-2-amine (I-185), (S)-5-(3-(1-cyclopropyl-1H-pyrazol-4-yl)-4,4-difluoropiperidin-1-yl)-7-(2-fluoro-4-(trifluoromethyl)phenyl)-N,N-dimethylthiazolo[4,5-d]pyrimidin-2-amine (I-199) and (R)-5-(3-(1-cyclopropyl-1H-pyrazol-4-yl)-4,4-difluoropiperidin-1-yl)-7-(2-fluoro-4-(trifluoromethyl)phenyl)-N,N-dimethylthiazolo[4,5-d]pyrimidin-2-amine (I-200)

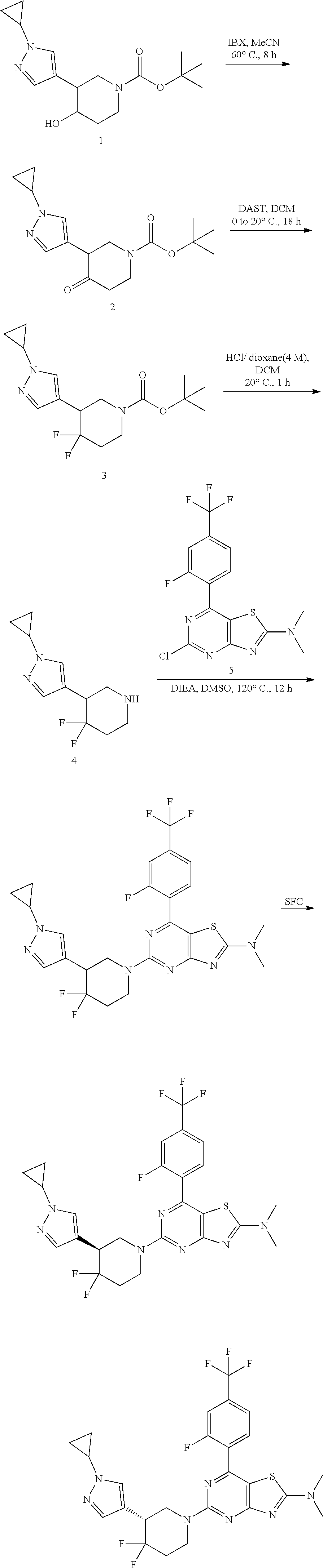

Step 1: To a solution of tert-butyl 3-(1-cyclopropyl-1H-pyrazol-4-yl-4-hydroxypiperidine-1-carboxylate (1.00 eq, 1000 mg, 3.25 mmol) in MeCN (20 mL) was added IBX (2.00 eq, 1718 mg, 6.51 mmol) at 20° C. and the mixture was stirred at 60° C. for 4 h. LCMS 2-P1A showed that 27% the starting material remained and 63% of the desired mass was detected (63%, Rt: 0.840 min; [M+H]⁺=306.0 at 220 nm). The mixture was stirred at 60° C. for 4 h. LCMS 2-P1B showed that 80% of the desired mass was detected (80%, Rt: 0.574 min; [M+H]⁺=306.1 at 220 nm). The reaction mixture (combined with) was filtered and concentrated under reduced pressure to give tert-butyl 3-(1-cyclopropyl-1H-pyrazol-4-yl)-4-oxopiperidine-1-carboxylate (1100 mg, 3.60 mmol, 110.73% yield) as yellow solid. (P1): [M+H]⁺=306.2; purity=76% (220 nm); Retention time=0.579 min. ¹H NMR (400 MHz, CDCl3) δ=7.48 (s, 1H), 7.40 (s, 1H), 4.31-3.94 (m, 2H), 3.69-3.48 (m, 4H), 2.61-2.48 (m, 2H), 1.51 (s, 9H), 1.14-1.08 (m, 2H), 1.04-0.98 (m, 2H).

Step 2: To a solution of tert-butyl 3-(1-cyclopropyl-1H-pyrazol-4-yl)-4-oxopiperidine-1-carboxylate (1.00 eq, 500 mg, 1.64 mmol) in DCM (10 mL) was added DAST (10.0 eq, 2639 mg, 16.4 mmol) at 0° C. under N₂, the mixture was stirred at 20° C. under N₂ for 12 h. LCMS 9-P1A showed that 21% of the intermediate and 45% of the desired mass was detected (45%, Rt: 0.581 min; [M+H]⁺=328.3 at 220 nm). The mixture was stirred at 20° C. under N₂ for 6 h. LCMS showed that 21% of the intermediate was consumed completely and 60% of the desired mass was detected (60%, Rt: 0.642 min; [M+H]⁺=328.4 at 220 nm). The reaction mixture was quenched by addition sat. NaHCO₃ (50 mL) at 0° C., and then extracted with DCM (40 mL×2). The combined organic layers were washed with brine (60 mL), dried over Na₂SO₄, filtered and concentrated under reduced pressure to give a residue. The crude product was purified by prep-HPLC (column: Phenomenex luna C18 150×25 mm×10 um; mobile phase: [water (0.1% FA)-ACN]; B %: 43%-73%, 10 min) to give tert-butyl 3-(1-cyclopropyl-1H-pyrazol-4-yl)-4,4-difluoropiperidine-1-carboxylate (200 mg, 0.611 mmol, 37.31% yield) as yellow oil, checked by LCMS and H NMR. (P1): [M+H]+=328.2; purity=97% (220 nm); Retention time=0.620 min. ¹H NMR (400 MHz, CDCl₃) δ=7.43 (s, 2H), 4.12-3.92 (m, 2H), 3.57 (tt, J=3.7, 7.3 Hz, 1H), 3.25 (br t, J=10.5 Hz, 2H), 3.15-2.99 (m, 1H), 2.17-2.07 (m, 1H), 2.01-1.84 (m, 1H), 1.48 (s, 9H), 1.17-1.09 (m, 2H), 1.06-0.96 (m, 2H).

Step 3: To a solution of tert-butyl 3-(1-cyclopropyl-1H-pyrazol-4-yl)-4,4-difluoropiperidine-1-carboxylate (1.00 eq, 190 mg, 0.580 mmol) in DCM (5 mL) was added HCl (6.89 eq, 1.0 mL, 4.00 mmol) in dioxane and stirred at 20° C. under N₂ for 1 h. LCMS 9-P1A1 showed that the starting material was consumed completely and the desired mass was major (90%, Rt: 0.417 min; [M+H]⁺=228.3 at 220 nm). The mixture was concentrated under reduced pressure to give 3-(1-cyclopropyl-1H-pyrazol-4-yl)-4,4-difluoropiperidine;hydrochloride (153 mg, 0.580 mmol, 99.97% yield, crude) as colorless oil. (P1): ¹H NMR (400 MHz, DMSO-d₆) δ=7.82 (s, 1H), 7.40 (s, 1H), 3.70 (tt, J=3.8, 7.4 Hz, 1H), 3.66-3.53 (m, 1H), 3.47 (br s, 2H), 3.28-2.97 (m, 2H), 2.43-2.32 (m, 2H), 1.04-0.98 (m, 2H), 0.97-0.91 (m, 2H).

Step 4: To a solution of 3-(1-cyclopropyl-1H-pyrazol-4-yl)-4,4-difluoropiperidine;hydrochloride (1.50 eq, 136 mg, 0.518 mmol) in DMSO (5 mL) was added DIEA (4.75 eq, 0.27 mL, 1.64 mmol) and 5-chloro-7-(2-fluoro-4-(trifluoromethyl)phenyl)-N,N-dimethylthiazolo[4,5-d]pyrimidin-2-amine (1.00 eq, 130 mg, 0.345 mmol) in one portion, then the mixture was stirred at 120° C. for 12 h. LCMS 1-P1A showed that 29% the starting material remained and 47% of the desired mass was detected (47%, Rt: 0.701 min; [M+H]⁺=568.5 at 220 nm). The mixture was diluted with water (40 mL) and extracted with ethyl acetate (40 mL) twice. The combined organic layers were washed with an aqueous solution with brine (30 mL) three times and dried over Na₂SO₄. The solvent was filtered and concentrated under reduced pressure. The residue was purified by prep-TLC (SiO₂, PE/EtOAc=1/1, the desired product R_{f}=0.3, showed by 254 nm) to give 5-(3-(1-cyclopropyl-1H-pyrazol-4-yl)-4,4-difluoropiperidin-1-yl)-7-(2-fluoro-4-(trifluoromethyl)phenyl)-N,N-dimethylthiazolo[4,5-d]pyrimidin-2-amine (150 mg, 0.254 mmol, 73.53% yield) as yellow solid. (P1, racemic): [M+H]⁺=568.2; purity=96% (220 nm); Retention time=0.708 min. ¹H NMR (400 MHz, CDCl₃) δ=7.86 (t, J=7.6 Hz, 1H), 7.55 (d, J=8.1 Hz, 1H), 7.50-7.42 (m, 3H), 4.86 (br t, J=15.2 Hz, 2H), 3.56 (tt, J=3.8, 7.3 Hz, 1H), 3.46 (br t, J=12.1 Hz, 2H), 3.40-3.04 (m, 7H), 2.27-2.12 (m, 1H), 2.11-1.93 (m, 1H), 1.15-1.07 (m, 2H), 1.05-0.96 (m, 2H). SFC showed two peaks ˜1:1.

Step 5: 5-(3-(1-cyclopropyl-1H-pyrazol-4-yl)-4,4-difluoropiperidin-1-yl)-7-(2-fluoro-4-(trifluoromethyl)phenyl)-N,N-dimethylthiazolo[4,5-d]pyrimidin-2-amine (1.00 eq, 146 mg, 0.257 mmol) was purified by SFC (Flow: 70 min/mL; column: DAICEL CHIRALPAK IC (250 mm×30 mm, 5 um); Mobile phase: Phase A for CO₂, and Phase B for 0.1% NH₃H₂O EtOH; Gradient elution: 0.1% NH₃H₂O EtOH in CO₂ in 40%, 10 min) to give (S)-5-(3-(1-cyclopropyl-1H-pyrazol-4-yl)-4,4-difluoropiperidin-1-yl)-7-(2-fluoro-4-(trifluoromethyl)phenyl)-N,N-dimethylthiazolo[4,5-d]pyrimidin-2-amine (68 mg, 0.119 mmol, 46.44% yield) as off-white solid.

(P1, peak 1 in SFC): [M+H]⁺=568.2; purity=100% (220 nm); Retention time=0.860 min. ¹H NMR (400 MHz, CDCl₃) δ=7.87 (t, J=7.6 Hz, 1H), 7.56 (d, J=8.3 Hz, 1H), 7.47 (d, J=7.6 Hz, 3H), 4.96-4.78 (m, 2H), 3.57 (tt, J=3.8, 7.3 Hz, 1H), 3.50-3.43 (m, 2H), 3.41-3.09 (m, 7H), 2.29-2.15 (m, 1H), 2.12-1.94 (m, 1H), 1.14-1.09 (m, 2H), 1.04-0.96 (m, 2H).

And (R)-5-(3-(1-cyclopropyl-1H-pyrazol-4-yl)-4,4-difluoropiperidin-1-yl)-7-(2-fluoro-4-(trifluoromethyl)phenyl)-N,N-dimethylthiazolo[4,5-d]pyrimidin-2-amine (65 mg, 0.113 mmol, 43.84% yield) as off-white solid.

(P2, peak 2 in SFC): [M+H]+=568.2; purity=99% (220 nm); Retention time=0.862 min. ¹H NMR (400 MHz, CDCl₃) δ=7.86 (t, J=7.5 Hz, 1H), 7.55 (d, J=8.1 Hz, 1H), 7.50-7.44 (m, 3H), 4.86 (br t, J=14.8 Hz, 2H), 3.57 (tt, J=3.7, 7.3 Hz, 1H), 3.46 (br t, J=12.0 Hz, 2H), 3.40-3.06 (m, 7H), 2.27-2.15 (m, 1H), 2.11-1.94 (m, 1H), 1.15-1.08 (m, 2H), 1.04-0.95 (m, 2H).

### Example 26—Synthesis of Compound I-210: 5-[(2R)-2-(1-cyclopropylpyrazol-4-yl)tetrahydropyran-4-yl]-N,N-dimethyl-7-phenoxy-thiazolo[4,5-d]pyrimidin-2-amine

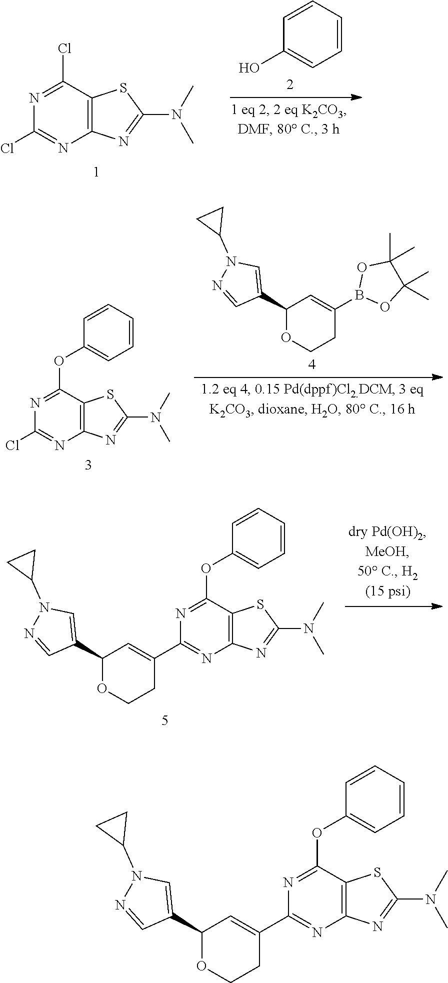 [SMILES: CN(C)c1nc2nc(Cl)nc(Cl)c2s1 ; Oc1ccccc1 ; CN(C)c1nc2nc(Cl)nc(Oc3ccccc3)c2s1 ; CC1(C)OB(C2=C[C@H](c3cnn(C4CC4)c3)OCC2)OC1(C)C ; CN(C)c1nc2nc(C3=C[C@H](c4cnn(C5CC5)c4)OCC3)nc(Oc3ccccc3)c2s1 ; CN(C)c1nc2nc(C3=C[C@H](c4cnn(C5CC5)c4)OCC3)nc(Oc3ccccc3)c2s1]

Step 1: To a solution of 5,7-dichloro-N,N-dimethyl-1,3-benzothiazol-2-amine (1.00 eq, 1000 mg, 4.05 mmol) and PHENOL (1.00 eq, 381 mg, 4.05 mmol) in DMF (2 mL) was added K₂CO₃ (2.00 eq, 1118 mg, 8.09 mmol). Then the reaction mixture was stirred at 80° C. for 12 hours. LCMS (0-P1A) (5-95AB/1.5 min): RT=0.890 min, 307.0=[M+H]⁺, ESI+ showed 85.8% of desired product. The reaction was diluted with water (80 mL) and then extracted with ethyl acetate (80 mL*3). The combined organic layers were washed with brine, dried over Na₂SO₄, filtered and concentrated under reduced pressure to give 5-chloro-N,N-dimethyl-7-phenoxy-1,3-benzothiazol-2-amine (1200 mg, 3.69 mmol, 91.27% yield) as yellow solid. RT=0.882 min, 307.0=[M+H]⁺, ESI+ purity=93.8%. ¹H NMR (400 MHz, DMSO-d6) δ ppm 3.09-3.32 (m, 6H) 7.28-7.36 (m, 3H) 7.45-7.51 (m, 2H).

Step 2: To a solution of 5-chloro-N,N-dimethyl-7-phenoxy-thiazolo[4,5-d]pyrimidin-2-amine (1.00 eq, 200 mg, 0.587 mmol), 1-cyclopropyl-4-[(6R)-4-(4,4,5,5-tetramethyl-1,3,2-dioxaborolan-2-yl)-3,6-dihydro-2H-pyran-6-yl]pyrazole (1.20 eq, 223 mg, 0.704 mmol) and K₂CO₃ (3.00 eq, 243 mg, 1.76 mmol) in 1,4-Dioxane (5 mL) and Water (0.5 mL) was added Pd(dppf)Cl₂·DCM (0.100 eq, 43 mg, 0.0587 mmol). The reaction mixture was stirred at 80° C. for 12 hours under N₂ atmosphere. LCMS (5-95AB/1.5 min): RT=0.954 min, 461.1=[M+H]⁺, ESI+ showed 60.5% of desired product. The reaction was diluted with water (50 mL) and then extracted with ethyl acetate (20 mL*3). The combined organic layers were washed with brine, dried over Na₂SO₄, filtered and concentrated under reduced pressure to give a residue. The residue was purified by prep-TLC (PE:EtOAc=2:1, Rf=0.25) to afford 5-[(6R)-6-(1-cyclopropylpyrazol-4-yl)-3,6-dihydro-2H-pyran-4-yl]-N,N-dimethyl-7-phenoxy-thiazolo[4,5-d]pyrimidin-2-amine (160 mg, 0.347 mmol, 59.21% yield) as white solid, which confirmed by LCMS (B) (5-95AB/1.5 min): RT=0.939 min, 461.1=[M+H]⁺, ESI+ showed 98.0% of desired product.

Step 3: To a solution of 5-[(6R)-6-(1-cyclopropylpyrazol-4-yl)-3,6-dihydro-2H-pyran-4-yl]-N,N-dimethyl-7-phenoxy-thiazolo[4,5-d]pyrimidin-2-amine (1.00 eq, 120 mg, 0.255 mmol) in Methanol (30 mL) was added dry-Pd(OH)₂ (3.35 eq, 120 mg, 0.855 mmol) under N₂ atmosphere. The mixture was purged with H₂ (15 psi) 3 times, then the mixture was stirred at 50° C. for 16 hours under H₂ (15 psi) atmosphere. LCMS (5-95AB/1.5 min): RT=0.897 min, 463.2=[M+H]⁺, ESI+ showed 76.5% of desired product. The reaction mixture was filtered through a pad of celite. The filter cake was washed with MeOH (40 mL). The filtrate was concentrated under reduced pressure to afford a residue (160 mg, NOTE: the residue has poor solubility in EA, ACN, PE, DMF). A part of residue (30 mg dissolved in 2 mL MeOH) was purified by prep-HPLC (Column, [Unisil 3-100 C18 Ultra 150*50 mm*3 um]; mobile phase: [ACN] and [H₂O](conditions: [water (0.225% FA)-ACN], B %: 48%-78%; Detector, UV 254 nm. RT: [7 min]) to afford 5-[(2R)-2-(1-cyclopropylpyrazol-4-yl)tetrahydropyran-4-yl]-N,N-dimethyl-7-phenoxy-thiazolo[4,5-d]pyrimidin-2-amine (17 mg, 0.0368 mmol, 14.39% yield) as white solid. P1, racemate): [M+H]⁺=463.2; purity=100% (220 nm); Retention time=0.946 min; ¹H NMR (400 MHz, CHLOROFORM-d) δ ppm 0.94-1.01 (m, 2H) 1.04-1.11 (m, 2H) 1.90-2.06 (m, 3H) 2.22 (br d, J=13.33 Hz, 1H) 2.37-2.46 (m, 1H) 3.04-3.17 (m, 1H) 3.27 (br s, 6H) 3.54 (tt, J=7.18, 3.76 Hz, 1H) 3.61-3.72 (m, 1H) 3.72-3.79 (m, 1H) 4.10-4.19 (m, 1H) 4.37-4.45 (m, 1H) 4.63 (dd, J=8.80, 2.81 Hz, 1H) 7.16-7.26 (m, 2H) 7.29-7.32 (m, 1H) 7.36-7.48 (m, 4H).

The rest part of residue (130 mg dissolved in 5 mL MeOH) was purified by prep-HPLC (Column, [Unisil 3-100 C18 Ultra 150*50 mm*3 um]; mobile phase: [ACN] and [H₂O](conditions: [water (0.225% FA)-ACN], B %: 49%-79%; Detector, UV 254 nm. RT: [7 min]) to afford 5-[(2R)-2-(1-cyclopropylpyrazol-4-yl)tetrahydropyran-4-yl]-N,N-dimethyl-7-phenoxy-thiazolo[4,5-d]pyrimidin-2-amine (37 mg, 0.0800 mmol, 31.33% yield) as white solid, which confirmed by LCMS (5-95AB/1.5 min): RT=0.939 min, 463.2=[M+H]⁺, ESI+ showed 100% of desired product. SFC (2_A9) showed the ratio was ˜3:1.

### Example 27—Synthesis of Compound I-216: 2S,6R)-2-(1-cyclopropyl-1H-pyrazol-4-yl)-4-(4-(2-fluoro-4-(trifluoromethyl)phenyl) imidazo[1,2-a][1,3,5]triazin-2-yl)-6-methylmorpholine

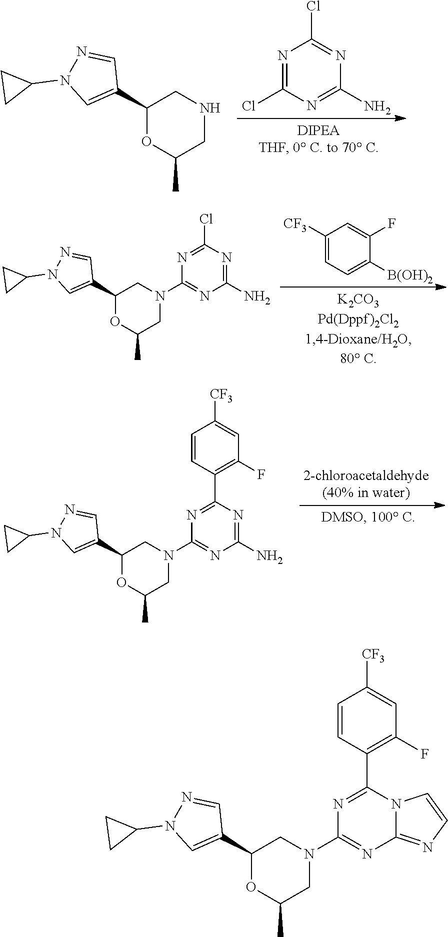 [SMILES: C[C@@H]1CNC[C@H](c2cnn(C3CC3)c2)O1 ; Nc1nc(Cl)nc(Cl)n1 ; C[C@@H]1CN(c2nc(N)nc(Cl)n2)C[C@H](c2cnn(C3CC3)c2)O1 ; OB(O)c1ccc(C(F)(F)F)cc1F ; C[C@@H]1CN(c2nc(N)nc(-c3ccc(C(F)(F)F)cc3F)n2)C[C@H](c2cnn(C3CC3)c2)O1 ; C[C@@H]1CN(c2nc(-c3ccc(C(F)(F)F)cc3F)n3ccnc3n2)C[C@H](c2cnn(C3CC3)c2)O1]

Step 1: To a 0° C. solution of (2S,6R)-2-(1-cyclopropylpyrazol-4-yl)-6-methyl-morpholine (1.00 eq, 200 mg, 0.97 mmol) in THF (4 ml) was added DIPEA (3.00 eq, 0.50 mL, 2.9 mmol) followed by 4,6-dichloro-1,3,5-triazin-2-amine (1.00 eq, 159 mg, 0.97 mmol). The mixture was then stirred at 70° C. for an hour. Upon completion, the reaction mixture was cooled down, and quenched with sat. NH₄Cl (aq.) and diluted with EtOAc. The reaction mixture was extracted with EtOAc and the combined organic layers were washed with water, brine, dried over MgSO₄, filtered, and concentrated under reduced pressure. The crude material was purified by silica gel chromatography on a 40 g pre-packed column eluting with DCM/MeOH (0% to 30%) to provide 4-chloro-6-((2S,6R)-2-(1-cyclopropyl-1H-pyrazol-4-yl)-6-methylmorpholino)-1,3,5-triazin-2-amine (300 mg, 0.89 mmol, 93% yield) as a white solid. ¹H NMR (400 MHz, Chloroform-d) δ_{H} 7.42 (2H, d, J 10.9 Hz), 4.59 (1H, t, J 14.7 Hz), 4.49 (1H, t, J 15.1 Hz), 4.39 (1H, dd, J 10.9, 2.6 Hz), 3.62 (1H, t, J 8.0 Hz), 3.44-3.50 (1H, m), 3.11 (3H, s), 2.81 (1H, d, J 12.5 Hz), 2.59 (1H, br s), 1.18 (3H, d, J 6.2 Hz), 0.99 (2H, s), 0.94 (2H, t, J 6.9 Hz).

Step 2: To a solution of 4-chloro-6-[(2S,6R)-2-(1-cyclopropylpyrazol-4-yl)-6-methyl-morpholin-4-yl]-1,3,5-triazin-2-amine (1.0 eq, 290 mg, 0.86 mmol) in 1,4-Dioxane (5 mL) was added [2-fluoro-4-(trifluoromethyl)phenyl]boronic acid (1.2 eq, 215 mg, 1.04 mmol) followed by potassium carbonate (3.00 eq, 358 mg, 2.6 mmol), water (1.5 mL) and 1,1′-Bis(diphenylphosphino)ferrocene palladium(II)dichloride dichloromethane complex (0.1 eq, 71 mg, 0.09 mmol). The mixture was then stirred at 80° C. for an hour. Upon completion, the reaction mixture was cooled down, quenched with sat. NH₄Cl (aq.) and diluted with EtOAc. The reaction mixture was extracted with EtOAc and the combined organic layers were washed with water, brine, dried over MgSO₄, filtered, and concentrated under reduced pressure. The crude material was purified by silica gel chromatography on a 40 g pre-packed column eluting with DCM/EtOAc (30% to 100%) to provide 4-((2S,6R)-2-(1-cyclopropyl-1H-pyrazol-4-yl)-6-methylmorpholino)-6-(2-fluoro-4-(trifluoromethyl)phenyl)-1,3,5-triazin-2-amine (294 mg, 0.63 mmol, 73% yield) as a white solid. ¹H NMR (400 MHz, Chloroform-d) SH 8.07 (1H, m), 7.50 (2H, s), 7.45 (1H, m), 7.38 (1H, d, J 10.4 Hz), 5.30 (2H, br s), 4.63-4.87 (2H, m), 4.49 (1H, dd, J 10.9, 2.6 Hz), 3.68-3.75 (1H, m), 3.54 (1H, m), 2.91 (1H, m), 2.68 (1H, m), 1.27 (3H, d, J 6.2 Hz), 1.06-1.10 (2H, m), 0.99 (2H, m).

Step 3: To a solution of 4-[(2S,6R)-2-(1-cyclopropylpyrazol-4-yl)-6-methyl-morpholin-4-yl]-6-[2-fluoro-4-(trifluoromethyl)phenyl]-1,3,5-triazin-2-amine (1.00 eq, 1.40 g, 3.0 mmol) in DMSO (20 mL) was added 2-chloroacetaldehyde (1.10 eq, 40% in water, 0.42 mL, 3.3 mmol). The mixture was then stirred at 100° C. for an hour, then cooled down, quenched with sat. NaHCO₃ (aq.) and diluted with EtOAc. The reaction mixture was extracted with EtOAc and the combined organic layers were washed with water, brine, dried over MgSO₄, filtered, and concentrated under reduced pressure. The crude material was purified by reverse phase chromatography on a 60 g C-18 pre-packed column eluting with H₂O (0.1% FA)/ACN (15% to 95%) to provide (2S,6R)-2-(1-cyclopropyl-1H-pyrazol-4-yl)-4-(4-(2-fluoro-4-(trifluoromethyl)phenyl)imidazo[1,2-a][1,3,5]triazin-2-yl)-6-methylmorpholine (330 mg, 0.68 mmol, 22% yield) as a yellow solid. ¹H NMR (400 MHz, Chloroform-d) SH 7.82-7.86 (1H, m), 7.65 (1H, d, J 8.1 Hz), 7.58 (1H, d, J 9.7 Hz), 7.50 (2H, s), 7.39 (1H, d, J 1.8 Hz), 6.98-7.00 (1H, m), 4.72-4.88 (2H, m), 4.51-4.56 (1H, m), 3.72-3.80 (1H, m), 3.50-3.58 (1H, m), 2.96-3.05 (1H, m), 2.76-2.82 (1H, m), 1.24-1.30 (3H, m), 1.06-1.12 (2H, m), 0.95-1.00 (2H, m). 19F NMR (Chloroform-d, 376 MHz): δ_{F} −63.3,-107.7. LC/MS (ESI+) m/z=488.2 [M+1]+

### Example 28—Synthesis of Compound I-221: (2S,6R)-2-(1-cyclopropyl-1H-pyrazol-4-yl)-4-(7-(2,4-difluorophenyl)-2-((R)-3-methoxypyrrolidin-1-yl)thiazolo[4,5-d]pyrimidin-5-yl)-6-methylmorpholine

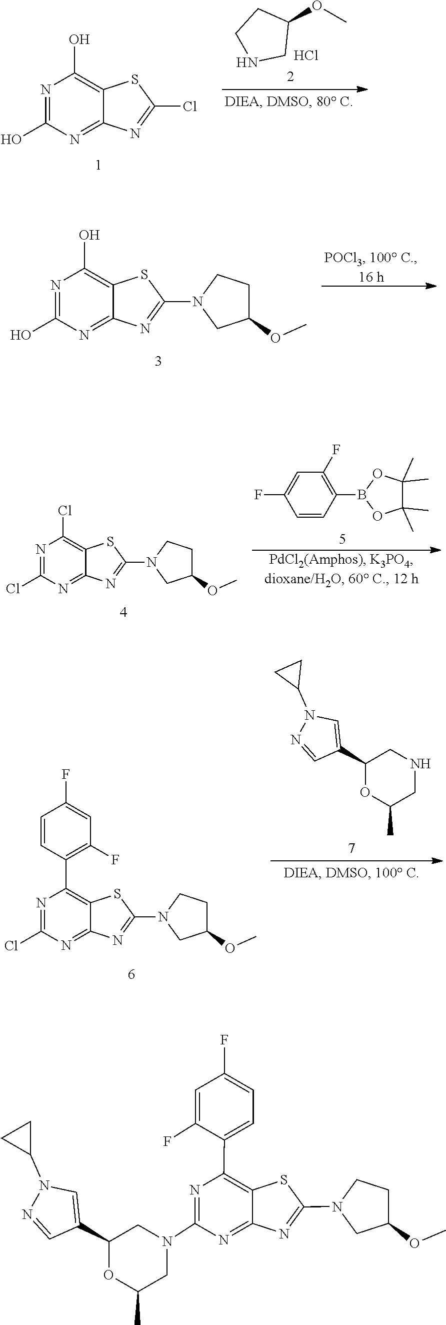

Step 1: To a solution of 2-chlorothiazolo[4,5-d]pyrimidine-5,7-diol (1.00 eq, 500 mg, 2.46 mmol) and (3R)-3-methoxypyrrolidine;hydrochloride (2.00 eq, 676 mg, 4.91 mmol) in DMSO (5 mL) was added DIEA (4.00 eq, 1.6 mL, 9.82 mmol). Then the reaction mixture was stirred at 80° C. for 1 hours. LCMS (5-95AB/1.5 min): RT=0.277 min, 269.1=[M+H]⁺, ESI+ showed 100% of desired product. The reaction mixture was adjusted to PH<7 with FA, purified by reversed-phase HPLC (0.1% FA condition) and lyophilized to give (R)-2-(3-methoxypyrrolidin-1-yl)thiazolo[4,5-d]pyrimidine-5,7-diol (530 mg, 1.98 mmol, 80.45% yield) as red solid. (M+H)=269.1; purity=99% (220 nm); Retention time=0.302 min ¹H NMR (400 MHz, CDCl₃-d) 6=8.26-8.15 (m, 1H), 7.38-7.28 (m, 1H), 3.38 (s, 4H), 2.35-2.24 (m, 2H), 2.21-2.11 (m, 2H), 1.48 (br d, J=6.0 Hz, 2H)

Step 2: A mixture of 2-[(3R)-3-methoxypyrrolidin-1-yl]thiazolo[4,5-d]pyrimidine-5,7-diol (1.00 eq, 530 mg, 1.98 mmol) in POCl₃ (1.00 eq, 5.0 mL, 1.98 mmol) was stirred at 100° C. for 12 hours. LCMS (5-95AB/1.5 min): RT=0.817 min, 305.0=[M+H]⁺, ESI+ showed 96% of desired product. The reaction mixture was concentrated under reduced pressure to give a residue, the residue was partitioned between DCM (80 mL*2) and NaHCO₃ (aq, 100 mL). The combined organic layers were dried over Na₂SO₄, filtered and concentrated under reduced pressure to give a residue, and purified by reversed-phase HPLC (0.1% FA condition) and lyophilized to give (R)-5,7-dichloro-2-(3-methoxypyrrolidin-1-yl)thiazolo[4,5-d]pyrimidine (480 mg, 1.57 mmol, 79.62% yield) as brown red solid. (M+H)+=304.9; purity=100% (220 nm); Retention time=0.495 min. 1H NMR (400 MHz, CDCl₃-d) 6=4.25-4.00 (m, 2H), 3.93-3.76 (m, 1H), 3.69-3.46 (m, 2H), 3.43-3.35 (m, 3H), 2.40-2.09 (m, 2H)

Step 3: Charge 5,7-dichloro-2-[(3R)-3-methoxypyrrolidin-1-yl]thiazolo[4,5-d]pyrimidine (1.10 eq, 475 mg, 1.56 mmol), 2-(2,4-difluorophenyl)-4,4,5,5-tetramethyl-1,3,2-dioxaborolane (1.00 eq, 340 mg, 1.42 mmol), K₃PO₄ (3.50 eq, 1052 mg, 4.96 mmol) and Pd(Amphos)Cl₂ (0.130 eq, 130 mg, 0.184 mmol) into sealed bottle under N₂ atmosphere and purged with N₂ for three times, then 1,4-Dioxane (5 mL) and Water (0.5 mL) was added in one portion at 15° C., then the mixture was stirred at 60° C. for 16 hours. LCMS (5-95AB/1.5 min): RT=0.857 min, 383.1=[M+H]⁺, ESI+ showed 53% of desired product. The reaction mixture was partitioned between ethyl acetate (60 mL*2) and water (80 mL). The combined organic layers were dried over Na₂SO₄, filtered and concentrated under reduced pressure to give a residue. The crude product was purified by reversed-phase HPLC (0.1% FA condition) and lyophilized to give (R)-5-chloro-7-(2,4-difluorophenyl)-2-(3-methoxypyrrolidin-1-yl)thiazolo[4,5-d]pyrimidine (340 mg, 0.888 mmol, 62.71% yield) as brown solid. (M+H)=383.2; purity=70% (220 nm); Retention time=0.848 min. ¹H NMR (400 MHz, CDCl₃-d) 6=7.82 (dt, J=6.4, 8.5 Hz, 1H), 7.09-7.03 (m, 1H), 6.97 (ddd, J=2.4, 8.6, 10.8 Hz, 1H), 4.08-3.75 (m, 2H), 3.67-3.50 (m, 2H), 3.37 (s, 4H), 2.30-2.07 (m, 2H)

Step 4: To a solution of 5-chloro-7-(2,4-difluorophenyl)-2-[(3R)-3-methoxypyrrolidin-1-yl]thiazolo[4,5-d]pyrimidine (1.00 eq, 240 mg, 0.627 mmol) and (2S,6R)-2-(1-cyclopropylpyrazol-4-yl)-6-methyl-morpholine (3.00 eq, 390 mg, 1.88 mmol) in DMSO (3 mL) was added DIEA (4.00 eq, 0.41 mL, 2.51 mmol) at 25° C. Then the reaction mixture was stirred at 100° C. for 1 h. LCMS (5-95AB/1.5 min): RT=0.967 min, 554.2=[M+H]⁺, ESI+ showed 60% of desired product. The reaction mixture was partitioned between ethyl acetate (50 mL*2) and water (80 mL). The combined organic layers were dried over Na₂SO₄, filtered and concentrated under reduced pressure to give a residue. The crude product was purified by prep-HPLC (Phenomenex luna C18 150*25 mm*10 um; mobile phase: [water (0.1% FA)-ACN]; B %: 58%-88%, 12 min) to give (2S,6R)-2-(1-cyclopropyl-1H-pyrazol-4-yl)-4-(7-(2,4-difluorophenyl)-2-((R)-3-methoxypyrrolidin-1-yl)thiazolo[4,5-d]pyrimidin-5-yl)-6-methylmorpholine (178 mg, 0.322 mmol, 51.34% yield) (SFC showed ee. is ˜100.0%) as yellow solid. (M+H)⁺=554.2; purity=100% (220 nm); Retention time=0.954 min. ¹H NMR (400 MHz, CDCl₃-d) δ=7.76 (dt, J=6.7, 8.3 Hz, 1H), 7.53 (d, J=4.4 Hz, 2H), 7.05-6.99 (m, 1H), 6.97-6.90 (m, 1H), 4.92 (br d, J=12.8 Hz, 1H), 4.80 (br d, J=12.9 Hz, 1H), 4.58 (dd, J=2.6, 10.8 Hz, 1H), 4.23-3.95 (m, 2H), 3.81 (ddd, J=2.6, 6.3, 10.5 Hz, 2H), 3.68-3.41 (m, 3H), 3.36 (s, 3H), 2.96 (dd, J=10.9, 13.2 Hz, 1H), 2.72 (dd, J=10.7, 13.2 Hz, 1H), 2.30-2.09 (m, 2H), 1.30 (d, J=6.3 Hz, 3H), 1.14-1.09 (m, 2H), 1.03-0.97 (m, 2H)

### Example 29—Synthesis of Compound I-224: (2S,6R)-2-(1-cyclopropylpyrazol-4-yl)-4-[7-(2,4-difluorophenyl)-2-[(3S)-3-methoxypyrrolidin-1-yl]thiazolo[4,5-d]pyrimidin-5-yl]-6-methyl-morpholine

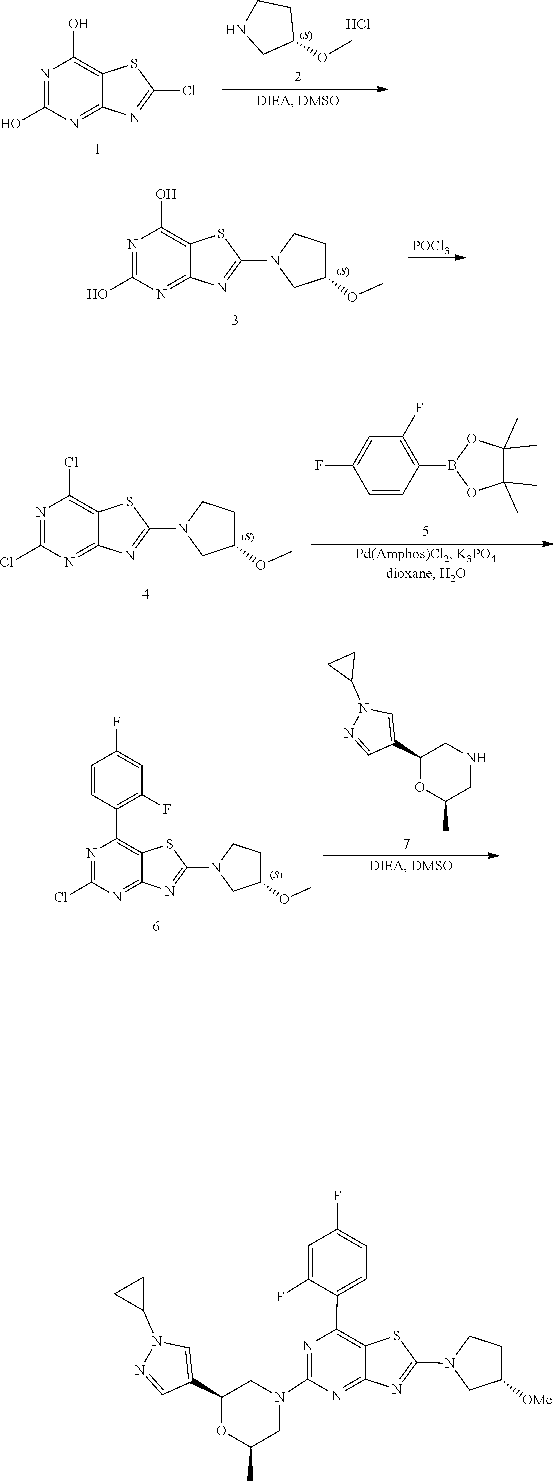

Step 1: To a solution of 2-chlorothiazolo[4,5-d]pyrimidine-5,7-diol (1.00 eq, 500 mg, 2.46 mmol) and (3S)-3-methoxypyrrolidine;hydrochloride (2.00 eq, 676 mg, 4.91 mmol) in DMSO (5 mL) was added DIEA (4.00 eq, 1.6 mL, 9.82 mmol). Then the reaction mixture was stirred at 80° C. for 1 hour. LCMS (1) showed the major peak with desired MS (M+H)+=269.0, purity=97.13%, uv=220 nm, Ret. Time=0.458 min. The reaction solution was adjusted pH<=7 with FA, then the solution purified by prep-HPLC (FA) and freeze-drying to give 2-[(3S)-3-methoxypyrrolidin-1-yl]thiazolo[4,5-d]pyrimidine-5,7-diol (430 mg, 1.60 mmol, 65.27% yield) as yellow solid which was confirmed by H NMR (1A). (P1): MS (M+H)⁺=269.0, purity=97.13%, uv=220 nm, Ret. Time=0.458 min. ¹H NMR (400 MHz, DMSO-d₆) δ ppm 3.26 (s, 3H) 3.34 (s, 6H) 10.86 (br s, 1H) 11.66-11.97 (m, 1H)

Step 2: To a solution of 2-[(3S)-3-methoxypyrrolidin-1-yl]thiazolo[4,5-d]pyrimidine-5,7-diol (1.00 eq, 430 mg, 1.60 mmol) in POCl₃ (1.00 eq, 5.0 mL, ?). Then the reaction mixture was stirred at 100° C. for 16 hours. LCMS (1A) showed the raw material was consumed and the major peak showed desired MS (M+H)+=304.9, purity=88.64%, uv=220 nm, Ret. Time=0.575 min. The reaction solution was concentration in vacuo to take away the POCl₃ and then the residue was dissolved in 10 mL ethyl acetate, poured into 30 mL (NaHCO₃, aq) slowly, then extracted with ethyl acetate (10 mL*2), the organics were then separated and dried (Na₂SO₄) before concentration to dryness to give 5,7-dichloro-2-[(3S)-3-methoxypyrrolidin-1-yl]thiazolo[4,5-d]pyrimidine (300 mg, 0.938 mmol, 58.51% yield) as yellow solid which was confirmed by LCMS (1A1). (P1): MS (M+H)+=304.9, purity=95.39%, uv=220 nm, Ret. Time=0.572 min.

Step 3: To a solution of 5,7-dichloro-2-[(3S)-3-methoxypyrrolidin-1-yl]thiazolo[4,5-d]pyrimidine (1.00 eq, 300 mg, 0.983 mmol) and 2-(2,4-difluorophenyl)-4,4,5,5-tetramethyl-1,3,2-dioxaborolane (1.00 eq, 236 mg, 0.983 mmol) in 1,4-Dioxane (10 mL) with Water (1 mL) was added K₃PO₄ (3.50 eq, 730 mg, 3.44 mmol) and Pd(Amphos)Cl₂ (0.100 eq, 70 mg, 0.0983 mmol) under N₂ atmosphere and the mixture was stirred for 16 h at 60° C. LCMS (1A) showed 44.9% raw material remained and the new peak showed desired MS (M+H)+=383.0, purity=37.13%, uv=220 nm, Ret. Time=0.611 min. The reaction was added water (20 mL) and then extracted with ethyl acetate (10 mL*2), the organics washed with 10 mL saturated brine solution. The organics were then separated and dried (Na₂SO₄) before concentration to dryness. The crude was then purified by silica gel column (PE/EA=1/1, Rf=0.5) to give 5-chloro-7-(2,4-difluorophenyl)-2-[(3S)-3-methoxypyrrolidin-1-yl]thiazolo[4,5-d]pyrimidine (300 mg, 0.374 mmol, 38.03% yield) as white solid which was confirmed by LCMS (lAl): (P1): MS (M+H)+=383.0, purity=47.7%, uv=220 nm, Ret. Time=0.9 min.

Step 4: To a solution of 5-chloro-7-(2,4-difluorophenyl)-2-[(3S)-3-methoxypyrrolidin-1-yl]thiazolo[4,5-d]pyrimidine (1.00 eq, 280 mg, 0.731 mmol) in DMSO (3 mL) was added (2S,6R)-2-(1-cyclopropylpyrazol-4-yl)-6-methyl-morpholine (3.00 eq, 455 mg, 2.19 mmol) and DIEA (5.00 eq, 473 mg, 3.66 mmol) then stirred for 1 h at 100° C. LCMS (1A) showed raw material consumed and the new peak showed desired MS (M+H)+=554.2, purity=28.69%, uv=220 nm, Ret. Time=0.651 min. The reaction was poured into water (20 mL) and then extracted with ethyl acetate (5 mL*3) and the organics washed with 10 mL saturated brine solution. The organics were then separated and dried (Na₂SO₄) before concentration to dryness. The crude was then purified by prep-HPLC (water (FA)-ACN, Phenomenex C18 75*30 mm*3 um) and freeze-drying to give 120 mg product, but H NMR (1A1) showed impurity. The product was purified by silica gel column (PE/EA=1/1, Rf=0.5) to give (2S,6R)-2-(1-cyclopropylpyrazol-4-yl)-4-[7-(2,4-difluorophenyl)-2-[(3S)-3-methoxypyrrolidin-1-yl]thiazolo[4,5-d]pyrimidin-5-yl]-6-methyl-morpholine (69 mg, 0.124 mmol, 16.98% yield) as white solid which was confirmed by QC. (P1): MS (M+H)+=554.2, purity=100%, uv=220 nm, Ret. Time=0.996 min. ¹H NMR (400 MHz, CHLOROFORM-d) δ ppm 0.94-1.03 (m, 2H) 1.07-1.15 (m, 2H) 1.30 (d, J=6.24 Hz, 3H) 2.09-2.32 (m, 2H) 2.72 (dd, J=13.20, 10.64 Hz, 1H) 2.96 (dd, J=13.20, 10.88 Hz, 1H) 3.37 (s, 3H) 3.41-3.70 (m, 3H) 3.74-3.88 (m, 2H) 3.92-4.21 (m, 2H) 4.58 (dd, J=10.88, 2.57 Hz, 1H) 4.81 (br d, J=12.96 Hz, 1H) 4.92 (br d, J=12.59 Hz, 1H) 6.89-7.06 (m, 2H) 7.53 (d, J=4.16 Hz, 2H) 7.71-7.80 (m, 1H)

### Example 30—Synthesis of Compound I-230: 7-(2,4-difluorophenyl)-5-[(2S,6R)-2-[1-(methoxymethyl)pyrazol-4-yl]-6-methyl-morpholin-4-yl]-N,N-dimethyl-thiazolo[4,5-d]pyrimidin-2-amine

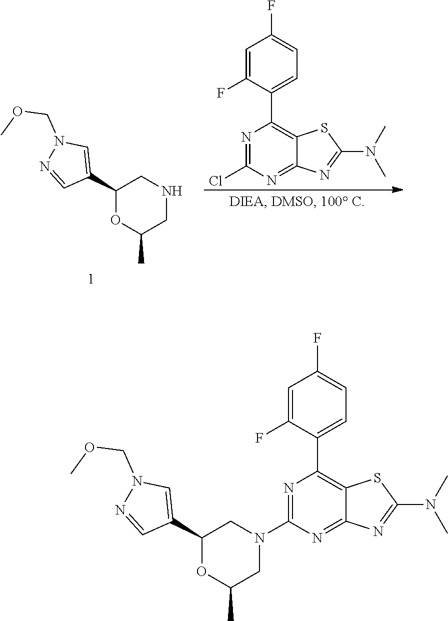

Step 1: To a solution of (2S,6R)-2-[1-(methoxymethyl)pyrazol-4-yl]-6-methyl-morpholine (1.00 eq, 207 mg, 0.245 mmol) in DMSO (2 mL) was added 5-chloro-7-(2,4-difluorophenyl)-N,N-dimethyl-thiazolo[4,5-d]pyrimidin-2-amine (1.00 eq, 80 mg, 0.245 mmol), DIEA (3.00 eq, 0.13 mL, 0.735 mmol) and stirred at 100° C. for 1 h. LCMS (1B2) showed ˜31% of desired product was detected (31%, Rt=0.630 min; [M+H]+=502.2 at 220 nm). The reaction (combined with) was quenched by 30 mL H₂O, extracted with EA (15 mL*3), the combine organic layers was dried over anhydrous Na₂SO₄, filtered and concentrated under reduced pressure to give the residue. The residue was purified by prep-HPLC (flow: 25 mL/min; gradient: from 47-73% water (0.1% FA)-ACN over 10 min; column: Phenomenex luna C18 150*25 mm*10 um) and lyophilized to afford 7-(2,4-difluorophenyl)-5-[(2S,6R)-2-[1-(methoxymethyl)pyrazol-4-yl]-6-methyl-morpholin-4-yl]-N,N-dimethyl-thiazolo[4,5-d]pyrimidin-2-amine (54 mg, 0.107 mmol, 43.84% yield) as white solid, checked by LCMS (1B), HPLC (1C), H NMR (1D(HNMR)), F NMR (1D(FNMR)). (P1): [M+H]+=502.2; purity=100% (220 nm); Retention time=0.948 min. HPLC: Retention time=2.279 min, 99.77% purity at 220 nm. 1H NMR (400 MHz, CHLOROFORM-d) δ=7.79-7.72 (m, 1H), 7.63 (s, 2H), 7.02 (br s, 1H), 6.97-6.89 (m, 1H), 5.36 (s, 2H), 4.93 (br d, J=13.3 Hz, 1H), 4.79 (br d, J=13.0 Hz, 1H), 4.62 (dd, J=2.6, 10.9 Hz, 1H), 3.86-3.75 (m, 1H), 3.37-3.17 (m, 9H), 2.96 (dd, J=10.9, 13.2 Hz, 1H), 2.73 (dd, J=10.7, 13.2 Hz, 1H), 1.30 (d, J=6.3 Hz, 3H).

### Example 31—Synthesis of Compound I-235: 5-((2S,6R)-2-(1-(difluoromethyl)-1H-pyrazol-4-yl)-6-methylmorpholino)-7-(2,4-difluorophenyl)-N,N-dimethylthiazolo[4,5-d]pyrimidin-2-amine

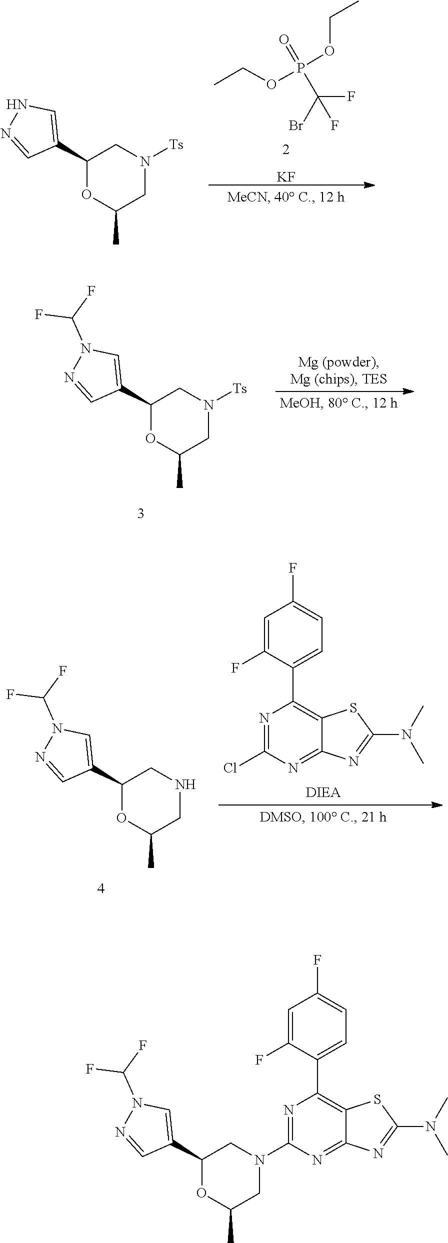

Step 1: A solution of (2R,6S)-2-methyl-4-(p-tolylsulfonyl)-6-(1H-pyrazol-4-yl)morpholine (1.00 eq, 500 mg, 1.56 mmol), KF (2.00 eq, 181 mg, 3.11 mmol) and 1-[[bromo(difluoro)methyl]-ethoxy-phosphoryl]oxyethane (1.50 eq, 623 mg, 2.33 mmol) in MeCN (5 mL) was stirred at 40° C. for 12 h. LCMS (0-P1A1)(5-95AB/1.5 min): RT=0.954 min, 372.1=[M+H]⁺, ESI+ showed 98% of desired product. The reaction mixture was purified by prep-HPLC (Phenomenex Luna C18 150*25 mm*10 um; mobile phase: [water (0.1% FA)-ACN]; B %: 45%-75%, 12 min) to give (2S,6R)-2-(1-(difluoromethyl)-1H-pyrazol-4-yl)-6-methyl-4-tosylmorpholine (500 mg, 1.35 mmol, 86.53% yield) as oil. (M+H)⁺=372.1; purity=100% (220 nm); Retention time=0.570 min. ¹H NMR (400 MHz, CDCl₃) δ=7.76 (s, 1H), 7.64 (d, J=8.4 Hz, 2H), 7.60 (s, 1H), 7.39-7.29 (m, 2H), 7.16-6.98 (m, 1H), 4.73-4.68 (m, 1H), 3.92-3.83 (m, 1H), 3.78 (td, J=2.1, 11.4 Hz, 1H), 3.66 (td, J=2.0, 11.4 Hz, 1H), 2.46 (s, 3H), 2.20 (t, J=11.0 Hz, 1H), 2.10-1.99 (m, 1H), 1.21 (d, J=6.3 Hz, 3H)

Step 2: To the mixture of (2S,6R)-2-[1-(difluoromethyl)pyrazol-4-yl]-6-methyl-4-(p-tolylsulfonyl)morpholine (1.00 eq, 500 mg, 1.35 mmol) and Et₃SiH (30.0 eq, 6.5 mL, 40.4 mmol) in Methanol (10 mL) was added Mg (30.0 eq, 969 mg, 40.4 mmol) (powder) and Mg (30.0 eq, 969 mg, 40.4 mmol) (chips) at 25° C. and the reaction mixture was stirred for 12 hours at 80° C. under N₂ atmosphere. LCMS (3-P1A) showed most of starting material was still remained. The reaction mixture was added Mg (20.0 eq, 646 mg, 26.9 mmol) (powder), Mg (20.0 eq, 646 mg, 26.9 mmol) (chips) and Et₃SiH (20.0 eq, 4.3 mL, 26.9 mmol), then stirred for 12 hours at 80° C. under N₂ atmosphere. LCMS (3-P1A1)(0-60AB/1.5 min): RT=0.224 min, 218.1=[M+H]⁺, ESI+ showed 35% of desired product. The reaction mixture was filtered and concentrated under reduced pressure to give a residue. The residue was used directly for next step. (P1): RT=0.224 min, 218.1=[M+H]⁺, ESI+.

Step 3: To a solution of (2S,6R)-2-[1-(difluoromethyl)pyrazol-4-yl]-6-methyl-morpholine;4-methylbenzenesulfonic acid (2.00 eq, 477 mg, 1.22 mmol) and 5-chloro-7-(2,4-difluorophenyl)-N,N-dimethyl-thiazolo[4,5-d]pyrimidin-2-amine (1.00 eq, 200 mg, 0.612 mmol) in DMSO (2 mL) was added DIEA (4.00 eq, 0.40 mL, 2.45 mmol) at 25° C. Then the reaction mixture was stirred at 100° C. for 1 h. LCMS (7-P1A)(5-95AB/1.5 min): RT=0.982 min, 508.1=[M+H]⁺, ESI+ showed 10.6% of desired product. The reaction mixture was stirred at 100° C. for 4 h. LCMS (7-P1A2)(5-95AB/1.5 min): RT=0.980 min, 508.1=[M+H]+, ESI+ showed 32% of desired product. The reaction mixture was stirred at 100° C. for 4 h. LCMS (7-P1A3)(5-95AB/1.5 min): RT=0.644 min, 508.1=[M+H]⁺, ESI+ showed 42% of desired product. The reaction mixture was stirred at 100° C. for 12 h. LCMS (7-P1A4)(5-95AB/1.5 min): RT=1.017 min, 508.1=[M+H]⁺, ESI+ showed 77% of desired product. The reaction mixture was partitioned between ethyl acetate (100 mL*2) and water (100 mL). The combined organic layers were dried over Na₂SO₄, filtered and concentrated under reduced pressure to give a residue. The crude product was purified by prep-HPLC (Phenomenex C18 75*30 mm*3 um; mobile phase: [water (0.1% FA)-ACN]; B %: 42%-72%, 9 min) to give 5-((2S,6R)-2-(1-(difluoromethyl)-1H-pyrazol-4-yl)-6-methylmorpholino)-7-(2,4-difluorophenyl)-N,N-dimethylthiazolo[4,5-d]pyrimidin-2-amine (91 mg, 0.162 mmol, 26.39% yield) (SFC showed ee. is ˜100.0%) as white solid. (P1): (M+H)⁺=508.2; purity=100% (220 nm); Retention time=0.999 min ¹H NMR (400 MHz, CDCl₃) δ=7.89 (s, 1H), 7.80-7.73 (m, 2H), 7.09-6.92 (m, 3H), 4.97 (br d, J=13.0 Hz, 1H), 4.82 (br d, J=13.1 Hz, 1H), 4.65 (dd, J=2.6, 10.9 Hz, 1H), 3.83 (ddd, J=2.6, 6.3, 10.6 Hz, 1H), 3.28 (br s, 6H), 2.94 (dd, J=10.9, 13.2 Hz, 1H), 2.74 (dd, J=10.6, 13.3 Hz, 1H), 1.32 (d, J=6.3 Hz, 3H)

### Example 32—Synthesis of Compound I-240: 5-[(2S,6R)-2-(1-cyclopropylpyrazol-4-yl)-6-methyl-morpholin-4-yl]-N,N-dimethyl-7-(2,4,6-trifluorophenyl)thiazolo[4,5-d]pyrimidin-2-amine

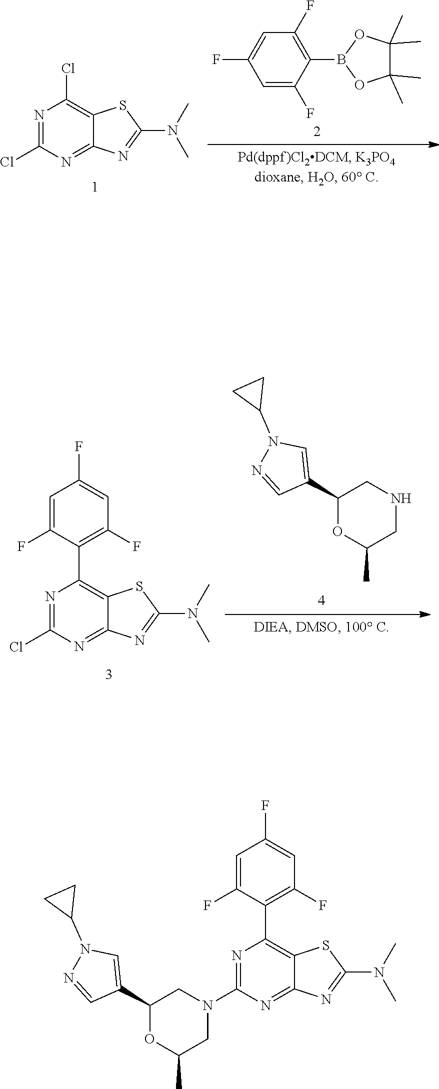 [SMILES: CN(C)c1nc2nc(Cl)nc(Cl)c2s1 ; CC1(C)OB(c2c(F)cc(F)cc2F)OC1(C)C ; CN(C)c1nc2nc(Cl)nc(-c3c(F)cc(F)cc3F)c2s1 ; C[C@@H]1CNC[C@H](c2cnn(C3CC3)c2)O1 ; C[C@@H]1CN(c2nc(-c3c(F)cc(F)cc3F)c3sc(N(C)C)nc3n2)C[C@H](c2cnn(C3CC3)c2)O1]

Step 1: To a solution of 5,7-dichloro-N,N-dimethyl-thiazolo[4,5-d]pyrimidin-2-amine (1.00 eq, 400 mg, 1.61 mmol) in 1,4-Dioxane (8 mL) and Water (0.8 mL) was added 4,4,5,5-tetramethyl-2-(2,4,6-trifluorophenyl)-1,3,2-dioxaborolane (1.10 eq, 456 mg, 1.77 mmol), K₃PO₄ (3.50 eq, 1193 mg, 5.62 mmol) and Pd(dppf)Cl₂·DCM (0.100 eq, 117 mg, 0.161 mmol) and stirred at 60° C. for 12 h. LCMS (1A1) showed ˜41% of desired product was detected (41%, Rt=0.590 min; [M+H]+=345.0 at 220 nm). The reaction (combined with &) was quenched by 80 mL H₂O, extracted with EA (30 mL*3), the combined organic layers was dried over anhydrous Na₂SO₄, filtered and concentrated under reduced pressure to give the residue. The residue was purified by Flash Column (PE:EA=0-30%, PE:EA=3:1, the desired product Rf=0.5) to give the crude product, the crude product was purified by prep-HPLC (flow: 45 mL/min; gradient: from 0-50% water (0.1% FA)-ACN) to afford 5-chloro-N,N-dimethyl-7-(2,4,6-trifluorophenyl)thiazolo[4,5-d]pyrimidin-2-amine (80 mg, 0.220 mmol, 13.73% yield) as white solid, checked by LCMS and HNMR. (P1): [M+H]+=345.0; purity=95.589% (220 nm); Retention time=0.593 min. 1H NMR (400 MHz, CHLOROFORM-d) 6=6.82 (t, J=8.1 Hz, 2H), 3.55-3.09 (m, 6H)

Step 2: To a solution of (2S,6R)-2-(1-cyclopropylpyrazol-4-yl)-6-methyl-morpholine (1.00 eq, 36 mg, 0.174 mmol) in DMSO (1.5 mL) was added 5-chloro-N,N-dimethyl-7-(2,4,6-trifluorophenyl)thiazolo[4,5-d]pyrimidin-2-amine (1.00 eq, 60 mg, 0.174 mmol), DIEA (3.00 eq, 0.091 mL, 0.522 mmol) and stirred at 100° C. for 1 h. LCMS (1A1) showed ˜40% of desired product was detected (40%, Rt=0.678 min; [M+H]⁺=516.2 at 220 nm). The reaction (combined with) was quenched by 30 mL H₂O, extracted with EA (15 mL*3), the combine organic layers was dried over anhydrous Na₂SO₄, filtered and concentrated under reduced pressure to give the residue. The residue was purified by prep-HPLC (flow: 25 mL/min; gradient: from 60-90% water (0.1% FA)-ACN over 10 min; column: Phenomenex luna C18 150*25 mm*10 um) and lyophilized to afford the crude product, the crude product was purified again by Flash Column (PE:EA=0˜50%, PE:EA=1:1, the desired product Rf=0.6) and lyophilized to afford 5-[(2S,6R)-2-(1-cyclopropylpyrazol-4-yl)-6-methyl-morpholin-4-yl]-N,N-dimethyl-7-(2,4,6-trifluorophenyl)thiazolo[4,5-d]pyrimidin-2-amine (22 mg, 0.0412 mmol, 23.67% yield) as white solid, checked by LCMS (1G), HPLC (1H), H NMR (1K (H NMR)), F NMR (1K (F NMR)). (P1): [M+H]+=516.1; purity=97.176% (220 nm); Retention time=0.643 min. HPLC: Retention time=2.417 min, 96.72% purity at 220 nm. 1H NMR (400 MHz, CHLOROFORM-d) 6=7.51 (d, J=2.8 Hz, 2H), 6.78 (t, J=8.1 Hz, 2H), 4.89-4.70 (m, 2H), 4.56 (dd, J=2.5, 10.8 Hz, 1H), 3.79 (ddd, J=2.4, 6.2, 10.4 Hz, 1H), 3.61-3.49 (m, 1H), 3.25 (br d, J=2.0 Hz, 6H), 2.94 (dd, J=11.0, 13.1 Hz, 1H), 2.70 (dd, J=10.7, 13.1 Hz, 1H), 1.28 (d, J=6.2 Hz, 3H), 1.12-1.07 (m, 2H), 1.01-0.95 (m, 2H)

### Example 33—Synthesis of Compound I-245: 5-(3-(1-cyclopropyl-1H-pyrazol-4-yl)-4,4-difluoropiperidin-1-yl)-7-(2,4-difluorophenyl)-N,N-dimethylthiazolo[4,5-d]pyrimidin-2-amine

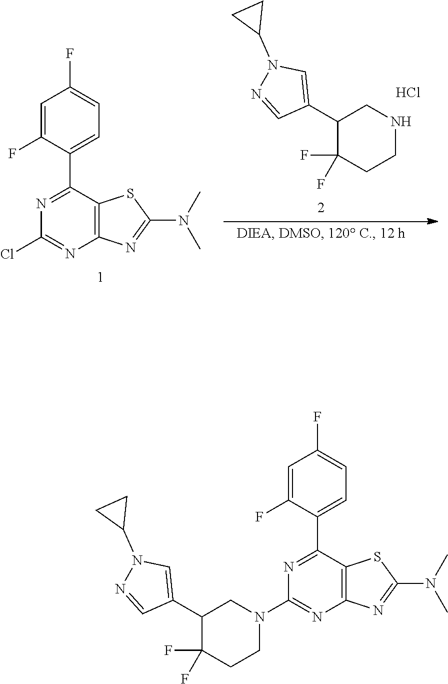 [SMILES: CN(C)c1nc2nc(Cl)nc(-c3ccc(F)cc3F)c2s1 ; FC1(F)CCNCC1c1cnn(C2CC2)c1 ; Cl ; CN(C)c1nc2nc(N3CCC(F)(F)C(c4cnn(C5CC5)c4)C3)nc(-c3ccc(F)cc3F)c2s1]

Step 1: To a solution of 3-(1-cyclopropyl-1H-pyrazol-4-yl)-4,4-difluoropiperidine;hydrochloride (1.30 eq, 157 mg, 0.597 mmol) in DMSO (4 mL) was added DIEA (5.00 eq, 297 mg, 2.30 mmol) and 5-chloro-7-(2,4-difluorophenyl)-N,N-dimethylthiazolo[4,5-d]pyrimidin-2-amine (1.00 eq, 150 mg, 0.459 mmol) in one portion, then the mixture was stirred at 120° C. for 12 h. LCMS 5-P1A showed that 35% the starting material remained and 25% the desired mass was detected (25%, Rt: 0.645 min; [M+H]⁺=518.2 at 220 nm). The mixture was diluted with water (20 mL) and extracted with DCM (30 mL) twice. The combined organic layers were washed with an aqueous solution with brine (40 mL) three times and dried over Na₂SO₄. The solvent was filtered and concentrated under reduced pressure. The residue was purified by prep-TLC (SiO₂, DCM/EtOAc=3/1, the desired product Rf=0.65) and prep-TLC (SiO2, PE/EtOAc=1/1, the desired product Rf=0.45) to give 5-(3-(1-cyclopropyl-1H-pyrazol-4-yl)-4,4-difluoropiperidin-1-yl)-7-(2,4-difluorophenyl)-N,N-dimethylthiazolo[4,5-d]pyrimidin-2-amine (30 mg, 0.0572 mmol, 12.46% yield) as white solid, checked by LCMS 5-P1B1, HPLC 5-P1C, HNMR 5-P1A and FNMR 5-P1A. (P1): [M+H]⁺=518.2; purity=98% (220 nm); Retention time=1.007 min. ¹H NMR (400 MHz, CDCl₃) δ=7.82-7.67 (m, 1H), 7.47 (d, J=7.1 Hz, 2H), 7.02 (dt, J=2.3, 8.1 Hz, 1H), 6.95 (ddd, J=2.4, 8.7, 10.7 Hz, 1H), 4.95-4.79 (m, 2H), 3.57 (tt, J=3.8, 7.3 Hz, 1H), 3.47 (br t, J=12.0 Hz, 2H), 3.38-3.10 (m, 7H), 2.28-1.92 (m, 2H), 1.17-1.08 (m, 2H), 1.06-0.96 (m, 2H).

### Example 34—Synthesis of Compound: 2-(azetidin-1-yl)-5-[(2R,4R)-2-(1-cyclopropylpyrazol-4-yl)tetrahydropyran-4-yl]-7-[2-fluoro-4-(trifluoromethyl)phenyl]thiazolo[4,5-d]pyrimidine (I-248) & 2-(azetidin-1-yl)-5-[(2R,4S)-2-(1-cyclopropylpyrazol-4-yl)tetrahydropyran-4-yl]-7-[2-fluoro-4-(trifluoromethyl)phenyl]thiazolo[4,5-d]pyrimidine (I-249)

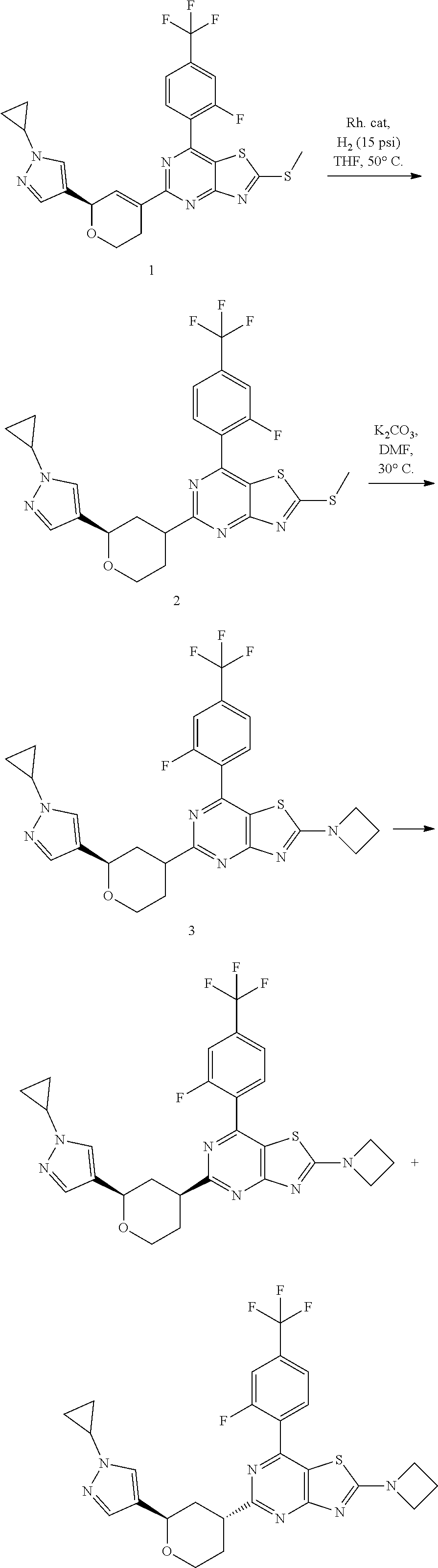 [SMILES: CSc1nc2nc(C3=C[C@H](c4cnn(C5CC5)c4)OCC3)nc(-c3ccc(C(F)(F)F)cc3F)c2s1 ; CSc1nc2nc(C3CCO[C@@H](c4cnn(C5CC5)c4)C3)nc(-c3ccc(C(F)(F)F)cc3F)c2s1 ; Fc1cc(C(F)(F)F)ccc1-c1nc(C2CCO[C@@H](c3cnn(C4CC4)c3)C2)nc2nc(N3CCC3)sc12 ; Fc1cc(C(F)(F)F)ccc1-c1nc([C@H]2CCO[C@@H](c3cnn(C4CC4)c3)C2)nc2nc(N3CCC3)sc12 ; Fc1cc(C(F)(F)F)ccc1-c1nc([C@@H]2CCO[C@@H](c3cnn(C4CC4)c3)C2)nc2nc(N3CCC3)sc12]

Step 1: A solution of 5-[(6R)-6-(1-cyclopropylpyrazol-4-yl)-3,6-dihydro-2H-pyran-4-yl]-7-[2-fluoro-4-(trifluoromethyl)phenyl]-2-methylsulfanyl-thiazolo[4,5-d]pyrimidine (1.00 eq, 300 mg, 0.416 mmol) in THF (10 mL) was added 1,1′-Bis(di-i-propylphosphino)ferrocene(1,5-cyclooctadiene)rhodium(I) tetrafluoroborate (0.400 eq, 119 mg, 0.166 mmol) under N₂ atmosphere, the mixture was purged by H₂ for 3 times, then stirred at 50° C. for 2 h under H₂ atmosphere (15 psi). LCMS (1A) showed part of raw material remained and the major peak showed desired MS (M+H)+=536.2, but the raw material MS signal was detected in the same peak. The reaction solution was filtered and then the filtrate was concentrated under vacuum to give 5-[(2R)-2-(1-cyclopropylpyrazol-4-yl)tetrahydropyran-4-yl]-7-[2-fluoro-4-(trifluoromethyl)phenyl]-2-methylsulfanyl-thiazolo[4,5-d]pyrimidine (300 mg, 0.560 mmol, 134.63% yield) as dark brown solid and used to next step without further purification. (P1): MS (M+H)⁺=536.1, purity=56.87%, uv=220 nm, Ret.Time=0.695 min.

Step 2: To a solution of 5-[(2R)-2-(1-cyclopropylpyrazol-4-yl)tetrahydropyran-4-yl]-7-[2-fluoro-4-(trifluoromethyl)phenyl]-2-methylsulfanyl-thiazolo[4,5-d]pyrimidine (1.00 eq, 300 mg, 0.560 mmol) in DMF (5 mL) was added K₂CO₃ (5.00 eq, 387 mg, 2.80 mmol) and azetidine; hydrochloride (3.00 eq, 157 mg, 1.68 mmol), then the mixture was stirred for 2 h at 30° C. LCMS (1B1) showed the most raw material consumed and the major peak showed desired MS (M+H)+=545.2 formed with BP-MS (M+H)+=543.2 in one peak. The reaction solution was poured into water (20 mL) and then was extracted with ethyl acetate (10 mL*2). The organic layer was washed with 10 mL CaCl₂ (aq.) and 10 mL saturated brine solution. The organic layer was then separated and dried (Na₂SO₄) before concentration to dryness. The residue was dissolved in MeOH and then purified by prep-HPLC (3_Phenomenex Luna C18 75*30 mm*3 um, water(FA)-CAN (61˜81%)) and freeze-drying to give 2-(azetidin-1-yl)-5-[(2R)-2-(1-cyclopropylpyrazol-4-yl)tetrahydropyran-4-yl]-7-[2-fluoro-4-(trifluoromethyl)phenyl]thiazolo[4,5-d]pyrimidine (60 mg, 0.110 mmol, 19.67% yield) as light yellow solid which was confirmed by LCMS (1C1): MS (M+H)⁺=545.3, and the −MS (M+H)+=543.2 in the same peak. HPLC (1C2) showed three peaks. (P1): MS (M+H)+=543.2, purity=75.75%, uv=220 nm, Ret.Time=0.649 min.

Step 3: The racemate product was purified by SFC (DAICEL CHIRALCEL OD (250 mm*30 mm, 10 um), 0.1% NH₃H₂O EtOH) and freeze-dryness after concentration in vacuum to give 2-(azetidin-1-yl)-5-[(2R,4R)-2-(1-cyclopropylpyrazol-4-yl)tetrahydropyran-4-yl]-7-[2-fluoro-4-(trifluoromethyl)phenyl]thiazolo[4,5-d]pyrimidine (19 mg, 0.0337 mmol, 30.61% yield) as white solid and 2-(azetidin-1-yl)-5-[(2R,4S)-2-(1-cyclopropylpyrazol-4-yl)tetrahydropyran-4-yl]-7-[2-fluoro-4-(trifluoromethyl)phenyl]thiazolo[4,5-d]pyrimidine (8.1 mg, 0.0147 mmol, 13.33% yield) as white solid.

(P1): SFC for 1A_D10 peak 1, Ret.Time=0.536 min. MS (M+H)⁺=545.2, purity=98.42%, uv=220 nm, Ret.Time=0.998 min. ¹H NMR (400 MHz, CHLOROFORM-d) δ ppm 0.94-1.03 (m, 2H), 1.06-1.14 (m, 2H), 2.05-2.16 (m, 1H), 2.25 (ddd, J=13.45, 8.31, 4.77 Hz, 1H), 2.35-2.45 (m, 1H), 2.62 (quin, J=7.64 Hz, 2H), 2.66-2.74 (m, 1H), 3.47-3.60 (m, 2H), 3.83-3.97 (m, 2H), 4.35 (br s, 4H), 4.86 (dd, J=8.13, 3.12 Hz, 1H), 7.44-7.52 (m, 3H), 7.59 (d, J=8.31 Hz, 1H), 7.92 (t, J=7.52 Hz, 1H).

(P2): SFC for 1A_D10 peak 2, Ret.Time=0.741 min. MS (M+H)+=545.2, purity=98.62%, uv=220 nm, Ret.Time=0.982 min. ¹H NMR (400 MHz, CHLOROFORM-d) δ ppm 0.95-1.01 (m, 2H), 1.05-1.11 (m, 2H), 2.04-2.21 (m, 3H), 2.29-2.38 (m, 1H), 2.61 (quin, J=7.64 Hz, 2H), 3.32 (tt, J=11.75, 4.02 Hz, 1H), 3.55 (tt, J=7.29, 3.77 Hz, 1H), 3.76 (td, J=11.65, 2.87 Hz, 1H), 4.18-4.26 (m, 1H), 4.34 (br s, 4H), 4.51 (dd, J=11.43, 1.90 Hz, 1H), 7.44-7.53 (m, 3H), 7.59 (d, J=8.31 Hz, 1H), 7.92 (t, J=7.46 Hz, 1H).

### Example 35—Synthesis of Compound I-253

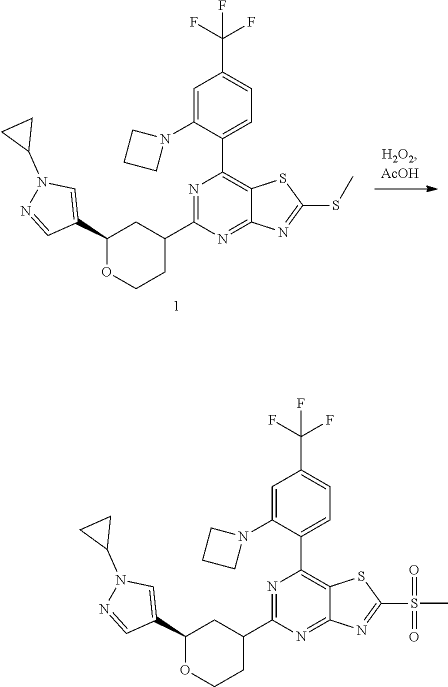 [SMILES: CSc1nc2nc(C3CCO[C@@H](c4cnn(C5CC5)c4)C3)nc(-c3ccc(C(F)(F)F)cc3N3CCC3)c2s1 ; CS(=O)(=O)c1nc2nc(C3CCO[C@@H](c4cnn(C5CC5)c4)C3)nc(-c3ccc(C(F)(F)F)cc3N3CCC3)c2s1]

Step 1: To a solution of 7-[2-(azetidin-1-yl)-4-(trifluoromethyl)phenyl]-5-[(2R,4S)-2-(1-cyclopropylpyrazol-4-yl)tetrahydropyran-4-yl]-2-methylsulfanyl-thiazolo[4,5-d]pyrimidine (1.00 eq, 20 mg, 0.0349 mmol) in Acetic acid (1 mL) was added H₂O₂ (50.5 eq, 0.20 mL, 1.76 mmol) and the solution was stirred for 16 h at 25° C. LCMS (1A) showed raw material was consumed and two peaks showed desired MS (M+H)+=621.2, purity=26.17%, uv=220 nm, Ret. Time=0.875 min and by-product MS (M+H)+=605.2, purity=34.53%, uv=220 nm, Ret. Time=0.920 min. The reaction solution with were added to the Na₂S₂O₃ (aq. 10 mL) and then the mixture was stirred for 1 h, then NaOH (1 N, 5 mL) was added to the reaction to adjust pH=7, and then extracted with Ethyl acetate (5 mL*2). The combined organic layers was washed with brine (10 mL), dried over Na₂SO₄, filtered and concentrated under reduced pressure to give 30 mg crude residue. Then the residue was dissolved in MeCN (2 mL) was added B₂Pin₂ (3.00 eq, 27 mg, 0.105 mmol) and stirred for 2 h. LCMS (1B2) showed the major peak with desired MS(M+H)⁺=605.2, purity=46.98%, uv=220 nm, Ret. Time=0.597 min. The reaction solution was concentration in vacuum and then purified by prep-HPLC (water (FA)-ACN, Phenomenex C18 75*30 mm*3 um) to give about 5 mg but H NMR (1A) showed impurity. The crude was purified by prep-TLC (PE/EA=0/1) to give 7-[2-(azetidin-1-yl)-4-(trifluoromethyl)phenyl]-5-[(2R,4S)-2-(1-cyclopropylpyrazol-4-yl)tetrahydropyran-4-yl]-2-methylsulfonyl-thiazolo[4,5-d]pyrimidine (0.79 mg, 0.00131 mmol, 3.74% yield) as white solid.

(P1): MS (M+H)⁺=605.2, purity=100%, uv=220 nm, Ret. Time=0.927 min. ¹H NMR (400 MHz, CHLOROFORM-d) δ ppm 0.96-1.01 (m, 2H), 1.07-1.11 (m, 2H), 1.99-2.05 (m, 2H), 2.11 (br d, J=12.01 Hz, 1H), 2.26-2.32 (m, 1H), 2.56-2.62 (m, 2H), 3.26 (tt, J=11.69, 4.13 Hz, 1H), 3.46 (s, 3H), 3.55 (tt, J=7.29, 3.78 Hz, 1H), 3.73 (td, J=11.66, 2.81 Hz, 1H), 4.18-4.23 (m, 1H), 4.30 (br s, 4H), 4.48 (dd, J=11.38, 1.88 Hz, 1H), 7.46 (d, J=4.13 Hz, 2H), 7.70 (d, J=7.88 Hz, 1H), 8.01 (br d, J=8.00 Hz, 1H), 8.51 (s, 1H).

### Example 36—Synthesis of Compound I-258: 5-[(2S, 6R)-2-(1-cyclopropylpyrazol-4-yl)-6-methyl-morpholin-4-yl]-7-[2-fluoro-4-(trifluoromethyl) phenyl]-N, N-dimethyl-thiazolo [4, 5-d]pyrimidin-2-amine

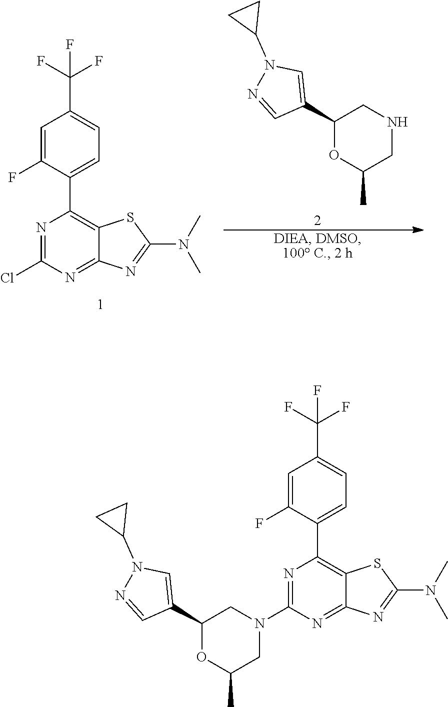 [SMILES: CN(C)c1nc2nc(Cl)nc(-c3ccc(C(F)(F)F)cc3F)c2s1 ; C[C@@H]1CNC[C@H](c2cnn(C3CC3)c2)O1 ; "2" ; "DIEA, DMSO,100° C., 2 h" ; C[C@@H]1CN(c2nc(-c3ccc(C(F)(F)F)cc3F)c3sc(N(C)C)nc3n2)C[C@H](c2cnn(C3CC3)c2)O1]

Step 1: A solution of (2S,6R)-2-(1-cyclopropylpyrazol-4-yl)-6-methyl-morpholine (1.50 eq, 25 mg, 0.119 mmol), 5-chloro-7-[2-fluoro-4-(trifluoromethyl)phenyl]-N,N-dimethyl-thiazolo[4,5-d]pyrimidin-2-amine (1.00 eq, 30 mg, 0.0796 mmol) in DMSO (1 mL) was added DIEA (3.00 eq, 0.039 mL, 0.239 mmol). The mixture was stirred at 100° C. for 2 hours. LCMS (2) (5-95AB/1.5 min): RT=0.718 min, 548.6=[M+H]+, ESI+ showed starting material was consumed completely and desired mass was detected. The crude reaction mixture was purified by prep-HPLC (Phenomenex luna C18 150*25 mm*10 um, water(FA)-ACN) to give 5-[(2S,6R)-2-(1-cyclopropylpyrazol-4-yl)-6-methyl-morpholin-4-yl]-7-[2-fluoro-4-(trifluoromethyl)phenyl]-N,N-dimethyl-thiazolo[4,5-d]pyrimidin-2-amine (47 mg, 0.0860 mmol, 107.98% yield) as yellow solid, which was confirmed by H NMR, F NMR, LCMS, HPLC. P1, single enantiomer of known absolute configuration), LCMS: (M+H)+=548.3; purity=100% (220 nm); Retention time=1.029 min. ¹H NMR (400 MHz, CHLOROFORM-d) δ ppm 0.93-1.05 (m, 2H) 1.06-1.15 (m, 2H) 1.30 (d, J=6.24 Hz, 3H) 2.73 (dd, J=13.02, 10.82 Hz, 1H) 2.97 (dd, J=12.96, 11.13 Hz, 1H) 3.28 (br s, 6H) 3.57 (dt, J=7.21, 3.61 Hz, 1H) 3.75-3.87 (m, 1H) 4.58 (dd, J=10.82, 2.14 Hz, 1H) 4.79 (br d, J=13.20 Hz, 1H) 4.90 (br d, J=12.96 Hz, 1H) 7.47 (br d, J=10.27 Hz, 1H) 7.51-7.59 (m, 3H) 7.89 (t, J=7.46 Hz, 1H)

### Example 37—Synthesis of Compound I-263: 7-[2-fluoro-4-(trifluoromethyl)phenyl]-N,N-dimethyl-5-[(2R,6S)-2-methyl-6-(2-methyl-4-pyridyl)morpholin-4-yl]thiazolo[4,5-d]pyrimidin-2-amine

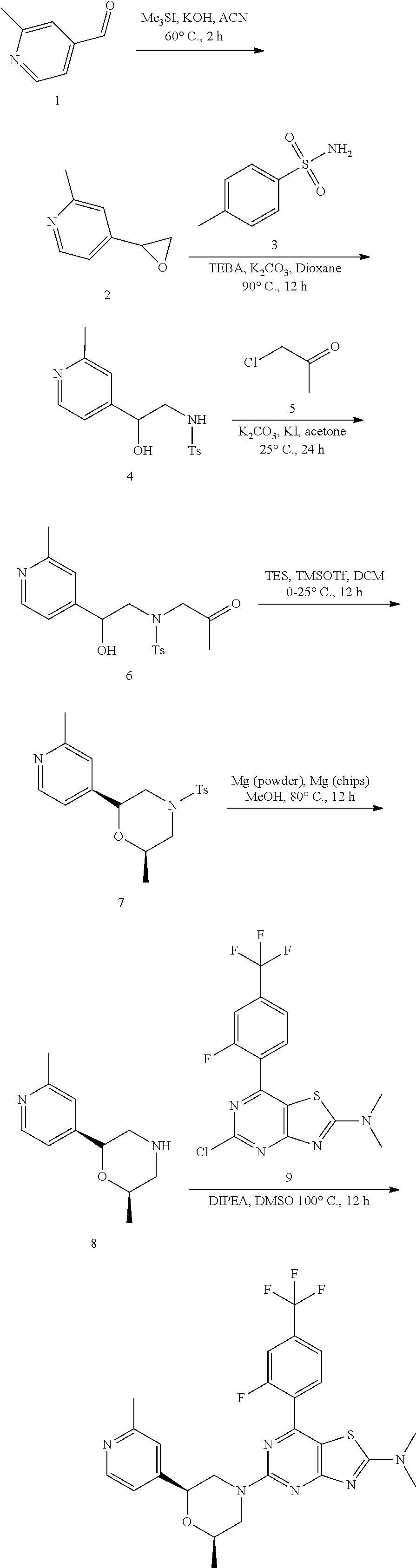

Step 1: A solution of 2-methylpyridine-4-carbaldehyde (1.00 eq, 10.00 g, 82.5 mmol) and trimethylsulfonium;iodide (2.40 eq, 40.43 g, 198 mmol) in MeCN (1000 mL) was added KOH (5.50 eq, 25.43 g, 454 mmol) and stirred at 60° C. for 2 h. LCMS did not show desired product due to lack of response, TLC (UV, PE:EtOAc=1:1, Rf=0.4) showed starting material was consumed and a new spot appeared. The reaction mixture was filtered and washed the cake with EtOAc (500 mL). The combined filtrate was poured into water (1000 mL), extracted with EtOAc (500 mL three times). The combined organic layers were washed by brine (200 mL), dried by Na₂SO₄, purified by flash column (PE to EtOAc condition, 30% to 70%, Rf=0.4 under PE:EtOAc=1:1) and concentrated to give 2-methyl-4-(oxiran-2-yl)pyridine (5.05 g, 37.4 mmol, 45.26% yield) as dark blue oil which confirmed by ¹HNMR. (P1): ¹H NMR (400 MHz, CDCl₃) δ=8.46 (d, J=5.1 Hz, 1H), 7.07 (s, 1H), 7.03-6.98 (m, 1H), 3.81 (dd, J=2.5, 4.1 Hz, 1H), 3.17 (dd, J=4.1, 5.6 Hz, 1H), 2.76 (dd, J=2.4, 5.6 Hz, 1H), 2.55 (s, 3H).

Step 2: A solution of 2-methyl-4-(oxiran-2-yl)pyridine (1.00 eq, 5.00 g, 37.0 mmol), P-TOLUENESULFONAMIDE (2.00 eq, 12.67 g, 74.0 mmol), benzyltriethylammonium chloride (0.100 eq, 0.84 g, 3.70 mmol), K₂CO₃ (0.100 eq, 0.51 g, 3.70 mmol) in 1,4-Dioxane (250 mL) was stirred at 90° C. for 12 h. LCMS (3-P1B) showed starting material consumed and desired product (97%, Rt: 0.405 min; [M+H]+=307.1 at 220 nm) formed. The reaction mixture was poured into water (300 mL), extracted with EtOAc (200 mL three times). The combined organic phase was washed by brine (100 mL), dried by Na₂SO₄, purified by reverse flash (Column: Welch Ultimate XB_C18 20-40 m 120 A; Retention time: 15-50% 40 min; 50% 20 min) and lyophilized to give N-[2-hydroxy-2-(2-methyl-4-pyridyl)ethyl]-4-methyl-benzenesulfonamide (550 mg, 1.80 mmol, 4.85% yield) as brown oil which confirmed by LCMS (3-P1B1). (P1): [M+H]⁺=307.1, purity=45% (220 nm); Retention time=0.403 min

Step 3: A solution of N-[2-hydroxy-2-(2-methyl-4-pyridyl)ethyl]-4-methyl-benzenesulfonamide (1.00 eq, 500 mg, 1.63 mmol), KI (1.10 eq, 298 mg, 1.80 mmol) and K₂CO₃ (3.00 eq, 677 mg, 4.90 mmol) in acetone (10 mL) was added 1-chloropropan-2-one (1.20 eq, 181 mg, 1.96 mmol) at 0° C. then stirred at 25° C. for 24 h. The color of reaction mixture turned from light yellow to orange, LCMS (1C) showed ˜19% of starting material remained and ˜16% of desired product was detected. The reaction mixture was poured into water (20 mL), extracted with EtOAc (10 mL three times). The combined organic phase was washed by brine (10 mL), dried over Na₂SO₄ and evaporated under reduced pressure to give the crude, which was then purified with reversed column (FA) and lyophilized to give N-acetonyl-N-[2-hydroxy-2-(2-methyl-4-pyridyl)ethyl]-4-methyl-benzenesulfonamide (160 mg, 0.441 mmol, 27.05% yield) as yellow solid. (P1): [M+H]⁺=363.1; Retention time=0.443 min

Step 4: A solution of methyl N-acetonyl-N-[2-hydroxy-2-(2-methyl-4-pyridyl)ethyl]-4-methyl-benzenesulfonamide (1.00 eq, 80 mg, 0.221 mmol) and TES (13.9 eq, 0.96 mL, 3.07 mmol) in DCM (3 mL) was added TMSOTf (24.1 eq, 0.96 mL, 5.31 mmol) at 0° C. then stirred at 20° C. for 12 h. LCMS (1A) showed 100% of desired product, the reaction solution was poured into saturated NaHCO₃ solution, extracted with EtOAc (20 mL), dried over Na₂SO₄ and evaporated under reduced pressure to give the residue, which was then purified with flash column (PE:EA=3:1, Rf=0.4) and evaporated under reduced pressure to give (2R,6S)-2-methyl-6-(2-methyl-4-pyridyl)-4-(p-tolylsulfonyl)morpholine (70 mg, 0.202 mmol, 91.54% yield) as yellow solid. (P1): [M+H]⁺=347.1; purity=100% (220 nm); Retention time=0.772 min

Step 5: To a solution of (2R,6S)-2-methyl-6-(2-methyl-4-pyridyl)-4-(p-tolylsulfonyl)morpholine (1.00 eq, 70 mg, 0.202 mmol) in Methanol (3 mL) was added Mg (chips) (15.0 eq, 73 mg, 3.03 mmol) at 25° C. and then the mixture was stirred at 80° C. for 16 h under N₂ atmosphere. LCMS (1A) showed reactant was consumed and traced desired mass, besides, major peak with unknown mass was detected. The mixture was filtered through celite and evaporated under reduced pressure to give (2R,6S)-2-methyl-6-(2-methyl-4-pyridyl)-4-(p-tolylsulfonyl)morpholine (1.00 eq, 70 mg, 0.202 mmol) as yellow solid.

Step 6: To a solution of 5-chloro-7-[2-fluoro-4-(trifluoromethyl)phenyl]-N,N-dimethyl-thiazolo[4,5-d]pyrimidin-2-amine (1.00 eq, 25 mg, 0.0664 mmol) and (2R,6S)-2-methyl-6-(2-methyl-4-pyridyl)morpholine; 4-methylbenzenesulfonic acid (1.50 eq, 36 mg, 0.0995 mmol) in DMSO (2 mL) was added DIEA (5.00 eq, 43 mg, 0.332 mmol), then the mixture was stirred at 100° C. for 12 h. LCMS (1B) showed ˜25% of desired product, the reaction solution was cooled to room temperature, diluted with H₂O (10 mL), extract with EtOAc (10 mL*3), the combined organic layers were dried over Na₂SO₄ and evaporated under reduced pressure to give the residue, which was then purified with Prep-HPLC (Instrument ACSWH-GX-Q; Method: Column Phenomenex luna C18 150*25 mm*10 um; Condition: water (FA)-ACN Begin B 31; End B 62 Gradient Time (min) 10; 100% B Hold Time (min) 2; FlowRate (mL/min) 25) and lyophilized to give 7-[2-fluoro-4-(trifluoromethyl)phenyl]-N,N-dimethyl-5-[(2R,6S)-2-methyl-6-(2-methyl-4-pyridyl)morpholin-4-yl]thiazolo[4,5-d]pyrimidin-2-amine (8.2 mg, 0.0151 mmol, 22.68% yield) as off-white solid. (P1): [M+H]⁺=533.2; purity=85.2% (220 nm); Retention time=0.899 min; HPLC showed ˜98% purity. ¹H NMR (400 MHz, CDCl₃) δ=8.49 (br d, J=4.9 Hz, 1H), 7.89 (t, J=7.6 Hz, 1H), 7.57 (d, J=8.1 Hz, 1H), 7.48 (br d, J=10.1 Hz, 1H), 7.30 (s, 1H), 7.23 (br d, J=4.8 Hz, 1H), 4.96 (br d, J=12.9 Hz, 1H), 4.83 (br d, J=13.1 Hz, 1H), 4.60 (dd, J=2.4, 10.7 Hz, 1H), 3.85 (ddd, J=2.5, 6.3, 10.5 Hz, 1H), 3.29 (br d, J=2.5 Hz, 6H), 2.78 (ddd, J=10.8, 13.3, 18.8 Hz, 2H), 2.60 (s, 3H), 1.36 (d, J=6.1 Hz, 3H).

### Example 38—Synthesis of Compound: 7-(2,4-difluorophenyl)-N,N-dimethyl-5-[(2R,4S)-2-(2-methyl-4-pyridyl)tetrahydropyran-4-yl]thiazolo[4,5-d]pyrimidin-2-amine (I-268) and 7-(2,4-difluorophenyl)-N,N-dimethyl-5-[(2R,4R)-2-(2-methyl-4-pyridyl)tetrahydropyran-4-yl]thiazolo[4,5-d]pyrimidin-2-amine (I-269)

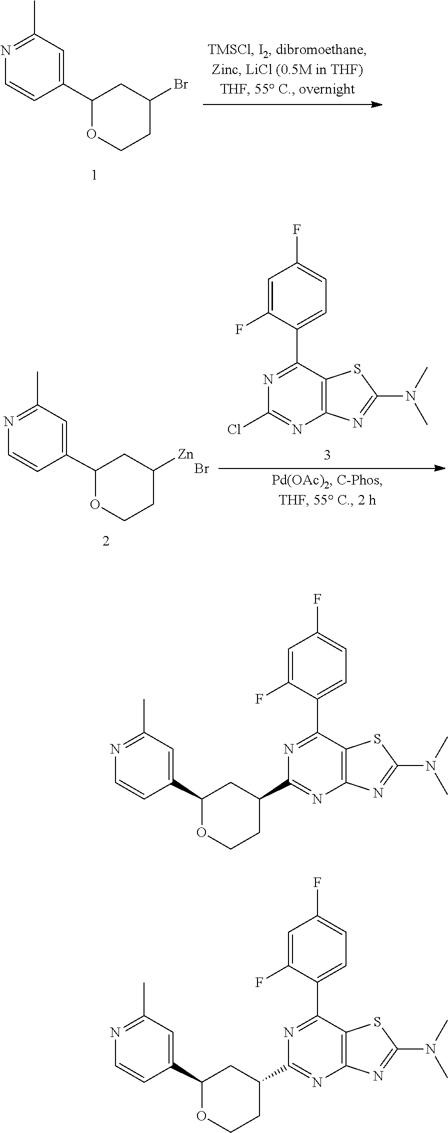

Step 1: Zinc (3.00 eq, 865 mg, 13.2 mmol) was suspended in LiCl (0.5 M in THF) (1.00 eq, 9.0 mL, 4.41 mmol). 1,2-Dibromoethane (0.0500 eq, 0.019 mL, 0.220 mmol) was added and the suspension was stirred at 55° C. for 20 min. Cooled down, then TMSCl (0.0500 eq, 0.028 mL, 0.220 mmol) was introduced and the mixture was stirred at 55° C. for additional 20 min. Cooled down, then iodine (0.0200 eq, 22 mg, 0.0882 mmol) in THF (0.2 mL) (99.5%, Extra Dry over Molecular Sieve, Stabilized, Acros) was introduced and the reaction was stirred at 55° C. for additional 20 min. 5-(4-bromotetrahydropyran-2-yl)-1-methyl-pyridin-2-one (1.00 eq, 1200 mg, 4.41 mmol) in THF (9 mL) (99.5%, Extra Dry over Molecular Sieve, Stabilized, Acros) was then added to the warm (55° C.) suspension of activated zinc. And the reaction was stirred at 55° C. overnight. LCMS (1; 2; 3) showed major peak with de-Br MS (a sample quenched by H₂O). The mixture was used directly for the next step. (M−Br+H)+=178.0; Retention time=0.1 min.

Step 2: To a solution of 5-chloro-7-(2,4-difluorophenyl)-N,N-dimethyl-thiazolo[4,5-d]pyrimidin-2-amine (1.00 eq, 100 mg, 0.306 mmol) and C-phos (0.100 eq, 13 mg, 0.0306 mmol) in THF (2 mL) (99.5%, Extra Dry over Molecular Sieve, Stabilized, Acros) was added PALLADIUM(II) ACETATE (0.0500 eq, 3.4 mg, 0.0153 mmol) under N₂. Then bromo-[(2R)-2-(2-methyl-4-pyridyl)tetrahydropyran-4-yl]zinc (1.20 eq, 118 mg, 0.367 mmol) was added and the mixture was stirred at 55° C. for 2 h. LCMS (1D) showed that ˜39% of desired product was detected. The mixture was cooled to rt, diluted with H₂O (10 mL), extracted with EtOAc (20 mL*3), The combined phase were washed with brine and dried over Na₂SO₄, filtered and concentrated in vacuo. The residue was purified with Prep-HPLC (Instrument: ACSWH-GX-Q; Method: Column Phenomenex luna C18 150*25 mm*10 um; Condition: water(FA)-ACN Begin B 21; End B 41 Gradient Time(min) 10; 100% B Hold Time(min) 20; FlowRate (ml/min) 25) and lyophilized to give 7-(2,4-difluorophenyl)-N,N-dimethyl-5-[(2R,4S)-2-(2-methyl-4-pyridyl)tetrahydropyran-4-yl]thiazolo[4,5-d]pyrimidin-2-amine (26 mg, 0.0563 mmol, 18.40% yield) as off-white solid and 7-(2,4-difluorophenyl)-N,N-dimethyl-5-[(2R,4R)-2-(2-methyl-4-pyridyl)tetrahydropyran-4-yl]thiazolo[4,5-d]pyrimidin-2-amine (13 mg, 0.0266 mmol, 8.68% yield) as off-white solid.

(P1, cis-mixture mixed with ˜3% trans-mixture): [M+H]+=468.1; purity=100% (220 nm); Retention time=0.810 min. ¹H NMR (400 MHz, CDCl₃) δ=8.46 (d, J=5.4 Hz, 1H), 7.80 (d, J=6.4 Hz, 1H), 7.18 (br d, J=4.9 Hz, 1H), 7.07 (dt, J=2.3, 8.3 Hz, 1H), 7.03-6.93 (m, 1H), 4.53 (dd, J=1.0, 12.2 Hz, 1H), 4.33 (br s, 1H), 3.81 (br d, J=2.4 Hz, 1H), 3.54-3.15 (m, 7H), 2.61 (s, 3H), 2.41-2.30 (m, 1H), 2.27-2.09 (m, 2H), 2.07-1.94 (m, 1H)

(P2, trans-mixture mixed with ˜9% cis-mixture): [M+H]+=468.1; purity=99.3% (220 nm); Retention time=0.816 min. ¹H NMR (400 MHz, CDCl₃) δ=8.45 (d, J=5.1 Hz, 1H), 7.83 (dt, J=6.6, 8.4 Hz, 1H), 7.25 (s, 1H), 7.21-6.93 (m, 3H), 4.85 (dd, J=2.1, 9.8 Hz, 1H), 4.07-3.85 (m, 2H), 3.59-3.14 (m, 7H), 2.79 (br d, J=13.6 Hz, 1H), 2.60-2.53 (m, 3H), 2.35-1.98 (m, 3H)

### Example 39—Synthesis of Compound I-273: 5-[(2S, 6R)-2-(1-cyclopropylpyrazol-4-yl)-6-methyl-morpholin-4-yl]-N, N-dimethyl-7-(2, 4, 5-trifluorophenyl) thiazolo[4, 5-d]pyrimidin-2-amine

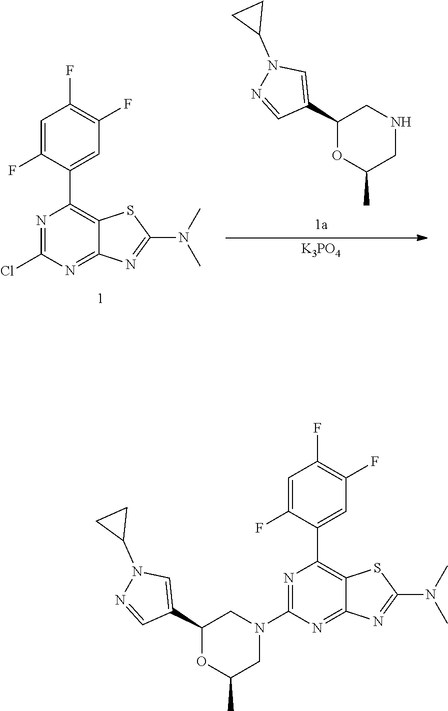 [SMILES: CN(C)c1nc2nc(Cl)nc(-c3cc(F)c(F)cc3F)c2s1 ; C[C@@H]1CNC[C@H](c2cnn(C3CC3)c2)O1 ; "K3PO4" ; C[C@@H]1CN(c2nc(-c3cc(F)c(F)cc3F)c3sc(N(C)C)nc3n2)C[C@H](c2cnn(C3CC3)c2)O1]

Step 1: To a solution of 5-chloro-N,N-dimethyl-7-(2,4,5-trifluorophenyl)thiazolo[4,5-d]pyrimidin-2-amine (1.00 eq, 350 mg, 1.02 mmol) and (2S,6R)-2-(1-cyclopropylpyrazol-4-yl)-6-methyl-morpholine (0.950 eq, 200 mg, 0.964 mmol) in 1,4-Dioxane (7 mL) was added K₃PO₄ (2.00 eq, 431 mg, 2.03 mmol). The mixture was stirred at 100° C. for 12 h. LCMS: (1A) showed a peak with desired MS (LCMS: (M+H)+=516.2; purity=51.3% (UV 220 nm); Retention time=0.843 min). The mixture was concentrated under vacuum to give a crude. The crude was purified by reversed phase-HPLC (FA, FA in water: ACN=100% to 0%, 220&254 nm) and lyophilized to give 5-[(2S,6R)-2-(1-cyclopropylpyrazol-4-yl)-6-methyl-morpholin-4-yl]-N,N-dimethyl-7-(2,4,5-trifluorophenyl)thiazolo[4,5-d]pyrimidin-2-amine (178 mg, 0.331 mmol, 95.77% yield) as off white solid. (P1): (M+H)+=516.2; purity=95.77% (UV 220 nm); Retention time=0.838 min. ¹H NMR (400 MHz, CHLOROFORM-d) 6=7.65-7.56 (m, 1H), 7.54-7.52 (m, 1H), 7.52-7.50 (m, 1H), 7.09-7.00 (m, 1H), 4.93-4.84 (m, 1H), 4.81-4.72 (m, 1H), 4.62-4.51 (m, 1H), 3.90-3.69 (m, 1H), 3.62-3.51 (m, 1H), 3.39-3.15 (m, 6H), 3.05-2.88 (m, 1H), 2.80-2.65 (m, 1H), 1.37-1.24 (m, 3H), 1.14-1.07 (m, 2H), 1.04-0.96 (m, 2H).

### Example 40—Synthesis of Compound: 2-cyclopropyl-5-[(2R,4R)-2-(1-cyclopropylpyrazol-4-yl)tetrahydropyran-4-yl]-7-[2-fluoro-4-(trifluoromethyl)phenyl]thiazolo[4,5-d]pyrimidine (I-278) & 2-cyclopropyl-5-[(2R,4S)-2-(1-cyclopropylpyrazol-4-yl)tetrahydropyran-4-yl]-7-[2-fluoro-4-(trifluoromethyl)phenyl]thiazolo[4,5-d]pyrimidine (I-279)

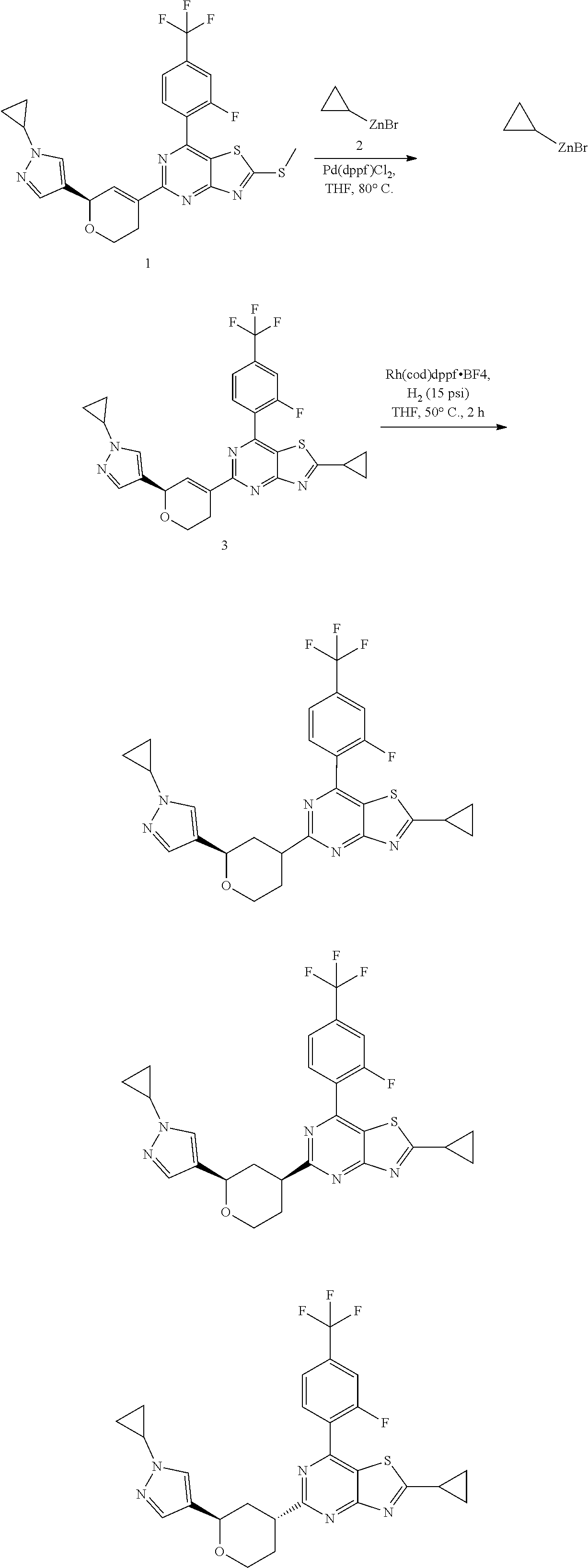

Step 1: To a mixture of 5-[(6R)-6-(1-cyclopropylpyrazol-4-yl)-3,6-dihydro-2H-pyran-4-yl]-7-[2-fluoro-4-(trifluoromethyl)phenyl]-2-methylsulfanyl-thiazolo[4,5-d]pyrimidine (1.00 eq, 200 mg, 0.375 mmol) and Pd(dppf)Cl₂·DCM (0.200 eq, 54 mg, 0.0750 mmol) in THF (5 mL) was added bromo(cyclopropyl)zinc (5.00 eq, 3.7 mL, 1.87 mmol) at 25° C. under N₂ atmosphere, then the reaction mixture was stirred at 80° C. for 12 hours under N₂ atmosphere. LCMS (5-95AB/1.5 min): RT=1.047 min, 528.2=[M+H]⁺, ESI+ showed 51% of desired product. The reaction mixture was diluted with water (50 mL) and then extracted with ethyl acetate (50 mL*3). The combined organic layers were washed with brine, dried over Na₂SO₄, filtered and concentrated under reduced pressure to give a residue. The residue was purified by flash silica gel chromatography (Silica Flash Column, Eluent of 50% Ethyl acetate/Petroleum ethergradient, Rf=0.35 product) to afford 2-cyclopropyl-5-[(2R)-2-(1-cyclopropylpyrazol-4-yl)tetrahydropyran-4-yl]-7-[2-fluoro-4-(trifluoromethyl)phenyl]thiazolo[4,5-d]pyrimidine (60 mg, 0.113 mmol, 30.23% yield) as brown oil. RT=1.050 min, 528.2=[M+H]+, ESI+ showed 87.8% purity.

Step 2: To a solution of 2-cyclopropyl-5-[(6R)-6-(1-cyclopropylpyrazol-4-yl)-3,6-dihydro-2H-pyran-4-yl]-7-[2-fluoro-4-(trifluoromethyl)phenyl]thiazolo[4,5-d]pyrimidine (1.00 eq, 60 mg, 0.0989 mmol) in THF (2 mL) was added 1,1′-Bis(di-i-propylphosphino)ferrocene(1,5-cyclooctadiene)rhodium(I) tetrafluoroborate (0.705 eq, 50 mg, 0.0697 mmol) under N₂, then the mixture was purged by H₂ for 3 times, then stirred at 50° C. for 2 h under H₂ atmosphere (15 psi). LCMS (5-95AB/1.5 min): RT=1.010 min, 530.2=[M+H]+, ESI+ showed 96.1% of desired product. The reaction solution was concentrated under reduced pressure to afford a residue. The residue was purified by prep-HPLC (Column, [Phenomenex Luna C18 150*25 mm*10 um]; mobile phase: [ACN] and [H₂O](conditions: [water (0.225% FA)-ACN], B %: 57%-87%; Detector, UV 254 nm. RT: [10 min]) to afford racemate (34 mg) as pink solid. The racemate was combined with the racemate (12 mg) of page to separate enantiomers. The combined racemates were separated by SFC (Sample preparation: Add CH₃OH 20 ml into sample; Instrument:Waters 80Q; Mobile Phase: 50% ETOH (0.1% NH₃·H₂O) in Supercritical CO₂; Flow Rate: 70 g/min; Cycle Time: 4.4 min, total time: 50 min; Single injection volume: 3.5 ml; Back Pressure: 100 bar to keep the CO₂ in Supercritical flow) to afford two enantiomers. 2-cyclopropyl-5-[(2R,4R)-2-(1-cyclopropylpyrazol-4-yl)tetrahydropyran-4-yl]-7-[2-fluoro-4-(trifluoromethyl)phenyl]thiazolo[4,5-d]pyrimidine (21 mg, 0.0377 mmol, 38.11% yield) was given as pink solid (Peak 1 in SFC).

(P1): [M+H]⁺=530.2; purity=96.6% (220 nm); Retention time=1.019 min; H NMR (400 MHz, CHLOROFORM-d) δ ppm 0.96-1.04 (m, 2H) 1.08-1.14 (m, 2H) 1.37-1.44 (m, 2H) 1.52-1.56 (m, 2H) 2.11-2.22 (m, 1H) 2.31 (ddd, J=13.41, 8.22, 4.75 Hz, 1H) 2.38-2.50 (m, 2H) 2.70-2.78 (m, 1H) 3.57 (tt, J=7.21, 3.67 Hz, 1H) 3.65 (quin, J=5.32 Hz, 1H) 3.87-3.96 (m, 2H) 4.87 (dd, J=8.00, 3.25 Hz, 1H) 7.47 (d, J=7.88 Hz, 2H) 7.55 (d, J=10.38 Hz, 1H) 7.63 (d, J=7.88 Hz, 1H) 7.97 (t, J=7.50 Hz, 1H); 2-cyclopropyl-5-[(2R,4S)-2-(1-cyclopropylpyrazol-4-yl)tetrahydropyran-4-yl]-7-[2-fluoro-4-(trifluoromethyl)phenyl]thiazolo[4,5-d]pyrimidine (9.5 mg, 0.0180 mmol, 18.21% yield) was given as pink solid (Peak 2 in SFC).

(P2): [M+H]⁺=530.2; purity=100% (220 nm); Retention time=1.016 min; H NMR (400 MHz, CHLOROFORM-d) δ ppm 0.95-1.03 (m, 2H) 1.07-1.13 (m, 2H) 1.37-1.43 (m, 2H) 1.54-1.57 (m, 2H) 2.11-2.25 (m, 3H) 2.35-2.48 (m, 2H) 3.46 (tt, J=11.49, 4.14 Hz, 1H) 3.56 (tt, J=7.32, 3.69 Hz, 1H) 3.79 (td, J=11.41, 3.31 Hz, 1H) 4.21-4.30 (m, 1H) 4.54 (dd, J=11.26, 1.88 Hz, 1H) 7.49 (s, 2H) 7.55 (br d, J=10.38 Hz, 1H) 7.63 (d, J=8.25 Hz, 1H) 7.97 (t, J=7.44 Hz, 1H).

### Example 41—Synthesis of Compound: 5-[4-[7-(2,4-difluorophenyl)-2-(dimethylamino)thiazolo[4,5-d]pyrimidin-5-yl]tetrahydropyran-2-yl]-1-methyl-pyridin-2-one (I-288) and 5-[(2R,4R)-4-[7-(2,4-difluorophenyl)-2-(dimethylamino)thiazolo[4,5-d]pyrimidin-5-yl]tetrahydropyran-2-yl]-1-methyl-pyridin-2-one (I-289)

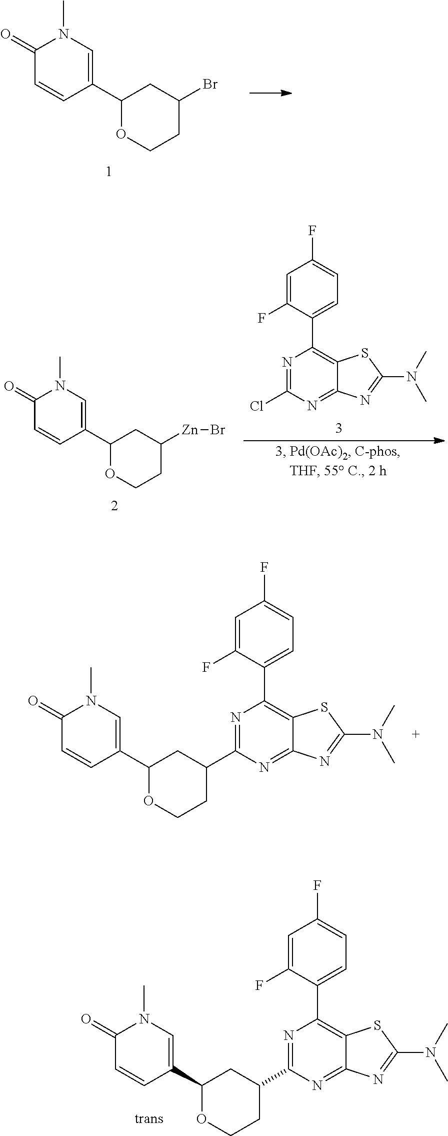

Step 1: Zinc (3.00 eq, 865 mg, 13.2 mmol) was suspended in LiCl (0.5 M in THF) (1.00 eq, 9.0 mL, 4.41 mmol). 1,2-Dibromoethane (0.0500 eq, 0.019 mL, 0.220 mmol) was added and the suspension was stirred at 55° C. for 20 min. Cooled down, then TMSCl (0.0500 eq, 0.028 mL, 0.220 mmol) was introduced and the mixture was stirred at 55° C. for additional 20 min. Cooled down, then iodine (0.0200 eq, 22 mg, 0.0882 mmol) in THF (0.2 mL) (99.5%, Extra Dry over Molecular Sieve, Stabilized, Acros) was introduced and the reaction was stirred at 55° C. for additional 20 min. 5-(4-bromotetrahydropyran-2-yl)-1-methyl-pyridin-2-one (1.00 eq, 1200 mg, 4.41 mmol) in THF (9 mL) (99.5%, Extra Dry over Molecular Sieve, Stabilized, Acros) was then added to the warm (55° C.) suspension of activated zinc. And the reaction was stirred at 55° C. overnight. LCMS (1; 2; 3) showed major peak with de-Br MS (a sample quenched by H₂O). The mixture was used directly for next step. (M−Br+H)⁺=178.0; Retention time=0.1 min.

Step 2: To a suspension of C-Phos (0.1000 eq, 13 mg, 0.0306 mmol) and 5-chloro-7-(2,4-difluorophenyl)-N,N-dimethyl-thiazolo[4,5-d]pyrimidin-2-amine (1.00 eq, 100 mg, 0.306 mmol) and Pd(OAc)₂ (0.0500 eq, 3.4 mg, 0.0153 mmol) in THF (3 mL) (99.5%, Extra Dry over Molecular Sieve, Stabilized, Acros) was added bromo-[2-(1-methyl-6-oxo-3-pyridyl)tetrahydropyran-4-yl]zinc (1.20 eq, 4 mL) under N₂ and the mixture was then stirred at 55° C. for 2 h. LCMS (1A) showed ˜37% of desired mass and excess Zinc reagents, the reaction solution was poured into H₂O (10 mL), extracted with EtOAc (10 mL*3), the combined organic layers were dried over Na₂SO₄ and evaporated under reduced pressure to give the residue, which was then purified with Prep-HPLC (FA) and lyophilized to give 5-[4-[7-(2,4-difluorophenyl)-2-(dimethylamino)thiazolo [4,5-d]pyrimidin-5-yl]tetrahydropyran-2-yl]-1-methyl-pyridin-2-one (40 mg, 0.0827 mmol, 27.03% yield) as off-white solid. (P1): (M+H)⁺=484.1; Retention time=0.575 min.

Step 3: The step was used for purification of. The residue was purified by prep-HPLC (Column, [Phenomenex luna C18 250*50 mm*10 um]; mobile phase: [ACN] and [H₂O](conditions: [water (0.225% FA)-ACN], B %: 65%-90%; Detector, UV 254 nm. RT: [22 min]) to afford 5-[4-[7-(2,4-difluorophenyl)-2-(dimethylamino)thiazolo[4,5-d]pyrimidin-5-yl]tetrahydropyran-2-yl]-1-methyl-pyridin-2-one (24 mg, 0.0487 mmol, 15.91% yield) as white solid and 5-[(2R,4R)-4-[7-(2,4-difluorophenyl)-2-(dimethylamino)thiazolo[4,5-d]pyrimidin-5-yl]tetrahydropyran-2-yl]-1-methyl-pyridin-2-one (12 mg, 0.0238 mmol, 7.76% yield) as white solid

(P1, racemate, 4 peaks): LCMS: [M+H]⁺=484.2; purity=100% (220 nm); Retention time=0.872 min. ¹H NMR (400 MHz, CDCl₃) δ=8.14 (s, 1H), 7.84-7.75 (m, 1H), 7.34 (s, 1H), 7.45-7.31 (m, 1H), 7.12-6.94 (m, 2H), 6.62-6.54 (m, 1H), 4.62 (br d, J=8.9 Hz, 1H), 4.34-4.19 (m, 2H), 3.98-3.86 (m, 1H), 3.75 (dt, J=2.8, 11.6 Hz, 1H), 3.55 (s, 4H), 3.46-3.16 (m, 6H), 2.73 (br d, J=13.5 Hz, 1H), 2.56-2.47 (m, 1H), 2.26 (br d, J=13.3 Hz, 1H), 2.21-1.98 (m, 4H)

(P2, trans-mixture): [M+H]⁺=484.2; purity=95.8% (220 nm); Retention time=0.889 min. ¹H NMR (400 MHz, CDCl₃) δ=7.79 (dt, J=6.6, 8.4 Hz, 1H), 7.40 (dd, J=2.4, 9.3 Hz, 1H), 7.33 (s, 1H), 7.14-6.92 (m, 2H), 6.57 (d, J=9.3 Hz, 1H), 4.62 (dd, J=1.9, 10.1 Hz, 1H), 3.99-3.87 (m, 3H), 3.54 (s, 6H), 2.73 (br d, J=13.6 Hz, 1H), 2.52 (br d, J=13.3 Hz, 1H), 2.28-1.94 (m, 5H).

### Example 42—Synthesis of I-293: 8-(azetidin-1-yl)-2-((2R,4S)-2-(1-cyclopropyl-1H-pyrazol-4-yl)tetrahydro-2H-pyran-4-yl)-6-(2,4-difluorophenyl)-7-methyl-7H-purine

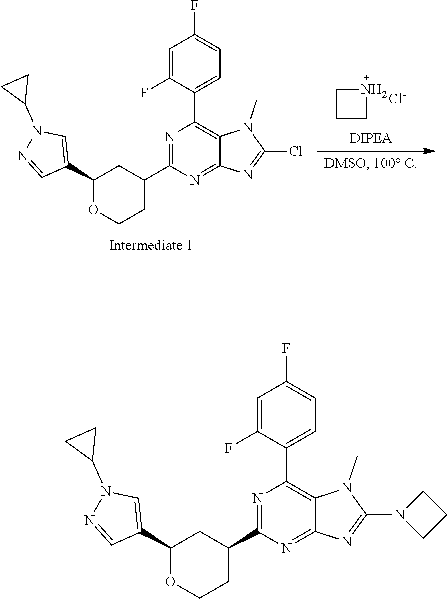

Step 1: To a solution of intermediate 1 (1.0 eq, 50 mg, 0.11 mmol) in DMSO (1.1 mL) was added azetidine hydrochloride (2.0 eq, 20 mg, 0.22 mmol) and DIPEA (5.0 eq, 93 μL, 0.53 mmol). The reaction was then stirred at 100° C. for 16 h. Following completion, the mixture was cooled down and diluted with saturated solution of NaHCO₃ (20 ml) and EtOAc (20 ml). The organic phase was extracted, washed with water (10 ml), brine (10 ml), dried over Na₂SO₄ and concentrated. The crude residue was then purified by reverse phase chromatography (Biotage C18 30 g) using aqueous 10 mM Ammonium Formate and ACN (10-60%). The mixture of diastereoisomers was then separated by Prep-HPLC purification (Gemini® 5 um NX—C18 110 Å, 100×30 mm) using aqueous 10 mM ammonium bicarbonate and ACN (30-50%) to afford 8-(azetidin-1-yl)-2-((2R,4S)-2-(1-cyclopropyl-1H-pyrazol-4-yl)tetrahydro-2H-pyran-4-yl)-6-(2,4-difluorophenyl)-7-methyl-7H-purine (5.9 mg, 0.012 mmol, 11% yield) as a white solid. ESI-MS (m/z+): 492.2 [M+H]. ¹H NMR (CHCl₃-d, 400 MHz): δ_{H} 7.65 (1H, td, J=8.4, 6.4 Hz), 7.45 (2H, s), 7.03-7.08 (1H, m), 6.93 (1H, ddd, J=9.9, 8.7, 2.5 Hz), 4.46 (1H, dd, J=11.4, 2.3 Hz), 4.38 (4H, t, J=7.6 Hz), 4.15-4.19 (1H, m), 3.72 (1H, td, J=11.9, 2.4 Hz), 3.49-3.54 (1H, m), 3.25-3.29 (1H, m), 3.23 (3H, d, J=1.6 Hz), 2.46-2.54 (2H, m), 1.96-2.27 (4H, m), 1.04-1.08 (2H, m), 0.92-0.97 (2H, m). ¹⁹F NMR (CHCl₃-d, 376 MHz): δ_{F} −107.4 (1F, d, J=15.5 Hz) −110.2 (1F, ddt, J=9.0 Hz).

I-298 is synthesized following the procedure for I-293, with starting material: 8-chloro-2-((2R,4S)-2-(1-cyclopropyl-1H-pyrazol-4-yl)tetrahydro-2H-pyran-4-yl)-6-(2,4-difluorophenyl)-7-methyl-7H-purine and N-methylcyclopropanamine. ESI-MS (m/z+): 506.2 [M+H]. ¹H NMR (CHCl₃-d, 400 MHz): δ_{H} 7.70-7.76 (1H, m), 7.45 (2H, s), 7.09 (1H, d, J=8.6 Hz), 6.92-6.97 (1H, m), 4.46 (1H, dd, J=11.3, 2.2 Hz), 4.15-4.18 (1H, m), 3.72 (1H, t, J=11.7 Hz), 3.50-3.54 (1H, m), 3.36 (3H, d, J=1.4 Hz), 3.27 (1H, d, J=4.0 Hz), 3.20 (3H, s), 2.99-3.02 (1H, m), 2.26 (1H, s), 2.08-2.19 (2H, m), 2.02 (1H, s), 1.04-1.07 (2H, m), 0.92-0.97 (2H, m), 0.90 (2H, d, J=6.6 Hz), 0.77 (2H, s). ¹⁹F NMR (CHCl₃-d, 376 MHz): δ_{F} −107.3 (1F, m) −110.4 (1F, m).

### Example 43—Synthesis of Compound I-303: 2-((2R,4S)-2-(1-cyclopropyl-1H-pyrazol-4-yl)tetrahydro-2H-pyran-4-yl)-6-(2,4-difluorophenyl)-N,N,7-trimethyl-7H-purin-8-amine

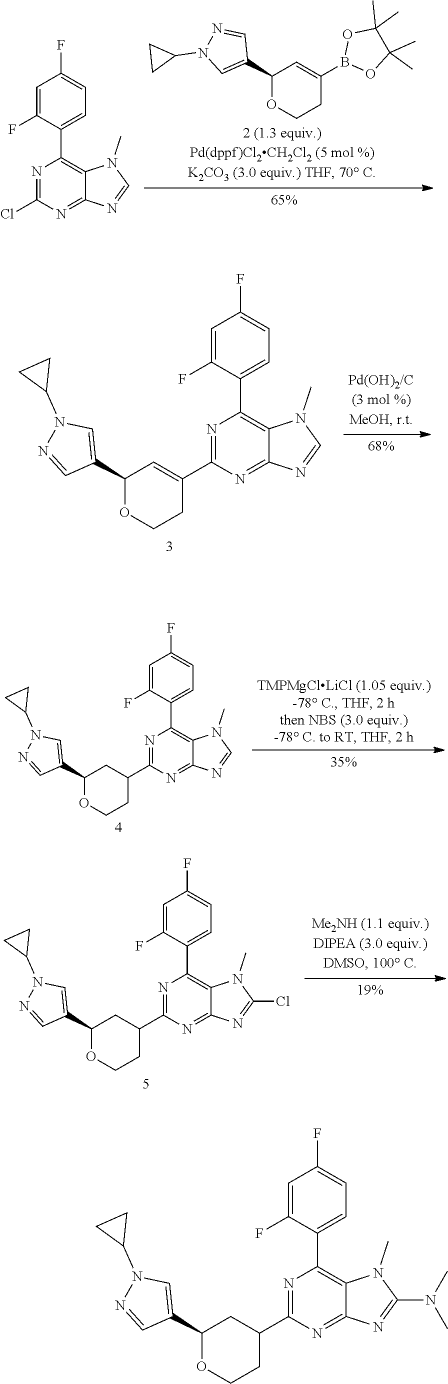

Step 1: A 100 mL round-bottom flask was charged with 2-chloro-6-(2,4-difluorophenyl)-7-methyl-7H-purine (1.0 equiv., 1.0 g, 3.56 mmol), Pd(dppf)Cl₂·CH₂Cl₂ (5 mol %, 145 mg, 0.18 mmol) and K₂CO₃ (3.0 eq, 1.16 g, 10.68 mmol) and the vial was cycled three times with vacuum/N₂. 1,4-Dioxane (14 mL), water (4 mL) and 1-cyclopropyl-4-[(6R)-4-(4,4,5,5-tetramethyl-1,3,2-dioxaborolan-2-yl)-3,6-dihydro-2H-pyran-6-yl]pyrazole 2 (1.3 eq, 1.5 g, 4.63 mmol) were added and the solution was degassed with N₂ for 5 min. The reaction flask was then capped, immersed in a pre-heated oil bath at 85° C. and stirred for 16 h. The reaction mixture was cooled down to room temperature, filtered over a pad of Celite, rinsed with EtOAc (2×50 mL), the volatiles were removed under reduced pressure and the crude material was purified by flash chromatography (Silicycle® column 80 g, using a gradient from 100% CH₂Cl₂ to 100% EtOAc). The selected fractions were evaporated to afford (R)-2-(6-(1-cyclopropyl-1H-pyrazol-4-yl)-3,6-dihydro-2H-pyran-4-yl)-6-(2,4-difluorophenyl)-7-methyl-7H-purine 3 as a white solid (1.0 g, 2.32 mmol, 65% yield, cis/trans ratio 2.5:1). ESI-MS (m/z+): 435.3 [M+1]+, LC-RT: 1.29 min.

Step 2: A 4-dram vial was charged with 2(R)-2-(6-(1-cyclopropyl-1H-pyrazol-4-yl)-3,6-dihydro-2H-pyran-4-yl)-6-(2,4-difluorophenyl)-7-methyl-7H-purine 3 (1.0 equiv., 1.0 g, 2.32 mmol), MeOH (2.7 mL) and 10% Pd(OH)₂/C (3 mol %, 100 mg, 0.07 mmol). The vial was capped with a screw cap with septa, fitted with a 20G needle and placed in a Parr reactor. The Parr reactor was purged three times with H₂ (g), pressurized under 100 psi of H₂ and stirred at 50° C. for 48 h. The reaction mixture was cooled down to room temperature, filtered through Celite and the pad of Celite was rinsed with 2×100 mL of MeOH. The combined organic fractions were evaporated under reduced pressure and the residue was purified by C18 chromatography (Biotage® column 60 g, using a gradient from 5% of MeCN in water (0.1% FA) to 90% MeCN in water (0.1% FA)). The selected fractions were evaporated to yield the desired product 2-((2R)-2-(1-cyclopropyl-1H-pyrazol-4-yl)tetrahydro-2H-pyran-4-yl)-6-(2,4-difluorophenyl)-7-methyl-7H-purine 4 (693 mg, 1.59 mmol, 68% yield, cis/trans ratio 2.5:1). ESI-MS (m/z+), Major diastereoisomer: 437.2 [M+1]+, LC-RT: 1.18 min. ESI-MS (m/z+), minor diastereoisomer: 437.2 [M+1]+, LC-RT: 1.20 min.

Step 3: To a solution of 2-((2R)-2-(1-cyclopropyl-1H-pyrazol-4-yl)tetrahydro-2H-pyran-4-yl)-6-(2,4-difluorophenyl)-7-methyl-7H-purine 4 (1.0 equiv., 693 mg, 1.59 mmol) in THF (16 mL) at −78° C. was slowly added TMPMgCl·LiCl (1.05 equiv., 1.7 mL, 1.67 mmol). The reaction was stirred at −78° C. for 2 hours then a solution of NBS (3.0 equiv., 848 mg, 4.76 mmol) in THF (5 mL) was added. The reaction was allowed to warm to room temperature then stirred at 50° C. for 16 hours. The reaction was quenched with saturated NH₄Cl solution, extracted with CH₂Cl₂ (2×20 mL). The combined organic extracts were washed with brine, dried over anhydrous Na₂SO₄ and concentrated under reduced pressure. The crude material was purified by flash chromatography (Biotage® 50 g column, using a gradient from 30% EtOAc in hexanes to 100% EtOAc) to afford 8-chloro-2-((2R)-2-(1-cyclopropyl-1H-pyrazol-4-yl)tetrahydro-2H-pyran-4-yl)-6-(2,4-difluorophenyl)-7-methyl-7H-purine 5 (235 mg, 0.56 mmol, 35% yield, cis/trans ratio 2.5:1). ESI-MS (m/z+): 473.3 [M+1]+, LC-RT: 1.47 min.

Step 4: Dimethylamine (2.0 equiv., 11 μL, 0.23 mmol) and DIPEA (3.0 equiv., 59 μL, 0.34 mmol) were added to a stirring solution of 8-chloro-2-((2R)-2-(1-cyclopropyl-1H-pyrazol-4-yl)tetrahydro-2H-pyran-4-yl)-6-(2,4-difluorophenyl)-7-methyl-7H-purine 5 (1.0 equiv., 53 mg, 0.11 mmol) in DMSO (1.0 mL) and the reaction vial was immersed in a pre-heated bath at 100° C. The reaction was stirred at 100° C. until complete conversion was observed by LCMS. The reaction mixture was cooled to RT and the resulting solution was directly loaded onto a 12 g Biotage® C18 column and purified by reverse phase flash chromatography using a gradient from 10% MeCN in water (0.1% FA) to 95% MeCN in water (0.1% FA). The selected fractions were evaporated to afford 2-((2R,4S)-2-(1-cyclopropyl-1H-pyrazol-4-yl)tetrahydro-2H-pyran-4-yl)-6-(2,4-difluorophenyl)-N,N,7-trimethyl-7H-purin-8-amine (10 mg, 0.02 mmol, 19% yield)). ¹H NMR (CHCl₃-d, 400 MHz): SH 7.72 (1H, td, J=8.4, 6.4 Hz), 7.45 (2H, s), 7.07 (1H, td, J=8.3, 2.4 Hz), 6.91-6.96 (1H, m), 4.46 (1H, dd, J=11.4, 2.2 Hz), 4.18 (1H, dd, J=11.4, 4.2 Hz), 3.72 (1H, td, J=11.8, 2.5 Hz), 3.52 (1H, tt, J=7.2, 3.8 Hz), 3.25-3.31 (4H, m), 3.16 (6H, s), 2.28 (1H, d, J=13.4 Hz), 2.06-2.18 (2H, m), 2.01 (1H, d, J=13.4 Hz), 1.04-1.08 (2H, m), 0.93-0.97 (2H, m). ESI-MS (m/z+): 480.4 [M+1]+, LC-RT: 1.13 min.

### Table B. Exemplary Compounds

The compounds disclosed below in Table 1 were made by a method of the present disclosure or a similar method. The appropriate reagents, starting materials and conditions necessary for synthesizing the compounds of Table 1 would be apparent to a person of ordinary skill in the art. Compounds designated with “(+/−)” were isolated as a mixture of diastereomers sharing the same relative stereochemistry (ie. cis or trans). Compounds designated with “(rac)” were isolated as a mixture of all possible stereoisomers of the shown compound. Compounds lacking either designation were isolated with the specific stereochemistry shown, such that the specific stereoisomer shown made up at least 90% of the isolated product.

| | | | Method used to |
|---|---|---|---|
| Ex # | Structure | Name | synthesize |
| 1 | 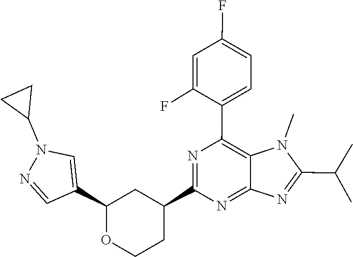 [SMILES: CC(C)c1nc2nc([C@H]3CCO[C@@H](c4cnn(C5CC5)c4)C3)nc(-c3ccc(F)cc3F)c2n1C] | 2-((2R,4S)-2-(1-cyclopropyl- 1H-pyrazol-4-yl)tetrahydro-2H- pyran-4-yl)-6-(2-fluoro-4- (trifluoromethyl)phenyl)-8- isopropyl-7-methyl-7H-purine | |
| 2 | 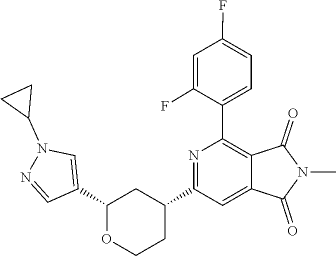 [SMILES: CN1C(=O)c2cc([C@@H]3CCO[C@H](c4cnn(C5CC5)c4)C3)nc(-c3ccc(F)cc3F)c2C1=O] | 6-((2S,4R)-2-(1-cyclopropyl- 1H-pyrazol-4-yl)tetrahydro-2H- pyran-4-yl)-4-(2-fluoro-4- (trifluoromethyl)phenyl)-2- methyl-1H-pyrrolo[3,4- c]pyridine-1,3(2H)-dione | |
| 3 | 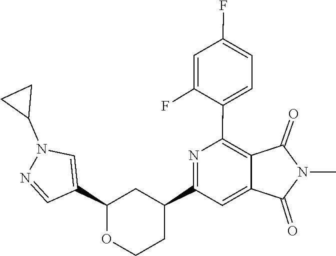 [SMILES: CN1C(=O)c2cc([C@H]3CCO[C@@H](c4cnn(C5CC5)c4)C3)nc(-c3ccc(F)cc3F)c2C1=O] | 6-((2R,4S)-2-(1-cyclopropyl- 1H-pyrazol-4-yl)tetrahydro-2H- pyran-4-yl)-4-(2-fluoro-4- (trifluoromethyl)phenyl)-2- methyl-1H-pyrrolo[3,4- c]pyridine-1,3(2H)-dione | |
| 4 | 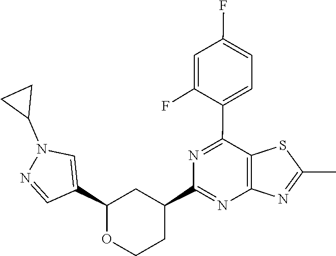 [SMILES: Cc1nc2nc([C@H]3CCO[C@@H](c4cnn(C5CC5)c4)C3)nc(-c3ccc(F)cc3F)c2s1] | 5-((2R,4S)-2-(1-cyclopropyl- 1H-pyrazol-4-yl)tetrahydro-2H- pyran-4-yl)-7-(2,4- difluorophenyl)-2- methylthiazolo[4,5-d] pyrimidine | |
| 5 | 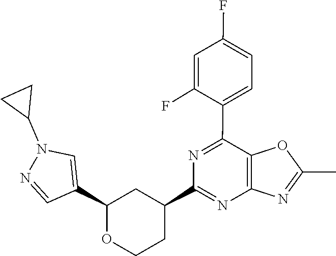 [SMILES: Cc1nc2nc([C@H]3CCO[C@@H](c4cnn(C5CC5)c4)C3)nc(-c3ccc(F)cc3F)c2o1] | 5-((2R,4S)-2-(1-cyclopropyl- 1H-pyrazol-4-yl)tetrahydro-2H- pyran-4-yl)-7-(2,4- difluorophenyl)-2- methyloxazolo[4,5-d] pyrimidine | |

### Example A3: In Vitro Assay Data

In vitro Measurement of Triggering Receptor Expressed on Myeloid Cells 2 activity using cellular phosphorylation of Spleen Tyrosine Kinase (“Syk”) Assays

Measurement of TREM2 agonist potency was done using a HEK cell line expressing human TREM2 and DAP12 (HEK293T-hTREM2 cells). Binding of small molecules to, and activation of, TREM2 increases the phosphorylation of Syk. The resultant levels of Syk phosphorylation are measured using a commercial AlphaLisa reagent kit. To perform the assay, HEK-hTREM2 cells were plated at 14,000 cells per well in a 384 well plate, in 25 L of complete growth media and incubated at 37° C., 5% CO₂ for 20-24 hours.

Prior to the assay, test compounds were diluted in the 384 well plates in assay buffer and allowed to equilibrate for 30 minutes. Growth media was removed from cell plates by inversion on blotting paper, and 25 μL of test articles in assay buffer was added to cells. Cells were incubated for 45 minutes at room temperature. After 45 minutes, assay buffer was removed and 10 μL of lysis buffer was added. Plates were shaken for 20 minutes at 350 RPM at room temperature. After complete lysis, AlphaLisa reagents were added to the lysate, and fluorescence intensity was measured using a Perkin Elmer Envision plate reader. Intensities were used to generate a standard curve, and % activation was calculated. Curve fitting was performed using Prism v9 software, log(agonist) vs response—variable slope (four parameters), and EC50s were calculated from the curve fit.

The results presented in Table D have been generated with the in vitro assay described above. This assay may be used to test any of the compounds described herein to assess and characterize a compound's ability to act as an agonist of TREM2.

Compounds designated as “A” demonstrated an EC50 of ≤0.05 M. Compounds designated as “B” demonstrated an EC50>0.05 M and ≤0.5 M. Compounds designated as “C” demonstrated an EC50>0.5 M and ≤3.0 M. Compounds designated as “D” demonstrated an EC50>3.0 M and ≤100 M. Compounds designated as “E” demonstrated an EC50>100 μM. Compounds designated as “-” had not been tested as of the filing of the present application, but can be tested using the methods described herein.

TABLE D
hTREM2 EC50 Data (HEK293 Cells)

| Ex # | hTREM2 EC50 μM |
|---|---|
| 1 | C |
| 2 | D |
| 3 | D |
| 4 | B |
| 5 | C |

TABLE D-2
hTREM2 EC50 Data (HEK293 Cells):

| I# | hTREM2 EC50 μM |
|---|---|
| I-14 | C |
| I-26 | — |
| I-22 | B |
| I-79 | A |
| I-84 | A |
| I-89 | B |
| I-94 | B |
| I-99 | B |
| I-104 | B |
| I-109 | B |
| I-114 | B |
| I-119 | C |
| I-124 | C |
| I-129 | B |
| I-134 | B |
| I-135 | C |
| I-139 | C |
| I-144 | A |
| I-149 | B |
| I-154 | C |
| I-159 | C |
| I-164 | B |
| I-165 | B |
| I-174 | C |
| I-179 | B |
| I-185 | B |
| I-188 | B |
| I-191 | B |
| I-196 | B |
| I-199 | B |
| I-200 | B |
| I-202 | B |
| I-203 | — |
| I-205 | C |
| I-210 | C |
| I-216 | — |
| I-221 | A |
| I-224 | A |
| I-230 | A |
| I-235 | A |
| I-240 | A |
| I-245 | A |
| I-248 | B |
| I-249 | A |
| I-253 | B |
| I-258 | A |
| I-263 | B |
| I-268 | B |
| I-269 | B |
| I-273 | A |
| I-278 | C |
| I-279 | A |
| I-283 | A |
| I-288 | A |
| I-289 | B |
| I-293 | B |
| I-298 | B |
| I-303 | B |

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of Formula I 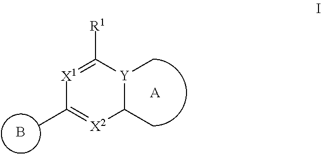
or a tautomer thereof, or a pharmaceutically acceptable salt of said compound or said tautomer, wherein
R¹ is an optionally substituted C₁₋₆ aliphatic group, C₁₋₆haloalkyl, optionally substituted OCH₂—(C₃₋₆cycloalkyl), optionally substituted O-phenyl, or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula 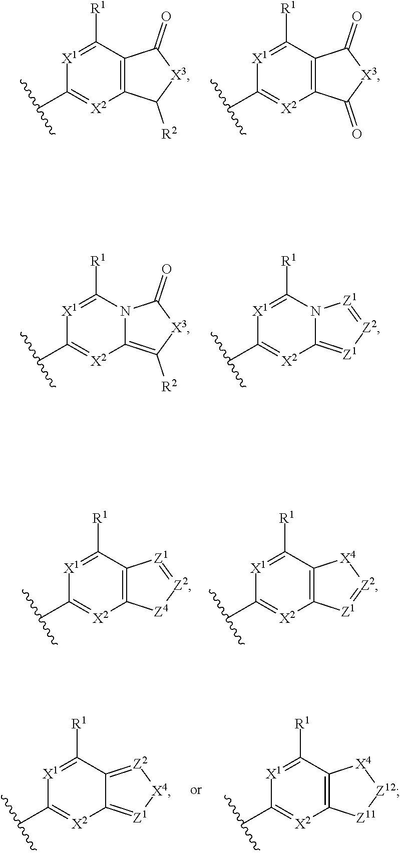
Y is C or N, as required by the bicyclic ring system formed by Ring A;
X³ is CHR³, or NR⁴;
X⁴ is CHR³, NR⁴, O or S;
each Z¹ is independently CR² or N;
Z² is CR³ or N;
Z¹¹ is CHR³, C(R³)₂, or NR⁴;
Z¹² is CHR², C(R²)₂, NR⁴, or C(═N—R⁴);
R² and R³ are each independently selected from hydrogen, an optionally substituted C₁₋₆ aliphatic group, halogen, —OR, —CN, —NR₂, —C(═O)R, —NR—C(O)—R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R⁴ is hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
R³ and R⁴ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
Ring B is 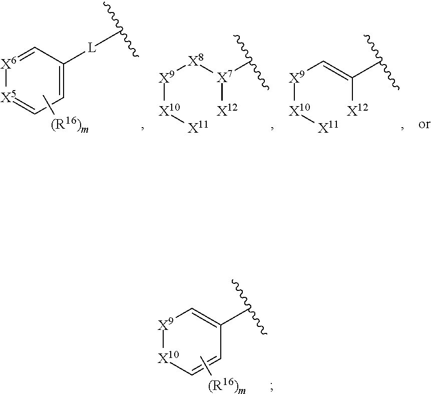
L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR, C(R)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁-₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³ and R¹⁴ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

2. The compound of claim 1, wherein R¹ is optionally substituted C₃₋₆cycloalkyl, optionally substituted spiro[3.3]heptanyl, optionally substituted spiro[5.2]octanyl, optionally substituted 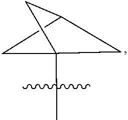 [SMILES: *C12CC(C1)C2]
optionally substituted cyclopent-1-en-1-yl, optionally substituted cyclohex-1-en-1-yl, optionally substituted phenyl, optionally substituted pyridinyl, optionally substituted aziridine-1-yl, optionally substituted pyrrolidine-1-yl, optionally substituted azabicyclo[3.1.0]hexan-3-yl, optionally substituted piperidine-1-yl, or optionally substituted —OCH₂—(C₃₋₄cycloalkyl).

3. The compound of claim 1, wherein R¹ is optionally substituted phenyl.

4. The compound of claim 1, wherein R is:
(A) —CH₂CH₂CF₃, 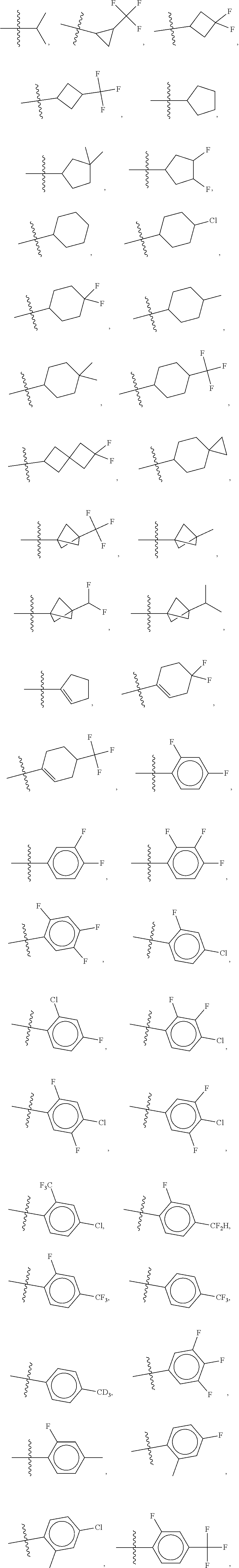 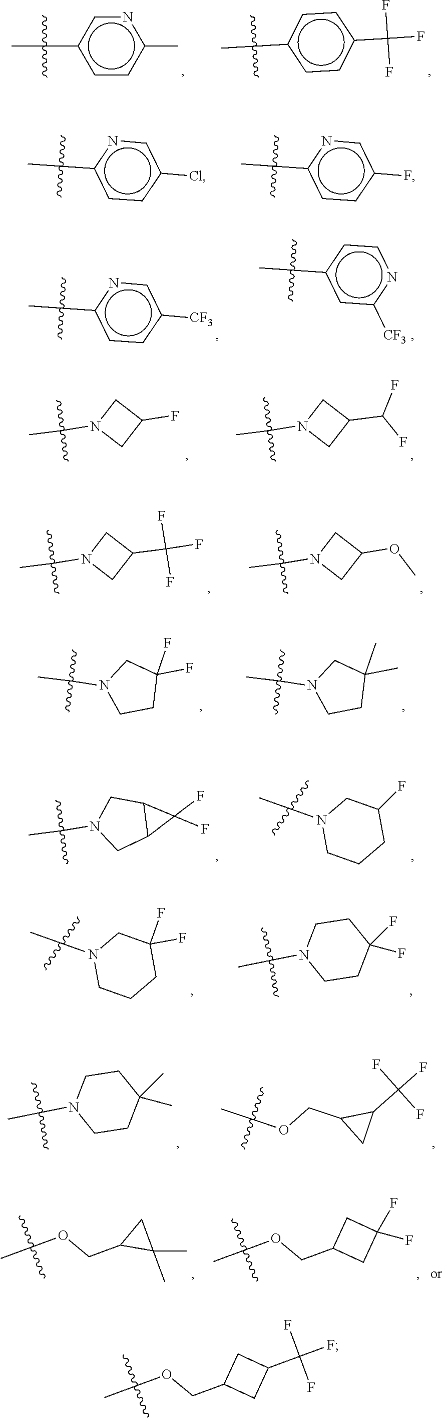
or (B) a substituent selected from:

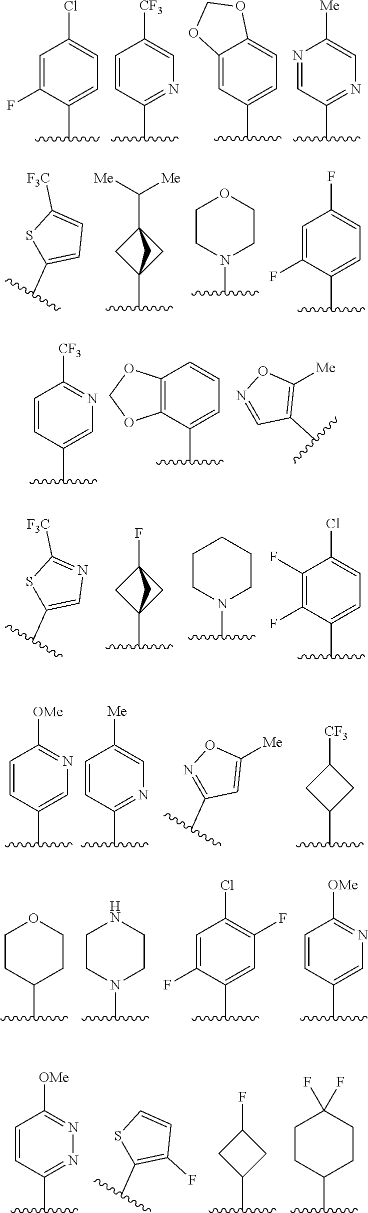

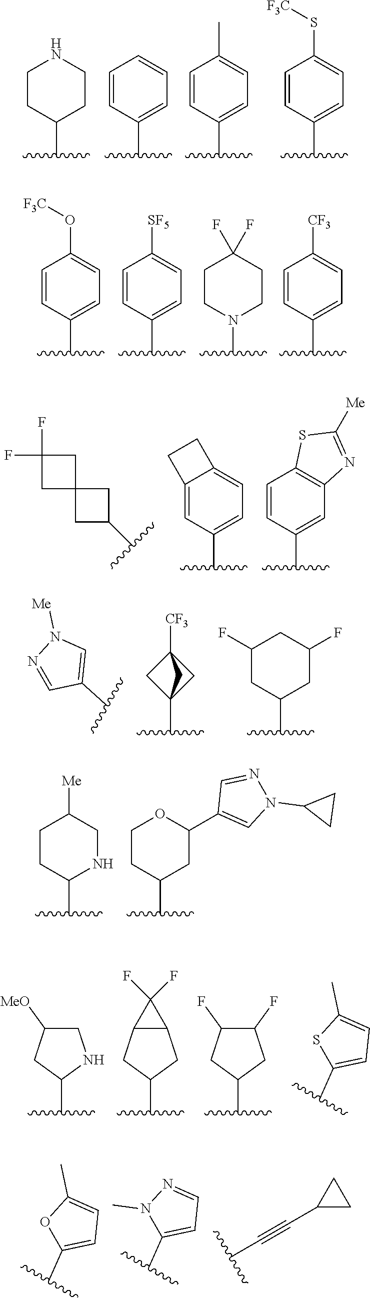

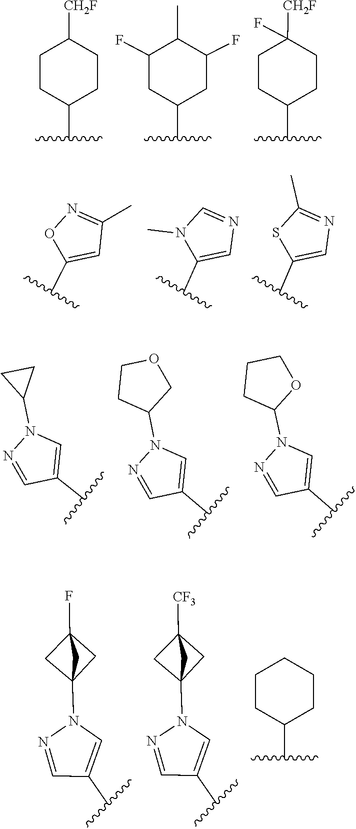

5. The compound of any one of claims 1-4, wherein Ring A together with the 6-membered ring system to which it is fused forms a bicyclic ring system of formula

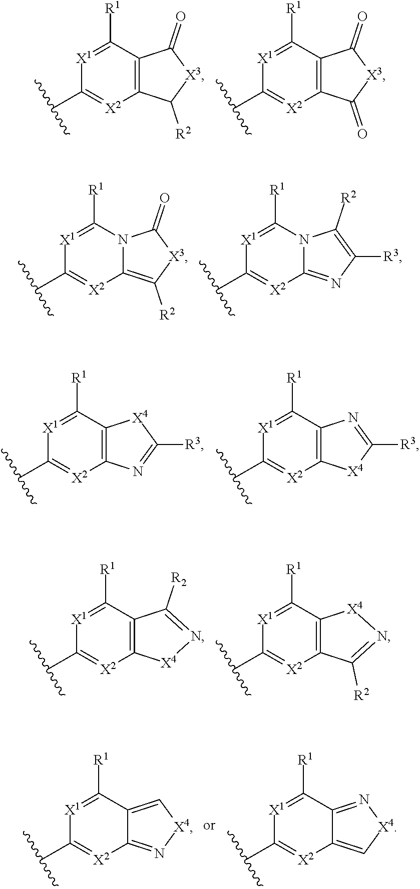

6. The compound of any one of claims 1-5, wherein X¹ is CH or N.

7. The compound of any one of claims 1-6, wherein X² is CH or N.

8. The compound of any one of claims 1-7, wherein X³ is NR⁴.

9. The compound of any one of claims 1-8, wherein X⁴ is NR⁴.

10. The compound of any one of claims 1-9, wherein Ring B is

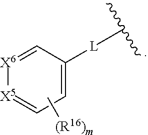

11. The compound of any one of claims 1-10, wherein L is a bond.

12. The compound of any one of claims 1-11, wherein Ring B is

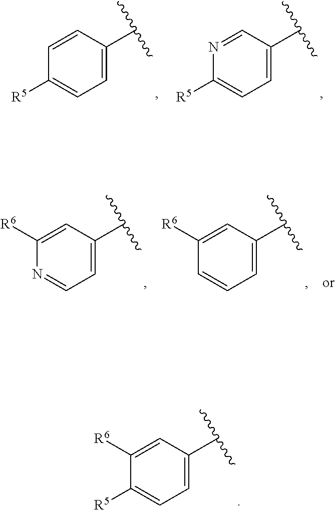

13. The compound of any one of claims 1-9, wherein Ring B is

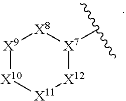

14. The compound of any one of claims 1-9, wherein Ring B is selected from:

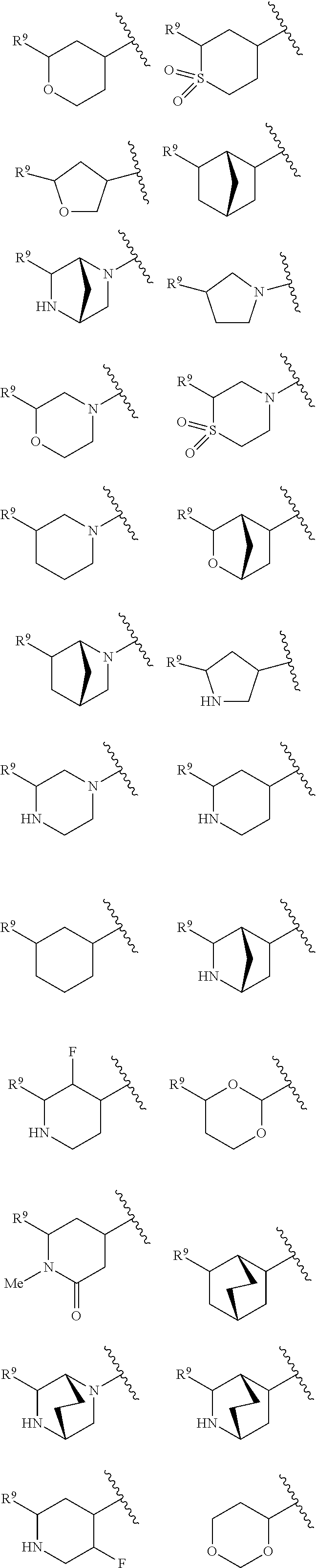

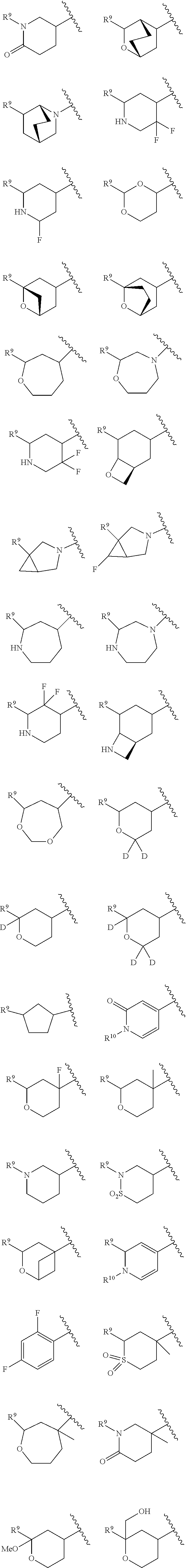

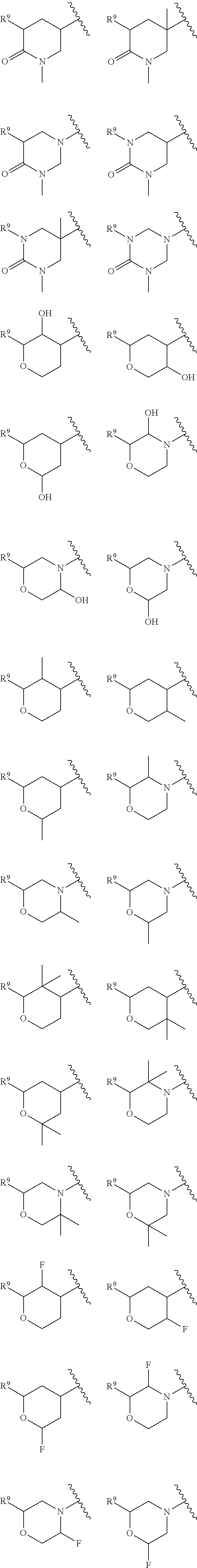

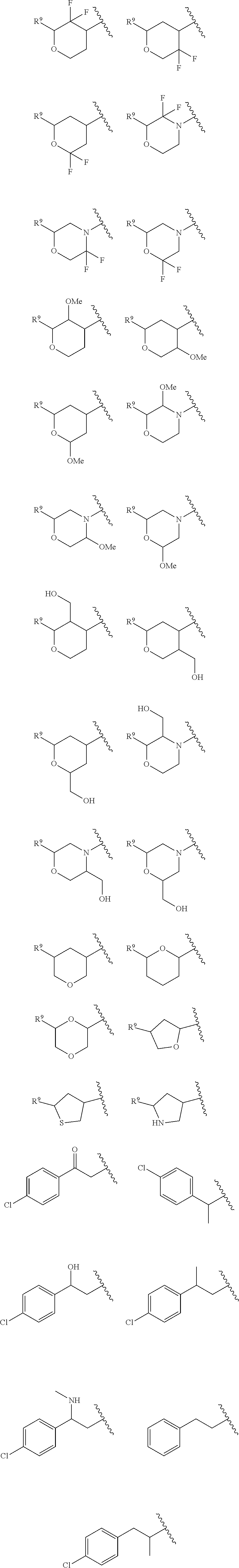

15. The compound of any one of claims 1-9, wherein Ring B is:

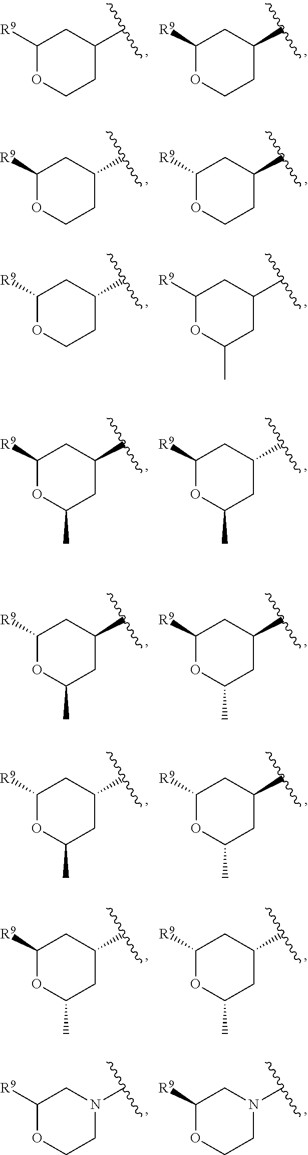

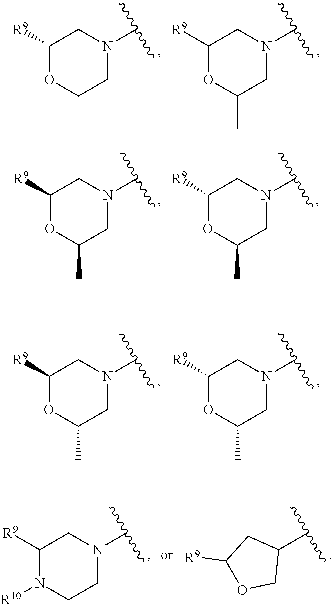

16. The compound of any one of claims 1-9, 13 and 14, wherein R⁹ is selected from:

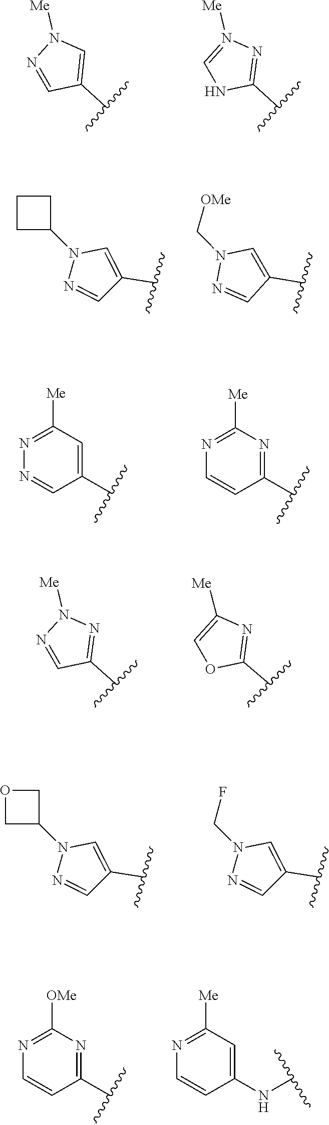

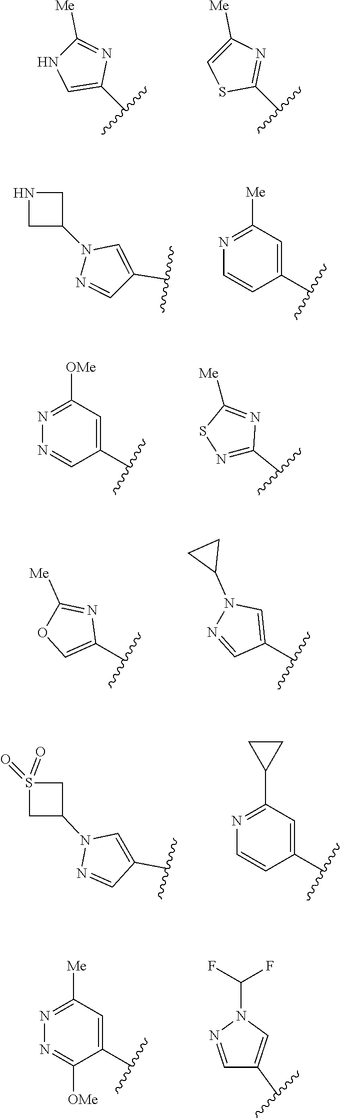

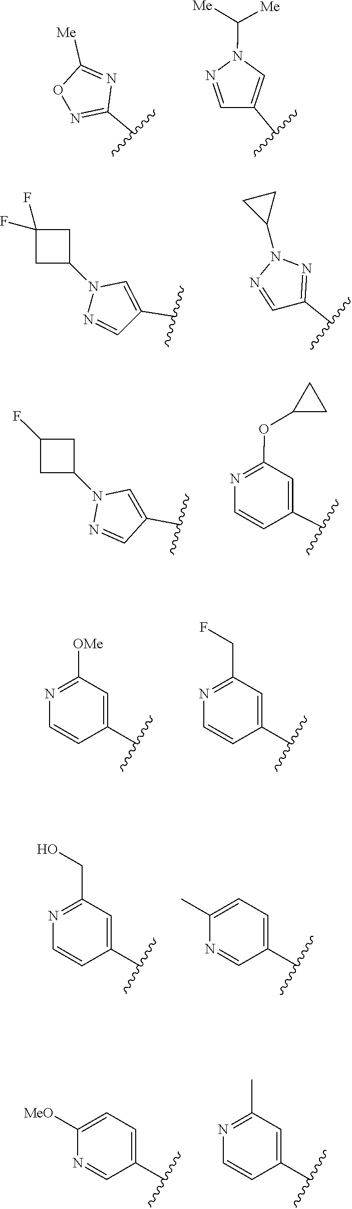

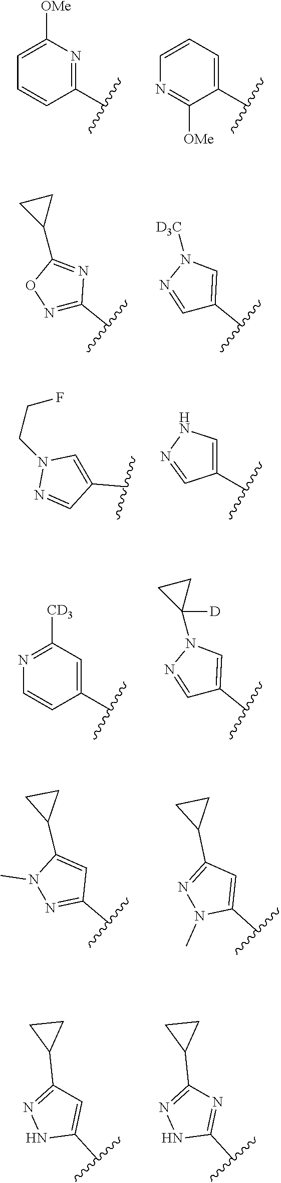

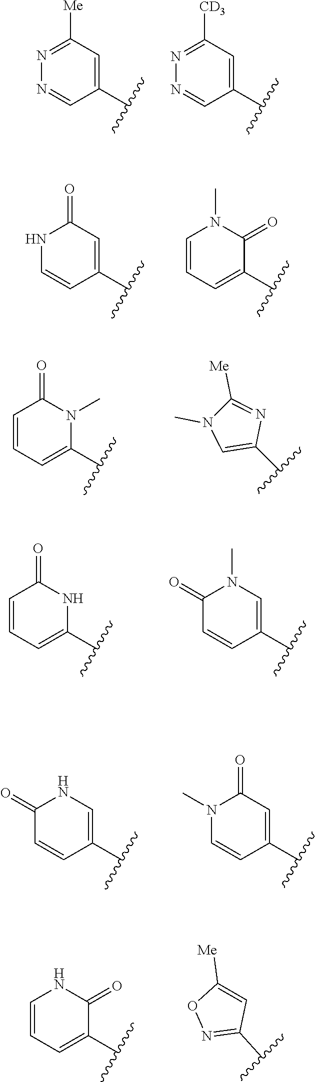

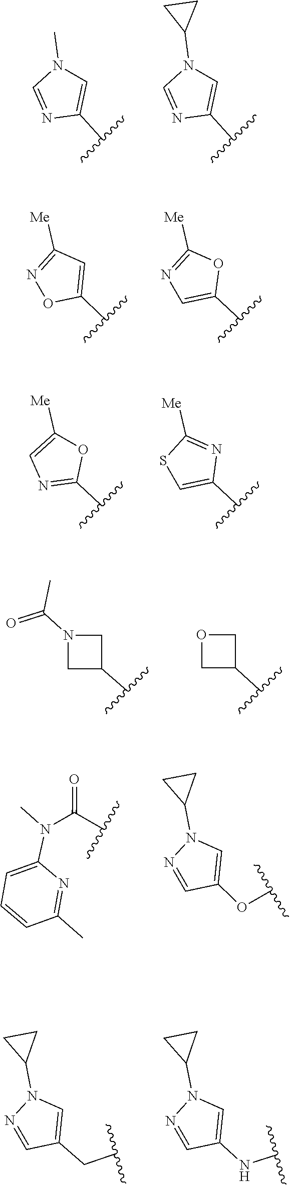

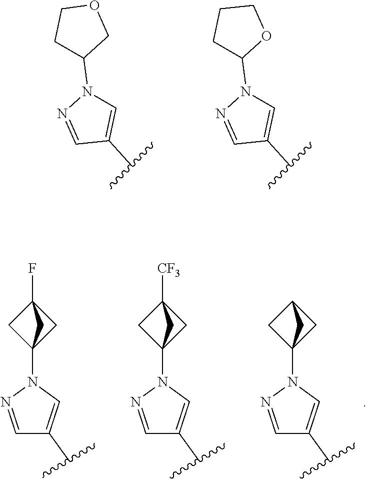

17. The compound of any one of claims 1-9, 13 and 14, wherein R⁹ is

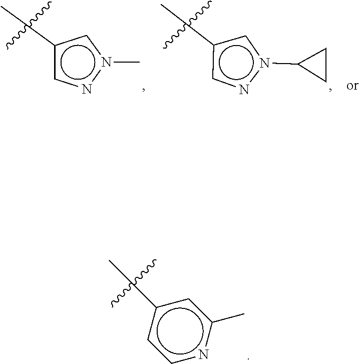

18. The compound of any one of claims 1-17, wherein the compound is a compound of Formula IIa, IIb, IIc, IIIa, IIIb, IIIc, IVa, IVb, IVc, Va, Vb, Vc, VIa, VIb, VIc, VIIa, VIIb, VIIc, VIIIa, VIIIb, or VIIIc.

19. A compound of Table A or Table A2, or a pharmaceutically acceptable salt thereof.

20. A pharmaceutical composition comprising the compound according to any one of claims 1-19, or a tautomer thereof, or a pharmaceutically acceptable salt of said compound or said tautomer, and a pharmaceutically acceptable excipient.

21. A compound according to any one of claims 1-19, or a tautomer thereof, or a pharmaceutically acceptable salt of said compound or said tautomer, or the pharmaceutical composition according to claim 20 for use as a medicament.

22. A compound according to any one of claims 1-19, or a tautomer thereof, or a pharmaceutically acceptable salt of said compound or said tautomer, or the pharmaceutical composition according to claim 20 for use in treating or preventing a condition associated with a loss of function of human TREM2.

23. A compound according to any one of claims 1-19, or a tautomer thereof, or a pharmaceutically acceptable salt of said compound or said tautomer, or the pharmaceutical composition according to claim 20 for use in treating or preventing Parkinson's disease, rheumatoid arthritis, Alzheimer's disease, Nasu-Hakola disease, frontotemporal dementia, multiple sclerosis, prion disease, or stroke.

24. Use of the compound according to any one of claims 1-19, or a tautomer thereof, or a pharmaceutically acceptable salt of said compound or said tautomer, or the pharmaceutical composition according to claim 20 in the preparation of a medicament for treating or preventing a condition associated with a loss of function of human TREM2.

25. Use of the compound according to any one of claims 1-19, or a tautomer thereof, or a pharmaceutically acceptable salt of said compound or said tautomer, or the pharmaceutical composition according to claim 20 in the preparation of a medicament for treating or preventing Parkinson's disease, rheumatoid arthritis, Alzheimer's disease, Nasu-Hakola disease, frontotemporal dementia, multiple sclerosis, prion disease, or stroke.

26. A method of treating or preventing a condition associated with a loss of function of human TREM2 in a subject in need thereof, the method comprising administering to the subject a therapeutically effective amount of the compound according to any one of claims 1-19, or a tautomer thereof, or a pharmaceutically acceptable salt of said compound or said tautomer.

27. A method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound according to any one of claims 1-19, or a tautomer thereof, or a pharmaceutically acceptable salt of said compound or said tautomer.
